(19) United States
(12) Patent Application Publication
(10) Pub. No.: US 2024/0342215 A1
(43) Pub. Date: Oct. 17, 2024

(54) ENGINEERED T CELL RECEPTORS FUSED TO BINDING DOMAINS FROM ANTIBODIES

(71) Applicant: Regeneron Pharmaceuticals, Inc., Tarrytown, NY (US)

(72) Inventor: Jordan Jarjour, Seattle, WA (US)

(21) Appl. No.: 18/578,367

(22) PCT Filed: Jul. 14, 2022
(86) PCT No.: PCT/US2022/073725

§ 371 Date: Jan. 11, 2024

Related U.S. Application Data
(60) Provisional application No. 63/221,819, filed on Jul. 14, 2021.

(51) Int. Cl. A61K 35/17; A61K 39/00; A61P 35/00; C07K 14/725; C07K 16/28; C07K 16/30; C12N 5/0783; C12N 15/86

(52) U.S. Cl. CPC A61K 35/17; A61K 39/4611; A61K 39/4632; A61K 39/464412; A61K 39/464417; A61K 39/464486; A61P 35/00; C07K 14/7051; C07K 16/2803; C07K 16/2851; C07K 16/2878; C07K 16/30; C12N 5/0636; C12N 15/86; A61K 2039/505; C07K 2317/22; C07K 2317/31; C07K 2317/569; C07K 2317/622; C07K 2319/02; C12N 2510/00; C12N 2740/15043

(57) ABSTRACT

The present disclosure provides improved T cell receptors, polynucleotides, polypeptides, vectors, cells, and methods of using the same. Particularly, the present invention relates to T cell receptor-based constructs engineered to comprise one or more additional binding domains, and methods of using the same. In certain embodiments, the one or more binding domains are fused to one or both TCR variable domains. In particular embodiments, the one or more additional binding domains are linked to the TCR with one or more polypeptide linkers.

### CROSS REFERENCE TO RELATED APPLICATIONS

This application claims the benefit under 35 U.S.C. § 119(e) of U.S. Provisional Application No. 63/221,819, filed Jul. 14, 2021, which is incorporated by reference herein in 5 its entirety.

### STATEMENT REGARDING SEQUENCE LISTING

The Sequence Listing associated with this application is provided in Sequence Listing XML format in lieu of a paper copy and is hereby incorporated by reference into the specification. The name of the XML file containing the Sequence Listing is 137080-03620_SL.xml. The text file is 198,833 bytes in size, created on Jul. 14, 2022, and is being submitted electronically via Patent Center, concurrent with the filing of the specification.

### BACKGROUND

### Technical Field

The present invention relates to engineered T cell receptors (TCRs). Particularly, the present invention relates to TCR-based constructs and complexes engineered to comprise one or more additional antigen-binding domains, and methods of using the same. In certain embodiments, the one or more antigen-binding domains are linked to the TCRα, TCRβ, TCRγ, and/or TCRδ variable domains. In particular embodiments, the one or more additional antigen-binding domains are linked to the TCR variable domain via one or more polypeptide linkers.

### Description of the Related Art

Adoptive T cell therapies can be engineered to target either cell surface antigens (via chimeric antigen receptors; CAR) or intracellular antigens (via engineered T cell receptors; TCR). CAR T cell activation and anti-tumor activity is achieved through linking targeting moieties to a compound intracellular signaling region comprising one or more costimulatory signaling domains fused to the CD3-zeta signaling domain. Conversely, engineered TCR T cells become activated through the natural intracellular signaling events coordinated by the CD3 complex and other proximal signaling molecules, resulting in increased sensitivity over CAR T cells.

While the response sensitivity of TCR T cells over CAR T cells is desirable, TCR T cells are limited by other characteristics. For example, since target recognition is governed by MHC-restriction, TCRs are usually developed toward HLA haplotypes that are present in less than 40% of the general population. This represents a ceiling for patient eligibility/recruitment, prior to standard cuts stemming from target expression and other exclusions and limitations. MHC-restriction also creates ample opportunity for target cells (e.g., tumors) to evolve escape routes via genetic mutation or suppression of antigen processing and presentation machinery.

Accordingly, there remains a need for improved TCR-based constructs and therapies to treat disease.

### BRIEF SUMMARY

The present disclosure generally relates, in part, to engineered T cell receptors, fusion proteins, polynucleotides, compositions, medicaments and uses thereof.

In one aspect an engineered T cell receptor (TCR) is provided, wherein the engineered TCR receptor comprises one or more antigen-binding domain(s) linked to one or both TCR variable domains.

In another aspect, an engineered T cell receptor (TCR) is provided comprising (a) a TCRα polypeptide comprising a TCRα variable domain; (b) a TCRβ polypeptide comprising a TCRβ variable domain; and (c) one or more antigen-binding domains linked to the TCRα variable domain and/or TCRβ variable domain.

In another aspect, an engineered T cell receptor (TCR) is provided comprising (a) a TCRγ polypeptide comprising a TCRγ variable domain; (b) a TCRδ polypeptide comprising a TCRδ variable domain; and (c) one or more antigen-binding domains linked to the TCRγ variable domain and/or TCRδ variable domain.

In another aspect a fusion polypeptide is provided comprising (a) a TCRβ polypeptide comprising a TCRβ variable domain; (b) a polypeptide cleavage signal; and (c) a TCRα polypeptide comprising one or more antigen-binding domains, a polypeptide linker, and a TCRα variable domain.

In another aspect a fusion polypeptide is provided comprising (a) a TCRβ polypeptide comprising one or more antigen-binding domains, a polypeptide linker, and a TCRβ variable domain; (b) a polypeptide cleavage signal; and (c) a TCRα polypeptide comprising a TCRα variable domain.

In another aspect a fusion polypeptide is provided comprising (a) a TCRβ polypeptide comprising one or more antigen-binding domains, a polypeptide linker, and a TCRβ variable domain; (b) a polypeptide cleavage signal; and (c) a TCRα polypeptide comprising one or more antigen-binding domains, a polypeptide linker, and a TCRα variable domain.

In another aspect a fusion polypeptide is provided comprising (a) a TCRγ polypeptide comprising a TCRγ variable domain; (b) a polypeptide cleavage signal; and (c) a TCRδ polypeptide comprising one or more antigen-binding domains, a polypeptide linker, and a TCRδ variable domain.

In another aspect a fusion polypeptide is provided comprising (a) a TCRγ polypeptide comprising one or more antigen-binding domains, a polypeptide linker, and a TCRγ variable domain; (b) a polypeptide cleavage signal; and (c) a TCRδ polypeptide comprising a TCRδ variable domain.

In another aspect a fusion polypeptide is provided comprising (a) a TCRγ polypeptide comprising one or more antigen-binding domains, a polypeptide linker, and a TCRγ variable domain; (b) a polypeptide cleavage signal; and (c) a TCRδ polypeptide comprising one or more antigen-binding domains, a polypeptide linker, and a TCRδ variable domain.

In various embodiments, the TCRα polypeptide comprises a TCRα constant domain and the TCRβ polypeptide comprises a TCRβ constant domain.

In various embodiments, the TCRγ polypeptide comprises a TCRγ constant domain and the TCRδ polypeptide comprises a TCRδ constant domain.

In various embodiments, the one or more antigen-binding domains comprises a first antigen-binding domain linked to the TCRα or TCRγ variable domain. In some embodiments, the one or more antigen-binding domains comprises a first antigen-binding domain linked to the TCRβ or TCRδ variable domain. In some embodiments, the one or more antigen-binding domains comprise: (i) a first antigen-binding domain linked to the TCRα or TCRγ variable domain, and (ii) a first antigen-binding domain linked to the TCRβ or TCRδ variable domain. In some embodiments, the first antigen-binding domains are linked to the N-terminus of the variable domains. In some embodiments, the first antigen-binding domains are the same or different, and/or bind to the same or different target antigens.

In various embodiments, the one or more antigen-binding domains comprises a second antigen-binding domain linked to the first antigen-binding domain. In various embodiments, the one or more antigen-binding domains comprises a second antigen-binding domain linked to the first antigen-binding domain linked to the TCRα or TCRγ variable domain. In some embodiments, the one or more antigen-binding domains comprises a second antigen-binding domain linked to the first antigen-binding domain linked to the TCRβ or TCRδ variable domain. In some embodiments, the one or more antigen-binding domains comprises: (i) a second antigen-binding domain linked to the first antigen-binding domain linked to the TCRα or TCRγ variable domain, and (ii) a second antigen-binding domain linked to the first antigen-binding domain linked to the TCRβ or TCRδ variable domain.

In various embodiments, the second antigen-binding domains are linked to the N-terminus of the first antigen-binding domain. In some embodiments, the second antigen-binding domains are the same or different, and/or bind to the same or different target antigens. In some embodiments, the first and second antigen-binding domains are the same or different, and/or bind to the same or different target antigens.

In various embodiments, the one or more antigen-binding domains bind a target antigen selected from the group consisting of: alpha folate receptor (FRα), αᵥβ₆ integrin, ADGRE2, BACE2, B cell maturation antigen (BCMA), B7-H3 (CD276), B7-H4, B7-H6, CA19.9, carbonic anhydrase IX (CAIX), CCR1, CD7, CD16, CD19, CD20, CD22, CD30, CD33, CD37, CD38, CD44, CD44v6, CD44v7/8, CD70, CD79a, CD79b, CD123, CD133, CD138, CD171, CD244, carcinoembryonic antigen (CEA), C-type lectin-like molecule-1 (CLL-1), CD2 subset 1 (CS-1), CLDN6, cMET, chondroitin sulfate proteoglycan 4 (CSPG4), CLDN18.2, cutaneous T cell lymphoma-associated antigen 1 (CTAGE1), DLL3, epidermal growth factor receptor (EGFR), epidermal growth factor receptor variant III (EGFRvIII), EGFR806, epithelial glycoprotein 2 (EGP2), epithelial glycoprotein 40 (EGP40), EPHB2, ERBB4, epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), FLT3, FN, FN-EDB, FRBeta, ganglioside G2 (GD2), ganglioside G3 (GD3), Glypican-3 (GPC3), EGFR family including ErbB2 (HER2), HER2p95, EGFRv3, IL-10Rα, IL-13Rα2, Kappa, cancer/testis antigen 2 (LAGE-1A), K-Ras, K-Ras G12C, K-Ras G12D, K-Ras G12V, Lambda, Lewis-Y (LeY), L1 cell adhesion molecule (L1-CAM), LILRB2, LY6G6GD, melanoma antigen recognized by T cells 1 (MelanA or MART1), Mesothelin (MSLN), MMP10, MUC1, MUC16, MHC class I chain related proteins A (MICA), MHC class I chain related proteins B (MICB), neural cell adhesion molecule (NCAM), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), transmembrane activator and CAML interactor (TACI), tumor endothelial marker 1 (TEM1/CD248), tumor endothelial marker 7-related (TEM7R), TIM3, trophoblast glycoprotein (TPBG), UL16-binding protein (ULBP) 1, ULBP2, ULBP3, ULBP4, ULBP5, ULBP6, and vascular endothelial growth factor receptor 2 (VEGFR2).

In various embodiments, the one or more antigen-binding domains bind a target polypeptide derived from a protein selected from the group consisting of: α-fetoprotein (AFP), ASCL2, B Melanoma Antigen (BAGE) family members, Brother of the regulator of imprinted sites (BORIS), Cancer-testis antigens, Cancer-testis antigen 83 (CT-83), Carbonic anhydrase IX (CAIX), Carcinoembryonic antigen (CEA), Cytomegalovirus (CMV) antigens, Cytotoxic T cell (CTL)-recognized antigen on melanoma (CAMEL), Epstein-Barr virus (EBV) antigens, EPHB2, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human papillomavirus (HPV)-E6, HPV-E7, Human telomerase reverse transcriptase (hTERT), IGF2BP3/A3, IGF2BP1, K-Ras, K-Ras G12C, K-Ras G12D, K-Ras G12V, Latent membrane protein 2 (LMP2), LY6G6D, Melanoma antigen family A, 1 (MAGE-A1), MAGE-A2, MAGE-A3, MAGE-A4, MAGE-A6, MAGE-A10, MAGE-A12, Melanoma antigen recognized by T cells (MART-1), Mesothelin (MSLN), Mucin 1 (MUC1), Mucin 16 (MUC16), New York esophageal squamous cell carcinoma-1 (NYESO-1), P53,P antigen (PAGE) family members, PAP, PIK3CA, PIK3CA H1047R, Placenta-specific 1 (PLAC1), Preferentially expressed antigen in melanoma (PRAME), Prostate specific antigen PSA, Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, TP53 R175H, Tyrosinase, Tyrosinase related protein (TRP)1, TRP2, UBD, Wilms tumor protein (WT-1), Wnt10A, X Antigen Family Member 1 (XAGE1), and X Antigen Family Member 2 (XAGE2).

In some embodiments, the one or more antigen-binding domains bind CD33, CLL1, CD19, CD20, CD22, CD79A, CD79B, or BCMA. In some embodiments, the one or more antigen-binding domains bind CD19, CD20, CD22, CD33, CD79A, CD79B, B7H3, Muc16, Her2, EGFR, FN-EDB, CLDN18.2, DLL3, FLT3, CLL1, CD123, or BCMA. In some embodiments, the one or more antigen-binding domains comprises an amino acid sequence at least 95% identical to an amino acid sequence as set forth in any one of SEQ ID NOs: 1-32.

In various embodiments, the one or more antigen-binding domains comprise an antibody or antigen binding fragment thereof selected from the group consisting of: a Camel Ig, a Llama Ig, an Alpaca Ig, Ig NAR, a Fab′ fragment, a F(ab′)₂ fragment, a bispecific Fab dimer (Fab2), a trispecific Fab trimer (Fab3), an Fv, an single chain Fv protein (“scFv”), a bis-scFv, (scFv)₂, a minibody, a diabody, a triabody, a tetrabody, a disulfide stabilized Fv protein (“dsFv”), and a single-domain antibody (sdAb, a camelid VHH, Nanobody). In some embodiments, the one or more antigen-binding domains comprise one or more single-chain variable fragments (scFv). In some embodiments, the one or more antigen-binding domains comprise one or more single domain antibodies (sdAb). In some embodiments, the sdAb is a camelid VHH, nanobody, or heavy chain-only antibody (HcAb). In some embodiments, the sdAb is a camelid VHH. In some embodiments, the antibody or antigen binding fragment thereof is human or humanized.

In various embodiments, the one or more antigen-binding domains comprise a ligand.

In various embodiments, the one or more antigen-binding domains are linked to the TCR variable domains by one or more polypeptide linkers. In some embodiments, the one or more polypeptide linkers comprise a linker from about 2 to about 25 amino acids long. In some embodiments, the one or more polypeptide linkers comprise a linker from about 4 to about 15 amino acids long. In some embodiments, the one or more polypeptide linkers comprise a linker from about 4 to about 10 amino acids long. In some embodiments, the one or more polypeptide linkers comprise a linker of about 4, about 5, about 6, about 7, about 8, about 9, about 10, about 11, about 12, about 13, about 14, or about 15 amino acids long. In some embodiments, the one or more polypeptide linkers comprise a linker of about 9 or about 10 amino acids long.

In various embodiments, the one or more polypeptide linkers comprise a linker selected from the group consisting of: GG, GS, SG, SS, GSS, SSG, GSG, SGS, SGG, GGS, GGGS (SEQ ID NO: 53), (GGGGS)₁₋₅ polypeptide (SEQ ID NOs: 35-39), a linker from a marsupial γμTCR (e.g., LEKT; SEQ ID NO: 33), and any combination thereof. In some embodiments, the one or more polypeptide linkers comprises a linker from a marsupial γμTCR, comprising an amino acid sequence as set forth in SEQ ID NO: 33. In some embodiments, the one or more polypeptide linkers comprise a GGGGS (SEQ ID NO: 35) linker (G4S). In some embodiments, the one or more polypeptide linkers comprise a marsupial γμTCR linker and a G4S linker as set forth in SEQ ID NO: 34. In some embodiments, the one or more polypeptide linkers comprise two GGGGS linkers (2×G4S) (SEQ ID NO: 36). In some embodiments, the one or more polypeptide linkers comprise three GGGGS linkers (3×G4S) (SEQ ID NO: 37). In particular embodiments, the one or more polypeptide linkers comprise an amino acid sequence as set forth in any one of SEQ ID NOs: 33-53.

In various embodiments, the first and second antigen-binding domains are separated by a second polypeptide linker. In some embodiments, the second polypeptide linker is about 2 to about 25 amino acids long. In some embodiments, the second polypeptide linker is about 4 to about 15 amino acids long.

In various embodiments, the second polypeptide linker comprises a linker selected from the group consisting of: GG, GS, SG, SS, GSS, SSG, GSG, SGS, SGG, GGS, GGGS (SEQ ID NO: 53), (GGGGS)₁₋₅ polypeptide (SEQ ID NOs: 35-39), and any combination thereof. In particular embodiments, the second polypeptide linker comprises an amino acid sequence as set forth in any one of SEQ ID NOs: 33-53.

In various embodiments, the TCR variable domains bind a target polypeptide presented by an MHC complex.

In various embodiments, the TCR variable domains bind a target polypeptide derived from a protein selected from the group consisting of: α-fetoprotein (AFP), ASCL2, B Melanoma Antigen (BAGE) family members, Brother of the regulator of imprinted sites (BORIS), Cancer-testis antigens, Cancer-testis antigen 83 (CT-83), Carbonic anhydrase IX (CAIX), Carcinoembryonic antigen (CEA), Cytomegalovirus (CMV) antigens, Cytotoxic T cell (CTL)-recognized antigen on melanoma (CAMEL), Epstein-Barr virus (EBV) antigens, EPHB2, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human papillomavirus (HPV)-E6, HPV-E7, Human telomerase reverse transcriptase (hTERT), IGF2BP3/A3, IGF2BP1, K-Ras, K-Ras G12C, K-Ras G12D, K-Ras G12V, Latent membrane protein 2 (LMP2), LY6G6D, Melanoma antigen family A, 1 (MAGE-A1), MAGE-A2, MAGE-A3, MAGE-A4, MAGE-A6, MAGE-A10, MAGE-A12, Melanoma antigen recognized by T cells (MART-1), Mesothelin (MSLN), Mucin 1 (MUC1), Mucin 16 (MUC16), New York esophageal squamous cell carcinoma-1 (NYESO-1), P53,P antigen (PAGE) family members, PAP, PIK3CA, PIK3CA H1047R, Placenta-specific 1 (PLAC1), Preferentially expressed antigen in melanoma (PRAME), Prostate specific antigen PSA, Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, TP53 R175H, Tyrosinase, Tyrosinase related protein (TRP)1, TRP2, UBD, Wilms tumor protein (WT-1), Wnt10A, X Antigen Family Member 1 (XAGE1), and X Antigen Family Member 2 (XAGE2). In some embodiments, the TCR variable domains bind a target polypeptide derived from MAGE-A4, PRAME, K-Ras, TP53R175H, PSA, or IGF2BP3. In some embodiments, the TCR variable domains bind a target polypeptide derived from MAGE-A4.

In various embodiments, the TCRα constant domain comprises an amino acid sequence at least 90% identical to an amino acid sequence as set forth in SEQ ID NOs: 82 or 88, and/or the TCRβ constant domain comprises an amino acid sequence at least 90% identical to an amino acid sequence as set forth in any one of SEQ ID NOs: 80, 81, 86, or 87.

In various embodiments, the TCRγ constant domain comprises an amino acid sequence at least 90% identical to an amino acid sequence as set forth in SEQ ID NO: 83 or 84, and/or the TCRδ constant domain comprises an amino acid sequence at least 90% identical to an amino acid sequence as set forth in any one of SEQ ID NO: 85.

In various embodiments, the TCRα or TCRγ polypeptide comprises (i) an amino acid sequence as set forth in any one of SEQ ID NOs: 105-111, or (ii) a TCRα or TCRγ variable domain comprising an amino acid sequence as set forth in any one of SEQ ID NOs: 62, 64, 66, 68, 70, 72, 74, 76, and 78.

In various embodiments, the TCRβ or TCRδ polypeptide comprises (i) an amino acid sequence as set forth in SEQ ID NO: 103 or 104, or (ii) a TCRβ or TCRδ variable domain comprising an amino acid sequence as set forth in any one of SEQ ID NOs: 63, 65, 67, 69, 71, 73, 75, 77, and 79.

In various embodiments, the polypeptide cleavage signal of the fusion polypeptide is a viral self-cleaving peptide or ribosomal skipping sequence. In some embodiments, the polypeptide cleavage signal is a viral 2A peptide. In some embodiments, the polypeptide cleavage signal is an aphthovirus 2A peptide, a potyvirus 2A peptide, or a cardiovirus 2A peptide. In some embodiments, the polypeptide cleavage signal is a viral 2A peptide selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide. In some embodiments, the polypeptide cleavage signal comprises a furin recognition site upstream of the self-cleaving peptide, optionally wherein the furin recognition site comprises the amino acid sequence as set forth in SEQ ID NO: 112. In some embodiments, the polypeptide cleavage signal comprises an amino acid sequence as set forth in any one of SEQ ID NOs: 113-137.

In various embodiments, the TCRβ or TCRδ polypeptide of the fusion polypeptide is N-terminal of the TCRα or TCRγ polypeptide.

In various embodiments, the TCRα or TCRγ polypeptide of the fusion polypeptide is N-terminal of the TCRβ or TCRδ polypeptide.

In various embodiments, the TCRα and TCRβ polypeptides each comprise an N-terminal signal sequence. In various embodiments, the TCRγ and TCRδ polypeptides each comprises an N-terminal signal sequence. In some embodiments, the signal sequences are the same or different. In some embodiments, the signal sequence is an IgK or TCRα signal sequence. In some embodiments, the signal sequence is an CD8a signal sequence.

In various embodiments, the fusion polypeptide comprises an amino acid sequence at least 90% identical to an amino acid sequence as set forth in any one of SEQ ID NOs: 91-97, 100, and 102.

In another aspect, a polynucleotide encoding an engineered TCR or fusion polypeptide contemplated herein is provided.

In another aspect, a vector comprising one or more polynucleotides contemplated herein is provided. In some embodiments, the vector is an expression vector, retroviral vector, or a lentiviral vector.

In another aspect, a cell comprising an engineered TCR, fusion polypeptide, polynucleotide, or vector contemplated herein is provided. In some embodiments, the cell is a hematopoietic cell. In some embodiments, the cell is a T cell, an αβ-T cell, or a γδ-T cell. In some embodiments, the cell is a CD3⁺, CD4⁺, and/or CD8⁺ cell. In some embodiments, the cell is an immune effector cell. In some embodiments, the cell is a cytotoxic T lymphocytes (CTLs), a tumor infiltrating lymphocytes (TILs), or a helper T cell. In some embodiments, the cell is a T cell, a natural killer (NK) cell, or a natural killer T (NKT) cell. In some embodiments, the source of the cell is peripheral blood mononuclear cells, bone marrow, lymph nodes tissue, cord blood, thymus issue, tissue from a site of infection, ascites, pleural effusion, spleen tissue, or tumors. In some embodiments, the cell is an isolated non-natural cell. In some embodiments, the cell is obtained from a subject. In some embodiments, the cell is a human cell.

In another aspect, a composition comprising an engineered TCR, fusion polypeptide, polynucleotide, vector, or cell contemplated herein is provided.

In another aspect, a pharmaceutical composition comprising an engineered TCR, fusion polypeptide, polynucleotide, vector, or cell contemplated herein is provided.

In another aspect, a method of treating a subject in need thereof is provided, comprising administering the subject an effective amount of a cell, composition, or a pharmaceutical composition contemplated herein.

In another aspect, a method of treating, preventing, or ameliorating at least one symptom of a cancer, infectious disease, autoimmune disease, inflammatory disease, and immunodeficiency, or condition associated therewith is provided, comprising administering to the subject an effective amount a cell, composition, or a pharmaceutical composition contemplated herein.

In another aspect, a method of treating a solid cancer is provided, comprising administering to the subject an effective amount of a cell, composition, or a pharmaceutical composition contemplated herein. In various embodiments, the solid cancer is selected from the group consisting of: lung cancer, squamous cell carcinoma, colorectal cancer, pancreatic cancer, breast cancer, thyroid cancer, bladder cancer, cervical cancer, esophageal cancer, ovarian cancer, gastric cancer endometrial cancer, brain cancer, or sarcoma. In some embodiments, the solid cancer is a non-small cell lung carcinoma (NSCLC), small cell lung cancer (SCLC), head and neck squamous cell carcinoma, colorectal cancer, pancreatic cancer, breast cancer, thyroid cancer, bladder cancer, cervical cancer, esophageal cancer, ovarian cancer, gastric cancer endometrial cancer, gliomas, glioblastomas, oligodendroglioma, sarcoma, or osteosarcoma.

In another aspect, a method of treating a hematological malignancy comprising administering to the subject an effective amount of a cell, composition, or a pharmaceutical composition contemplated herein. In various embodiments, the hematological malignancy is a leukemia, lymphoma, or multiple myeloma. In some embodiments, the hematological malignancy is selected from the group consisting of non-Hodgkin's lymphoma, acute myeloid leukemia (AML), acute lymphoblastic leukemia (ALL).

### BRIEF DESCRIPTION OF SEVERAL VIEWS OF THE DRAWINGS

FIG. 1A shows illustrative MAGE TCR, CD33 DARIC, and engineered TCR (VHH-TCR) construct designs.

FIG. 1B shows an illustrative engineered TCR having a VHH linked to a TCR.

FIG. 2A shows VHH expression on immune effector cells.

FIG. 2B shows engineered TCR/receptor cytokine response against A549.CD33 cells.

FIG. 2C shows engineered TCR/receptor cytotoxicity against A549.CD33 cells.

FIG. 3A shows engineered TCR/receptor expression on immune effector cells.

FIG. 3B shows engineered TCR/receptor cytokine response against A549.A2.MAGEA4 cells.

FIG. 3C shows engineered TCR/receptor cytotoxicity against A549.A2.MAGEA4 cells.

FIG. 4A shows engineered TCR cytokine response against MAGEA4 peptide.

FIGS. 4B and 4C show engineered TCR cytokine response against cells electroporated with varying amounts of CD33 mRNA.

FIGS. 5A-5C show engineered TCR and DARIC cytotoxicity against HL-60, Kasumi1, and OCI-AML3 cells.

FIG. 6 shows illustrative engineered TCR constructs.

FIG. 7A shows VHH expression on immune effector cells.

FIG. 7B shows engineered TCR/receptor cytokine response against A549.CD33 cells.

FIG. 7C shows engineered TCR/receptor cytotoxicity against A549.CD33 cells.

FIG. 8A shows VHH expression on immune effector cells.

FIG. 8B shows engineered TCR/receptor cytokine response against A549.MAGEA4.A2 cells.

FIG. 8C shows engineered TCR/receptor cytotoxicity against A549.MAGEA4.A2 cells.

FIG. 9 shows illustrative engineered TCR constructs.

FIG. 10A shows VHH expression on immune effector cells.

FIG. 10B shows engineered TCR/receptor cytokine response against A549.CD33 cells.

FIG. 10C shows engineered TCR/receptor cytotoxicity against A549.CD33 cells.

FIG. 11A shows VHH expression on immune effector cells.

FIG. 11B shows engineered TCR cytokine response against A549.MAGEA4.A2 cells.

FIG. 11C shows engineered TCR/receptor cytotoxicity against A549.MAGEA4.A2 cells.

FIG. 12A shows illustrative MAGE TCR, CD33 DARIC, CLL1 DARIC, CLL1-CD33 DARIC, and engineered TCR (CLL1-CD33 VHH TCR) construct designs.

FIG. 12B shows an illustrative engineered TCR having two VHHs linked to the TCR.

FIG. 13A shows CD33-based receptor expression on immune effector cells.

FIG. 13B shows engineered TCR/receptor cytokine response against A549.CD33 cells.

FIG. 14A shows CLL1-based receptor expression on immune effector cells.

FIG. 14B shows engineered TCR/receptor cytokine response against A549.CLL1 cells.

FIG. 15A shows TCR expression on immune effector cells.

FIG. 15B shows engineered TCR/receptor cytokine response against A549.MAGEA4 cells.

FIG. 16 shows TCR and CAR expression on immune effector cells.

FIG. 17 shows engineered TCR and CAR cytokine responses against A375.NLR (MAGEA4+; BCMA−) cells.

FIG. 18A shows engineered TCR and CAR IFNg cytokine response against Toledo cells.

FIG. 18B shows engineered TCR and CAR IL-2 cytokine response against Toledo cells.

FIG. 19 shows antigen-independent IFNg cytokine response with engineered TCR and CAR T cells alone.

FIG. 20A shows illustrative MAGEA4 TCR, scFv CAR, and engineered TCR (scFv TCR) construct designs.

FIG. 20B shows an illustrative engineered TCR having an scFv linked to the TCR.

FIG. 21A shows BCMA-based receptor expression on immune effector cells.

FIG. 21B shows engineered TCR/receptor IFNg cytokine response against HT1080.BCMA, RPMI-8226, and Toledo cells.

FIG. 21C shows engineered TCR/receptor IL-2 cytokine response against HT1080.BCMA, RPMI-8226, and Toledo cells.

FIG. 21D shows engineered TCR/receptor TNFα cytokine response against HT1080.BCMA, RPMI-8226, and Toledo cells.

FIG. 21E shows engineered TCR/receptor cytotoxicity against HT1080.BCMA cells.

FIG. 22A shows TCR expression on immune effector cells.

FIG. 22B shows engineered TCR/receptor IFNg, IL2, and TNFα cytokine response against A375 cells.

FIG. 23 shows HL-60.FP (CD33+ MAGEA4−) tumor growth in an NGS systemic tumor model treated with UTD T cells, CD33 DARIC T cells, MAGEA4 TCR T cells, or VHH-TCR T cells.

FIG. 24 shows NCI-H2023 (CD33− MAGEA4+) tumor growth in an NGS subcutaneous tumor model treated with UTD T cells, CD33 DARIC T cells, MAGEA4 TCR T cells, or VHH-TCR T cells.

FIG. 25A shows TCR/ATOMIC expression on immune effector cells.

FIG. 25B shows engineered TCR/ATOMIC IFNg cytokine response against RPMI-8226 cells.

FIG. 25C shows engineered TCR/ATOMIC IFNg cytokine response against K562.CD19 cells.

### BRIEF DESCRIPTION OF THE SEQUENCE IDENTIFIERS

SEQ ID NOs: 1-32 set forth the amino acid sequences for representative target antigen binding domains.

SEQ ID NOs: 33-53 set forth the amino acid sequences for representative polypeptide linkers.

SEQ ID NOs: 54-79 set forth the amino acid sequences for representative TCR components (e.g., TCR variable regions).

SEQ ID NOs: 80-88 set forth the amino acid sequences for representative TCR constant domains.

SEQ ID NO: 89 sets forth the amino acid sequence for a representative MAGEA4-targeting TCR.

SEQ ID NOs: 90, 98, and 99 set forth the amino acid sequences for representative DARICs.

SEQ ID NO: 91-97, 100, and 102 set forth the amino acid sequences for representative engineered TCR constructs/ATOMICs.

SEQ ID NO: 101 sets forth the amino acid sequences for a representative anti-BCMA CAR.

SEQ ID NOs: 103-111 set forth the amino acid sequences for representative TRA or TRB polypeptides.

SEQ ID NO: 112 sets forth the amino acid sequence for a representative furin cleavage site.

SEQ ID NO: 113-137 set forth the amino acid sequences for representative polypeptide cleavage signal (e.g., self-cleaving peptides).

In the foregoing sequences, X, if present, refers to any amino acid or the absence of an amino acid.

### DETAILED DESCRIPTION

### A. Overview

The present disclosure generally relates to, in part, TCR-based constructs engineered to comprise one or more additional binding domains (e.g., antigen-binding domains), and methods of using the same. Without wishing to be bound by any particular theory, the inventors have unexpectedly discovered that TCRs engineered to comprise both a TCR binding domain (e.g., a TCR variable domain) and one or more additional antigen-binding domains are surprisingly effective at cell killing, and can target cells expressing either a TCR antigen, a non-TCR antigen, or both.

The multi-chain architecture of the TCR poses significant structural hurdles to grafting secondary binders into the TCR architecture, and success has primarily been achieved through co-expressing scFv-CD3 chain fusions or replacing the TCR variable regions with antibody-based binders. Overall, the complexity and MHC-restricted nature of the TCR architecture has stymied the development of broadly applicable technologies that achieve high levels of sensitivity and/or multiplexing. At minimum, there are very few potential solutions to these important challenges that do not consume the majority of available vector (e.g., lentiviral) payload space.

Thus, disclosed herein is an efficient and effective engineered/hybrid TCR architecture that enables concurrent TCR targeting and secondary binder targeting. Specifically, an antigen-binding domain (e.g., a VHH or scFv) is linked to a TCR component, e.g., a TCRα, TCRβ, TCRγ, and/or TCRδ variable domain/chain, in a manner that preserves TCR function. In certain embodiments, the engineered TCRs comprise a linker between the antigen-binding domain and the TCR component, such that the function of each targeting molecule (i.e., the TCR component and secondary antigen-binding domain) is preserved. Accordingly, the invention enables simultaneous targeting of intracellular and extracellular antigens.

In various embodiments, the engineered/hybrid TCR comprises one or more additional antigen-binding domains. In some embodiments, the engineered/hybrid TCR comprises two or more additional antigen-binding domains. In some embodiments, the two or more additional antigen-binding domains target the same or different antigens.

In various embodiments, the one or more antigen-binding domains are selected from the group consisting of: a Camel Ig, a Llama Ig, an Alpaca Ig, Ig NAR, a Fab′ fragment, a F(ab′)₂ fragment, a bispecific Fab dimer (Fab2), a trispecific Fab trimer (Fab3), an Fv, an single chain Fv protein (“scFv”), a bis-scFv, (scFv)₂, a minibody, a diabody, a triabody, a tetrabody, a disulfide stabilized Fv protein (“dsFv”), and a single-domain antibody (sdAb, a camelid VHH, Nanobody). In particular embodiments, the one or more antigen-binding domains comprise one or more single-chain variable fragments (scFv) or single domain antibodies (sdAb, e.g., camelid VHHs).

In various embodiments, the linker is a polypeptide linker from about 2 to about 25 amino acids long. In some embodiments, the linker is selected from the group consisting of: GG, GS, SG, SS, GSS, SSG, GSG, SGS, SGG, GGS, GGGS (SEQ ID NO: 53), (GGGGS)₁₋₅ polypeptide (SEQ ID NOs: 35-39), a linker from a marsupial γμTCR (e.g., LEKT; SEQ ID NO: 33), and any combination thereof. In particular embodiments, the linker comprises an amino acid sequence as set forth in any one of SEQ ID NOs: 33-53.

In various embodiments, the engineered TCR comprises one or more TCR components comprising one or more TCR variable domains that bind a target polypeptide presented by an MHC complex.

In various embodiments, the TCR component of the engineered TCR comprises a TCR constant region. In some embodiments, the TCR constant region is selected from a TCRα, TCRβ, TCRγ, or TCRδ constant region. In some embodiments, the TCR constant domain comprises an amino acid sequence at least 90% identical to an amino acid sequence as set forth in any one of SEQ ID NOs: 80-88.

In some embodiments, a non-functioning TCR can be used if antibody-based targeting alone is sufficient.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3^{rd} Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and C C Blackwell, eds., 1986); Roitt, Essential Immunology, 6^{th} Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

Methods and techniques for generating and modifying novel TCRs are also known in the art, see, e.g., Linnemann, C. et al., Nat. Med., 19, 1534-1541 (2013); Scheper, W. et al., Nat. Med., 25, 89-94 (2019); Yossef, R. et al., JCI Insight, 3, 122467 (2018); Hu, Z. et al., Blood, 132, 1911-1921 (2018); Li, Y. et al., Nat. Biotechnol, 23, 349-354 (2005); Wagner, E. K. et al. J. Biol. Chem, 294, 5790-5804 (2019); Guo, X.-Z. J. et al., Mol. Ther. Methods Clin. Dev, 3, 15054 (2016); Azizi, E. et al., Cell, 174, 1293-1308 (2018); Kieke, M. C. et al., Proc. Natl Acad. Sci. USA, 96, 5651-5656 (1999); Smith, S. N. et al, 1319, 95-141 (Springer, 2015); Tsuji, T. et al., Cancer Immunol. Res, 6, 594-604 (2018); and Spindler, M. J., et al., Nat Biotechnol, 38, 609-619 (2020).

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

As used herein, the term “TCR complex” refers to a complex formed by the association of CD3 with a TCR. For example, a TCR complex can be composed of a CD3γ chain, a CD3δ chain, two CD3ε chains, a homodimer of CD3ζ chains, a TCRα chain, and a TCRβ chain. In some embodiments, a TCR complex can be composed of a CD37 chain, a CD3δ chain, two CD3ε chains, a homodimer of CD3ζ chains, a TCRγ chain, and a TCRδ chain.

A “component of a TCR complex,” as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

As used herein, the terms, “binding domain,” “extracellular domain,” “antigen binding domain,” “extracellular binding domain,” “extracellular antigen binding domain,” “antigen-specific binding domain,” “extracellular antigen specific binding domain,” “binder,” and “antigen binder” are used interchangeably and provide a polypeptide with the ability to specifically bind to the target antigen of interest. The binding domain may be derived either from a natural, synthetic, semi-synthetic, or recombinant source.

The term “antibody” refers to a binding agent that is a polypeptide comprising at least a light chain or heavy chain immunoglobulin variable region or fragment thereof which specifically recognizes and binds one or more epitopes of an antigen, such as a peptide, lipid, polysaccharide, or nucleic acid containing an antigenic determinant, such as those recognized by an immune cell.

The term “antibody” encompasses any naturally-occurring, recombinant, modified or engineered immunoglobulin or immunoglobulin-like structure or antigen-binding fragment or portion thereof, or derivative thereof, as further described elsewhere herein. Thus, the term refers to an immunoglobulin molecule that specifically binds to a target antigen, and includes, for instance, chimeric, humanized, fully human, and bispecific antibodies. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies. Antibodies, or antigen-binding portions thereof, can be produced in hybridomas, by recombinant DNA techniques, or by enzymatic or chemical cleavage of intact antibodies.

The term “antigen binding fragment” or “antigen binding portion” refers to one or more fragments of an antibody that retain the ability to specifically bind to an antigen. Antigen binding fragments include, but are not limited to, any naturally occurring, enzymatically obtainable, synthetic, or genetically engineered polypeptide or glycoprotein that specifically binds an antigen to form a complex. In some embodiments, an antigen-binding portion of an antibody may be derived, e.g., from full antibody molecules using any suitable standard techniques such as proteolytic digestion or recombinant genetic engineering techniques involving the manipulation and expression of DNA encoding antibody variable and optionally constant domains.

A “Single-chain Fv” or “scFv” antibody fragments comprise the VH and VL domains of antibody, wherein these domains are present in a single polypeptide chain and in either orientation (e.g., VL-VH or VH-VL). For example, in some embodiments, the scFv variable light chain is positioned c-terminal to that of the variable heavy chain. In some embodiments, the scFv variable heavy chain is positioned c-terminal to that of the variable light chain. Generally, the scFv polypeptide further comprises a polypeptide linker between the VH and VL domains which enables the scFv to form the desired structure for antigen binding. For a review of scFv, see, e.g., Pluckthun, in The Pharmacology of Monoclonal Antibodies, vol. 113, Rosenburg and Moore eds., (Springer-Verlag, New York, 1994), pp. 269-315.

A “VHH,” “VHH antibody,” or “VHH domain” as used herein refers an antibody fragment that contains the smallest known antigen-binding unit of the variable region of a heavy chain antibody (Koch-Nolte, et al, FASEB J., 21: 3490-3498 (2007)).

An “isolated antibody or antigen binding fragment thereof” refers to an antibody or antigen binding fragment thereof which has been identified and separated and/or recovered from a component of its natural environment.

The terms “Antigen (Ag),” “target antigen,” and “polypeptide antigen” are used interchangeably and broadly include any molecules comprising an antigenic determinant within a binding region(s) to which an TCR or antibody or a fragment specifically binds. In particular embodiments, an “antigen (Ag)” refers to a compound, composition, or substance that can stimulate the production of antibodies or a T cell response in an animal, including compositions (such as one that includes a cancer-specific protein) that are injected or absorbed into an animal. An antigen reacts with the products of specific humoral or cellular immunity, including those induced by heterologous antigens, such as the disclosed antigens.

An antigen can be a single-unit molecule (such as a protein monomer or a fragment) or a complex comprised of multiple components. An antigen provides an epitope, e.g., a molecule or a portion of a molecule, or a complex of molecules or portions of molecules, capable of being bound by a selective binding agent, such as an antigen-binding protein (including, e.g., an antibody and/or a TCR). Thus, a selective binding agent may specifically bind to an antigen that is formed by two or more components in a complex. In some embodiments, the antigen is capable of being used in an animal to produce antibodies capable of binding to that antigen. An antigen can possess one or more epitopes that are capable of interacting with different antigen-binding proteins, e.g., antibodies. In preferred TCR-related embodiments, the terms “antigen (Ag),” “target antigen,” and “polypeptide antigen” are collective refer to a naturally processed or synthetically produced portion of an antigenic protein, e.g., a tumor associated antigen (TAA) or tumor specific antigen (TSA), ranging in length from about 7 amino acids to about 15 amino acids, which can form a complex with a MHC (e.g., HLA) molecule forming a target antigen:MHC (e.g., HLA) complex.

A “target antigen” or “target antigen of interest” refers to a molecule expressed on the cell surface of a target cell that a binding domain contemplated herein, is designed to bind. In particular embodiments, the target antigen is an epitope of a polypeptide expressed on the surface of a cancer cell. An “epitope” or “antigenic determinant” refers to the region of an antigen to which a binding agent binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation.

The terms “selectively binds” or “selectively bound” or “selectively binding” or “selectively targets”, “specific binding affinity” or “specifically binds” or “specifically bound” or “specific binding” or “specifically targets” as used herein, describes preferential binding of one molecule to a target molecule (on-target binding) in the presence of a plurality of off-target molecules. In particular embodiments, the terms refer to binding of a TCR, antibody, or antigen binding fragment thereof to an antigen at greater binding affinity than background binding. A binding domain “specifically binds” to an antigen if it binds to or associates with the antigen With an affinity or Kₐ (i.e., an equilibrium association constant of a particular binding interaction with units of 1/M) of, for example, greater than or equal to about 105 M⁻¹. In certain embodiments, a binding domain (or a fusion protein thereof) binds to a target with a Ka greater than or equal to about 10⁶ M⁻¹, 10⁷ M⁻¹, 10⁸ M⁻¹, 10⁹ M⁻¹, 10¹⁰ M⁻¹, 10¹¹ M¹, 10¹² M⁻¹, or 10¹³ M⁻¹. “High affinity” binding domains (or single chain fusion proteins thereof) refers to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10³ M⁻¹, or greater.

Alternatively, affinity may be defined as an equilibrium dissociation constant (K_{d}) of a particular binding interaction with units of M (e.g., 10⁻⁵ M to 10⁻¹³ M, or less). Affinities of binding domain polypeptides and CAR proteins according to the present disclosure can be readily determined using conventional techniques, e.g., by competitive ELISA (enzyme-linked immunosorbent assay), or by binding association, or displacement assays using labeled ligands, or using a surface-plasmon resonance device such as the Biacore T100, which is available from Biacore, Inc., Piscataway, NJ, or optical biosensor technology such as the EPIC system or EnSpire that are available from Corning and Perkin Elmer respectively (see also, e.g., Scatchard et al. (1949) Ann. N.Y. Acad. Sci. 51:660; and U.S. Pat. Nos. 5,283,173; 5,468,614, or the equivalent).

In one embodiment, the affinity of specific binding is about 2 times greater than background binding, about 5 times greater than background binding, about 10 times greater than background binding, about 20 times greater than background binding, about 50 times greater than background binding, about 100 times greater than background binding, or about 1000 times greater than background binding or more.

In particular embodiments, the engineered/hybrid TCR comprises an antibody or antigen binding fragment thereof. In the context of an engineered TCR, an “antibody” refers to a binding agent that is a polypeptide comprising at least a light chain or heavy chain immunoglobulin variable region which specifically recognizes and binds an epitope of an antigen, such as a peptide, lipid, polysaccharide, or nucleic acid containing an antigenic determinant, such as those recognized by an immune cell.

As would be understood by the skilled person and as described elsewhere herein, a complete antibody comprises two heavy chains and two light chains. Each heavy chain consists of a variable region and a first, second, and third constant region, while each light chain consists of a variable region and a constant region.

Light and heavy chain variable regions contain a “framework” region interrupted by three hypervariable regions, also called “complementarity-determining regions” or “CDRs.” The CDRs can be defined or identified by conventional methods, such as by sequence according to Kabat et al (Wu, TT and Kabat, E. A., J Exp Med. 132(2):211-50, (1970); Borden, P. and Kabat E. A., PNAS, 84: 2440-2443 (1987); (see, Kabat et al., Sequences of Proteins of Immunological Interest, U.S. Department of Health and Human Services, 1991, which is hereby incorporated by reference), or by structure according to Chothia et al (Chothia, C. and Lesk, A. M., J Mol. Biol., 196(4): 901-917 (1987), Chothia, C. et al, Nature, 342: 877-883 (1989)).

Other boundaries defining CDRs overlapping with the Kabat CDRs have been described by Padlan (1995) FASEB J. 9: 133-139 and MacCallum (1996) J. Mol. Biol. 262(5): 732-45. Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen-binding. For example, the CDRs of an antibody can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.).

Additionally, the CDRs of an antibody can be determined according to the IMGT numbering system as described in Lefranc M-P, (1999) The Immunologist 7: 132-136 and Lefranc M-P et al., (1999) Nucleic Acids Res 27: 209-212.

Still other methods of CDR determination are disclosed in MacCallum R M et al., (1996) J Mol Biol 262: 732-745. See also, e.g., Martin A. “Protein Sequence and Structure Analysis of Antibody Variable Domains,” in Antibody Engineering, Kontermann and Dubel, eds., Chapter 31, pp. 422-439, Springer-Verlag, Berlin (2001). Proprietary and publicly available programs are known to those skilled in the art which can be used to determine CDRs base on any of the CDR definitions described herein, for example, abYsis (abysis.org/abysis/) and IMGT/V-QUEST (imgt.org/IMGT_vquest).

Illustrative examples of rules for predicting light chain CDRs include: CDRL1 starts at about residue 24, is preceded by a Cys, is about 10-17 residues, and is followed by a Trp (typically Trp-Tyr-Gln, but also, Trp-Leu-Gln, Trp-Phe-Gln, Trp-Tyr-Leu); CDRL2 starts about 16 residues after the end of CDRL1, is generally preceded by Ile-Tyr, but also, Val-Tyr, Ile-Lys, Ile-Phe, and is 7 residues; and CDRL3 starts about 33 residues after the end of CDRL2, is preceded by a Cys, is 7-11 residues, and is followed by Phe-Gly-XXX-Gly (XXX is any amino acid).

Illustrative examples of rules for predicting heavy chain CDRs include: CDRH1 starts at about residue 26, is preceded by Cys-XXX-XXX-XXX, is 10-12 residues and is followed by a Trp (typically Trp-Val, but also, Trp-Ile, Trp-Ala); CDRH2 starts about 15 residues after the end of CDRH1, is generally preceded by Leu-Glu-Trp-Ile-Gly (SEQ ID NO: 138), or a number of variations, is 16-19 residues, and is followed by Lys/Arg-Leu/Ile/Val/Phe/Thr/Ala-Thr/Ser/Ile/Ala, AbM definition ends 7 residues earlier; and CDRH3 starts about 33 residues after the end of CDRH2, is preceded by Cys-XXX-XXX (typically Cys-Ala-Arg), is 3 to 25 residues, and is followed by Trp-Gly-XXX-Gly.

References to “V_{H}” or “VH” refer to the variable region of an immunoglobulin heavy chain, including that of an antibody, Fv, scFv, dsFv, Fab, or other antibody fragment as disclosed herein. References to “V_{L}” or “VL” refer to the variable region of an immunoglobulin light chain, including that of an antibody, Fv, scFv, dsFv, Fab, or other antibody fragment as disclosed herein.

Additional definitions are set forth throughout this disclosure.

### C. Engineered T Cell Receptors

T cell receptors (TCRs) recognize a peptide fragment of a target antigen when it is presented by a major histocompatibility complex (MHC) molecule. There are two different classes of MHC molecules, MHC I and MHC II, that deliver peptides from different cellular compartments to the cell surface. Engagement of the TCR with antigen and MHC results in immune effector cell activation through a series of biochemical events mediated by associated enzymes, co-receptors, and specialized accessory molecules.

A TCR contemplated herein is a heterodimeric complex comprising a TCR alpha (TCRα) polypeptide/chain and a TCR beta (TCRβ) polypeptide/chain; or a TCR gamma (TCRγ) polypeptide/chain and a TCR delta (TCRδ) polypeptide/chain.

The human TCRα locus is located on chromosome 14 (14q11.2). The mature TCRα chain comprises a variable domain derived from recombination of a variable (V) segment and a joining (J) segment, and a constant (C) domain. The term “variable TCRα region” or “TCRα variable region” or “variable TCRα chain” or “TCRα variable chain” or “variable TCRα domain” or “TCRα variable domain” refers to the variable region of a TCRα chain.

The human TCRβ locus is located on chromosome 7 (7q34). The mature TCRβ chain comprises a variable domain derived from recombination of a variable (V) segment, a diversity (D) segment, and a joining (J) segment, and one of two constant (C) domains. The term “variable TCRβ region” or “TCRβ variable region” or “variable TCRβ chain” or “TCRβ variable chain” or “variable TCRβ domain” or “TCRβ variable domain” refers to the variable region of a TCRβ chain.

The human TCRγ locus is located on chromosome 7 (7p14.1). The mature TCRγ chain comprises a variable domain derived from recombination of a variable (V) segment and a joining (J) segment, and a constant (C) domain. The term “variable TCRγ region” or “TCRγ variable region” or “variable TCRγ chain” or “TCRγ variable chain” or “variable TCRγ domain” or “TCRγ variable domain” refers to the variable region of a TCRγ chain.

The human TCRδ locus is located on chromosome 14 (14q11.2). The mature TCRδ chain comprises a variable domain derived from recombination of a variable (V) segment, a diversity (D) segment, and a joining (J) segment, and one of two constant (C) domains. The term “variable TCRδ region” or “TCRδ variable region” or “variable TCRδ chain” or “TCRδ variable chain” or “variable TCRδ domain” or “TCRδ variable domain” refers to the variable region of a TCRδ chain.

The rearranged V(D)J regions of both the TCRα, TCRβ, TCRγ, and TCRδ chains each contain three hypervariable regions known as complementarity determining regions (CDRs). CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. Framework regions (FRs) are positioned between the CDRs. These regions provide the structure of the TCR variable region.

The constant domain or constant region of the TCR chain also contributes to TCR structure and consists of an extracellular domain, a transmembrane domain and a short cytoplasmic domain. The TCR structure allows the formation of a TCR complex that includes the TCRα or TCRγ chain, the TCRβ or TCRδ chain, and accessory molecules CD3γ, CD3δ, CD3ε, and CD3ζ. The signal from the T cell complex is enhanced by simultaneous binding of the MHC molecules by a specific co-receptor. CD4 is the co-receptor for MHC II molecules expressed on helper T cells and CD8 is the co-receptor for MHC I molecules expressed on cytotoxic T cells. The co-receptor not only ensures the specificity of the TCR for an antigen, but also allows prolonged engagement between the antigen presenting cell and the T cell and recruits essential molecules (e.g., LCK) inside the cell involved in the signaling of the activated T lymphocyte.

Engineered TCRs contemplated herein can be used to redirect immune effector cells to target cells. Additionally, the TCRs contemplated herein are engineered to comprise a functional antigen binding domain. In particular embodiments, the engineered TCR comprises both functional TCR binding domains (e.g., functional TCR variable regions) and one or more separate antigen-binding domains linked to one or both of the TCR polypeptides/chains. Accordingly, in some embodiments, the engineered TCR variable domains and the additional antigen binding domains can bind the same antigen or two different antigens, or more. In some embodiments, the engineered TCR can bind both an intracellular antigen presented on MHC molecules and a second antigen (e.g., receptor, ligand, or cancer antigen). In some embodiments, the engineered TCR can bind three different antigens.

The TCRs contemplated herein are sometimes referred to as engineered TCRs, hybrid TCRs, dual targeting TCRs, multi-targeting TCRs, or ATOMICs (Antibody Tethered Orthogonal Multiplexing Compatible) and comprise one or more antigen-binding domain components (“A” component) and one or more TCR components (“C” component), with or without one or more linkers (“B” component), each of which are described in more detail in the subsections below.

The data in the Examples demonstrate that the engineered TCRs and fusion proteins disclosed herein may comprise an antigen-binding domain (“A” component) and/or TCR component (“C” component) specific for any antigen(s). One of ordinary skill in the art would readily understand that an antigen-binding domain component and TCR component, irrespective of the antigen specificity or any specific sequence, e.g., of its variable domain or CDR sequences, may be linked to produce an engineered TCR or fusion protein meeting the characteristics of the engineered TCRs disclosed herein.

This is because the inventors have unexpectedly discovered that the disclosed engineered TCRs and fusion proteins, which comprise an antigen-binding domain (“A” component) linked to one or more TCR binding domains (“C” component), have an efficient and effective architecture that enables concurrent TCR targeting and secondary antigen-binder targeting, in a manner that preserves the function of both components. The antigen specificity of the component, as well as the sequences of the component, e.g., variable domain or CDR sequences, can be varied by one of ordinary skill in the art using the illustrative general engineered TCR formulas provided herein. Therefore, although the present disclosure and Examples provide a plethora of engineered TCRs and fusion proteins comprising (i) antigen-binding domain components and TCR components to different antigens, as well as (ii) different antigen-binding domains directed to the same antigen, one of ordinary skill in the art would understand that the engineered TCRs and fusion proteins disclosed and claimed herein should not be limited by antigen-specificity or by sequence, e.g., variable region sequences or CDR sequences.

### 1. Antigen-Binding Domain Component (“A” Component)

Provided herein are engineered TCRs, and related fusion polypeptides, comprising (a) a TCRα or TCRγ polypeptide comprising a TCRα or TCRγ variable domain; (b) a TCRβ or TCRδ polypeptide comprising a TCRβ or TCRδ variable domain; and (c) one or more antigen-binding domains (“A” components) linked to the TCRα, TCRβ, TCRγ and/or TCRδ variable domains.

In various embodiments, the one or more antigen-binding domains (also referred to herein as binders or antigen-binders) comprises one or more, two or more, or three or more antigen-binding domains. In some embodiments, the one or more antigen-binding domains comprises one or more first antigen-binding domains linked to any one or more of the TCRα, TCRβ, TCRγ, and/or TCRδ variable domains. In some embodiments, the one or more antigen-binding domains comprises a first antigen-binding domain linked to the TCRα variable domain. In some embodiments, the one or more antigen-binding domains comprises a first antigen-binding domain linked to the TCRβ variable domain. In some embodiments, the one or more antigen-binding domains comprises a first antigen-binding domain linked to the TCRγ variable domain. In some embodiments, the one or more antigen-binding domains comprises a first antigen-binding domain linked to the TCRδ variable domain. In some embodiments, the one or more antigen-binding domains comprise: (i) a first antigen-binding domain linked to the TCRα variable domain, and (ii) a first antigen-binding domain linked to the TCRβ variable domain. In some embodiments, the one or more antigen-binding domains comprise: (i) a first antigen-binding domain linked to the TCRγ variable domain, and (ii) a first antigen-binding domain linked to the TCRδ variable domain. In some embodiments, the first antigen-binding domains are the same or different, and/or bind to the same or different target antigens.

In various embodiments, the first antigen-binding domains are linked to the N-terminus of the variable domains.

In various embodiments, the one or more antigen-binding domains comprises a second antigen-binding domain linked to the first antigen-binding domain. In some embodiments, the second antigen-binding domain is N-terminal of the first antigen-binding domain. In some embodiments, the one or more antigen-binding domains comprises a second antigen-binding domain linked to the first antigen-binding domain which is linked to the TCRα variable domain. In some embodiments, the one or more antigen-binding domains comprises a second antigen-binding domain linked to the first antigen-binding domain which is linked to the TCRβ variable domain. In some embodiments, the one or more antigen-binding domains comprises a second antigen-binding domain linked to the first antigen-binding domain which is linked to the TCRγ variable domain. In some embodiments, the one or more antigen-binding domains comprises a second antigen-binding domain linked to the first antigen-binding domain which is linked to the TCRδ variable domain.

In various embodiments, the one or more antigen-binding domains comprises: (i) a second antigen-binding domain linked to the first antigen-binding domain linked to the TCRα variable domain, and (ii) a second antigen-binding domain linked to the first antigen-binding domain linked to the TCRβ variable domain.

In various embodiments, the one or more antigen-binding domains comprises: (i) a second antigen-binding domain linked to the first antigen-binding domain linked to the TCRγ variable domain, and (ii) a second antigen-binding domain linked to the first antigen-binding domain linked to the TCRδ variable domain.

In various embodiments, the second antigen-binding domains are the same or different, and/or bind to the same or different target antigens. In some embodiments, the second antigen-binding domains are the same. In some embodiments, the second antigen-binding domains are different.

In various embodiments, the one or more antigen-binding domains (e.g., the first and/or second antigen-binding domains) bind a target antigen selected from the group consisting of: alpha folate receptor (FRα), αᵥβ₆ integrin, ADGRE2, BACE2, B cell maturation antigen (BCMA), B7-H3 (CD276), B7-H4, B7-H6, CA19.9, carbonic anhydrase IX (CAIX), CCR1, CD7, CD16, CD19, CD20, CD22, CD30, CD33, CD37, CD38, CD44, CD44v6, CD44v7/8, CD70, CD79a, CD79b, CD123, CD133, CD138, CD171, CD244, carcinoembryonic antigen (CEA), C-type lectin-like molecule-1 (CLL-1), CD2 subset 1 (CS-1), CLDN6, cMET, chondroitin sulfate proteoglycan 4 (CSPG4), CLDN18.2, cutaneous T cell lymphoma-associated antigen 1 (CTAGE1), DLL3, epidermal growth factor receptor (EGFR), epidermal growth factor receptor variant III (EGFRvIII), EGFR806, epithelial glycoprotein 2 (EGP2), epithelial glycoprotein 40 (EGP40), EPHB2, ERBB4, epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), FLT3, FN, FN-EDB, FRBeta, ganglioside G2 (GD2), ganglioside G3 (GD3), Glypican-3 (GPC3), EGFR family including ErbB2 (HER2), HER2p95, EGFRv3, IL-10Rα, IL-13Rα2, Kappa, cancer/testis antigen 2 (LAGE-1A), K-Ras, K-Ras G12C, K-Ras G12D, K-Ras G12V, Lambda, Lewis-Y (LeY), L1 cell adhesion molecule (L1-CAM), LILRB2, LY6G6GD, melanoma antigen recognized by T cells 1 (MelanA or MART1), Mesothelin (MSLN), MMP10, MUC1, MUC16, MHC class I chain related proteins A (MICA), MHC class I chain related proteins B (MICB), neural cell adhesion molecule (NCAM), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), transmembrane activator and CAML interactor (TACI), tumor endothelial marker 1 (TEM1/CD248), tumor endothelial marker 7-related (TEM7R), TIM3, trophoblast glycoprotein (TPBG), UL16-binding protein (ULBP) 1, ULBP2, ULBP3, ULBP4, ULBP5, ULBP6, and vascular endothelial growth factor receptor 2 (VEGFR2).

In various embodiments, the one or more antigen-binding domains (e.g., the first and/or second antigen-binding domains) bind a target polypeptide derived from a protein selected from the group consisting of: α-fetoprotein (AFP), ASCL2, B Melanoma Antigen (BAGE) family members, Brother of the regulator of imprinted sites (BORIS), Cancer-testis antigens, Cancer-testis antigen 83 (CT-83), Carbonic anhydrase IX (CAIX), Carcinoembryonic antigen (CEA), Cytomegalovirus (CMV) antigens, Cytotoxic T cell (CTL)-recognized antigen on melanoma (CAMEL), Epstein-Barr virus (EBV) antigens, EPHB2, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human papillomavirus (HPV)-E6, HPV-E7, Human telomerase reverse transcriptase (hTERT), IGF2BP3/A3, IGF2BP1, K-Ras, K-Ras G12C, K-Ras G12D, K-Ras G12V, Latent membrane protein 2 (LMP2), LY6G6D, Melanoma antigen family A, 1 (MAGE-A1), MAGE-A2, MAGE-A3, MAGE-A4, MAGE-A6, MAGE-A10, MAGE-A12, Melanoma antigen recognized by T cells (MART-1), Mesothelin (MSLN), Mucin 1 (MUC1), Mucin 16 (MUC16), New York esophageal squamous cell carcinoma-1 (NYESO-1), P53, P antigen (PAGE) family members, PAP, PIK3CA, PIK3CA H1047R, Placenta-specific 1 (PLAC1), Preferentially expressed antigen in melanoma (PRAME), Prostate specific antigen PSA, Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, TP53 R175H, Tyrosinase, Tyrosinase related protein (TRP)1, TRP2, UBD, Wilms tumor protein (WT-1), Wnt10A, X Antigen Family Member 1 (XAGE1), and X Antigen Family Member 2 (XAGE2).

In various embodiments, the one or more antigen-binding domains bind CD33, CLL1, CD19, CD20, CD22, CD79A, CD79B, or BCMA. In some embodiments, the one or more antigen-binding domains bind CD19, CD20, CD22, CD33, CD79A, CD79B, B7H3, Muc16, Her2, EGFR, FN-EDB, CLDN18.2, DLL3, FLT3, CLL1, CD123, or BCMA.

In various embodiments, the one or more antigen-binding domains comprises an amino acid sequence at least 85% identical to an amino acid sequence as set forth in any one of SEQ ID NOs: 1-32. In various embodiments, the one or more antigen-binding domains comprises an amino acid sequence at least 90% identical to an amino acid sequence as set forth in any one of SEQ ID NOs: 1-32. In various embodiments, the one or more antigen-binding domains comprises an amino acid sequence at least 95% identical to an amino acid sequence as set forth in any one of SEQ ID NOs: 1-32. In some embodiments, the one or more antigen-binding domains comprises an amino acid sequence at least 96% identical to an amino acid sequence as set forth in any one of SEQ ID NOs: 1-32. In some embodiments, the one or more antigen-binding domains comprises an amino acid sequence at least 97% identical to an amino acid sequence as set forth in any one of SEQ ID NOs: 1-32. In some embodiments, the one or more antigen-binding domains comprises an amino acid sequence at least 98% identical to an amino acid sequence as set forth in any one of SEQ ID NOs: 1-32. In some embodiments, the one or more antigen-binding domains comprises an amino acid sequence at least 99% identical to an amino acid sequence as set forth in any one of SEQ ID NOs: 1-32. In some embodiments, the one or more antigen-binding domains comprises an amino acid sequence as set forth in any one of SEQ ID NOs: 1-32.

In various embodiments, the one or more antigen-binding domains comprise an antibody or antigen binding fragment thereof selected from the group consisting of: a Camel Ig, a Llama Ig, an Alpaca Ig, Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, a camelid VHH, Nanobody).

In various embodiments, the one or more antigen-binding domains comprise one or more single-chain variable fragments (scFv).

In various embodiments, the one or more antigen-binding domains comprise one or more single domain antibodies (sdAb). In some embodiments, the sdAb is a camelid VHH, nanobody, or heavy chain-only antibody (HcAb). In particular embodiments, the sdAb is a camelid VHH.

In various embodiments, the antibody or antigen binding fragment thereof is human or humanized.

Numerous methods may be used for obtaining antibodies, or antigen-binding fragments thereof. For example, antibodies can be produced using recombinant DNA methods. Monoclonal antibodies may also be produced by generation of hybridomas (see e.g., Kohler and Milstein (1975) Nature, 256: 495-499) in accordance with known methods. Hybridomas formed in this manner are then screened using standard methods, such as enzyme-linked immunosorbent assay (ELISA) and surface plasmon resonance (e.g., OCTET or BIACORE) analysis, to identify one or more hybridomas that produce an antibody that specifically binds to a specified antigen. Any form of the specified antigen may be used as the immunogen, e.g., recombinant antigen, naturally occurring forms, any variants or fragments thereof, as well as antigenic peptide thereof (e.g., any of the epitopes described herein as a linear epitope or within a scaffold as a conformational epitope). One exemplary method of making antibodies includes screening protein expression libraries that express antibodies or fragments thereof (e.g., scFv), e.g., phage or ribosome display libraries. Phage display is described, for example, in Ladner et al., U.S. Pat. No. 5,223,409; Smith (1985) Science 228:1315-1317; Clackson et al. (1991) Nature, 352: 624-628; Marks et al. (1991) J. Mol. Biol., 222: 581-597; WO 92/18619; WO 91/17271; WO92/20791; WO92/15679; WO93/01288; WO92/01047; WO92/09690; and WO90/02809.

In some embodiments, a monoclonal antibody is obtained from the non-human animal, and then modified, e.g., chimeric, using suitable recombinant DNA techniques. A variety of approaches for making chimeric antibodies have been described. See e.g., Morrison et al., Proc. Natl. Acad. Sci. U.S.A. 81:6851, 1985; Takeda et al., Nature 314:452, 1985; Cabilly et al., U.S. Pat. No. 4,816,567; Boss et al., U.S. Pat. No. 4,816,397; Tanaguchi et al., European Patent Publication EP171496; European Patent Publication 0173494; and United Kingdom Patent GB 2177096B.

For additional antibody production techniques, see Antibodies: A Laboratory Manual, eds. Harlow et al., Cold Spring Harbor Laboratory, 1988. The present disclosure is not necessarily limited to any particular source, method of production, or other special characteristics of an antibody.

In various embodiments, the one or more antigen-binding domains comprise a ligand.

### 2. Linkers (“B” Component)

As contemplated herein, the engineered TCRs may or may not comprise linker residues (“B” component) between the various domains, e.g., added for appropriate spacing and conformation of the molecule. Particularly, the engineered TCRs comprise a linker between the one or more antigen-binding domains and the TCR component, e.g., TCR variable domain. In various embodiments, the one or more antigen-binding domains are linked to the TCR component, e.g., TCR variable domains, by one or more polypeptide linkers. In some embodiments, the TCR comprises two or more linkers between the antigen-binding domain and the TCR variable domain. In some embodiments, the engineered TCR does not comprise a polypeptide linker (“B” component) between the antigen-binding domain and the TCR component.

A “linker” or “polypeptide linker” or “linker polypeptide” is an amino acid sequence that connects adjacent domains of a polypeptide or fusion polypeptide. Illustrative examples of linkers include glycine polymers (G)ₙ; glycine-serine polymers (G₁₋₅S₁₅)ₙ, where n is an integer of at least one, two, three, four, or five; glycine-alanine polymers; alanine-serine polymers; and other flexible linkers known in the art. Glycine accesses significantly more phi-psi space than even alanine, and is much less restricted than residues with longer side chains (see Scheraga, Rev. Computational Chem. 11173-142 (1992)). A linker may be 1, 2, 3, 4, 5, 6, 7, 8, 9, 10, 11, 12, 13, 14, 15, 16, 17, 18, 19, 20, 21, 22, 23, 24, 25, or more amino acids long.

In particular embodiments, the engineered TCRs and/or antigen-binding domains comprise one, two, three, four, or five or more linkers. The linker may be between the TCR variable domain and the antigen-binding domain, between two or more antigen binding domains, or between VH and VL sequences within an antigen binding domain (e.g., scFv). In particular embodiments, the length of a linker is about 2 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

In various embodiments, the one or more polypeptide linkers comprise a linker from about 2 to about 25 amino acids long. In some embodiments, the one or more polypeptide linkers comprise a linker from about 3 to about 20 amino acids long. In some embodiments, the one or more polypeptide linkers comprise a linker from about 4 to about 15 amino acids long. In some embodiments, the one or more polypeptide linkers comprise a linker from about 4 to about 10 amino acids long. In some embodiments, the one or more polypeptide linkers comprise a linker of about 4 amino acids long. In some embodiments, the one or more polypeptide linkers comprise a linker of about 5 amino acids long. In some embodiments, the one or more polypeptide linkers comprise a linker of about 6 amino acids long. In some embodiments, the one or more polypeptide linkers comprise a linker of about 7 amino acids long. In some embodiments, the one or more polypeptide linkers comprise a linker of about 8 amino acids long. In some embodiments, the one or more polypeptide linkers comprise a linker of about 9 amino acids long. In some embodiments, the one or more polypeptide linkers comprise a linker of about amino acids long. In some embodiments, the one or more polypeptide linkers comprise a linker of about 11 amino acids long. In some embodiments, the one or more polypeptide linkers comprise a linker of about 12 amino acids long. In some embodiments, the one or more polypeptide linkers comprise a linker of about 13 amino acids long. In some embodiments, the one or more polypeptide linkers comprise a linker of about 14 amino acids long. In some embodiments, the one or more polypeptide linkers comprise a linker of about 15 amino acids long.

Illustrative examples of linkers include glycine polymers (G)ₙ; glycine-serine polymers (G₁₋₅S₁₋₅)ₙ, where n is an integer of at least one, two, three, four, or five; glycine-alanine polymers; alanine-serine polymers; and other flexible linkers are known in the art. Glycine and glycine-serine polymers are relatively unstructured, and therefore may be able to serve as a neutral tether between domains of fusion proteins such as the engineered/hybrid TCRs described herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of an engineered/hybrid TCR in particular embodiments can include linkers that are all or partially flexible, such that the linker can include a flexible linker as well as one or more portions that confer less flexible structure to provide for a desired TCR/hybrid structure.

Other illustrative linkers include, but are not limited to the following amino acid sequences: DGGGS (SEQ ID NO: 40); TGEKP (SEQ ID NO: 41) (see, e.g., Liu et al., PNAS 5525-5530 (1997)); GGRR (SEQ ID NO: 42) (Pomerantz et al. 1995, supra); (GGGGS)ₙ where n=1, 2, 3, 4 or 5 (SEQ ID NOs: 35-39) (Kim et al., PNAS 93, 1156-1160 (1996.); EGKSSGSGSESKVD (SEQ ID NO: 43) (Chaudhary et al., 1990, Proc. Natl. Acad. Sci. U.S.A. 87:1066-1070); KESGSVSSEQLAQFRSLD (SEQ ID NO: 44) (Bird et al., 1988, Science 242:423-426), GGRRGGGS (SEQ ID NO: 45); LRQRDGERP (SEQ ID NO: 46); LRQKDGGGSERP (SEQ ID NO: 47); LRQKD(GGGS)₂ ERP (SEQ ID NO: 48). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods. In particular embodiments, a linker comprises the amino acid sequence: GSTSGSGKPGSGEGSTKG (SEQ ID NO: 49) or GSTSGSGKSSEGSGSTKG (SEQ ID NO: 50) (Cooper et al., Blood, 101(4): 1637-1644 (2003) and Whitlow et al., Protein Eng., 6(8): 989-95 (1993)). Other linkers include GSTSGSGKSSEGKG (SEQ ID NO: 51), GSTSGSGKPGSGEGS (SEQ ID NO: 52), or GGGS (SEQ ID NO: 53).

In various embodiments, the one or more polypeptide linkers comprise a linker selected from the group consisting of: GG, GS, SG, SS, GSS, SSG, GSG, SGS, SGG, GGS, GGGS (SEQ ID NO: 53), (GGGGS)₁₋₅ polypeptide (SEQ ID NOs: 35-39), a linker from a marsupial YTCR (e.g., LEKT; SEQ ID NO: 33), and any combination thereof.

In various embodiments, the one or more polypeptide linkers comprise a linker from a marsupial γμTCR. In particular embodiments, the marsupial γμTCR linker is a μLNK comprising an amino acid sequence as set forth in SEQ ID NO: 33. In various embodiments, the one or more polypeptide linkers comprise a marsupial γμTCR linker and a G4S linker as set forth in SEQ ID NO: 34.

In various embodiments, the one or more polypeptide linkers comprise a GGGGS (SEQ ID NO: 35) linker (G4S). In various embodiments, the one or more polypeptide linkers comprise two GGGGS linkers (2×G4S) (SEQ ID NO: 36). In various embodiments, the one or more polypeptide linkers comprise three GGGGS linkers (3×G4S) (SEQ ID NO: 37). In various embodiments, the one or more polypeptide linkers comprise four GGGGS linkers (4×G4S) (SEQ ID NO: 38). In various embodiments, the one or more polypeptide linkers comprise five GGGGS linkers (5×G4S) (SEQ ID NO: 39).

In various embodiments, the one or more polypeptide linkers comprise an amino acid sequence as set forth in any one of SEQ ID NOs: 33-53.

In particular embodiments, the first and second antigen-binding domains are separated by a second polypeptide linker. In some embodiments, the second polypeptide linker comprises a linker from about 2 to about 25 amino acids long. In some embodiments, the second polypeptide linker comprises a linker from about 3 to about 20 amino acids long. In some embodiments, the second polypeptide linker comprises a linker from about 4 to about 15 amino acids long. In some embodiments, the second polypeptide linker comprises a linker from about 4 to about 10 amino acids long. In some embodiments, the second polypeptide linker comprises a linker of about 4 amino acids long. In some embodiments, the second polypeptide linker comprises a linker of about 5 amino acids long. In some embodiments, the second polypeptide linker comprises a linker of about 6 amino acids long. In some embodiments, the second polypeptide linker comprises a linker of about 7 amino acids long. In some embodiments, the second polypeptide linker comprises a linker of about 8 amino acids long. In some embodiments, the second polypeptide linker comprises a linker of about 9 amino acids long. In some embodiments, the second polypeptide linker comprises a linker of about 10 amino acids long. In some embodiments, the second polypeptide linker comprises a linker of about 11 amino acids long. In some embodiments, the second polypeptide linker comprises a linker of about 12 amino acids long. In some embodiments, the second polypeptide linker comprises a linker of about 13 amino acids long. In some embodiments, the second polypeptide linker comprises a linker of about 14 amino acids long. In some embodiments, the second polypeptide linker comprises a linker of about 15 amino acids long.

In various embodiments, the second polypeptide linker comprises a linker selected from the group consisting of: GG, GS, SG, SS, GSS, SSG, GSG, SGS, SGG, GGS, GGGS (SEQ ID NO: 53), (GGGGS)₁₋₅ polypeptide (SEQ ID NOs: 35-39), and any combination thereof. In some embodiments, the second polypeptide linker comprises a GGGGS (SEQ ID NO: 35) linker (G4S). In some embodiments, the one or more polypeptide linkers comprise two GGGGS linkers (2×G4S) (SEQ ID NO: 36). In some embodiments, the second polypeptide linker comprises three GGGGS linkers (3×G4S) (SEQ ID NO: 37). In some embodiments, the second polypeptide linker comprises four GGGGS linkers (4×G4S) (SEQ ID NO: 38). In some embodiments, the second polypeptide linker comprises five GGGGS linkers (5×G4S) (SEQ ID NO: 39).

In various embodiments, the second polypeptide linker comprises an amino acid sequence as set forth in any one of SEQ ID NOs: 33-53, or combination thereof.

### 3. T Cell Receptor Component (“C” Component)

The engineered T cell receptors (TCRs) contemplated herein bind a polypeptide antigen presented by a major histocompatibility complex (MHC) class I or MHC class II molecule, preferentially a polypeptide antigen presented by an MHC class I molecule.

“Major histocompatibility complex” (MHC) refers to glycoproteins that deliver peptide antigens to a cell surface. MHC class I molecules are heterodimers having a membrane spanning α chain (with three α domains) and a non-covalently associated β2 microglobulin. MHC class II molecules are composed of two transmembrane glycoproteins, α and β, both of which span the membrane. Each chain has two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where they are recognized by CD4⁺ T cells. Human MHC is referred to as human leukocyte antigen (HLA).

Principles of antigen processing by antigen presenting cells (APC) (such as dendritic cells, macrophages, lymphocytes or other cell types), and of antigen presentation by APC to T cells, including major histocompatibility complex (MHC)-restricted presentation between immunocompatible (e.g., sharing at least one allelic form of an MHC gene that is relevant for antigen presentation) APC and T cells, are well established (see, e.g., Murphy, Janeway's Immunobiology (8th Ed.) 2011 Garland Science, NY; chapters 6, 9 and 16). For example, processed antigen peptides originating in the cytosol (e.g., tumor antigen, intracellular pathogen) are generally from about 7 amino acids to about 11 amino acids in length and will associate with class I MHC molecules, whereas peptides processed in the vesicular system (e.g., bacterial, viral) will generally vary in length from about 10 amino acids to about 25 amino acids and associate with class II MHC molecules.

In particular embodiments, an engineered TCR contemplated herein binds a tumor antigen, e.g., a TAA or TSA. “Tumor associate antigens” or “TAAs” include but are not limited to oncofetal antigens, overexpressed antigens, lineage restricted antigens, and cancer-testis antigens. TAAs are relatively restricted to tumor cells. TAAs have elevated expression levels on tumor cells, but are also expressed at lower levels on healthy cells. “Tumor-specific antigens” or “TSAs” include but are not limited to neoantigens and oncoviral antigens. TSAs are unique to tumor cells. TSAs are expressed in cancer cells and not normal cells.

In particular embodiments, engineered TCRs contemplated herein bind an antigenic portion of a polypeptide selected from the group consisting of: α-fetoprotein (AFP), ASCL2, B Melanoma Antigen (BAGE) family members, Brother of the regulator of imprinted sites (BORIS), Cancer-testis antigens, Cancer-testis antigen 83 (CT-83), Carbonic anhydrase IX (CAIX), Carcinoembryonic antigen (CEA), Cytomegalovirus (CMV) antigens, Cytotoxic T cell (CTL)-recognized antigen on melanoma (CAMEL), Epstein-Barr virus (EBV) antigens, EPHB2, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human papillomavirus (HPV)-E6, HPV-E7, Human telomerase reverse transcriptase (hTERT), IGF2BP3/A3, IGF2BP1, K-Ras, K-Ras G12C, K-Ras G12D, K-Ras G12V, Latent membrane protein 2 (LMP2), LY6G6D, Melanoma antigen family A, 1 (MAGE-A1), MAGE-A2, MAGE-A3, MAGE-A4, MAGE-A6, MAGE-A10, MAGE-A12, Melanoma antigen recognized by T cells (MART-1), Mesothelin (MSLN), Mucin 1 (MUC1), Mucin 16 (MUC16), New York esophageal squamous cell carcinoma-1 (NYESO-1), P53, P antigen (PAGE) family members, PAP, PIK3CA, PIK3CA H1047R, Placenta-specific 1 (PLAC1), Preferentially expressed antigen in melanoma (PRAME), Prostate specific antigen PSA, Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, TP53 R175H, Tyrosinase, Tyrosinase related protein (TRP)1, TRP2, UBD, Wilms tumor protein (WT-1), Wnt10A, X Antigen Family Member 1 (XAGE1), and X Antigen Family Member 2 (XAGE2). In some embodiments, the TCR variable domains bind a target polypeptide derived from MAGE-A4, PRAME, K-Ras, TP53R175H, PSA, or IGF2BP3. In some embodiments, the TCR variable domains bind a target polypeptide derived from MAGE-A4.

As contemplated herein, the engineered TCRs comprise a TCR component (“C” component). In some embodiments, the TCR component comprises a TCRα polypeptide comprising a TCRα variable domain. In some embodiments, the TCR component comprises a TCRβ polypeptide comprising a TCRβ variable domain. In some embodiments, the TCR component comprises a TCRγ polypeptide comprising a TCRγ variable domain. In some embodiments, the TCR component comprises a TCRδ polypeptide comprising a TCRδ variable domain.

In one embodiment, the TCR component (“C” component) comprises a TCRα polypeptide comprising a TCRα variable domain; and a TCRβ polypeptide comprising a TCRβ variable domain. In a particular embodiment, the TCR component comprises a TCRα polypeptide comprising a TCRα variable domain; a TCRβ polypeptide comprising a TCRβ variable domain; and one or more antigen-binding domains linked to the TCRγ variable domain and/or TCRδ variable domain.

In one embodiment, the TCR component (“C” component) a TCRγ polypeptide comprising a TCRγ variable domain; and a TCRδ polypeptide comprising a TCRδ variable domain. In a particular embodiment, the TCR component comprises a TCRγ polypeptide comprising a TCRγ variable domain; a TCRδ polypeptide comprising a TCRδ variable domain; and one or more antigen-binding domains linked to the TCRγ variable domain and/or TCRδ variable domain.

In various embodiments, the TCR component (“C” component) comprises a TCR constant domain. One of skill in the art would understand that a given TCR variable domains can be paired with any one of several different constant domains. For example, any one of the TCRα, TCRβ, TCRγ, or TCRδ variable domains can be paired with any one of the TCRα, TCRβ, TCRγ, or TCRδ constant domains. In some embodiments, the TCRα polypeptide comprises a TCRα constant domain. In some embodiments, the TCRβ polypeptide comprises a TCRβ constant domain. In some embodiments, the TCRγ polypeptide comprises a TCRγ constant domain. In some embodiments, the TCRδ polypeptide comprises a TCRδ constant domain. In some embodiments a TCRα variable domain is paired with a TCRγ constant domain. In some embodiments a TCRα variable domain is paired with a TCRδ constant domain. In some embodiments a TCRβ variable domain is paired with a TCRγ constant domain. In some embodiments a TCRβ variable domain is paired with a TCRδ constant domain. In some embodiments a TCRγ variable domain is paired with a TCRα constant domain. In some embodiments a TCRγ variable domain is paired with a TCRβ constant domain. In some embodiments a TCRδ variable domain is paired with a TCRα constant domain. In some embodiments a TCRδ variable domain is paired with a TCRβ constant domain.

The constant domains can be derived from native constant domains or mutated to enhance pairing with each other over pairing with native TCRs when expressed, or to increase stability. Such pairing and stability enhanced TCR are known, see, e.g., WO2021195503A1 and WO2018102795A1, which is incorporated by reference herein, in its entirety.

In various embodiments, the TCRα constant domain comprises an amino acid sequence at least 85% identical to an amino acid sequence as set forth in SEQ ID NOs: 82 or 88. In some embodiments, the TCRα constant domain comprises an amino acid sequence at least 90% identical to an amino acid sequence as set forth in SEQ ID NOs: 82 or 88. In some embodiments, the TCRα constant domain comprises an amino acid sequence at least 95% identical to an amino acid sequence as set forth in SEQ ID NOs: 82 or 88. In some embodiments, the TCRα constant domain comprises an amino acid sequence at least 96% identical to an amino acid sequence as set forth in SEQ ID NOs: 82 or 88. In some embodiments, the TCRα constant domain comprises an amino acid sequence at least 97% identical to an amino acid sequence as set forth in SEQ ID NOs: 82 or 88. In some embodiments, the TCRα constant domain comprises an amino acid sequence at least 98% identical to an amino acid sequence as set forth in SEQ ID NOs: 82 or 88. In some embodiments, the TCRα constant domain comprises an amino acid sequence at least 99% identical to an amino acid sequence as set forth in SEQ ID NOs: 82 or 88. In some embodiments, the TCRα constant domain comprises an amino acid sequence as set forth in SEQ ID NOs: 82 or 88.

In various embodiments, the TCRβ constant domain comprises an amino acid sequence at least 85% identical to an amino acid sequence as set forth in any one of SEQ ID NOs: 80, 81, 86, or 87. In some embodiments, the TCRβ constant domain comprises an amino acid sequence at least 90% identical to an amino acid sequence as set forth in any one of SEQ ID NOs: 80, 81, 86, or 87. In some embodiments, the TCRβ constant domain comprises an amino acid sequence at least 95% identical to an amino acid sequence as set forth in any one of SEQ ID NOs: 80, 81, 86, or 87. In some embodiments, the TCRβ constant domain comprises an amino acid sequence at least 96% identical to an amino acid sequence as set forth in any one of SEQ ID NOs: 80, 81, 86, or 87. In some embodiments, the TCRβ constant domain comprises an amino acid sequence at least 97% identical to an amino acid sequence as set forth in any one of SEQ ID NOs: 80, 81, 86, or 87. In some embodiments, the TCRβ constant domain comprises an amino acid sequence at least 98% identical to an amino acid sequence as set forth in any one of SEQ ID NOs: 80, 81, 86, or 87. In some embodiments, the TCRβ constant domain comprises an amino acid sequence at least 99% identical to an amino acid sequence as set forth in any one of SEQ ID NOs: 80, 81, 86, or 87. In some embodiments, the TCRβ constant domain comprises an amino acid sequence as set forth in any one of SEQ ID NOs: 80, 81, 86, or 87.

In various embodiments, the TCRγ constant domain comprises an amino acid sequence at least 85% identical to an amino acid sequence as set forth in SEQ ID NO: 83 or 84. In some embodiments, the TCRγ constant domain comprises an amino acid sequence at least 90% identical to an amino acid sequence as set forth in SEQ ID NO: 83 or 84. In some embodiments, the TCRγ constant domain comprises an amino acid sequence at least 95% identical to an amino acid sequence as set forth in SEQ ID NO: 83 or 84. In some embodiments, the TCRγ constant domain comprises an amino acid sequence at least 96% identical to an amino acid sequence as set forth in SEQ ID NO: 83 or 84. In some embodiments, the TCRγ constant domain comprises an amino acid sequence at least 97% identical to an amino acid sequence as set forth in SEQ ID NO: 83 or 84. In some embodiments, the TCRγ constant domain comprises an amino acid sequence at least 98% identical to an amino acid sequence as set forth in SEQ ID NO: 83 or 84. In some embodiments, the TCRγ constant domain comprises an amino acid sequence at least 99% identical to an amino acid sequence as set forth in SEQ ID NO: 83 or 84. In some embodiments, the TCRγ constant domain comprises an amino acid sequence as set forth in SEQ ID NO: 83 or 84.

In various embodiments, the TCRδ constant domain comprises an amino acid sequence at least 85% identical to an amino acid sequence as set forth in SEQ ID NO: 85. In some embodiments, the TCRδ constant domain comprises an amino acid sequence at least 90% identical to an amino acid sequence as set forth in SEQ ID NO: 85. In some embodiments, the TCRδ constant domain comprises an amino acid sequence at least 95% identical to an amino acid sequence as set forth in SEQ ID NO: 85. In some embodiments, the TCRδ constant domain comprises an amino acid sequence at least 96% identical to an amino acid sequence as set forth in SEQ ID NO: 85. In some embodiments, the TCRδ constant domain comprises an amino acid sequence at least 97% identical to an amino acid sequence as set forth in SEQ ID NO: 85. In some embodiments, the TCRδ constant domain comprises an amino acid sequence at least 98% identical to an amino acid sequence as set forth in SEQ ID NO: 85. In some embodiments, the TCRδ constant domain comprises an amino acid sequence at least 99% identical to an amino acid sequence as set forth in SEQ ID NO: 85. In some embodiments, the TCRδ constant domain comprises an amino acid sequence as set forth in SEQ ID NO: 85.

In various embodiments, the TCRα polypeptide comprises an amino acid sequence as set forth in any one of SEQ ID NOs: 105-111. In some embodiments, the TCRα polypeptide comprises an amino acid sequence as set forth in SEQ ID NO: 105. In some embodiments, the TCRα polypeptide comprises an amino acid sequence as set forth in SEQ ID NO: 106. In some embodiments, the TCRα polypeptide comprises an amino acid sequence as set forth in SEQ ID NO: 107. In some embodiments, the TCRα polypeptide comprises an amino acid sequence as set forth in SEQ ID NO: 108. In some embodiments, the TCRα polypeptide comprises an amino acid sequence as set forth in SEQ ID NO: 109. In some embodiments, the TCRα polypeptide comprises an amino acid sequence as set forth in SEQ ID NO: 110. In some embodiments, the TCRα polypeptide comprises an amino acid sequence as set forth in SEQ ID NO: 111.

In various embodiments, the TCRα polypeptide comprises an amino acid sequence as set forth in any one of SEQ ID NOs: 62, 64, 66, 68, 70, 72, 74, and 76. In some embodiments, the TCRα polypeptide comprises an amino acid sequence as set forth in SEQ ID NO: 62. In some embodiments, the TCRα polypeptide comprises an amino acid sequence as set forth in SEQ ID NO: 64. In some embodiments, the TCRα polypeptide comprises an amino acid sequence as set forth in SEQ ID NO: 66. In some embodiments, the TCRα polypeptide comprises an amino acid sequence as set forth in SEQ ID NO: 68. In some embodiments, the TCRα polypeptide comprises an amino acid sequence as set forth in SEQ ID NO: 70. In some embodiments, the TCRα polypeptide comprises an amino acid sequence as set forth in SEQ ID NO: 72. In some embodiments, the TCRα polypeptide comprises an amino acid sequence as set forth in SEQ ID NO: 74. In some embodiments, the TCRα polypeptide comprises an amino acid sequence as set forth in SEQ ID NO: 76.

In various embodiments, the TCRγ polypeptide comprises an amino acid sequence as set forth in any one of SEQ ID NO: 78.

In various embodiments, the TCRβ polypeptide comprises an amino acid sequence as set forth in SEQ ID NO: 103 or 104. In some embodiments, the TCRβ polypeptide comprises an amino acid sequence as set forth in SEQ ID NO: 103. In some embodiments, the TCRβ polypeptide comprises an amino acid sequence as set forth in SEQ ID NO: 104.

In various embodiments, the TCRβ polypeptide comprises an amino acid sequence as set forth in any one of SEQ ID NOs: 63, 65, 67, 69, 71, 73, 75, and 77. In some embodiments, the TCRβ polypeptide comprises an amino acid sequence as set forth in SEQ ID NO: 63. In some embodiments, the TCRβ polypeptide comprises an amino acid sequence as set forth in SEQ ID NO: 65. In some embodiments, the TCRβ polypeptide comprises an amino acid sequence as set forth in SEQ ID NO: 67. In some embodiments, the TCRβ polypeptide comprises an amino acid sequence as set forth in SEQ ID NO: 69. In some embodiments, the TCRβ polypeptide comprises an amino acid sequence as set forth in SEQ ID NO: 71. In some embodiments, the TCRβ polypeptide comprises an amino acid sequence as set forth in SEQ ID NO: 73. In some embodiments, the TCRβ polypeptide comprises an amino acid sequence as set forth in SEQ ID NO: 75. In some embodiments, the TCRβ polypeptide comprises an amino acid sequence as set forth in SEQ ID NO: 77.

In various embodiments, the TCRδ polypeptide comprises an amino acid sequence as set forth in any one of SEQ ID NO: 79.

As discussed herein, one or more antigen-binding domains are linked to one or both TCR variable domains of the TCR component. For example, one or more antigen-binding domains can be linked to any one or more of the TCRα, TCRβ, TCRγ, or TCRδ variable domains as the case may be. Various antigen-binding domain/TCR component configurations are contemplated herein. For example, a first antigen binding domain can be linked to the N-terminus of one or both TCR polypeptides (e.g., TCRα/β or TCRγ/δ variable regions). Additionally, a second antigen binding domain can be linked to the N-terminus of the first antigen binding domain, thus generating a tandem antigen binding domain. The first and second antigen-binding domains can be targeted to bind the same or different antigens. Similarly, multiple first binding domains can be targeted to bind the same or different antigens, and multiple second binding domains can be targeted to bind the same or different antigens.

In various embodiments, the TCR component further comprises a signal sequence/peptide. In some embodiments, the TCRα, TCRβ, TCRγ, or TCRδ polypeptides comprise an N-terminal signal sequence.

In some embodiments, the TCRα polypeptide comprises an N-terminal TCRα, TCRβ, TCRγ, TCRδ, CD8α, or IgK signal sequence. In some embodiments, the TCRα polypeptide comprises an N-terminal TCRα signal sequence. In some embodiments, the TCRα polypeptide comprises an N-terminal IgK signal sequence. In some embodiments, the TCRα polypeptide comprises an N-terminal CD8α signal sequence.

In some embodiments, the TCRβ polypeptide comprises an N-terminal TCRα, TCRβ, TCRγ, TCRδ, CD8α, or IgK signal sequence. In some embodiments, the TCRβ polypeptide comprises an N-terminal TCRβ signal sequence. In some embodiments, the TCRβ polypeptide comprises an N-terminal IgK signal sequence. In some embodiments, the TCRβ polypeptide comprises an N-terminal CD8α signal sequence.

In some embodiments, the TCRγ polypeptide comprises an N-terminal TCRα, TCRβ, TCRγ, TCRδ, CD8α, or IgK signal sequence. In some embodiments, the TCRγ polypeptide comprises an N-terminal TCRγ signal sequence. In some embodiments, the TCRγ polypeptide comprises an N-terminal IgK signal sequence. In some embodiments, the TCRγ polypeptide comprises an N-terminal CD8α signal sequence.

In some embodiments, the TCRδ polypeptide comprises an N-terminal TCRα, TCRβ, TCRγ, TCRδ, CD8α, or IgK signal sequence. In some embodiments, the TCRδ polypeptide comprises an N-terminal TCRδ signal sequence. In some embodiments, the TCRδ polypeptide comprises an N-terminal IgK signal sequence. In some embodiments, the TCRδ polypeptide comprises an N-terminal CD8α signal sequence.

### D. Illustrative Engineered TCR Polypeptides and Complexes

Various engineered TCR polypeptides and their related variants and complexes are contemplated herein. As discussed above, the engineered TCRs surprising have multi-specificity, the ability to simultaneously target both intracellular and extracellular targets, and increased sensitivity to non-MHC presented targets.

The engineered TCRs can be constructed in multiple formats, and can be designed and constructed using known components (e.g., antigen-binding domains, polypeptide linkers, and TCRα and TCRβ chains) and techniques. For example, one or more antigen-binding domains (e.g., one or more “A” components) can be linked to one or more TCR components (e.g., one or more “C” components) with or without one or more polypeptide linkers (e.g., with or without one or more “B” components) using standard cloning techniques. The “A” component can be linked to either the TCRα or TCRβ polypeptide/chain or both; or the TCRγ or TCRδ or both; of the “C” component, as the case may be. Illustrative engineered TCR formulas are provided below:

A-C

A-B-C

Illustrative antigen-binding domains, linkers, and TCRs can be found in Tables 3-5 below. Additionally, Table 6 provides an illustrative list of engineered TCR/ATOMIC polypeptides and complexes based on the antigen-binding domains, linkers, and TCRs provided in Tables 3, 4, and 5 (see Example 10). One of skill in the art would understand that other combinations are possible, including combinations using other antigen-binding domains, linkers, and TCRs either known to or newly developed by the skilled artisan.

In various embodiments, the TCRα polypeptide comprises an amino acid sequence as set forth in SEQ ID NO: 105. In various embodiments, the TCRα polypeptide comprises an amino acid sequence as set forth in SEQ ID NO: 106. In various embodiments, the TCRα polypeptide comprises an amino acid sequence as set forth in SEQ ID NO: 107. In various embodiments, the TCRα polypeptide comprises an amino acid sequence as set forth in SEQ ID NO: 108. In various embodiments, the TCRα polypeptide comprises an amino acid sequence as set forth in SEQ ID NO: 109. In various embodiments, the TCRα polypeptide comprises an amino acid sequence as set forth in SEQ ID NO: 110. In various embodiments, the TCRα polypeptide comprises an amino acid sequence as set forth in SEQ ID NO: 111.

In various embodiments, the TCRβ polypeptide comprises an amino acid sequence as set forth in SEQ ID NO: 103. In various embodiments, the TCRβ polypeptide comprises an amino acid sequence as set forth in SEQ ID NO: 104.

### E. Polypeptides

Various polypeptides, fusion polypeptides, and polypeptide variants are contemplated herein, including, but not limited to, TCR polypeptides, TCRα chain polypeptides, TCRβ chain polypeptides, TCR fusion polypeptides, and fragments thereof.

“Polypeptide,” “peptide” and “protein” are used interchangeably, unless specified to the contrary, and according to conventional meaning, i.e., as a sequence of amino acids. Polypeptides are not limited to a specific length, e.g., they may comprise a full-length polypeptide or a polypeptide fragment, and may include one or more post-translational modifications of the polypeptide, for example, glycosylations, acetylations, phosphorylations and the like, as well as other modifications known in the art, both naturally occurring and non-naturally occurring.

An “isolated polypeptide” and the like, as used herein, refer to in vitro synthesis, isolation, and/or purification of a peptide or polypeptide molecule from a cellular environment, and from association with other components of the cell, i.e., it is not significantly associated with in vivo substances. In particular embodiments, an isolated polypeptide is a synthetic polypeptide, a recombinant polypeptide, or a semi-synthetic polypeptide, or a polypeptide obtained or derived from a recombinant source.

Polypeptides include “polypeptide variants.” Polypeptide variants may differ from a naturally occurring polypeptide in one or more amino acid substitutions, deletions, additions and/or insertions. Such variants may be naturally occurring or may be synthetically generated, for example, by modifying one or more of the polypeptide sequences contemplated herein. For example, in particular embodiments, it may be desirable to improve the binding affinity, stability, expression, specific pairing, functional avidity and/or other biological properties of a TCR by introducing one or more substitutions, deletions, additions and/or insertions into any one or more of the TCRα, TCRβ, TCRγ, and/or TCRδ polypeptides, variable domains, and/or constant regions. In particular embodiments, polypeptides include polypeptides having at least about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity to any of the polypeptide sequences contemplated herein, typically where the variant maintains at least one biological activity of the reference sequence.

Polypeptides include “polypeptide fragments.” Polypeptide fragments refer to a polypeptide, which can be monomeric or multimeric that has an amino-terminal deletion, a carboxyl-terminal deletion, and/or an internal deletion or substitution of a naturally-occurring or recombinantly-produced polypeptide. As used herein, the term “biologically active fragment” or “minimal biologically active fragment” refers to a polypeptide fragment that retains at least 100%, at least 90%, at least 80%, at least 70%, at least 60%, at least 50%, at least 40%, at least 30%, at least 20%, at least 10%, or at least 5% of the naturally occurring polypeptide activity. In certain embodiments, a polypeptide fragment can comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long.

As noted above, in particular embodiments, polypeptides may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In certain embodiments, a polypeptide variant comprises one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides contemplated in particular embodiments and still obtain a functional molecule that encodes a variant or derivative polypeptide with desirable characteristics. When it is desired to alter the amino acid sequence of a polypeptide to create an equivalent, or even an improved, variant polypeptide, one skilled in the art, for example, can change one or more of the codons of the encoding DNA sequence, e.g., according to Table 1.

TABLE 1
Amino Acid Codons

| | One | Three | | | | | | |
|---|---|---|---|---|---|---|---|---|
| | letter | letter | | | | | | |
| Amino Acids | code | code | Codons | | | | | |
| Alanine | A | Ala | GCA | GCC | GCG | GCU | | |
| Cysteine | C | Cys | UGC | UGU | | | | |
| Aspartic acid | D | Asp | GAC | GAU | | | | |
| Glutamic acid | E | Glu | GAA | GAG | | | | |
| Phenylalanine | F | Phe | UUC | UUU | | | | |
| Glycine | G | Gly | GGA | GGC | GGG | GGU | | |
| Histidine | H | His | CAC | CAU | | | | |
| Isoleucine | I | Iso | AUA | AUC | AUU | | | |
| Lysine | K | Lys | AAA | AAG | | | | |
| Leucine | L | Leu | UUA | UUG | CUA | CUC | CUG | CUU |
| Methionine | M | Met | AUG | | | | | |
| Asparagine | N | Asn | AAC | AAU | | | | |
| Proline | P | Pro | CCA | CCC | CCG | CCU | | |
| Glutamine | Q | Gln | CAA | CAG | | | | |
| Arginine | R | Arg | AGA | AGG | CGA | CGC | CGG | CGU |
| Serine | S | Ser | AGC | AGU | UCA | UCC | UCG | UCU |

| | | | | | | |
|---|---|---|---|---|---|---|
| Threonine | T | Thr | ACA | ACC | ACG | ACU |
| Valine | V | Val | GUA | GUC | GUG | GUU |

| Tryptophan | W | Trp | UGG | |
|---|---|---|---|---|
| Tyrosine | Y | Tyr | UAC | UAU |

Guidance in determining which amino acid residues can be substituted, inserted, or deleted without abolishing biological activity can be found using computer programs well known in the art, such as DNASTAR, DNA Strider, Geneious, Mac Vector, or Vector NTI software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224).

As outlined above, amino acid substitutions may be based on the relative similarity of the amino acid side-chain substituents, for example, their hydrophobicity, hydrophilicity, charge, size, and the like.

Polypeptide variants further include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

In particular embodiments, expression of both TCRα and TCRβ polypeptides, or TCRγ and TCRδ polypeptides, in the same cell is desired. Polynucleotide sequences encoding TCR polypeptides can be separated by an IRES sequence as discussed elsewhere herein.

In preferred embodiments, fusion polypeptides are contemplated herein. Fusion polypeptides and fusion proteins refer to a polypeptide having at least two, three, four, five, six, seven, eight, nine, or ten or more polypeptide segments. Fusion polypeptides are typically linked C-terminus to N-terminus, although they can also be linked C-terminus to C-terminus, N-terminus to N-terminus, or N-terminus to C-terminus. In particular embodiments, polypeptides of the fusion protein can be in any order or a specified order.

In a particular preferred embodiment, a TCR polypeptides (i.e., TCRα, TCRβ, TCRγ, and/or TCRδ polypeptides) can be expressed as a fusion polypeptide that comprises one or more self-cleaving polypeptide sequences that separate TCR polypeptides.

In particular embodiments, a TCR contemplated herein (e.g., an engineered TCR) is expressed as a fusion polypeptide that comprises a TCRα polypeptide, a polypeptide linker (e.g., self cleaving polypeptide), and a TCRβ polypeptide. In particular embodiments, a TCR contemplated herein (e.g., an engineered TCR) is expressed as a fusion polypeptide that comprises a TCRγ polypeptide, a polypeptide linker (e.g., self cleaving polypeptide), and a TCRδ polypeptide.

In some embodiments, a TCR (e.g., an engineered TCR) is expressed as a fusion protein that comprises from N-terminus to C-terminus, a TCRα polypeptide, a polypeptide linker (e.g., self cleaving polypeptide), and a TCRβ polypeptide. In some embodiments, a TCR (e.g., an engineered TCR) is expressed as a fusion protein that comprises from N-terminus to C-terminus, a TCRβ polypeptide, a polypeptide linker (e.g., self cleaving polypeptide), and a TCRα polypeptide.

In some embodiments, a TCR (e.g., an engineered TCR) is expressed as a fusion protein that comprises from N-terminus to C-terminus, a TCRγ polypeptide, a polypeptide linker (e.g., self cleaving polypeptide), and a TCRδ polypeptide. In some embodiments, a TCR (e.g., an engineered TCR) is expressed as a fusion protein that comprises from N-terminus to C-terminus, a TCRδ polypeptide, a polypeptide linker (e.g., self cleaving polypeptide), and a TCRγ polypeptide.

In particular embodiments, an engineered TCR (e.g., an engineered TCR complex) contemplated herein is expressed as a fusion polypeptide comprising: (a) a TCRβ polypeptide comprising a TCRβ variable domain; (b) a polypeptide cleavage signal; and (c) a TCRα polypeptide comprising one or more antigen-binding domains, a polypeptide linker, and a TCRα variable domain.

In particular embodiments, an engineered TCR (e.g., an engineered TCR complex)contemplated herein is expressed as a fusion polypeptide comprising (a) a TCRβ polypeptide comprising one or more antigen-binding domains, a polypeptide linker, and a TCRβ variable domain; (b) a polypeptide cleavage signal; and (c) a TCRα polypeptide comprising a TCRα variable domain.

In particular embodiments, an engineered TCR (e.g., an engineered TCR complex)contemplated herein is expressed as a fusion polypeptide comprising: (a) a TCRβ polypeptide comprising one or more antigen-binding domains, a polypeptide linker, and a TCRβ variable domain; (b) a polypeptide cleavage signal; and (c) a TCRα polypeptide comprising one or more antigen-binding domains, a polypeptide linker, and a TCRα variable domain.

In particular embodiments, an engineered TCR (e.g., an engineered TCR complex)contemplated herein is expressed as a fusion polypeptide comprising: (a) a TCRγ polypeptide comprising a TCRγ variable domain; (b) a polypeptide cleavage signal; and (c) a TCRδ polypeptide comprising one or more antigen-binding domains, a polypeptide linker, and a TCRδ variable domain.

In particular embodiments, an engineered TCR (e.g., an engineered TCR complex)contemplated herein is expressed as a fusion polypeptide comprising (a) a TCRγ polypeptide comprising one or more antigen-binding domains, a polypeptide linker, and a TCRγ variable domain; (b) a polypeptide cleavage signal; and (c) a TCRδ polypeptide comprising a TCRδ variable domain.

In particular embodiments, an engineered TCR (e.g., an engineered TCR complex)contemplated herein is expressed as a fusion polypeptide comprising: (a) a TCRγ polypeptide comprising one or more antigen-binding domains, a polypeptide linker, and a TCRγ variable domain; (b) a polypeptide cleavage signal; and (c) a TCRδ polypeptide comprising one or more antigen-binding domains, a polypeptide linker, and a TCRδ variable domain.

The fusion polypeptides can comprise any of the TCR polypeptides contemplated herein.

The fusion proteins contemplated herein also comprise a polypeptide cleavage signal between the TCR polypeptides. Illustrative examples of polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S), for example, ENLYFQG (SEQ ID NO: 114) and ENLYFQS (SEQ ID NO: 115), wherein X represents any amino acid (cleavage by TEV occurs between Q and G or Q and S).

In particular embodiments, the polypeptide cleavage signal is a viral self-cleaving peptide or ribosomal skipping sequence.

Illustrative examples of ribosomal skipping sequences include, but are not limited to: a 2A or 2A-like site, sequence or domain (Donnelly et al., 2001. J. Gen. Virol. 82:1027-1041).

In a particular embodiment, the viral 2A peptide is an aphthovirus 2A peptide, a potyvirus 2A peptide, or a cardiovirus 2A peptide. In one embodiment, the viral 2A peptide is selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

Illustrative examples of 2A sites are provided in Table 1.

TABLE 2

| | |
|---|---|
| SEQ ID NO: 116 | GSGATNFSLLKQAGDVEENPGP |
| SEQ ID NO: 117 | ATNFSLLKQAGDVEENPGP |
| SEQ ID NO: 118 | LLKQAGDVEENPGP |
| SEQ ID NO: 119 | GSGEGRGSLLTCGDVEENPGP |
| SEQ ID NO: 120 | EGRGSLLTCGDVEENPGP |
| SEQ ID NO: 121 | LLTCGDVEENPGP |
| SEQ ID NO: 122 | GSGQCTNYALLKLAGDVESNPGP |
| SEQ ID NO: 123 | QCTNYALLKLAGDVESNPGP |
| SEQ ID NO: 124 | LLKLAGDVESNPGP |
| SEQ ID NO: 125 | GSGVKQTLNFDLLKLAGDVESNPGP |
| SEQ ID NO: 126 | VKQTLNFDLLKLAGDVESNPGP |
| SEQ ID NO: 127 | LLKLAGDVESNPGP |
| SEQ ID NO: 128 | LLNFDLLKLAGDVESNPGP |
| SEQ ID NO: 129 | TLNFDLLKLAGDVESNPGP |
| SEQ ID NO: 130 | LLKLAGDVESNPGP |
| SEQ ID NO: 131 | NFDLLKLAGDVESNPGP |
| SEQ ID NO: 132 | QLLNFDLLKLAGDVESNPGP |
| SEQ ID NO: 133 | APVKQTLNFDLLKLAGDVESNPGP |
| SEQ ID NO: 134 | VTELLYRMKRAETYCPRPLLAIHPTEARHKQ |
| | KIVAPVKQT |
| SEQ ID NO: 135 | LNFDLLKLAGDVESNPGP |
| SEQ ID NO: 136 | LLAIHPTEARHKQKIVAPVKQTLNFDLLKLA |
| | GDVESNPGP |
| SEQ ID NO: 137 | EARHKQKIVAPVKQTLNFDLLKLAGDVESNP |
| | GP |

In particular embodiments, the fusion protein comprises a polypeptide cleavage signal that is a viral self-cleaving peptide or ribosomal skipping sequence.

In particular embodiments, the fusion protein comprises a polypeptide cleavage signal that is a viral 2A peptide. In particular embodiments, the fusion protein comprises a polypeptide cleavage signal that is an aphthovirus 2A peptide, a potyvirus 2A peptide, or a cardiovirus 2A peptide. In particular embodiments, the fusion protein comprises a polypeptide cleavage signal that is a viral 2A peptide is selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

In particular embodiments, the polypeptide cleavage signal is a viral self-cleaving peptide or ribosomal skipping sequence. In some embodiments, the polypeptide cleavage signal is a viral 2A peptide. In some embodiments, the polypeptide cleavage signal is an aphthovirus 2A peptide, a potyvirus 2A peptide, or a cardiovirus 2A peptide. In some embodiments, the polypeptide cleavage signal is a viral 2A peptide selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

In various embodiments, the polypeptide cleavage signal comprises a self-cleaving peptide (e.g., 2A peptide) and a GSG amino acid sequence immediately upstream (i.e., N-term) of the 2A peptide.

In various embodiments, the polypeptide cleavage signal further comprises a furin recognition site upstream of the polypeptide cleavage signal (e.g., self-cleaving 2A peptide). In particular embodiments, the furin recognition site comprises the amino acid sequence as set forth in SEQ ID NO: 112.

In various embodiments, the polypeptide cleavage signal comprises an amino acid sequence as set forth in any one of SEQ ID NOs: 113-137. In some embodiments, the polypeptide cleavage signal comprises an amino acid sequence as set forth in SEQ ID NO: 113. In some embodiments, the polypeptide cleavage signal comprises an amino acid sequence as set forth in SEQ ID NO: 114. In some embodiments, the polypeptide cleavage signal comprises an amino acid sequence as set forth in SEQ ID NO: 115. In some embodiments, the polypeptide cleavage signal comprises an amino acid sequence as set forth in SEQ ID NO: 116. In some embodiments, the polypeptide cleavage signal comprises an amino acid sequence as set forth in SEQ ID NO: 117. In some embodiments, the polypeptide cleavage signal comprises an amino acid sequence as set forth in SEQ ID NO: 118. In some embodiments, the polypeptide cleavage signal comprises an amino acid sequence as set forth in SEQ ID NO: 119. In some embodiments, the polypeptide cleavage signal comprises an amino acid sequence as set forth in SEQ ID NO: 120. In some embodiments, the polypeptide cleavage signal comprises an amino acid sequence as set forth in SEQ ID NO: 121. In some embodiments, the polypeptide cleavage signal comprises an amino acid sequence as set forth in SEQ ID NO: 122. In some embodiments, the polypeptide cleavage signal comprises an amino acid sequence as set forth in SEQ ID NO: 123. In some embodiments, the polypeptide cleavage signal comprises an amino acid sequence as set forth in SEQ ID NO: 124. In some embodiments, the polypeptide cleavage signal comprises an amino acid sequence as set forth in SEQ ID NO: 125. In some embodiments, the polypeptide cleavage signal comprises an amino acid sequence as set forth in SEQ ID NO: 126. In some embodiments, the polypeptide cleavage signal comprises an amino acid sequence as set forth in SEQ ID NO: 127. In some embodiments, the polypeptide cleavage signal comprises an amino acid sequence as set forth in SEQ ID NO: 128. In some embodiments, the polypeptide cleavage signal comprises an amino acid sequence as set forth in SEQ ID NO: 129. In some embodiments, the polypeptide cleavage signal comprises an amino acid sequence as set forth in SEQ ID NO: 130. In some embodiments, the polypeptide cleavage signal comprises an amino acid sequence as set forth in SEQ ID NO: 131. In some embodiments, the polypeptide cleavage signal comprises an amino acid sequence as set forth in SEQ ID NO: 132. In some embodiments, the polypeptide cleavage signal comprises an amino acid sequence as set forth in SEQ ID NO: 133. In some embodiments, the polypeptide cleavage signal comprises an amino acid sequence as set forth in SEQ ID NO: 134. In some embodiments, the polypeptide cleavage signal comprises an amino acid sequence as set forth in SEQ ID NO: 135. In some embodiments, the polypeptide cleavage signal comprises an amino acid sequence as set forth in SEQ ID NO: 136. In some embodiments, the polypeptide cleavage signal comprises an amino acid sequence as set forth in SEQ ID NO: 137.

In various embodiments, the TCRβ or TCRδ polypeptide is N-terminal of the TCRα or TCRγ polypeptide.

In various embodiments, the TCRα or TCRγ polypeptide is N-terminal of the TCRβ or TCRδ polypeptide.

In particular embodiments, the fusion polypeptide comprises an amino acid sequence at least 85%, 90%, 95%, 96%, 97%, 98%, or 99% identical to an amino acid sequence as set forth in any one of SEQ ID NOs: 91-97, 100, and 102. In some embodiments, the fusion polypeptide comprises an amino acid sequence at least 85%, identical to an amino acid sequence as set forth in any one of SEQ ID NOs: 91-97, 100, and 102. In some embodiments, the fusion polypeptide comprises an amino acid sequence at least 90%, identical to an amino acid sequence as set forth in any one of SEQ ID NOs: 91-97, 100, and 102. In some embodiments, the fusion polypeptide comprises an amino acid sequence at least 95%, identical to an amino acid sequence as set forth in any one of SEQ ID NOs: 91-97, 100, and 102. In some embodiments, the fusion polypeptide comprises an amino acid sequence at least 96%, identical to an amino acid sequence as set forth in any one of SEQ ID NOs: 91-97, 100, and 102. In some embodiments, the fusion polypeptide comprises an amino acid sequence at least 97%, identical to an amino acid sequence as set forth in any one of SEQ ID NOs: 91-97, 100, and 102. In some embodiments, the fusion polypeptide comprises an amino acid sequence at least 98%, identical to an amino acid sequence as set forth in any one of SEQ ID NOs: 91-97, 100, and 102. In some embodiments, the fusion polypeptide comprises an amino acid sequence at least 99%, identical to an amino acid sequence as set forth in any one of SEQ ID NOs: 91-97, 100, and 102.

In particular embodiments, the fusion polypeptide comprises an amino acid sequence as set forth in any one of SEQ ID NOs: 91-97, 100, and 102. In some embodiments, the fusion polypeptide comprises an amino acid sequence as set forth in SEQ ID NO: 91. In some embodiments, the fusion polypeptide comprises an amino acid sequence as set forth in SEQ ID NO: 92. In some embodiments, the fusion polypeptide comprises an amino acid sequence as set forth in SEQ ID NO: 93. In some embodiments, the fusion polypeptide comprises an amino acid sequence as set forth in SEQ ID NO: 94. In some embodiments, the fusion polypeptide comprises an amino acid sequence as set forth in SEQ ID NO: 95. In some embodiments, the fusion polypeptide comprises an amino acid sequence as set forth in SEQ ID NO: 96. In some embodiments, the fusion polypeptide comprises an amino acid sequence as set forth in SEQ ID NO: 97. In some embodiments, the fusion polypeptide comprises an amino acid sequence as set forth in SEQ ID NO: 100. In some embodiments, the fusion polypeptide comprises an amino acid sequence as set forth in SEQ ID NO: 102.

### F. Polynucleotides

In particular embodiments, one or more polynucleotides encoding one or more TCR polypeptides, TCRα polypeptides, TCRβ polypeptides, TCRγ polypeptides, TCRδ polypeptides, TCR fusion polypeptides, and fragments thereof is provided. As used herein, the terms “polynucleotide” or “nucleic acid” refer to deoxyribonucleic acid (DNA), ribonucleic acid (RNA) and DNA/RNA hybrids. Polynucleotides may be monocistronic or polycistronic, single-stranded or double-stranded, and either recombinant, synthetic, or isolated. Polynucleotides include, but are not limited to: pre-messenger RNA (pre-mRNA), messenger RNA (mRNA), RNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

As used herein, “isolated polynucleotide” refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” also refers to a complementary DNA (cDNA), a recombinant DNA, or other polynucleotide that does not exist in nature and that has been made by the hand of man. In particular embodiments, an isolated polynucleotide is a synthetic polynucleotide, a recombinant polynucleotide, a semi-synthetic polynucleotide, or a polynucleotide obtained or derived from a recombinant source.

In various embodiments, a polynucleotide comprises an mRNA encoding a polypeptide contemplated herein. In certain embodiments, the mRNA comprises a cap, one or more nucleotides, and a poly(A) tail.

In various embodiments, the polynucleotide is an mRNA that is introduced into a cell in order to transiently express a desired polypeptide.

As used herein, “transient” refers to expression of a non-integrated transgene for a period of hours, days or weeks, wherein the period of time of expression is less than the period of time for expression of the polynucleotide if integrated into the genome or contained within a stable plasmid replicon in the cell.

In particular embodiments, the mRNA encoding a polypeptide is an in vitro transcribed mRNA. As used herein, “in vitro transcribed RNA” refers to RNA, preferably mRNA that has been synthesized in vitro. Generally, the in vitro transcribed RNA is generated from an in vitro transcription vector. The in vitro transcription vector comprises a template that is used to generate the in vitro transcribed RNA.

In particular embodiments, mRNAs may further comprise a comprise a 5′ cap or modified 5′ cap and/or a poly(A) sequence. As used herein, a 5′ cap (also termed an RNA cap, an RNA 7-methylguanosine cap or an RNA m^{7G} cap) is a modified guanine nucleotide that has been added to the ‘front” or 5′ end of a eukaryotic messenger RNA shortly after the start of transcription. The 5′ cap comprises a terminal group which is linked to the first transcribed nucleotide and recognized by the ribosome and protected from RNases. The capping moiety can be modified to modulate functionality of mRNA such as its stability or efficiency of translation. In a particular embodiment, the mRNA comprises a poly(A) sequence of between about 50 and about 5000 adenines. In one embodiment, the mRNA comprises a poly(A) sequence of between about 100 and about 1000 bases, between about 200 and about 500 bases, or between about 300 and about 400 bases. In one embodiment, the mRNA comprises a poly(A) sequence of about 65 bases, about 100 bases, about 200 bases, about 300 bases, about 400 bases, about 500 bases, about 600 bases, about 700 bases, about 800 bases, about 900 bases, or about 1000 or more bases. poly(A) sequences can be modified chemically or enzymatically to modulate mRNA functionality such as localization, stability or efficiency of translation. In particular embodiments, polynucleotides may be codon-optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, (x) systematic variation of codon sets for each amino acid, and/or (xi) isolated removal of spurious translation initiation sites.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

Polynucleotide variants include polynucleotide fragments that encode biologically active polypeptide fragments or variants. As used herein, the term “polynucleotide fragment” refers to a polynucleotide fragment at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200,250, 300, 350, 400, 450, 500, 550, 600, 650, 700, 750, 800, 850, 900, 950, 1000, 1100, 1200, 1300, 1400, 1500, 1600, 1700 or more nucleotides in length that encodes a polypeptide variant that retains at least 100%, at least 90%, at least 80%, at least 70%, at least 60%, at least 50%, at least 40%, at least 30%, at least 20%, at least 10%, or at least 5% of the naturally occurring polypeptide activity. Polynucleotide fragments refer to a polynucleotide that encodes a polypeptide that has an amino-terminal deletion, a carboxyl-terminal deletion, and/or an internal deletion or substitution of one or more amino acids of a naturally-occurring or recombinantly-produced polypeptide.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any of the reference sequences described herein, typically where the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the premessenger (premRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

Moreover, it will be appreciated by those of ordinary skill in the art that, as a result of the degeneracy of the genetic code, there are many nucleotide sequences that encode a polypeptide, or fragment of variant thereof, as described herein. Some of these polynucleotides bear minimal homology to the nucleotide sequence of any native gene. Nonetheless, polynucleotides that vary due to differences in codon usage are specifically contemplated in particular embodiments, for example polynucleotides that are optimized for human and/or primate codon selection. Further, alleles of the genes comprising the polynucleotide sequences provided herein may also be used. Alleles are endogenous genes that are altered as a result of one or more mutations, such as deletions, additions and/or substitutions of nucleotides.

The term “nucleic acid cassette” or “expression cassette” as used herein refers to genetic sequences within the vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly(A) sequence, and a gene(s)-of-interest, e.g., a polynucleotide(s)-of-interest. Vectors may comprise 1, 2, 3, 4, 5, 6, 7, 8, 9 or 10 or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In a preferred embodiment, the nucleic acid cassette encodes one or more chains of a TCR. The cassette can be removed and inserted into a plasmid or viral vector as a single unit.

Polynucleotides include polynucleotide(s)-of-interest. As used herein, the term “polynucleotide-of-interest” refers to a polynucleotide encoding a polypeptide, polypeptide variant, or fusion polypeptide. A vector may comprise 1, 2, 3, 4, 5, 6, 7, 8, 9, or 10 polynucleotides-of-interest. In certain embodiments, the polynucleotide-of-interest encodes a polypeptide that provides a therapeutic effect in the treatment or prevention of a disease or disorder. Polynucleotides-of-interest, and polypeptides encoded therefrom, include both polynucleotides that encode wild-type polypeptides, as well as functional variants and fragments thereof. In particular embodiments, a functional variant has at least 80%, at least 90%, at least 95%, or at least 99% identity to a corresponding wild-type reference polynucleotide or polypeptide sequence. In certain embodiments, a functional variant or fragment has at least 50%, at least 60%, at least 70%, at least 80%, or at least 90% of a biological activity of a corresponding wild-type polypeptide.

The polynucleotides contemplated herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed in particular embodiments, with the total length preferably being limited by the ease of preparation and use in the intended recombinant DNA protocol.

Polynucleotides can be prepared, manipulated and/or expressed using any of a variety of well-established techniques known and available in the art. In order to express a desired polypeptide, a nucleotide sequence encoding the polypeptide, can be inserted into appropriate vector as discussed further below.

Illustrative examples of vectors include, but are not limited to plasmid, autonomously replicating sequences, and transposable elements, e.g., piggyBac, Sleeping Beauty, Mos1, Tc1/mariner, Tol2, mini-Tol2, Tc3, MuA, Himar I, Frog Prince, and derivatives thereof.

Additional Illustrative examples of vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses.

Illustrative examples of viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40).

Illustrative examples of expression vectors include, but are not limited to, pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

The “control elements” or “regulatory sequences” present in an expression vector are those non-translated regions of the vector-origin of replication, selection cassettes, promoters, enhancers, translation initiation signals (Shine Dalgarno sequence or Kozak sequence) introns, a polyadenylation sequence, 5′ and 3′ untranslated regions-which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, vectors include, but are not limited to expression vectors and viral vectors, and will include exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), 0-kinesin (0-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, d1587rev primer-binding site substituted (MND) U3 promoter (Haas et al. Journal of Virology. 2003; 77(17): 9439-9450).

In one embodiment, a vector comprises an MNDU3 promoter.

In one embodiment, a vector comprises an EF1a promoter comprising the first intron of the human EF1a gene.

In one embodiment, a vector comprises an EF1a promoter that lacks the first intron of the human EF1a gene.

In a particular embodiment, it may be desirable to express a polynucleotide comprising an engineered TCR from a T cell specific promoter.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site-specific DNA recombinase. According to certain embodiments the vector comprises at least one (typically two) site(s) for recombination mediated by a site-specific recombinase. As used herein, the terms “recombinase” or “site specific recombinase” include excusive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular embodiments include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

The vectors may comprise one or more recombination sites for any of a wide variety of site-specific recombinases. It is to be understood that the target site for a site-specific recombinase is in addition to any site(s) required for integration of a vector, e.g., a retroviral vector or lentiviral vector. As used herein, the terms “recombination sequence,” “recombination site,” or “site specific recombination site” refer to a particular nucleic acid sequence to which a recombinase recognizes and binds.

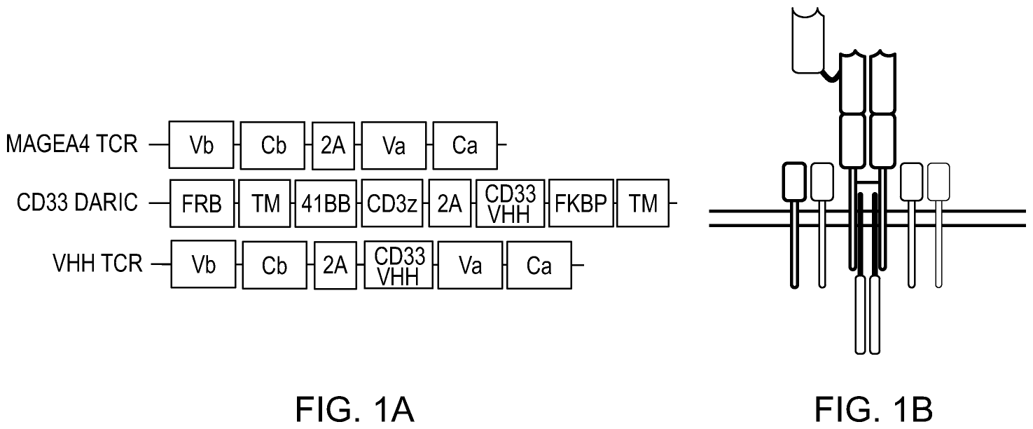

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other exemplary loxP sites include, but are not limited to: lox511 (Hoess et al., 1996; Bethke and Sauer, 1997), lox5171 (Lee and Saito, 1998), lox2272 (Lee and Saito, 1998), m2 (Langer et al., 2002), lox71 (Albert et al., 1995), and lox66 (Albert et al., 1995).

Suitable recognition sites for the FLP recombinase include, but are not limited to: FRT (McLeod, et al., 1996), F₁, F₂, F₃ (Schlake and Bode, 1994), F₄, F₅ (Schlake and Bode, 1994), FRT(LE) (Senecoff et al., 1988), FRT(RE) (Senecoff et al., 1988).

Other examples of recognition sequences are the attB, attP, attL, and attR sequences, which are recognized by the recombinase enzyme λ Integrase, e.g., phi-c31. The φC31 SSR mediates recombination only between the heterotypic sites attB (34 bp in length) and attP (39 bp64aposiength) (Groth et al., 2000). attB and attP, named for the attachment sites for the phage integrase on the bacterial and phage genomes, respectively, both contain imperfect inverted repeats that are likely bound by φC3164aposidimers (Groth et al., 2000). The product sites, attL and attR, are effectively inert to further φC31-mediated recombination (Belteki et al., 2003), making the reaction irreversible. For catalyzing insertions, it has been found that attB-bearing DNA inserts into a genomic attP site more readily than an attP site into a genomic attB site (Thyagarajan et al., 2001; Belteki et al., 2003). Thus, typical strategies position by homologous recombination an attP-bearing “docking site” into a defined locus, which is then partnered with an attB-bearing incoming sequence for insertion.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. In particular embodiments, vectors include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides. In one embodiment, the IRES used in polynucleotides contemplated herein is an EMCV IRES.

As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO: 139), where R is a purine (A or G) (Kozak, 1986. Cell. 44(2):283-92, and Kozak, 1987. Nucleic Acids Res. 15(20):8125-48). In particular embodiments, the vectors comprise polynucleotides that have a consensus Kozak sequence and that encode a desired polypeptide, e.g., a TCR.

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal polyA sequence (e.g., AATAAA, ATTAAA, AGTAAA). In particular embodiments, the poly(A) sequence is an SV40 polyA sequence, a bovine growth hormone polyA sequence (BGHpA), a rabbit β-globin polyA sequence (rpgpA), variants thereof, or another suitable heterologous or endogenous polyA sequence known in the art.

In some embodiments, a polynucleotide or cell harboring the polynucleotide utilizes a suicide gene, including an inducible suicide gene to reduce the risk of direct toxicity and/or uncontrolled proliferation. In specific aspects, the suicide gene is not immunogenic to the host harboring the polynucleotide or cell. A certain example of a suicide gene that may be used is caspase-9 or caspase-8 or cytosine deaminase. Caspase-9 can be activated using a specific chemical inducer of dimerization (CID).

In certain embodiments, a polycistronic polynucleotide encoding a fusion protein encoding a TCR is contemplated herein. In some embodiments, a polycistronic polynucleotide encoding a TCR comprising a TCRα polypeptide/chain and a TCRβ polypeptide/chain is introduced into a cell. In some embodiments, a polycistronic polynucleotide encoding a TCR comprising a TCRγ polypeptide/chain and a TCRδ polypeptide/chain is introduced into a cell.

In particular embodiments, the polycistronic polynucleotide comprises the TCRα polypeptide/chain 5′ to the TCRβ polypeptide/chain. In other embodiments, the polycistronic polynucleotide comprises the TCRβ polypeptide/chain 5′ to the TCRα polypeptide/chain. In other embodiments, the polycistronic polynucleotide comprises the TCRδ polypeptide/chain 5′ to the TCRγ polypeptide/chain. In other embodiments, the polycistronic polynucleotide comprises the TCRγ polypeptide/chain 5′ to the TCRδ polypeptide/chain.

### G. Vectors

In particular embodiments, one or more polynucleotides encoding a TCRα polypeptide/chain and a TCRβ polypeptide/chain are introduced into a cell (e.g., an immune effector cell) by non-viral or viral vectors.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, non-viral vectors are used to deliver one or more polynucleotides contemplated herein to a T cell.

Illustrative examples of non-viral vectors include, but are not limited to mRNA, plasmids (e.g., DNA plasmids or RNA plasmids), transposons, cosmids, and bacterial artificial chromosomes. Other non-viral vectors are discussed above.

Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, polycation or lipid:nucleic acid conjugates, naked DNA, artificial virions, DEAE-dextran-mediated transfer, gene gun, and heat-shock.

Illustrative examples of non-viral/polynucleotide delivery systems suitable for use in particular embodiments contemplated in particular embodiments include, but are not limited to those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al. (2003) Gene Therapy. 10:180-187; and Balazs et al. (2011) Journal of Drug Delivery. 2011:1-12. Antibody-targeted, bacterially derived, non-living nanocell-based delivery is also contemplated in particular embodiments.

In various embodiments, the polynucleotide is an mRNA that is introduced into a cell in order to transiently express a desired polypeptide. As used herein, “transient” refers to expression of a non-integrated transgene for a period of hours, days or weeks, wherein the period of time of expression is less than the period of time for expression of the polynucleotide if integrated into the genome or contained within a stable plasmid replicon in the cell.

In particular embodiments, viral vectors are used to deliver one or more polynucleotides contemplated herein to a T cell.

Viral vectors comprising polynucleotides contemplated in particular embodiments can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, viral vectors comprising nuclease variants and/or donor repair templates are administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Illustrative examples of viral vector systems suitable for use in particular embodiments contemplated in particular embodiments include, but are not limited to adeno-associated virus (AAV), retrovirus, herpes simplex virus, adenovirus, and vaccinia virus vectors.

In certain embodiments, a polycistronic polynucleotide encoding a TCR comprising a TCRα polypeptide/chain and a TCRβ polypeptide/chain is introduced into a cell by a non-viral or viral vector. In particular embodiments, the polycistronic polynucleotide comprises the TCRα polypeptide/chain 5′ to the TCRβ polypeptide/chain. In other embodiments, the polycistronic polynucleotide comprises the TCRβ polypeptide/chain 5′ to the TCRα polypeptide/chain.

In some embodiments, a polycistronic polynucleotide encoding a TCR comprising a TCRα polypeptide/chain and a TCRβ polypeptide/chain is introduced into a cell by a non-viral or viral vector. In some embodiments, a polycistronic polynucleotide encoding a TCR comprising a TCRγ polypeptide/chain and a TCRδ polypeptide/chain is introduced into a cell by a non-viral or viral vector.

In certain embodiments, the polycistronic polynucleotide comprises the TCRα polypeptide/chain 5′ to the TCRβ polypeptide/chain. In other embodiments, the polycistronic polynucleotide comprises the TCRβ polypeptide/chain 5′ to the TCRα polypeptide/chain. In other embodiments, the polycistronic polynucleotide comprises the TCRδ polypeptide/chain 5′ to the TCRγ polypeptide/chain. In other embodiments, the polycistronic polynucleotide comprises the TCRγ polypeptide/chain 5′ to the TCRδ polypeptide/chain.

In various embodiments, one or more polynucleotides are introduced into an immune effector cell, e.g., T cell, by transducing the cell with a recombinant adeno-associated virus (rAAV), comprising the one or more polynucleotides.

AAV is a small (˜26 nm) replication-defective, primarily episomal, non-enveloped virus. AAV can infect both dividing and non-dividing cells and may incorporate its genome into that of the host cell. Recombinant AAV (rAAV) are typically composed of, at a minimum, a transgene and its regulatory sequences, and 5′ and 3′ AAV inverted terminal repeats (ITRs). The ITR sequences are about 145 bp in length. In particular embodiments, the rAAV comprises ITRs and capsid sequences isolated from AAV1, AAV2, AAV3, AAV4, AAV5, AAV6, AAV7, AAV8, AAV9, or AAV10.

In some embodiments, a chimeric rAAV is used the ITR sequences are isolated from one AAV serotype and the capsid sequences are isolated from a different AAV serotype. For example, a rAAV with ITR sequences derived from AAV2 and capsid sequences derived from AAV6 is referred to as AAV2/AAV6. In particular embodiments, the rAAV vector may comprise ITRs from AAV2, and capsid proteins from any one of AAV1, AAV2, AAV3, AAV4, AAV5, AAV6, AAV7, AAV8, AAV9, or AAV10. In a preferred embodiment, the rAAV comprises ITR sequences derived from AAV2 and capsid sequences derived from AAV6. In a preferred embodiment, the rAAV comprises ITR sequences derived from AAV2 and capsid sequences derived from AAV2.

In some embodiments, engineering and selection methods can be applied to AAV capsids to make them more likely to transduce cells of interest.

Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety.

In various embodiments, one or more polynucleotides are introduced into an immune effector cell, e.g., T cell, by transducing the cell with a retrovirus, e.g., lentivirus, comprising the one or more polynucleotides.

As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

In various embodiments, a lentiviral vector contemplated herein comprises one or more LTRs, and one or more, or all, of the following accessory elements: a cPPT/FLAP, a Psi (Ψ) packaging signal, an export element, poly (A) sequences, and may optionally comprise a WPRE or HPRE, an insulator element, a selectable marker, and a cell suicide gene, as discussed elsewhere herein.

In particular embodiments, lentiviral vectors contemplated herein may be integrative or non-integrating or integration defective lentivirus. As used herein, the term “integration defective lentivirus” or “IDLV” refers to a lentivirus having an integrase that lacks the capacity to integrate the viral genome into the genome of the host cells. Integration-incompetent viral vectors have been described in patent application WO 2006/010834, which is herein incorporated by reference in its entirety.

Illustrative mutations in the HIV-1 pol gene suitable to reduce integrase activity include, but are not limited to: H12N, H12C, H16C, H16V, S81 R, D41A, K42A, H51A, Q53C, D55V, D64E, D64V, E69A, K71A, E85A, E87A, D116N, D1161, D116A, N120G, N1201, N120E, E152G, E152A, D35E, K156E, K156A, E157A, K159E, K159A, K160A, R166A, D167A, E170A, H171A, K173A, K186Q, K186T, K188T, E198A, R199c, R199T, R199A, D202A, K211A, Q214L, Q216L, Q221 L, W235F, W235E, K236S, K236A, K246A, G247W, D253A, R262A, R263A and K264H.

In one embodiment, the HIV-1 integrase deficient pol gene comprises a D64V, D116I, D116A, E152G, or E152A mutation; D64V, D116I, and E152G mutations; or D64V, D116A, and E152A mutations.

In one embodiment, the HIV-1 integrase deficient pol gene comprises a D64V mutation.

The term “long terminal repeat (LTR)” refers to domains of base pairs located at the ends of retroviral DNAs which, in their natural sequence context, are direct repeats and contain U3, R and U5 regions.

As used herein, the term “FLAP element” or “cPPT/FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173. In another embodiment, a lentiviral vector contains a FLAP element with one or more mutations in the cPPT and/or CTS elements. In yet another embodiment, a lentiviral vector comprises either a cPPT or CTS element. In yet another embodiment, a lentiviral vector does not comprise a cPPT or CTS element.

As used herein, the term “packaging signal” or “packaging sequence” refers to psi [Ψ] sequences located within the retroviral genome which are required for insertion of the viral RNA into the viral capsid or particle, see e.g., Clever et al., 1995. J. of Virology, Vol. 69, No. 4; pp. 2101-2109.

The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE).

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766).

Lentiviral vectors preferably contain several safety enhancements as a result of modifying the LTRs. “Self-inactivating” (SIN) vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. Self-inactivation is preferably achieved through in the introduction of a deletion in the U3 region of the 3′ LTR of the vector DNA, i.e., the DNA used to produce the vector RNA. Thus, during reverse transcription, this deletion is transferred to the 5′ LTR of the proviral DNA. In particular embodiments, it is desirable to eliminate enough of the U3 sequence to greatly diminish or abolish altogether the transcriptional activity of the LTR, thereby greatly diminishing or abolishing the production of full-length vector RNA in transduced cells. In the case of HIV based lentivectors, it has been discovered that such vectors tolerate significant U3 deletions, including the removal of the LTR TATA box (e.g., deletions from −418 to −18), without significant reductions in vector titers.

An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters.

The terms “pseudotype” or “pseudotyping” as used herein, refer to a virus whose viral envelope proteins have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4⁺ presenting cells.

In certain embodiments, lentiviral vectors are produced according to known methods. See e.g., Kutner et al., BMC Biotechnol. 2009; 9:10. doi: 10.1186/1472-6750-9-10; Kutner et al. Nat. Protoc. 2009; 4(4):495-505. doi: 10.1038/nprot.2009.22.

According to certain specific embodiments contemplated herein, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid contemplated herein.

In various embodiments, one or more polynucleotides are introduced into an immune effector cell, by transducing the cell with an adenovirus comprising the one or more polynucleotides.

Adenoviral based vectors are capable of very high transduction efficiency in many cell types and do not require cell division. With such vectors, high titer and high levels of expression have been obtained. This vector can be produced in large quantities in a relatively simple system. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Generation and propagation of the current adenovirus vectors, which are replication deficient, may utilize a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). Adenovirus vectors have been used in eukaryotic gene expression (Levrero et al., 1991; Gomez-Foix et al., 1992) and vaccine development (Grunhaus & Horwitz, 1992; Graham & Prevec, 1992). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993). An example of the use of an Ad vector in a clinical trial involved polynucleotide therapy for antitumor immunization with intramuscular injection (Sterman et al., Hum. Gene Ther. 7:1083-9 (1998)).

In various embodiments, one or more polynucleotides are introduced into an immune effector cell by transducing the cell with a herpes simplex virus, e.g., HSV-1, HSV-2, comprising the one or more polynucleotides.

The mature HSV virion consists of an enveloped icosahedral capsid with a viral genome consisting of a linear double-stranded DNA molecule that is 152 kb. In one embodiment, the HSV based viral vector is deficient in one or more essential or non-essential HSV genes. In one embodiment, the HSV based viral vector is replication deficient. Most replication deficient HSV vectors contain a deletion to remove one or more intermediate-early, early, or late HSV genes to prevent replication. For example, the HSV vector may be deficient in an immediate early gene selected from the group consisting of: ICP4, ICP22, ICP27, ICP47, and a combination thereof. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, and 5,804,413, and International Patent Applications WO 91/02788, WO 96/04394, WO 98/15637, and WO 99/06583, each of which are incorporated by reference herein in its entirety.

### H. Genetically Modified Cells

In various embodiments, cells genetically modified to express an engineered TCR are contemplated herein. In some embodiments, the immune effector cells genetically modified to express an engineered TCR as contemplated herein are used in preparation or manufacture of a medicament for the treatment of cancer.

As used herein, the term “genetically engineered” or “genetically modified” refers to the addition of extra genetic material in the form of DNA or RNA into the total genetic material in a cell. The terms, “genetically modified cells,” “modified cells,” and, “redirected cells,” are used interchangeably. As used herein, the term “gene therapy” refers to the introduction of extra genetic material in the form of DNA or RNA into the total genetic material in a cell that restores, corrects, or modifies expression of a gene, or for the purpose of expressing a therapeutic polypeptide, e.g., an engineered TCR.

In particular embodiments, a polynucleotide encoding an engineered TCR contemplated herein is introduced into immune effector cells so as express the engineered TCR and to redirect the immune effector cells to target cells expressing a target antigen. An “immune effector cell,” is any cell of the immune system that has one or more effector functions (e.g., cytotoxic cell killing activity, secretion of cytokines, induction of ADCC and/or CDC). Illustrative immune effector cells contemplated herein are T lymphocytes, including but not limited to cytotoxic T cells (CTLs; CD8⁺ T cells), TILs, and helper T cells (HTLs; CD4⁺ T cells. In a particular embodiment, the cells comprise αβ T cells. In a particular embodiment, the cells comprise 76 T cells modified to express an αβ TCR. In one embodiment, immune effector cells include natural killer (NK) cells. In one embodiment, immune effector cells include natural killer T (NKT) cells.

Immune effector cells can be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells are autologous.

Illustrative immune effector cells used with the engineered TCRs contemplated in particular embodiments include T lymphocytes. The terms “T cell” or “T lymphocyte” are art-recognized and are intended to include thymocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), CD4⁺CD8⁺ T cell, CD4⁻CD8⁻ T cell, or any other subset of T cells. Other illustrative populations of T cells suitable for use in particular embodiments include naïve T cells (T_{N}), T memory stem cells (T_{SCM}), central memory T cells (T_{CM}), effector memory T cells (T_{EM}), and effector T cells (T_{EFF}).

As would be understood by the skilled person, other cells may also be used as immune effector cells with the engineered TCRs contemplated herein. In particular, immune effector cells also include NK cells, NKT cells, neutrophils, and macrophages. Immune effector cells also include progenitors of effector cells wherein such progenitor cells can be induced to differentiate into an immune effector cells in vivo or in vitro. Thus, in particular embodiments, immune effector cell includes progenitors of immune effectors cells such as hematopoietic stem cells (HSCs) contained within the CD34⁺ population of cells derived from cord blood, bone marrow or mobilized peripheral blood which upon administration in a subject differentiate into mature immune effector cells, or which can be induced in vitro to differentiate into mature immune effector cells.

The term, “CD34⁺ cell,” as used herein refers to a cell expressing the CD34 protein on its cell surface. “CD34,” as used herein refers to a cell surface glycoprotein (e.g., sialomucin protein) that often acts as a cell-cell adhesion factor and is involved in T cell entrance into lymph nodes. The CD34⁺ cell population contains hematopoietic stem cells (HSC), which upon administration to a patient differentiate and contribute to all hematopoietic lineages, including T cells, NK cells, NKT cells, neutrophils and cells of the monocyte/macrophage lineage.

Methods for making the immune effector cells that express an engineered TCR contemplated herein are provided in particular embodiments. In one embodiment, the method comprises transfecting or transducing immune effector cells isolated from an individual such that the immune effector cells express a polynucleotide or polycistronic message encoding an engineered TCR as contemplated herein or a fusion protein encoding an engineered TCR contemplated herein. In particular embodiments, the transduced cells are subsequently cultured for expansion, prior to administration to a subject.

In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express an engineered TCR contemplated herein. In this regard, the immune effector cells may be cultured before and/or after being genetically modified.

In particular embodiments, prior to in vitro manipulation or genetic modification of the immune effector cells described herein, the source of cells is obtained from a subject. In particular embodiments, modified immune effector cells comprise T cells.

In particular embodiments, PBMCs may be directly genetically modified to express a polycistronic message encoding an engineered TCR contemplated herein. In certain embodiments, after isolation of PBMC, T lymphocytes are further isolated and in certain embodiments, both cytotoxic and helper T lymphocytes can be sorted into naïve, memory, and effector T cell subpopulations either before or after genetic modification and/or expansion.

The immune effector cells, such as T cells, can be genetically modified following isolation using known methods, or the immune effector cells can be activated and expanded (or differentiated in the case of progenitors) in vitro prior to being genetically modified. In a particular embodiment, the immune effector cells, such as T cells, are activated and stimulated for expansion and then genetically modified with the TCRs contemplated herein (e.g., transduced with a viral vector comprising a nucleic acid encoding a polycistronic message encoding an engineered TCR contemplated herein comprising. In various embodiments, T cells can be activated and expanded before or after genetic modification, using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7, 175,843; 5,883,223; 6,905,874; 6,797,514; 6,867,041; and U.S. Patent Application Publication No. 20060121005.

In one embodiment, CD34⁺ cells are transduced with a nucleic acid construct contemplated herein. In certain embodiments, the transduced CD34⁺ cells differentiate into mature immune effector cells in vivo following administration into a subject, generally the subject from whom the cells were originally isolated. In another embodiment, CD34⁺ cells may be stimulated in vitro prior to exposure to or after being genetically modified with one or more of the following cytokines: Flt-3 ligand (FLT3), stem cell factor (SCF), megakaryocyte growth and differentiation factor (TPO), IL-3 and IL-6 according to the methods described previously (Asheuer et al., 2004; Imren, et al., 2004).

In particular embodiments, a population of modified immune effector cells for the treatment of cancer comprises an engineered TCR contemplated herein. For example, a population of modified immune effector cells are prepared from peripheral blood mononuclear cells (PBMCs) obtained from a patient diagnosed with B cell malignancy described herein (autologous donors). The PBMCs form a heterogeneous population of T lymphocytes that can be CD4⁺, CD8⁺, or CD4⁺ and CD8⁺.

The PBMCs also can include other cytotoxic lymphocytes such as NK cells or NKT cells. An expression vector carrying the coding sequence of an engineered TCR contemplated in particular embodiments is introduced into a population of human donor T cells, NK cells or NKT cells. In particular embodiments, successfully transduced T cells that carry the expression vector can be sorted using flow cytometry to isolate CD3 positive T cells and then further propagated to increase the number of these CAR protein expressing T cells in addition to cell activation using anti-CD3 antibodies and or anti-CD28 antibodies and IL-2 or any other methods known in the art as described elsewhere herein. Standard procedures are used for cryopreservation of T cells expressing the CAR protein T cells for storage and/or preparation for use in a human subject. In one embodiment, the in vitro transduction, culture and/or expansion of T cells are performed in the absence of non-human animal derived products such as fetal calf serum and fetal bovine serum. Since a heterogeneous population of PBMCs is genetically modified, the resultant transduced cells are a heterogeneous population of modified cells comprising a BCMA targeting CAR as contemplated herein.

In a further embodiment, a mixture of, e.g., one, two, three, four, five or more, different expression vectors can be used in genetically modifying a donor population of immune effector cells wherein each vector encodes a different chimeric antigen receptor protein as contemplated herein. The resulting modified immune effector cells forms a mixed population of modified cells.

Genetically engineered cells, including T cells, can be manufactured using various methods known in the art, see, e.g., WO 2016/094304 which is incorporated herein by reference in its entirety.

### I. Compositions and Formulations

The compositions contemplated herein may comprise one or more engineered TCR polypeptides, TCRα polypeptides, TCRβ polypeptides, TCRγ polypeptides, TCRδ polypeptides, TCR fusion polypeptides, polynucleotides, vectors comprising same, genetically modified immune effector cells, etc., as contemplated herein. Compositions include, but are not limited to pharmaceutical compositions. In preferred embodiments, a composition comprises one or more cells modified to express an engineered TCR contemplated herein.

A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy. In preferred embodiments, a pharmaceutical composition comprises a pharmaceutically acceptable carrier, diluent or excipient, and one or more cells modified to express an engineered TCR as contemplated herein.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular embodiments, formulation of pharmaceutically-acceptable carrier solutions is well-known to those of skill in the art, as is the development of suitable dosing and treatment regimens for using the particular compositions described herein in a variety of treatment regimens, including e.g., enteral and parenteral, e.g., intravascular, intravenous, intrarterial, intrarterial, intraosseously, intraventricular, intracerebral, intracranial, intraspinal, intrathecal, and intramedullary administration and formulation. It would be understood by the skilled artisan that particular embodiments contemplated herein may comprise other formulations, such as those that are well known in the pharmaceutical art, and are described, for example, in Remington: The Science and Practice of Pharmacy, volume I and volume II. 22^{nd} Edition. Edited by Loyd V. Allen Jr. Philadelphia, PA: Pharmaceutical Press; 2012, which is incorporated by reference herein, in its entirety.

In particular embodiments, compositions comprise an amount of immune effector cells expressing an engineered TCR contemplated herein. As used herein, the term “amount” refers to “an amount effective” or “an effective amount” of a genetically modified therapeutic cell, e.g., T cell, to achieve a beneficial or desired prophylactic or therapeutic result, including clinical results.

A “prophylactically effective amount” refers to an amount of a genetically modified therapeutic cells effective to achieve the desired prophylactic result. Typically, but not necessarily, since a prophylactic dose is used in subjects prior to or at an earlier stage of disease, the prophylactically effective amount is less than the therapeutically effective amount.

A “therapeutically effective amount” of a genetically modified therapeutic cell may vary according to factors such as the disease state, age, sex, and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient). When a therapeutic amount is indicated, the precise amount of the compositions to be administered can be determined by a physician with consideration of individual differences in age, weight, tumor size, extent of infection or metastasis, and condition of the patient (subject).

It can generally be stated that a pharmaceutical composition comprising the T cells described herein may be administered at a dosage of 10⁶ to 10¹³ cells/kg body weight, preferably 10⁸ to 10¹³ cells/kg body weight, including all integer values within those ranges. The number of cells will depend upon the ultimate use for which the composition is intended as will the type of cells included therein. For uses provided herein, the cells are generally in a volume of a liter or less, can be 500 mLs or less, even 250 mLs or 100 mLs or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁶, 10⁷, 10⁸, 10⁹, 10¹⁰, 10¹¹, 10¹² or 10¹³ cells. Compositions may be administered multiple times at dosages within these ranges. The cells may be allogeneic, syngeneic, xenogeneic, or autologous to the patient undergoing therapy. If desired, the treatment may also include administration of mitogens (e.g., PHA) or lymphokines, cytokines, and/or chemokines (e.g., IFN-γ, IL-2, IL-12, TNF-alpha, IL-18, and TNF-beta, GM-CSF, IL-4, IL-13, Flt3-L, RANTES, MIP1α, etc.) as contemplated herein to enhance induction of the immune response.

Generally, compositions comprising the cells activated and expanded as contemplated herein may be utilized in the treatment and prevention of diseases that arise in individuals who are immunocompromised. In particular embodiments, compositions comprising immune effector cells modified to express an engineered TCR contemplated herein are used in the treatment of cancer. The modified immune effector cells may be administered either alone, or as a pharmaceutical composition in combination with carriers, diluents, excipients, and/or with other components such as IL-2 or other cytokines or cell populations. In particular embodiments, pharmaceutical compositions comprise an amount of genetically modified T cells, in combination with one or more pharmaceutically or physiologically acceptable carriers, diluents or excipients.

Pharmaceutical compositions comprising an immune effector cell population modified to express an engineered TCR (e.g., T cells), may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions are preferably formulated for parenteral administration, e.g., intravascular (intravenous or intraarterial), intraperitoneal or intramuscular administration.

The liquid pharmaceutical compositions, whether they be solutions, suspensions or other like form, may include one or more of the following: sterile diluents such as water for injection, saline solution, preferably physiological saline, Ringer's solution, or isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In one embodiment, the T cell compositions contemplated herein are formulated in a pharmaceutically acceptable cell culture medium. Such compositions are suitable for administration to human subjects. In particular embodiments, the pharmaceutically acceptable cell culture medium is a serum free medium.

Serum-free medium has several advantages over serum containing medium, including a simplified and better-defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions includes, but is not limited to QBSF-60 (Quality Biological, Inc.), StemPro-34 (Life Technologies), and X-VIVO 10.

In one preferred embodiment, compositions comprising immune effector cells contemplated herein are formulated in a solution comprising PlasmaLyte A.

In another preferred embodiment, compositions comprising immune effector cells contemplated herein are formulated in a solution comprising a cryopreservation medium. For example, cryopreservation media with cryopreservation agents may be used to maintain a high cell viability outcome post-thaw. Illustrative examples of cryopreservation media used in particular compositions includes, but is not limited to, CryoStor CS10, CryoStor CS5, and CryoStor CS2.

In a more preferred embodiment, compositions comprising immune effector cells contemplated herein are formulated in a solution comprising 50:50 PlasmaLyte A to CryoStor CS10.

In a particular embodiment, compositions comprise an effective amount of genome edited immune effector cells modified to express an engineered TCR contemplated herein. Thus, the immune effector cell compositions may be administered alone or in combination with other known cancer treatments, such as radiation therapy, chemotherapy, transplantation, immunotherapy, hormone therapy, photodynamic therapy, etc. The compositions may also be administered in combination with antibiotics. Such therapeutic agents may be accepted in the art as a standard treatment for a particular disease state as described herein, such as a particular cancer. Exemplary therapeutic agents contemplated in particular embodiments include cytokines, growth factors, steroids, NSAIDs, DMARDs, anti-inflammatories, chemotherapeutics, radiotherapeutics, therapeutic antibodies, or other active and ancillary agents.

In certain embodiments, compositions comprising genome edited immune effector cells modified to express an engineered TCR contemplated herein may be administered in conjunction with any number of chemotherapeutic agents. A variety of other therapeutic agents may be used in conjunction with the compositions contemplated herein. In one embodiment, the composition comprising immune effector cells expressing an engineered TCR is administered with an anti-inflammatory agent.

In particular embodiments, a composition comprising immune effector modified to express an engineered TCR contemplated herein is administered with a therapeutic antibody (e.g., mono or bispecific antibody or fragment thereof) and/or an immune cell engager (NK engager). Illustrative examples of therapeutic antibodies suitable for combination with the CAR modified T cells contemplated in particular embodiments, include but are not limited to, atezolizumab, avelumab, bavituximab, bevacizumab (avastin), bivatuzumab, blinatumomab, conatumumab, crizotinib, daratumumab, duligotumab, dacetuzumab, dalotuzumab, durvalumab, elotuzumab (HuLuc63), gemtuzumab, ibritumomab, indatuximab, inotuzumab, ipilimumab, lorvotuzumab, lucatumumab, milatuzumab, moxetumomab, nivolumab, ocaratuzumab, ofatumumab, pembrolizumab, rituximab, siltuximab, teprotumumab, and ublituximab.

### J. Therapeutic Methods

The genetically modified immune effector cells expressing an engineered TCR contemplated herein provide improved methods of adoptive immunotherapy for use in the prevention, treatment, and amelioration cancers or for preventing, treating, or ameliorating at least one symptom associated with cancer.

In various embodiments, the genetically modified immune effector cells contemplated herein provide improved methods of adoptive immunotherapy for use in increasing the cytotoxicity in cancer cells in a subject or for use in decreasing the number of cancer cells in a subject.

In particular embodiments, the specificity of a primary immune effector cell is redirected to cells expressing a particular antigen, e.g., cancer cells, by genetically modifying the primary immune effector cell with an engineered TCR as contemplated herein. In various embodiments, a viral vector is used to genetically modify an immune effector cell with a particular polynucleotide encoding an engineered TCR. In some embodiments, the engineered TCR comprises (a) a TCRα polypeptide comprising a TCRα variable domain; (b) a TCRβ polypeptide comprising a TCRβ variable domain; and (c) one or more antigen-binding domains linked to the TCRα variable domain and/or TCRβ variable domain. In some embodiments, the engineered TCR comprises (a) a TCRγ polypeptide comprising a TCRγ variable domain; (b) a TCRδ polypeptide comprising a TCRδ variable domain; and (c) one or more antigen-binding domains linked to the TCRγ variable domain and/or TCRδ variable domain. In certain embodiments, the linker is a polypeptide linker. In particular embodiments, the polypeptide linker comprises an amino acid sequence as set forth in any one or more of SEQ ID NOs: 33-53.

In one embodiment, a type of cellular therapy where T cells are genetically modified to express an engineered TCR contemplated herein are infused to a recipient in need thereof is provided. The infused cell is able to kill disease causing cells in the recipient. Unlike antibody therapies, T cell therapies are able to replicate in vivo resulting in long-term persistence that can lead to sustained cancer therapy.

In one embodiment, T cells that express an engineered TCR contemplated herein can undergo robust in vivo T cell expansion and can persist for an extended amount of time. In another embodiment, T cells that express an engineered TCR contemplated herein evolve into specific memory T cells or stem cell memory T cells that can be reactivated to inhibit any additional tumor formation or growth.

In particular embodiments, modified immune effector cells that express an engineered TCR contemplated herein are used in the treatment of solid tumors or cancers.

In particular embodiments, the modified immune effector cells contemplated herein are used in the treatment of solid tumors or cancers including, but not limited to: adrenal cancer, adrenocortical carcinoma, anal cancer, appendix cancer, astrocytoma, atypical teratoid/rhabdoid tumor, basal cell carcinoma, bile duct cancer, bladder cancer, bone cancer, brain/CNS cancer, breast cancer, bronchial tumors, cardiac tumors, cervical cancer, cholangiocarcinoma, chondrosarcoma, chordoma, colon cancer, colorectal cancer, craniopharyngioma, ductal carcinoma in situ (DCIS) endometrial cancer, ependymoma, esophageal cancer, esthesioneuroblastoma, Ewing's sarcoma, extracranial germ cell tumor, extragonadal germ cell tumor, eye cancer, fallopian tube cancer, fibrous histiosarcoma, fibrosarcoma, gallbladder cancer, gastric cancer, gastrointestinal carcinoid tumors, gastrointestinal stromal tumor (GIST), germ cell tumors, glioma, glioblastoma, head and neck cancer, hemangioblastoma, hepatocellular cancer, hypopharyngeal cancer, intraocular melanoma, kaposi sarcoma, kidney cancer, laryngeal cancer, leiomyosarcoma, lip cancer, liposarcoma, liver cancer, lung cancer, non-small cell lung cancer, lung carcinoid tumor, malignant mesothelioma, medullary carcinoma, medulloblastoma, menangioma, melanoma, Merkel cell carcinoma, midline tract carcinoma, mouth cancer, myxosarcoma, myelodysplastic syndrome, myeloproliferative neoplasms, nasal cavity and paranasal sinus cancer, nasopharyngeal cancer, neuroblastoma, oligodendroglioma, oral cancer, oral cavity cancer, oropharyngeal cancer, osteosarcoma, ovarian cancer, pancreatic cancer, pancreatic islet cell tumors, papillary carcinoma, paraganglioma, parathyroid cancer, penile cancer, pharyngeal cancer, pheochromocytoma, pinealoma, pituitary tumor, pleuropulmonary blastoma, primary peritoneal cancer, prostate cancer, rectal cancer, retinoblastoma, renal cell carcinoma, renal pelvis and ureter cancer, rhabdomyosarcoma, salivary gland cancer, sebaceous gland carcinoma, skin cancer, soft tissue sarcoma, squamous cell carcinoma, small cell lung cancer, small intestine cancer, stomach cancer, sweat gland carcinoma, synovioma, testicular cancer, throat cancer, thymus cancer, thyroid cancer, urethral cancer, uterine cancer, uterine sarcoma, vaginal cancer, vascular cancer, vulvar cancer, and Wilms Tumor.

In particular embodiments, the modified immune effector cells contemplated herein are used in the treatment of solid tumors or cancers including, without limitation, non-small cell lung carcinoma, head and neck squamous cell carcinoma, colorectal cancer, pancreatic cancer, breast cancer, thyroid cancer, bladder cancer, cervical cancer, esophageal cancer, ovarian cancer, gastric cancer endometrial cancer, gliomas, glioblastomas, and oligodendroglioma.

In particular embodiments, the modified immune effector cells contemplated herein are used in the treatment of solid tumors or cancers including, without limitation, non-small-cell lung cancer, metastatic colorectal cancer, glioblastoma, head and neck cancer, pancreatic cancer, and breast cancer.

In particular embodiments, the modified immune effector cells contemplated herein are used in the treatment of glioblastoma.

In particular embodiments, the modified immune effector cells that express an engineered TCR contemplated herein are used in the treatment of liquid cancers or hematological cancers.

In particular embodiments, the modified immune effector cells contemplated herein are used in the treatment of B-cell malignancies, including but not limited to: leukemias, lymphomas, and multiple myeloma.

In particular embodiments, the modified immune effector cells contemplated herein are used in the treatment of liquid cancers including, but not limited to leukemias, lymphomas, and multiple myelomas: acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CML), chronic myelomonocytic leukemia (CMML) and polycythemia vera, Hodgkin lymphoma, nodular lymphocyte-predominant Hodgkin lymphoma, Burkitt lymphoma, small lymphocytic lymphoma (SLL), diffuse large B-cell lymphoma, follicular lymphoma, immunoblastic large cell lymphoma, precursor B-lymphoblastic lymphoma, mantle cell lymphoma, marginal zone lymphoma, mycosis fungoides, anaplastic large cell lymphoma, Sézary syndrome, precursor T-lymphoblastic lymphoma, multiple myeloma, overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

In certain embodiments, the liquid or hematological cancer is selected from the group consisting of: acute lymphocytic leukemia (ALL), chronic lymphocytic leukemia (CLL), hairy cell leukemia (HCL), multiple myeloma (MM), acute myeloid leukemia (AML), or chronic myeloid leukemia (CML).

In preferred embodiments, the liquid or hematological cancer is multiple myeloma (MM).

In preferred embodiments, the liquid or hematological cancer is relapsed/refractory multiple myeloma (MM).

In particular embodiments, the modified immune effector cells contemplated herein are used in the treatment of acute myeloid leukemia (AML).

In particular embodiments, the modified immune effector cells contemplated herein are used in the treatment of lymphoma (e.g., non-hogkin's lymphoma or DLBCL).

As used herein, the terms “individual” and “subject” are often used interchangeably and refer to any animal that exhibits a symptom of a disease, disorder, or condition that can be treated with the gene therapy vectors, cell-based therapeutics, and methods contemplated elsewhere herein. In preferred embodiments, a subject includes any animal that exhibits symptoms of a disease, disorder, or condition related to cancer that can be treated with the gene therapy vectors, cell-based therapeutics, and methods contemplated elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

As used herein, the term “patient” refers to a subject that has been diagnosed with a particular disease, disorder, or condition that can be treated with the gene therapy vectors, cell-based therapeutics, and methods disclosed elsewhere herein.

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, the phrase “ameliorating at least one symptom of” refers to decreasing one or more symptoms of the disease or condition for which the subject is being treated. In particular embodiments, the disease or condition being treated is a cancer, wherein the one or more symptoms ameliorated include, but are not limited to, weakness, fatigue, shortness of breath, easy bruising and bleeding, frequent infections, enlarged lymph nodes, distended or painful abdomen (due to enlarged abdominal organs), bone or joint pain, fractures, unplanned weight loss, poor appetite, night sweats, persistent mild fever, and decreased urination (due to impaired kidney function).

By “enhance” or “promote,” or “increase” or “expand” refers generally to the ability of a composition contemplated herein, e.g., genetically modified T cells that express an engineered TCR contemplated herein, to produce, elicit, or cause a greater physiological response (i.e., downstream effects) compared to the response caused by either vehicle or a control molecule/composition. A measurable physiological response may include an increase in T cell expansion, activation, persistence, and/or an increase in cancer cell killing ability, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. A “decrease” or “reduced” amount is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

By “maintain,” or “preserve,” or “maintenance,” or “no change,” or “no substantial change,” or “no substantial decrease” refers generally to the ability of a composition contemplated herein to produce, elicit, or cause a similar physiological response (i.e., downstream effects) in a cell, as compared to the response caused by either vehicle, a control molecule/composition, or the response in a particular cell lineage. A comparable response is one that is not significantly different or measurable different from the reference response.

In one embodiment, a method of treating cancer in a subject in need thereof comprises administering an effective amount, e.g., therapeutically effective amount of a composition comprising genetically modified immune effector cells contemplated herein. The quantity and frequency of administration will be determined by such factors as the condition of the patient, and the type and severity of the patient's disease, although appropriate dosages may be determined by clinical trials.

In one embodiment, the amount of immune effector cells, e.g., T cells that express an engineered TCR, in the composition administered to a subject is at least 1×10⁷ cells, at least 0.5×10⁸ cells, at least 1×10⁸ cells, at least 0.5×10⁹ cells, at least 1×10⁹ cells, at least 1×10¹⁰ cells, at least 1×10¹¹ cells, at least 1×10¹² cells, at least 5×10¹² cells, or at least 1×10¹³ cells.

In particular embodiments, about 1×10⁷ T cells to about 1×10¹³ T cells, about 1×10⁸ T cells to about 1×10¹³ T cells, about 1×10⁹ T cells to about 1×10¹³ T cells, about 1×10¹⁰ T cells to about 1×10¹³ T cells, about 1×10¹¹ T cells to about 1×10¹³ T cells, or about 1×10¹² T cells to about 1×10¹³ T cells are administered to a subject.

In one embodiment, the amount of immune effector cells, e.g., T cells that express an engineered TCR, in the composition administered to a subject is at least 0.1×10⁴ cells/kg of bodyweight, at least 0.5×10⁴ cells/kg of bodyweight, at least 1×10⁴ cells/kg of bodyweight, at least 5×10⁴ cells/kg of bodyweight, at least 1×10⁵ cells/kg of bodyweight, at least 0.5×10⁶ cells/kg of bodyweight, at least 1×10⁶ cells/kg of bodyweight, at least 0.5×10⁷ cells/kg of bodyweight, at least 1×10⁷ cells/kg of bodyweight, at least 0.5×10⁸ cells/kg of bodyweight, at least 1×10⁸ cells/kg of bodyweight, at least 2×10⁸ cells/kg of bodyweight, at least 3×10⁸ cells/kg of bodyweight, at least 4×10⁸ cells/kg of bodyweight, at least 5×10⁸ cells/kg of bodyweight, or at least 1×10⁹ cells/kg of bodyweight.

In particular embodiments, about 1×10⁶ T cells/kg of bodyweight to about 1×10⁸ T cells/kg of bodyweight, about 2×10⁶ T cells/kg of bodyweight to about 0.9×10⁸ T cells/kg of bodyweight, about 3×10⁶ T cells/kg of bodyweight to about 0.8×10⁸ T cells/kg of bodyweight, about 4×10⁶ T cells/kg of bodyweight to about 0.7×10⁸ T cells/kg of bodyweight, about 5×10⁶ T cells/kg of bodyweight to about 0.6×10⁸ T cells/kg of bodyweight, or about 5×10⁶ T cells/kg of bodyweight to about 0.5×10⁸ T cells/kg of bodyweight are administered to a subject.

One of ordinary skill in the art would recognize that multiple administrations of the compositions contemplated herein may be required to effect the desired therapy. For example a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

In certain embodiments, it may be desirable to administer activated immune effector cells to a subject and then subsequently redraw blood (or have an apheresis performed), activate immune effector cells therefrom, and reinfuse the patient with these activated and expanded immune effector cells. This process can be carried out multiple times every few weeks. In certain embodiments, immune effector cells can be activated from blood draws of from 10 cc to 400 cc. In certain embodiments, immune effector cells are activated from blood draws of 20 cc, 30 cc, 40 cc, 50 cc, 60 cc, 70 cc, 80 cc, 90 cc, 100 cc, 150 cc, 200 cc, 250 cc, 300 cc, 350 cc, or 400 cc or more. Not to be bound by theory, using this multiple blood draw/multiple reinfusion protocol may serve to select out certain populations of immune effector cells.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

In one embodiment, a subject in need thereof is administered an effective amount of a composition to increase a cellular immune response to a B cell related condition in the subject. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

In one embodiment, a method of treating a subject diagnosed with a cancer is provided comprising removing immune effector cells from the subject, genetically modifying said immune effector cells with a vector comprising a nucleic acid encoding an engineered TCR contemplated herein, thereby producing a population of modified immune effector cells, and administering the population of modified immune effector cells to the same subject. In a preferred embodiment, the immune effector cells comprise T cells.

In certain embodiments, methods for stimulating an immune effector cell mediated immune modulator response to a target cell population in a subject are provided comprising the steps of administering to the subject an immune effector cell population expressing a nucleic acid construct encoding an engineered TCR contemplated herein.

The methods for administering the cell compositions contemplated in particular embodiments includes any method which is effective to result in reintroduction of ex vivo genetically modified immune effector cells that either directly express an engineered TCR contemplated herein in the subject or on reintroduction of the genetically modified progenitors of immune effector cells that on introduction into a subject differentiate into mature immune effector cells that express the TCR. One method comprises transducing peripheral blood T cells ex vivo with a nucleic acid construct contemplated herein and returning the transduced cells into the subject.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

SEQUENCE LISTING

| SEQ | | |
|---|---|---|
| ID | | |
| NO | NAME | SEQUENCE |
| 1 | anti-BCMA.1 scFV | DIVLTQSPPSLAMSLGKRATISCRASESVTILGSHLIHWYQQKP |
| | | GQPPTLLIQLASNVQTGVPARFSGSGSRTDFTLTIDPVEEDDVA |
| | | VYYCLQSRTIPRTFGGGTKLEIKGSTSGSGKPGSGEGSTKGQIQ |
| | | LVQSGPELKKPGETVKISCKASGYTFTDYSINWVKRAPGKGLKW |
| | | MGWINTETREPAYAYDFRGRFAFSLETSASTAYLQINNLKYEDT |
| | | ATYFCALDYSYAMDYWGQGTSVTVSS |
| 2 | anti-BCMA.2 scFV | DIVLTQSPASLAVSLGERATINCRASESVSVIGAHLIHWYQQKP |
| | | GQPPKLLIYLASNLETGVPARFSGSGSGTDFTLTISSLQAEDAA |
| | | IYYCLQSRIFPRTFGQGTKLEIKGSTSGSGKPGSGEGSTKGQVQ |
| | | LVQSGSELKKPGASVKVSCKASGYTFTDYSINWVRQAPGQGLEW |
| | | MGWINTETREPAYAYDFRGRFVFSLDTSVSTAYLQISSLKAEDT |
| | | AVYYCARDYSYAMDYWGQGTLVTVSS |
| 3 | anti-BCMA. 3 scFV | DIQMTQSPSSLSASVGDRITITCRASQDIRNYLGWYQQKPGKAP |
| | | KVLIFAASSLQSGVPSRFSGSGSGTDFTLTISSLQPEDFATYYC |
| | | LQDYIYPWTFAQGTKVEIKGGGGSGGGGSGGGGSQVQLVESGGG |
| | | VVQPGRSLRLSCAASGFTFSSYGMHWVRQAPGKGLEWVAVISYD |
| | | GRNKNYADSVKGRFTISRDNSKNTLYLQMNSLRAEDTAVYYCAR |
| | | EGEATYYDILTGPFDYWGQGTLVTVSS |
| 4 | anti-CD19 scFV | DIQMTQTTSSLSASLGDRVTISCRASQDISKYLNWYQQKPDGTV |
| | | KLLIYHTSRLHSGVPSRFSGSGSGTDYSLTISNLEQEDIATYFC |
| | | QQGNTLPYTFGGGTKLEITGGGGSGGGGSGGGGSEVKLQESGPG |
| | | LVAPSQSLSVTCTVSGVSLPDYGVSWIRQPPRKGLEWLGVIWGS |
| | | ETTYYNSALKSRLTIIKDNSKSQVFLKMNSLQTDDTAIYYCAKH |
| | | YYYGGSYAMDYWGQGTSVTVSS |
| 5 | anti-CD20.1 scFV | EVQLQQSGAELVKPGASVKMSCKASGYTFTSYNMHWVKQTPGQG |
| | | LEWIGAIYPGNGDTSYNQKFKGKATLTADKSSSTAYMQLSSLTS |
| | | EDSADYYCARSNYYGSSYWFFDVWGAGTTVTVSSGGGGSGGGGS |
| | | GGGGSDIVLTQSPAILSASPGEKVTMTCRASSSVNYMDWYQKKP |
| | | GSSPKPWIYATSNLASGVPARFSGSGSGTSYSLTISRVEAEDAA |
| | | TYYCQQWSFNPPTFGGGTKLEIK |
| 6 | anti-CD20.2 scFV | QVQLQQPGAELVKPGASVKMSCKASGYTFTSYNMHWVKQTPGRG |
| | | LEWIGAIYPGNGDTSYNQKFKGKATLTADKSSSTAYMQLSSLTS |
| | | EDSAVYYCARSTYYGGDWYFNVWGAGTTVTVSAGGGGGGGGSG |
| | | GGGSQIVLSQSPAILSASPGEKVTMTCRASSSVSYIHWFQQKPG |
| | | SSPKPWIYATSNLASGVPVRFSGSGSGTSYSLTISRVEAEDAAT |
| | | YYCQQWTSNPPTFGGGTKLEIKR |
| 7 | anti-CD20.3 scFV | QVQLVQSGAEVKKPGSSVKVSCKASGYAFSYSWINWVRQAPGQG |
| | | LEWMGRIFPGDGDTDYNGKFKGRVTITADKSTSTAYMELSSLRS |
| | | EDTAVYYCARNVFDGYWLVYWGQGTLVTVSSGGGGSGGGGSGGG |
| | | GSDIVMTQTPLSLPVTPGEPASISCRSSKSLLHSNGITYLYWYL |
| | | QKPGQSPQLLIYQMSNLVSGVPDRFSGSGSGTDFTLKISRVEAE |
| | | DVGVYYCAQNLELPYTFGGGTKVEIKRTV |
| 8 | anti-CD22 scFV | QVQLQQSGPGLVKPSQTLSLTCAISGDSVSSNSAAWNWIRQSPS |
| | | RGLEWLGRTYYRSKWYNDYAVSVKSRITINPDTSKNQFSLQLNS |
| | | VTPEDTAVYYCAREVTGDLEDAFDIWGQGTMVTVSSGGGGSDIQ |
| | | MTQSPSSLSASVGDRVTITCRASQTIWSYLNWYQQRPGKAPNLL |
| | | IYAASSLQSGVPSRFSGRGSGTDFTLTISSLQAEDFATYYCQQS |
| | | YSIPQTFGQGTKLEIK |
| 9 | anti-CD33 scFV | EIVLTQSPGSLAVSPGERVTMSCKSSQSVFFSSSQKNYLAWYQQ |
| | | IPGQSPRLLIYWASTRESGVPDRFTGSGSGTDFTLTISSVQPED |
| | | LAIYYCHQYLSSRTFGQGTKLEIKGGGGSGGGGSGGGGSQVQLQ |
| | | QPGAEVVKPGASVKMSCKASGYTFTSYYIHWIKQTPGQGLEWVG |
| | | VIYPGNDDISYNQKFQGKATLTADKSSTTAYMQLSSLTSEDSAV |
| | | YYCAREVRLRYFDVWGQGTTVTVSS |
| 10 | anti-CD79A scFV | DVLMTQIPLSLPVSLGDQASISCRSSQSIVHSNGNTYLEWYLQK |
| | | PGQSPKLLIYKVSNRFSGVPDRFSGSGSGTDFTLKISRVEAEDL |
| | | GVYYCFQGSHVPFTFGSGTKLEIKRGGGGSGGGGSGGGGSQVQL |
| | | QQSGPELVKPGASVKISCKASGYTFSTSWMNWVKQRPGQGLEWI |
| | | GRIYPGDGDTNYNGKFKGKATLTADKSSNTAYMQLSSLTSVDSA |
| | | VYFCERFYYGNTFAMDYWGQGTSVTVSS |
| 11 | anti-CD79B.1 | QIQLVQSGPELKKPGETVKISCKASGYTFTDYSMHWVKQAPGEG |
| | scFV | LKWMGWINTETGEPTYADDFKGRFAFSLETSASTAYLQINNLKN |
| | | EDTATYFCYYGSYWGQGTLVTVSAGGGGSGGGGSGGGGSDIVLT |
| | | QSPASLAVSLGQRATISCKASQSVDYDGDGYMDWYQQKPGQPPK |
| | | LLIFAASNLKSGIPARFSGSGSGTDFTLNIHPVEEEDAATYYCQ |
| | | QTNEYPWTFGGGTKLEIK |
| 12 | anti-CD79B.2 | DIQLTQSPSSLSASVGDRVTITCKASQSVDYEGDSFLNWYQQKP |
| | scFV | GKAPKLLIYAASNLESGVPSRFSGSGSGTDFTLTISSLQPEDFA |
| | | TYYCQQSNEDPLTFGQGTKVEIKRGGGGSGGGGSGGGGSEVQLV |
| | | ESGGGLVQPGGSLRLSCAASGYTFSSYWIEWVRQAPGKGLEWIG |
| | | EILPGGGDTNYNEIFKGRATFSADTSKNTAYLQMNSLRAEDTAV |
| | | YYCTRRVPIRLDYWGQGTLVTVSS |
| 13 | anti-B7H3 scFV | DIVMTQSHKFMSTSIGARVSITCKASQDVRTAVAWYQQKPGQSP |
| | | KLLIYSASYRYTGVPDRFTGSGSGTDFTFTISSVQAEDLAVYYC |
| | | QQHYGTPPWTFGGGTKLEIKGGGGSGGGGSGGGGSEVQLVESGG |
| | | GLVKPGGSLKLSCEASRFTFSSYAMSWVRQTPEKRLEWVAAISG |
| | | GGRYTYYPDSMKGRFTISRDNAKNFLYLQMSSLRSEDTAMYYCA |
| | | RHYDGYLDYWGQGTTLTVSSTR |
| 14 | anti-Muc16 scFV | VKLQESGGGFVKPGGSLKVSCAASGFTFSSYAMSWVRLSPEMRL |
| | | EWVATISSAGGYIFYSDSVQGRFTISRDNAKNTLHLQMGSLRSG |
| | | DTAMYYCARQGFGNYGDYYAMDYWGQGTTVTVSSGGGGSGGGGS |
| | | GGGGSDIELTQSPSSLAVSAGEKVTMSCKSSQSLLNSRTRKNQL |
| | | AWYQQKPGQSPELLIYWASTRQSGVPDRFTGSGSGTDFTLTISS |
| | | VQAEDLAVYYCQQSYNLLTFGPGTKLEVKR |
| 15 | anti-HER2 scFV | DIQMTQSPSSLSASVGDRVTITCRASQDVNTAVAWYQQKPGKAP |
| | | KLLIYSASFLYSGVPSRFSGSRSGTDFTLTISSLQPEDFATYYC |
| | | QQHYTTPPTFGQGTKVEIKGSTSGSGKPGSGEGSGEVQLVESGG |
| | | GLVQPGGSLRLSCAASGFNIKDTYIHWVRQAPGKGLEWVARIYP |
| | | TNGYTRYADSVKGRFTISADTSKNTAYLQMNSLRAEDTAVYYCS |
| | | RWGGDGFYAMDVWGQGTLVTVSS |
| 16 | anti-EGFR scFV | DILLTQSPVILSVSPGERVSFSCRASQSIGTNIHWYQQRTNGSP |
| | | RLLIKYASESISGIPSRFSGSGSGTDFTLSINSVESEDIADYYC |
| | | QQNNNWPTTFGAGTKLELKGGGGSGGGGSGGGGSQVQLKQSGPG |
| | | LVQPSQSLSITCTVSGFSLTNYGVHWVRQSPGKGLEWLGVIWSG |
| | | GNTDYNTPFTSRLSINKDNSKSQVFFKMNSLQSNDTAIYYCARA |
| | | LTYYDYEFAYWGQGTLVTVSS |
| 17 | anti-FN-EDB scFV | EVQLLESGGGLVQPGGSLRLSCAASGFTFSSFSMSWVRQAPGKG |
| | | LEWVSSISGSSGTTYYADSVKGRFTISRDNSKNTLYLQMNSLRA |
| | | EDTAVYYCAKPFPYFDYWGQGTLVTVSSGDGSSGGSGGASEIVL |
| | | TQSPGTLSLSPGERATLSCRASQSVSSSFLAWYQQKPGQAPRLL |
| | | IYYASSRATGIPDRFSGSGSGTDFTLTISRLEPEDFAVYYCQQT |
| | | GRIPPTFGQGTKVEIK |
| 18 | anti-CLDN18.2 | QIQLVQSGPELKKPGETVKISCKASGYTFTNYGMNWVKQAPGKG |
| | scFV | LKWMGWINTNTGEPTYAEEFKGRFAFSLETSASTAYLQINNLKN |
| | | EDTATYFCARLGFGNAMDYWGQGTSVTVSSGGGGSGGGGSGGGG |
| | | SDIVMTQSPSSLTVTAGEKVTMSCKSSQSLLNSGNQKNYLTWYQ |
| | | QKPGQPPKLLIYWASTRESGVPDRFTGSGSGTDFTLTISSVQAE |
| | | DLAVYYCQNDYSYPLTFGAGTKLELK |
| 19 | anti-DLL3 scFV | QVQLQESGPGLVKPSETLSLTCTVSGDSISSYYWTWIRQPPGKG |
| | | LEWIGYIYYSGTTNYNPSLKSRVTISVDTSKSQFSLKLSSVTAA |
| | | DTAVYYCASIAVRGFFFDYWGQGTLVTVSSGGGGSGGGGSGGGG |
| | | SEIVLTQSPGTLSLSPGERATLSCRASQSVSSSYLAWYQQKPGQ |
| | | APRLLIYGASTRATGIPDRFSGSGSGTDFTLTISRLEPEDFAVY |
| | | YCQQYGTSPLTFGGGTKVEIK |
| 20 | anti-FLT3.1 scFV | QVTLKESGPVLVKPTETLTLTCTVSGFSLINARMGVSWIRQPPG |
| | | KALEWLAHIFSNAEKSYRTSLKSRLTISKDTSKSQVVLTMTNMD |
| | | PVDTATYYCARIPGYGGNGDYHYYGMDVWGQGTTVTVSSGGGGS |
| | | GGGGSGGGGSDIQMTQSPSSLSASLGDRVTITCRASQGIRNDLG |
| | | WYQQKPGKAPKRLIYASSTLQSGVPSRFSGSGSGTEFTLTISSL |
| | | QPEDFATYYCLQHNNFPWTFGQGTKVEIK |
| 21 | anti-FLT3.2 scFV | EVQLVESGGGLVQPGGSLRLSCAASGFTFSSYGMHWVRQAPGKG |
| | | LEWVAVISYDGSNKYYADSVKGRFTISRDNSKNTLYLQMNSLRA |
| | | EDTAVYYCANLAPWAAYWGQGTLVTVSSGGGGSGGGGSGGGGSE |
| | | IVLTQSPLSLPVTPGEPASISCRSSQSLLHSNGYNYLDWYLQKP |
| | | GQSPQLLIYLGSNRASGVPDRFSGSGSGTDFTLKISRVEAEDVG |
| | | VYYCMQALQTPHTFGQGTKLEIK |
| 22 | anti-CD33.1 VHH | EVQLVESGGGEVQPGGSLRLSCAASRSSGIDVMGWYRQAPGKER |
| | | ELVAEISGVGDTNYAASLADRFTVSRDNAKNTVYLQMSSLRAED |
| | | TAVYYCNAHSFLDLVGAWGQGTLVTVKP |
| 23 | anti-CD33.2 VHH | EVQLVESGGGEVQPGGSLRLSCAASGRTFSGYIMGWFRQAPGKE |
| | | RELVARISGNNLSTEYAESVKGRFTISRDNAKNTLYLQMSSLRA |
| | | EDTAVYYCAAEYDYSSGDFVYWGQGTLVTVKP |
| 24 | anti-CD33.3 VHH | EVQLVESGGGEVQPGGSLRLSCAASGSTLNIDHIGWYRQAPGKE |
| | | RELVGVISSGAGPNYAESVKGRFTISRDNAKNTVYLQMSSLRAE |
| | | DTAVYYCNAWIDYGSGLPQNYWGQGTLVTVKP |
| 25 | anti-CLL1.1 VHH | EVQLVESGGGEVQPGGSLRLSCAASGFLFSIYDMNWYRQAPGKE |
| | | REWVAGITNNGYSTAYAESVKGRFTISRDNAKNTIYLQMSSLRA |
| | | EDTAVYYCHADLTKAYDVEYAWGQGTLVTVKP |
| 26 | anti-CLL1.2 VHH | EVQLVESGGGEVQPGGSLRLSCAASGLLFSIYDMNWYRQAPGKE |
| | | REWVAGITNNGYSTAYAESVKGRFTISRDNAKNTVYLQMSSLRA |
| | | EDTAVYYCHTDEWGREYWGQGTLVTVKP |
| 27 | anti-CD123.1 VHH | EVQLVESGGGEVQPGGSLRLSCTASGRAINMYAMGWFRQAPGKE |
| | | REFVAAINWNGAYTQYAESVKGRFTISRDNAKNTLYLQMSSLRA |
| | | EDTAVYYCSADADYNTYVSPNKRVSYWGQGTLVTVKP |
| 28 | anti-CD123.2 VHH | EVQLVESGGGLVQPGGSLRLSCAASGRAINAYNMGWFRQAPGKG |
| | | REFVSAINWNAARTYYAESVKGRFTISRDNAKNTLYLQMSSLRA |
| | | EDTAVYYCAASGRWSAAVPSGEDQYNFWGQGTLVTVKP |
| 29 | anti-CD20 VHH | QVQLQESGGGLVQAGGSLRLSCAASGRTFSNYNMGWFRQAPGKE |
| | | REFVAAIDWSGGSPYYAASVRGRFTISRDNAENTVYLQMNSLKP |
| | | EDTAVYYCAAPLSYGSTWLADYWGQGTQVTVSS |
| 30 | anti-EGFR VHH | QVKLEESGGGSVQTGGSLRLTCAASGRTSRSYGMGWFRQAPGKE |
| | | REFVSGISWRGDSTGYADSVKGRFTISRDNAKNTVDLQMNSLKP |
| | | EDTAIYYCAAAAGSAWYGTLYEYDYWGQGTQVTVSS |
| 31 | anti-BCMA.1 VHH | QVKLEESGGGLVQAGRSLRLSCAASEHTFSSHVMGWFRQAPGKE |
| | | RESVAVIGWRDISTSYADSVKGRFTISRDNAKKTLYLQMNSLKP |
| | | EDTAVYYCAARRIDAAD FDSWGQGTQVTVSS |
| 32 | anti-BCMA.2 VHH | EVQLVESGGGLVQAGGSLRLSCAASGRTFTMGWFRQAPGKEREF |
| | | VAAISLSPTLAYYAESVKGRFTISRDNAKNTVVLQMNSLKPEDT |
| | | ALYYCAADRKSVMSIRPDYWGQGTQVTVSS |
| 33 | yuTCR Tinker | LEKT |
| | (uLNK) | |
| 34 | uLNK + G4S | LEKTGGGGS |
| | linker | |
| 35 | 1xG4S linker | GGGGS |
| 36 | 2xG4S linker | GGGGSGGGGS |
| 37 | 3xG4s linker | GGGGSGGGGSGGGGS |
| 38 | 4xG4s linker | GGGGSGGGGSGGGGSGGGGS |
| 39 | 5xG4S Tinker | GGGGSGGGGSGGGGSGGGGSGGGGS |
| 40 | Tinker | DGGGS |
| 41 | Tinker | TGEKP |
| 42 | Tinker | GGRR |
| 43 | Tinker | EGKSSGSGSESKVD |
| 44 | Tinker | KESGSVSSEQLAQFRSLD |
| 45 | Tinker | GGRRGGGS |
| 46 | Tinker | LRQRDGERP |
| 47 | Tinker | LRQKDGGGSERP |
| 48 | Tinker | LRQKDGGGSGGGSERP |
| 49 | Tinker | GSTSGSGKPGSGEGSTKG |
| 50 | Tinker | GSTSGSGKSSEGSGSTKG |
| 51 | Tinker | GSTSGSGKSSEGKG |
| 52 | Tinker | GSTSGSGKPGSGEGS |
| 53 | Tinker | GGGS |
| 54 | NY-ESO-1.1 TCRα | QEVTQIPAALSVPEGENLVLNCSFTDSAIYNLQWFRQDPGKGLT |
| | | SLLLIQSSQREQTSGRLNASLDKSSGRSTLYIAASQPGDSATYL |
| | | CAVRPTSGGSYIPTFGRGTSLIVHPY |
| 55 | NY-ESO-1.1 TCRβ | GVTQTPKFQVLKTGQSMTLQCAQDMNHEYMSWYRQDPGMGLRLI |
| | | HYSVGAGITDQGEVPNGYNVSRSTTEDFPLRLLSAAPSQTSVYF |
| | | CASSYVGNTGELFFGEGSRLTVLE |
| 56 | NY-ESO-1.2 TCRα | QEVTQIPAALSVPEGENLVLNCSFTDSAIYNLQWFRQDPGKGLT |
| | | SLLLIQSSQREQTSGRLNASLDKSSGRSTLYIAASQPGDSATYL |
| | | CAVRPLYGGSYIPTFGRGTSLIVH |
| 57 | NY-ESO-1.2 TCRβ | GVTQTPKFQVLKTGQSMTLQCAQDMNHEYMSWYRQDPGMGLRLI |
| | | HYSVGAGITDQGEVPNGYNVSRSTTEDFPLRLLSAAPSQTSVYF |
| | | CASSYVGNTGELFFGEGSRLTVLE |
| 58 | PRAME TCRα | VEQSPQSLHVQEGDSTNFTCSFPSSNFYALHWYRWETAKSPEAL |
| | | FVMTLNGDEKKKGRISATLNTKEGYSYLYIKGSQPEDSATYLCA |
| | | LYNNNDMRFGAGTRLTVKPN |
| 59 | PRAME TCRβ | GVIQSPRHEVTEMGQEVTLRCKPISGHNSLFWYRQTMMRGLELL |
| | | IYFNNNVPIDDSGMPEDRFSAKMPNASFSTLKIQPSEPRDSAVY |
| | | FCASSPGSTDTQYFGPGTRLTVLE |
| 60 | TP53R175H TCRα | QRKEVEQDPGPFNVPEGATVAFNCTYSNSASQSFFWYRQDCRKE |
| | | PKLLMSVYSSGNEDGRFTAQLNRASQYISLLIRDSKLSDSATYL |
| | | CVVQPGGYQKVTFGTGTKLQVIPD |
| 61 | TP53R175H TCRβ | GVTQTPKFQVLKTGQSMTLQCAQDMNHNSMYWYRQDPGMGLRLI |
| | | YYSASEGTTDKGEVPNGYNVSRLNKREFSLRLESAAPSQTSVYF |
| | | CASSEGLWQVGDEQYFGPGTRLTVTE |
| 62 | MAGE-A4 TCRα | QKITQTQPGMFVQEKEAVTLDCTYDTSDPSYGLFWYKQPSSGEM |
| | | IFLIYQGSYDQQNATEGRYSLNFQKARKSANLVISASQLGDSAM |
| | | YFCAMSGGYTGGFKTIFGAGTRLFVKAN |
| 63 | MAGE-A4 TCRβ | GVTQTPKHLITATGQRVTLRCSPRSGDLSVYWYQQSLDQGLQFL |
| | | IQYYNGEERAKGNILERFSAQQFPDLHSELNLSSLELGDSALYF |
| | | CASSGGDGDEQFFGPGTRLTVLE |
| 64 | WT1 TCRα | VEQHPSTLSVQEGDSAVIKCTYSDSASNYFPWYKQELGKRPQLI |
| | | IDIRSNVGEKKDQRIAVTLNKTAKHFSLHITETQPEDSAVYFCA |
| | | ATEDYQLIWGAGTKLIIKP |
| 65 | WT1 TCRβ | GITQSPKYLFRKEGQNVTLSCEQNLNHDAMYWYRQDPGQGLRLI |
| | | YYSQIVNDFQKGDIAEGYSVSREKKESFPLTVTSAQKNPTAFYL |
| | | CASSPGALYEQYFGPGTRLTVT |
| 66 | MR1.1 TCRα | GQNIDQPTEMTATEGAIVQINCTYQTSGFNGLFWYQQHAGEAPT |
| | | FLSYNVLDGLEEKGRFSSFLSRSKGYSYLLLKELQMKDSASYLC |
| | | AVKDSNYQLIWGAGTKLIIKPD |
| 67 | MR1.1 TCRβ | NAGVTQTPKFQVLKTGQSMTLQCAQDMNHNSMYWYRQDPGMGLR |
| | | LIYYSASEGTTDKGEVPNGYNVSRLNKREFSLRLESAAPSQTSV |
| | | YFCASSVWTGEGSGELFFGEGSRLTVLE |
| 68 | MR1.2 TCRα | QTVTQSQPEMSVQEAETVTLSCTYDTSESDYYLFWYKQPPSRQM |
| | | ILVIRQEAYKQQNATENRFSVNFQKAAKSFSLKISDSQLGDAAM |
| | | YFCAYRSAVNARLMFGDGTQLVVKPN |
| 69 | MR1.2 TCRβ | DIYQTPRYLVIGTGKKITLECSQTMGHDKMYWYQQDPGMELHLI |
| | | HYSYGVNSTEKGDLSSESTVSRIRTEHFPLTLESARPSHTSQYL |
| | | CASSEARGLAEFTDTQYFGPGTRLTVLE |
| 70 | CD1d + aGalCer | QVEQSPQSLIILEGKNCTLQCNYTVSPFSNLRWYKQDTGRGPVS |
| | TCRα | LTIMTFSENTKSNGRYTATLDADTKQSSLHITASQLSDSASYIC |
| | | VVSDRGSTLGRLYFGRGTQLTVWPD |
| 71 | CD1d + aGalCer | DIYQTPRYLVIGTGKKITLECSQTMGHDKMYWYQQDPGMELHLI |
| | TCRß | HYSYGVNSTEKGDLSSESTVSRIRTEHFPLTLESARPSHTSQYL |
| | | CASSGLRDRGLYEQYFGPGTRLTVTE |
| 72 | HPV16E7 TCRα | MLLEHLLIILWMQLTWVSGQQLNQSPQSMFIQEGEDVSMNCTSS |
| | | SIFNTWLWYKQDPGEGPVLLIALYKAGELTSNGRLTAQFGITRK |
| | | DSFLNISASIPSDVGIYFCAGHPSSNSGYALNFGKGTSLLVTPH |
| 73 | HPV16E7 TCRβ | MGTSLLCWVVLGFLGTDHTGAGVSQSPRYKVTKRGQDVALRCDP |
| | | ISGHVSLYWYRQALGQGPEFLTYFNYEAQQDKSGLPNDRFSAER |
| | | PEGSISTLTIQRTEQRDSAMYRCASSSQTGARTDTQYFGPGTRL |
| | | TVLE |
| 74 | GP100 TCRα | MVKIRQFLLAILWLQLSCVSAAKNEVEQSPQNLTAQEGEFITIN |
| | | CSYSVGISALHWLQQHPGGGIVSLFMLSSGKKKHGRLIATINIQ |
| | | EKHSSLHITASHPRDSAVYICAASLIQGAQKLVFGQGTRLTINP |
| | | N |
| 75 | GP100 TCRβ | MDSWTFCCVSLCILVAKHTDAGVIQSPRHEVTEMGQEVTLRCKP |
| | | ISGHNSLFWYRQTMMRGLELLIYFNNNVPIDDSGMPEDRFSAKM |
| | | PNASFSTLKIQPSEPRDSAVYFCASSPGGNEQFFGPGTRLTVLE |
| 76 | MART-1 TCRα | MLLEHLLIILWMQLTWVSGQQLNQSPQSMFIQEGEDVSMNCTSS |
| | | SIFNTWLWYKQDPGEGPVLLIALYKAGELTSNGRLTAQFGITRK |
| | | DSFLNISASIPSDVGIYFCAGGTGNQFYFGTGTSLTVIPN |
| 77 | MART-1 TCRβ | MGTRLFFYVALCLLWTGHMDAGITQSPRHKVTETGTPVTLRCHQ |
| | | TENHRYMYWYRQDPGHGLRLIHYSYGVKDTDKGEVSDGYSVSRS |
| | | KTEDFLLTLESATSSQTSVYFCAISEVGVGQPQHFGDGTRLSIL |
| | | E |
| 78 | allo-HLA TCRγ | MGWALLVLLAFLSPASQKSSNLEGGTKSVTRPTRSSAEITCDLT |
| | | VINAFYIHWYLHQEGKAPQRLLYYDVSNSKDVLESGLSPGKYYT |
| | | HTPRRWSWILILRNLIENDSGVYYCATWDRPEIYYKKLFGSGTT |
| | | LVVT |
| 79 | allo-HLA TCRδ | MVFSSLLCVFVAFSYSGSSVAQKVTQAQSSVSMPVRKAVTLNCL |
| | | YETSWWSYYIFWYKQLPSKEMIFLIRQGSDEQNAKSGRYSVNFK |
| | | KAAKSVALTISALQLEDSAKYFCALGDSYGGGPLYTDKLIFGKG |
| | | TRVTVEPR |
| 80 | huTRBC1 | DLNKVFPPEVAVFEPSEAEISHTQKATLVCLATGFFPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSVSYQQGVLSATILYEILLGKATLYAVLVSALVLMAMVKRKDF |
| 81 | huTRBC2 | DLKNVFPPEVAVFEPSEAEISHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGF |
| | | TSESYQQGVLSATILYEILLGKATLYAVLVSALVLMAMVKRKDS |
| | | RG |
| 82 | huTRAC | IQNPDPAVYQLRDSKSSDKSVCLFTDFDSQTNVSQSKDSDVYIT |
| | | DKTVLDMRSMDFKSNSAVAWSNKSDFACANAFNNSIIPEDTFFP |
| | | SPESSCDVKLVEKSFETDTNLNFQNLSVIGFRILLLKVAGFNLL |
| | | MTLRLWSS |
| 83 | huTRGC1 | DKQLDADVSPKPTIFLPSIAETKLQKAGTYLCLLEKFFPDVIKI |
| | | HWQEKKSNTILGSQEGNTMKTNDTYMKFSWLTVPEKSLDKEHRC |
| | | IVRHENNKNGVDQEIIFPPIKTDVITMDPKDNCSKDANDTLLLQ |
| | | LTNTSAYYMYLLLLLKSVVYFAIITCCLLRRTAFCCNGEKS |
| 84 | huTRGC2 | DKQLDADVSPKPTIFLPSIAETKLQKAGTYLCLLEKFFPDIIKI |
| | | HWQEKKSNTILGSQEGNTMKTNDTYMKFSWLTVPEESLDKEHRC |
| | | IVRHENNKNGIDQEIIFPPIKTDVTTVDPKYNYSKDANDVITMD |
| | | PKDNWSKDANDTLLLQLTNTSAYYTYLLLLLKSVVYFAIITCCL |
| | | LRRTAFCCNGEKS |
| 85 | huTRDC | SQPHTKPSVFVMKNGTNVACLVKEFYPKDIRINLVSSKKITEFD |
| | | PAIVISPSGKYNAVKLGKYEDSNSVTCSVQHDNKTVHSTDFEVK |
| | | TDSTDHVKPKETENTKQPSKSCHKPKAIVHTEKVNMMSLTVLGL |
| | | RMLFAKTVAVNFLLTAKLFFL |
| 86 | mmTRBC1 | DLNKVFPPEVAVFEPSKAEIAHTQKATLVCLATGFFPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDF |
| 87 | mmTRBC2 | DLKNVFPPEVAVFEPSKAEIAHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDS |
| | | RG |
| 88 | mmTRAC | IQNPDPAVYQLRDSKSSDKSVCLFTDFDSQTNVSQSKDSDVYIT |
| | | DKTVLDMRSMDFKSNSAVAWSNKSDFACANAFNNSIIPEDTFFP |
| | | SSDVPCDVKLVEKSFETDTNLNFQNLLVIVLRILLLKVAGENLL |
| | | MTLRLWSS |
| 89 | MAGEA4 TCR | MGFRLLCCVAFCLLGAGPVDSGVTQTPKHLITATGQRVTLRCSP |
| | | RSGDLSVYWYQQSLDQGLQFLIQYYNGEERAKGNILERFSAQQF |
| | | PDLHSELNLSSLELGDSALYFCASSGGDGDEQFFGPGTRLTVLE |
| | | DLKNVFPPEVAVFEPSKAEIAHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDS |
| | | RGRAKRGSGATNFSLLKQAGDVEENPGPMSLSSLLKVVTASLWL |
| | | GPGIAQKITQTQPGMFVQEKEAVTLDCTYDTSDPSYGLFWYKQP |
| | | SSGEMIFLIYQGSYDQQNATEGRYSLNFQKARKSANLVISASQL |
| | | GDSAMYFCAMSGGYTGGFKTIFGAGTRLFVKANIQNPDPAVYQL |
| | | RDSKSSDKSVCLFTDFDSQTNVSQSKDSDVYITDKTVLDMRSMD |
| | | FKSNSAVAWSNKSDFACANAFNNSIIPEDTFFPSSDVPCDVKLV |
| | | EKSFETDTNLNFQNLLVIVLRILLLKVAGFNLLMTLRLWSS |
| 90 | CD33 DARIC | MALPVTALLLPLALLLHAARPGSILWHEMWHEGLEEASRLYFGE |
| | | RNVKGMFEVLEPLHAMMERGPQTLKETSFNQAYGRDLMEAQEWC |
| | | RKYMKSGNVKDLLQAWDLYYHVFRRISKASAGTGSDIYIWAPLA |
| | | GTCGVLLLSLVITMHKRGRKKLLYIFKQPFMRPVQTTQEEDGCS |
| | | CRFPEEEEGGCELRVKFSRSADAPAYQQGQNQLYNELNLGRREE |
| | | YDVLDKRRGRDPEMGGKPRRKNPQEGLYNELQKDKMAEAYSEIG |
| | | MKGERRRGKGHDGLYQGLSTATKDTYDALHMQALPPRSGSGATN |
| | | FSLLKQAGDVEENPGPSMETDTLLLWVLLLWVPGSTGEVQLVES |
| | | GGGEVQPGGSLRLSCAASRSSGIDVMGWYRQAPGKERELVAEIS |
| | | GVGDTNYAASLADRFTVSRDNAKNTVYLQMSSLRAEDTAVYYCN |
| | | AHSFLDLVGAWGQGTLVTVKPGGGGSGVQVETISPGDGRTFPKR |
| | | GQTCVVHYTGMLEDGKKFDSSRDRNKPFKFMLGKQEVIRGWEEG |
| | | VAQMSVGQRAKLTISPDYAYGATGHPGIIPPHATLVFDVELLKL |
| | | EGGRMALIVLGGVAGLLLFIGLGIFFCVRCRHRRRQ |
| 91 | CD33. VHH-mLNK- | METPAQLLFLLLLWLPDTTGEVQLVESGGGEVQPGGSLRLSCAA |
| | TRB-P2A-TRA | SRSSGIDVMGWYRQAPGKERELVAEISGVGDTNYAASLADRFTV |
| | | SRDNAKNTVYLQMSSLRAEDTAVYYCNAHSFLDLVGAWGQGTLV |
| | | TVKPLEKTDSGVTQTPKHLITATGQRVTLRCSPRSGDLSVYWYQ |
| | | QSLDQGLQFLIQYYNGEERAKGNILERFSAQQFPDLHSELNLSS |
| | | LELGDSALYFCASSGGDGDEQFFGPGTRLTVLEDLKNVFPPEVA |
| | | VFEPSKAEIAHTQKATLVCLATGFYPDHVELSWWVNGKEVHSGV |
| | | STDPQPLKEQPALNDSRYCLSSRLRVSATFWQNPRNHFRCQVQF |
| | | YGLSENDEWTQDRAKPVTQIVSAEAWGRADCGITSASYHQGVLS |
| | | ATILYEILLGKATLYAVLVSALVLMAMVKRKDSRGRAKRGSGAT |
| | | NFSLLKQAGDVEENPGPMSLSSLLKVVTASLWLGPGIAQKITQT |
| | | QPGMFVQEKEAVTLDCTYDTSDPSYGLFWYKQPSSGEMIFLIYQ |
| | | GSYDQQNATEGRYSLNFQKARKSANLVISASQLGDSAMYFCAMS |
| | | GGYTGGFKTIFGAGTRLFVKANIQNPDPAVYQLRDSKSSDKSVC |
| | | LFTDFDSQTNVSQSKDSDVYITDKTVLDMRSMDFKSNSAVAWSN |
| | | KSDFACANAFNNSIIPEDTFFPSSDVPCDVKLVEKSFETDTNLN |
| | | FQNLLVIVLRILLLKVAGFNLLMTLRLWSS |
| 92 | CD33.VHH- | METPAQLLFLLLLWLPDTTGEVQLVESGGGEVQPGGSLRLSCAA |
| | mLNK.1xG4S-TRB- | SRSSGIDVMGWYRQAPGKERELVAEISGVGDTNYAASLADRFTV |
| | P2A-TRA | SRDNAKNTVYLQMSSLRAEDTAVYYCNAHSFLDLVGAWGQGTLV |
| | | TVKPLEKTGGGGSDSGVTQTPKHLITATGQRVTLRCSPRSGDLS |
| | | VYWYQQSLDQGLQFLIQYYNGEERAKGNILERFSAQQFPDLHSE |
| | | LNLSSLELGDSALYFCASSGGDGDEQFFGPGTRLTVLEDLKNVF |
| | | PPEVAVFEPSKAEIAHTQKATLVCLATGFYPDHVELSWWVNGKE |
| | | VHSGVSTDPQPLKEQPALNDSRYCLSSRLRVSATFWQNPRNHFR |
| | | CQVQFYGLSENDEWTQDRAKPVTQIVSAEAWGRADCGITSASYH |
| | | QGVLSATILYEILLGKATLYAVLVSALVLMAMVKRKDSRGRAKR |
| | | GSGATNFSLLKQAGDVEENPGPMSLSSLLKVVTASLWLGPGIAQ |
| | | KITQTQPGMFVQEKEAVTLDCTYDTSDPSYGLFWYKQPSSGEMI |
| | | FLIYQGSYDQQNATEGRYSLNFQKARKSANLVISASQLGDSAMY |
| | | FCAMSGGYTGGFKTIFGAGTRLFVKANIQNPDPAVYQLRDSKSS |
| | | DKSVCLFTDFDSQTNVSQSKDSDVYITDKTVLDMRSMDFKSNSA |
| | | VAWSNKSDFACANAFNNSIIPEDTFFPSSDVPCDVKLVEKSFET |
| | | DTNLNFQNLLVIVLRILLLKVAGFNLLMTLRLWSS |
| 93 | TRB-P2A- | MGFRLLCCVAFCLLGAGPVDSGVTQTPKHLITATGQRVTLRCSP |
| | CD33.VHH-mLNK- | RSGDLSVYWYQQSLDQGLQFLIQYYNGEERAKGNILERFSAQQF |
| | TRA | PDLHSELNLSSLELGDSALYFCASSGGDGDEQFFGPGTRLTVLE |
| | | DLKNVFPPEVAVFEPSKAEIAHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDS |
| | | RGRAKRGSGATNFSLLKQAGDVEENPGPMETPAQLLFLLLLWLP |
| | | DTTGEVQLVESGGGEVQPGGSLRLSCAASRSSGIDVMGWYRQAP |
| | | GKERELVAEISGVGDTNYAASLADRFTVSRDNAKNTVYLQMSSL |
| | | RAEDTAVYYCNAHSFLDLVGAWGQGTLVTVKPLEKTAQKITQTQ |
| | | PGMFVQEKEAVTLDCTYDTSDPSYGLFWYKQPSSGEMIFLIYQG |
| | | SYDQQNATEGRYSLNFQKARKSANLVISASQLGDSAMYFCAMSG |
| | | GYTGGFKTIFGAGTRLFVKANIQNPDPAVYQLRDSKSSDKSVCL |
| | | FTDFDSQTNVSQSKDSDVYITDKTVLDMRSMDFKSNSAVAWSNK |
| | | SDFACANAFNNSIIPEDTFFPSSDVPCDVKLVEKSFETDTNLNF |
| | | QNLLVIVLRILLLKVAGFNLLMTLRLWSS |
| 94 | TRB-P2A- | MGFRLLCCVAFCLLGAGPVDSGVTQTPKHLITATGQRVTLRCSP |
| | CD33.VHH- | RSGDLSVYWYQQSLDQGLQFLIQYYNGEERAKGNILERFSAQQF |
| | mLNK.1xG4S-TRA | PDLHSELNLSSLELGDSALYFCASSGGDGDEQFFGPGTRLTVLE |
| | | DLKNVFPPEVAVFEPSKAEIAHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDS |
| | | RGRAKRGSGATNFSLLKQAGDVEENPGPMETPAQLLFLLLLWLP |
| | | DTTGEVQLVESGGGEVQPGGSLRLSCAASRSSGIDVMGWYRQAP |
| | | GKERELVAEISGVGDTNYAASLADRFTVSRDNAKNTVYLQMSSL |
| | | RAEDTAVYYCNAHSFLDLVGAWGQGTLVTVKPLEKTGGGGSAQK |
| | | ITQTQPGMFVQEKEAVTLDCTYDTSDPSYGLFWYKQPSSGEMIF |
| | | LIYQGSYDQQNATEGRYSLNFQKARKSANLVISASQLGDSAMYF |
| | | CAMSGGYTGGFKTIFGAGTRLFVKANIQNPDPAVYQLRDSKSSD |
| | | KSVCLFTDFDSQTNVSQSKDSDVYITDKTVLDMRSMDFKSNSAV |
| | | AWSNKSDFACANAFNNSIIPEDTFFPSSDVPCDVKLVEKSFETD |
| | | TNLNFQNLLVIVLRILLLKVAGFNLLMTLRLWSS |
| 95 | TRB-P2A- | MGFRLLCCVAFCLLGAGPVDSGVTQTPKHLITATGQRVTLRCSP |
| | CD33.VHH-1xG4S- | RSGDLSVYWYQQSLDQGLQFLIQYYNGEERAKGNILERFSAQQF |
| | TRA | PDLHSELNLSSLELGDSALYFCASSGGDGDEQFFGPGTRLTVLE |
| | | DLKNVFPPEVAVFEPSKAEIAHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDS |
| | | RGRAKRGSGATNFSLLKQAGDVEENPGPMETPAQLLFLLLLWLP |
| | | DTTGEVQLVESGGGEVQPGGSLRLSCAASRSSGIDVMGWYRQAP |
| | | GKERELVAEISGVGDTNYAASLADRFTVSRDNAKNTVYLQMSSL |
| | | RAEDTAVYYCNAHSFLDLVGAWGQGTLVTVKPGGGGSAQKITQT |
| | | QPGMFVQEKEAVTLDCTYDTSDPSYGLFWYKQPSSGEMIFLIYQ |
| | | GSYDQQNATEGRYSLNFQKARKSANLVISASQLGDSAMYFCAMS |
| | | GGYTGGFKTIFGAGTRLFVKANIQNPDPAVYQLRDSKSSDKSVC |
| | | LFTDFDSQTNVSQSKDSDVYITDKTVLDMRSMDFKSNSAVAWSN |
| | | KSDFACANAFNNSIIPEDTFFPSSDVPCDVKLVEKSFETDTNLN |
| | | FQNLLVIVLRILLLKVAGFNLLMTLRLWSS |
| 96 | TRB-P2A- | MGFRLLCCVAFCLLGAGPVDSGVTQTPKHLITATGQRVTLRCSP |
| | CD33.VHH-2xG4S- | RSGDLSVYWYQQSLDQGLQFLIQYYNGEERAKGNILERFSAQQF |
| | TRA | PDLHSELNLSSLELGDSALYFCASSGGDGDEQFFGPGTRLTVLE |
| | | DLKNVFPPEVAVFEPSKAEIAHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDS |
| | | RGRAKRGSGATNFSLLKQAGDVEENPGPMETPAQLLFLLLLWLP |
| | | DTTGEVQLVESGGGEVQPGGSLRLSCAASRSSGIDVMGWYRQAP |
| | | GKERELVAEISGVGDTNYAASLADRFTVSRDNAKNTVYLQMSSL |
| | | RAEDTAVYYCNAHSFLDLVGAWGQGTLVTVKPGGGGSGGGGSAQ |
| | | KITQTQPGMFVQEKEAVTLDCTYDTSDPSYGLFWYKQPSSGEMI |
| | | FLIYQGSYDQQNATEGRYSLNFQKARKSANLVISASQLGDSAMY |
| | | FCAMSGGYTGGFKTIFGAGTRLFVKANIQNPDPAVYQLRDSKSS |
| | | DKSVCLFTDFDSQTNVSQSKDSDVYITDKTVLDMRSMDFKSNSA |
| | | VAWSNKSDFACANAFNNSIIPEDTFFPSSDVPCDVKLVEKSFET |
| | | DTNLNFQNLLVIVLRILLLKVAGFNLLMTLRLWSS |
| 97 | TRB-P2A-CLL1- | MGFRLLCCVAFCLLGAGPVDSGVTQTPKHLITATGQRVTLRCSP |
| | CD33.VHH- | RSGDLSVYWYQQSLDQGLQFLIQYYNGEERAKGNILERFSAQQF |
| | mLNK.1xG4S-TRA | PDLHSELNLSSLELGDSALYFCASSGGDGDEQFFGPGTRLTVLE |
| | | DLKNVFPPEVAVFEPSKAEIAHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDS |
| | | RGRAKRGSGATNFSLLKQAGDVEENPGPMETPAQLLFLLLLWLP |
| | | DTTGEVQLVESGGGEVQPGGSLRLSCAASGFLFSIYDMNWYRQA |
| | | PGKEREWVAGITNNGYSTAYAESVKGRFTISRDNAKNTIYLQMS |
| | | SLRAEDTAVYYCHADLTKAYDVEYAWGQGTLVTVKPGGGGSGGG |
| | | GSEVQLVESGGGEVQPGGSLRLSCAASRSSGIDVMGWYRQAPGK |
| | | ERELVAEISGVGDTNYAASLADRFTVSRDNAKNTVYLQMSSLRA |
| | | EDTAVYYCNAHSFLDLVGAWGQGTLVTVKPLEKTGGGGSAQKIT |
| | | QTQPGMFVQEKEAVTLDCTYDTSDPSYGLFWYKQPSSGEMIFLI |
| | | YQGSYDQQNATEGRYSLNFQKARKSANLVISASQLGDSAMYFCA |
| | | MSGGYTGGFKTIFGAGTRLFVKANIQNPDPAVYQLRDSKSSDKS |
| | | VCLFTDFDSQTNVSQSKDSDVYITDKTVLDMRSMDFKSNSAVAW |
| | | SNKSDFACANAFNNSIIPEDTFFPSSDVPCDVKLVEKSFETDTN |
| | | LNFQNLLVIVLRILLLKVAGFNLLMTLRLWSS |
| 98 | CLL1 DARIC | MALPVTALLLPLALLLHAARPGSILWHEMWHEGLEEASRLYFGE |
| | | RNVKGMFEVLEPLHAMMERGPQTLKETSFNQAYGRDLMEAQEWC |
| | | RKYMKSGNVKDLLQAWDLYYHVFRRISKASAGTGSDIYIWAPLA |
| | | GTCGVLLLSLVITMHKRGRKKLLYIFKQPFMRPVQTTQEEDGCS |
| | | CRFPEEEEGGCELRVKFSRSADAPAYQQGQNQLYNELNLGRREE |
| | | YDVLDKRRGRDPEMGGKPRRKNPQEGLYNELQKDKMAEAYSEIG |
| | | MKGERRRGKGHDGLYQGLSTATKDTYDALHMQALPPRSGSGATN |
| | | FSLLKQAGDVEENPGPSMETDTLLLWVLLLWVPGSTGEVQLVES |
| | | GGGEVQPGGSLRLSCAASGFLFSIYDMNWYRQAPGKEREWVAGI |
| | | TNNGYSTAYAESVKGRFTISRDNAKNTIYLQMSSLRAEDTAVYY |
| | | CHADLTKAYDVEYAWGQGTLVTVKPGGGGSGVQVETISPGDGRT |
| | | FPKRGQTCVVHYTGMLEDGKKFDSSRDRNKPFKFMLGKQEVIRG |
| | | WEEGVAQMSVGQRAKLTISPDYAYGATGHPGIIPPHATLVEDVE |
| | | LLKLEGGRMALIVLGGVAGLLLFIGLGIFFCVRCRHRRRQ |
| 99 | CLL1-CD33 tandem | MALPVTALLLPLALLLHAARPGSILWHEMWHEGLEEASRLYFGE |
| | DARIC | RNVKGMFEVLEPLHAMMERGPQTLKETSFNQAYGRDLMEAQEWC |
| | | RKYMKSGNVKDLLQAWDLYYHVFRRISKASAGTGSDIYIWAPLA |
| | | GTCGVLLLSLVITMHKRGRKKLLYIFKQPFMRPVQTTQEEDGCS |
| | | CRFPEEEEGGCELRVKFSRSADAPAYQQGQNQLYNELNLGRREE |
| | | YDVLDKRRGRDPEMGGKPRRKNPQEGLYNELQKDKMAEAYSEIG |
| | | MKGERRRGKGHDGLYQGLSTATKDTYDALHMQALPPRSGSGATN |
| | | FSLLKQAGDVEENPGPSMETDTLLLWVLLLWVPGSTGEVQLVES |
| | | GGGEVQPGGSLRLSCAASGFLFSIYDMNWYRQAPGKEREWVAGI |
| | | TNNGYSTAYAESVKGRFTISRDNAKNTIYLQMSSLRAEDTAVYY |
| | | CHADLTKAYDVEYAWGQGTLVTVKPGGGGSGGGGSGGGGSEVQL |
| | | VESGGGEVQPGGSLRLSCAASRSSGIDVMGWYRQAPGKERELVA |
| | | EISGVGDTNYAASLADRFTVSRDNAKNTVYLQMSSLRAEDTAVY |
| | | YCNAHSFLDLVGAWGQGTLVTVKPGGGGSGVQVETISPGDGRTF |
| | | PKRGQTCVVHYTGMLEDGKKFDSSRDRNKPFKFMLGKQEVIRGW |
| | | EEGVAQMSVGQRAKLTISPDYAYGATGHPGIIPPHATLVFDVEL |
| | | LKLEGGRMALIVLGGVAGLLLFIGLGIFFCVRCRHRRRQ |
| 100 | TRB-P2A- | MGFRLLCCVAFCLLGAGPVDSGVTQTPKHLITATGQRVTLRCSP |
| | BCMA.scFv-2xG4S- | RSGDLSVYWYQQSLDQGLQFLIQYYNGEERAKGNILERFSAQQF |
| | TRA | PDLHSELNLSSLELGDSALYFCASSGGDGDEQFFGPGTRLTVLE |
| | | DLKNVFPPEVAVFEPSKAEIAHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDS |
| | | RGRAKRGSGATNFSLLKQAGDVEENPGPMETPAQLLFLLLLWLP |
| | | DTTGDIVLTQSPPSLAMSLGKRATISCRASESVTILGSHLIHWY |
| | | QQKPGQPPTLLIQLASNVQTGVPARFSGSGSRTDFTLTIDPVEE |
| | | DDVAVYYCLQSRTIPRTFGGGTKLEIKGSTSGSGKPGSGEGSTK |
| | | GQIQLVQSGPELKKPGETVKISCKASGYTFTDYSINWVKRAPGK |
| | | GLKWMGWINTETREPAYAYDFRGRFAFSLETSASTAYLQINNLK |
| | | YEDTATYFCALDYSYAMDYWGQGTSVTVSSGGGGSGGGGSAQKI |
| | | TQTQPGMFVQEKEAVTLDCTYDTSDPSYGLFWYKQPSSGEMIFL |
| | | IYQGSYDQQNATEGRYSLNFQKARKSANLVISASQLGDSAMYFC |
| | | AMSGGYTGGFKTIFGAGTRLFVKANIQNPDPAVYQLRDSKSSDK |
| | | SVCLFTDFDSQTNVSQSKDSDVYITDKTVLDMRSMDFKSNSAVA |
| | | WSNKSDFACANAFNNSIIPEDTFFPSSDVPCDVKLVEKSFETDT |
| | | NLNFQNLLVIVLRILLLKVAGFNLLMTLRLWSS |
| 101 | BCMA scFv CAR | MALPVTALLLPLALLLHAARPDIVLTQSPPSLAMSLGKRATISC |
| | | RASESVTILGSHLIHWYQQKPGQPPTLLIQLASNVQTGVPARFS |
| | | GSGSRTDFTLTIDPVEEDDVAVYYCLQSRTIPRTFGGGTKLEIK |
| | | GSTSGSGKPGSGEGSTKGQIQLVQSGPELKKPGETVKISCKASG |
| | | YTFTDYSINWVKRAPGKGLKWMGWINTETREPAYAYDFRGRFAF |
| | | SLETSASTAYLQINNLKYEDTATYFCALDYSYAMDYWGQGTSVT |
| | | VSSAAATTTPAPRPPTPAPTIASQPLSLRPEACRPAAGGAVHTR |
| | | GLDFACDIYIWAPLAGTCGVLLLSLVITLYCKRGRKKLLYIFKQ |
| | | PFMRPVQTTQEEDGCSCRFPEEEEGGCELRVKFSRSADAPAYQQ |
| | | GQNQLYNELNLGRREEYDVLDKRRGRDPEMGGKPRRKNPQEGLY |
| | | NELQKDKMAEAYSEIGMKGERRRGKGHDGLYQGLSTATKDTYDA |
| | | LHMQALPPR |
| 102 | TRB-P2A- | MGFRLLCCVAFCLLGAGPVDSGVTQTPKHLITATGQRVTLRCSP |
| | CD19.scFv-2xG4S- | RSGDLSVYWYQQSLDQGLQFLIQYYNGEERAKGNILERFSAQQF |
| | TRA | PDLHSELNLSSLELGDSALYFCASSGGDGDEQFFGPGTRLTVLE |
| | | DLKNVFPPEVAVFEPSKAEIAHTQKATLVCLATGFYPDHVELSW |
| | | WVNGKEVHSGVSTDPQPLKEQPALNDSRYCLSSRLRVSATFWQN |
| | | PRNHFRCQVQFYGLSENDEWTQDRAKPVTQIVSAEAWGRADCGI |
| | | TSASYHQGVLSATILYEILLGKATLYAVLVSALVLMAMVKRKDS |
| | | RGRAKRGSGATNFSLLKQAGDVEENPGPMETPAQLLFLLLLWLP |
| | | DTTGDIQMTQTTSSLSASLGDRVTISCRASQDISKYLNWYQQKP |
| | | DGTVKLLIYHTSRLHSGVPSRFSGSGSGTDYSLTISNLEQEDIA |
| | | TYFCQQGNTLPYTFGGGTKLEITGGGGSGGGGSGGGGSEVKLQE |
| | | SGPGLVAPSQSLSVTCTVSGVSLPDYGVSWIRQPPRKGLEWLGV |
| | | IWGSETTYYNSALKSRLTIIKDNSKSQVFLKMNSLQTDDTAIYY |
| | | CAKHYYYGGSYAMDYWGQGTSVTVSSGGGGSGGGGSAQKITQTQ |
| | | PGMFVQEKEAVTLDCTYDTSDPSYGLFWYKQPSSGEMIFLIYQG |
| | | SYDQQNATEGRYSLNFQKARKSANLVISASQLGDSAMYFCAMSG |
| | | GYTGGFKTIFGAGTRLFVKANIQNPDPAVYQLRDSKSSDKSVCL |
| | | FTDFDSQTNVSQSKDSDVYITDKTVLDMRSMDFKSNSAVAWSNK |
| | | SDFACANAFNNSIIPEDTFFPSSDVPCDVKLVEKSFETDTNLNF |
| | | QNLLVIVLRILLLKVAGFNLLMTLRLWSS |
| 103 | CD33.VHH-MLNK- | EVQLVESGGGEVQPGGSLRLSCAASRSSGIDVMGWYRQAPGKER |
| | TRB | ELVAEISGVGDTNYAASLADRFTVSRDNAKNTVYLQMSSLRAED |
| | | TAVYYCNAHSFLDLVGAWGQGTLVTVKPLEKTDSGVTQTPKHLI |
| | | TATGQRVTLRCSPRSGDLSVYWYQQSLDQGLQFLIQYYNGEERA |
| | | KGNILERFSAQQFPDLHSELNLSSLELGDSALYFCASSGGDGDE |
| | | QFFGPGTRLTVLEDLKNVFPPEVAVFEPSKAEIAHTQKATLVCL |
| | | ATGFYPDHVELSWWVNGKEVHSGVSTDPQPLKEQPALNDSRYCL |
| | | SSRLRVSATFWQNPRNHFRCQVQFYGLSENDEWTQDRAKPVTQI |
| | | VSAEAWGRADCGITSASYHQGVLSATILYEILLGKATLYAVLVS |
| | | ALVLMAMVKRKDSRG |
| 104 | CD33.VHH- | EVQLVESGGGEVQPGGSLRLSCAASRSSGIDVMGWYRQAPGKER |
| | mLNK.1xG4S-TRB | ELVAEISGVGDTNYAASLADRFTVSRDNAKNTVYLQMSSLRAED |
| | | TAVYYCNAHSFLDLVGAWGQGTLVTVKPLEKTGGGGSDSGVTQT |
| | | PKHLITATGQRVTLRCSPRSGDLSVYWYQQSLDQGLQFLIQYYN |
| | | GEERAKGNILERFSAQQFPDLHSELNLSSLELGDSALYFCASSG |
| | | GDGDEQFFGPGTRLTVLEDLKNVFPPEVAVFEPSKAEIAHTQKA |
| | | TLVCLATGFYPDHVELSWWVNGKEVHSGVSTDPQPLKEQPALND |
| | | SRYCLSSRLRVSATFWQNPRNHFRCQVQFYGLSENDEWTQDRAK |
| | | PVTQIVSAEAWGRADCGITSASYHQGVLSATILYEILLGKATLY |
| | | AVLVSALVLMAMVKRKDSRG |
| 105 | CD33.VHH-MLNK- | EVQLVESGGGEVQPGGSLRLSCAASRSSGIDVMGWYRQAPGKER |
| | TRA | ELVAEISGVGDTNYAASLADRFTVSRDNAKNTVYLQMSSLRAED |
| | | TAVYYCNAHSFLDLVGAWGQGTLVTVKPLEKTAQKITQTQPGMF |
| | | VQEKEAVTLDCTYDTSDPSYGLFWYKQPSSGEMIFLIYQGSYDQ |
| | | QNATEGRYSLNFQKARKSANLVISASQLGDSAMYFCAMSGGYTG |
| | | GFKTIFGAGTRLFVKANIQNPDPAVYQLRDSKSSDKSVCLFTDF |
| | | DSQTNVSQSKDSDVYITDKTVLDMRSMDFKSNSAVAWSNKSDFA |
| | | CANAFNNSIIPEDTFFPSSDVPCDVKLVEKSFETDTNLNFQNLL |
| | | VIVLRILLLKVAGFNLLMTLRLWSS |
| 106 | CD33.VHH- | EVQLVESGGGEVQPGGSLRLSCAASRSSGIDVMGWYRQAPGKER |
| | mLNK.1xG4S-TRA | ELVAEISGVGDTNYAASLADRFTVSRDNAKNTVYLQMSSLRAED |
| | | TAVYYCNAHSFLDLVGAWGQGTLVTVKPLEKTGGGGSAQKITQT |
| | | QPGMFVQEKEAVTLDCTYDTSDPSYGLFWYKQPSSGEMIFLIYQ |
| | | GSYDQQNATEGRYSLNFQKARKSANLVISASQLGDSAMYFCAMS |
| | | GGYTGGFKTIFGAGTRLFVKANIQNPDPAVYQLRDSKSSDKSVC |
| | | LFTDFDSQTNVSQSKDSDVYITDKTVLDMRSMDFKSNSAVAWSN |
| | | KSDFACANAFNNSIIPEDTFFPSSDVPCDVKLVEKSFETDTNLN |
| | | FQNLLVIVLRILLLKVAGFNLLMTLRLWSS |
| 107 | CD33. | EVQLVESGGGEVQPGGSLRLSCAASRSSGIDVMGWYRQAPGKER |
| | VHH-1xG4S- | ELVAEISGVGDTNYAASLADRFTVSRDNAKNTVYLQMSSLRAED |
| | TRA | TAVYYCNAHSFLDLVGAWGQGTLVTVKPGGGGSAQKITQTQPGM |
| | | FVQEKEAVTLDCTYDTSDPSYGLFWYKQPSSGEMIFLIYQGSYD |
| | | QQNATEGRYSLNFQKARKSANLVISASQLGDSAMYFCAMSGGYT |
| | | GGFKTIFGAGTRLFVKANIQNPDPAVYQLRDSKSSDKSVCLFTD |
| | | FDSQTNVSQSKDSDVYITDKTVLDMRSMDFKSNSAVAWSNKSDF |
| | | ACANAFNNSIIPEDTFFPSSDVPCDVKLVEKSFETDTNLNFQNL |
| | | LVIVLRILLLKVAGFNLLMTLRLWSS |
| 108 | CD33.VHH-2xG4S- | EVQLVESGGGEVQPGGSLRLSCAASRSSGIDVMGWYRQAPGKER |
| | TRA | ELVAEISGVGDTNYAASLADRFTVSRDNAKNTVYLQMSSLRAED |
| | | TAVYYCNAHSFLDLVGAWGQGTLVTVKPGGGGSGGGGSAQKITQ |
| | | TQPGMFVQEKEAVTLDCTYDTSDPSYGLFWYKQPSSGEMIFLIY |
| | | QGSYDQQNATEGRYSLNFQKARKSANLVISASQLGDSAMYFCAM |
| | | SGGYTGGFKTIFGAGTRLFVKANIQNPDPAVYQLRDSKSSDKSV |
| | | CLFTDFDSQTNVSQSKDSDVYITDKTVLDMRSMDFKSNSAVAWS |
| | | NKSDFACANAFNNSIIPEDTFFPSSDVPCDVKLVEKSFETDTNL |
| | | NFQNLLVIVLRILLLKVAGFNLLMTLRLWSS |
| 109 | CLL1-CD33.VHH- | EVQLVESGGGEVQPGGSLRLSCAASGFLFSIYDMNWYRQAPGKE |
| | mLNK.1xG4S-TRA | REWVAGITNNGYSTAYAESVKGRFTISRDNAKNTIYLQMSSLRA |
| | | EDTAVYYCHADLTKAYDVEYAWGQGTLVTVKPGGGGSGGGGSEV |
| | | QLVESGGGEVQPGGSLRLSCAASRSSGIDVMGWYRQAPGKEREL |
| | | VAEISGVGDTNYAASLADRFTVSRDNAKNTVYLQMSSLRAEDTA |
| | | VYYCNAHSFLDLVGAWGQGTLVTVKPLEKTGGGGSAQKITQTQP |
| | | GMFVQEKEAVTLDCTYDTSDPSYGLFWYKQPSSGEMIFLIYQGS |
| | | YDQQNATEGRYSLNFQKARKSANLVISASQLGDSAMYFCAMSGG |
| | | YTGGFKTIFGAGTRLFVKANIQNPDPAVYQLRDSKSSDKSVCLF |
| | | TDFDSQTNVSQSKDSDVYITDKTVLDMRSMDFKSNSAVAWSNKS |
| | | DFACANAFNNSIIPEDTFFPSSDVPCDVKLVEKSFETDTNLNFQ |
| | | NLLVIVLRILLLKVAGFNLLMTLRLWSS |
| 110 | BCMA.scFv-2xG4S- | DIVLTQSPPSLAMSLGKRATISCRASESVTILGSHLIHWYQQKP |
| | TRA | GQPPTLLIQLASNVQTGVPARFSGSGSRTDFTLTIDPVEEDDVA |
| | | VYYCLQSRTIPRTFGGGTKLEIKGSTSGSGKPGSGEGSTKGQIQ |
| | | LVQSGPELKKPGETVKISCKASGYTFTDYSINWVKRAPGKGLKW |
| | | MGWINTETREPAYAYDFRGRFAFSLETSASTAYLQINNLKYEDT |
| | | ATYFCALDYSYAMDYWGQGTSVTVSSGGGGSGGGGSAQKITQTQ |
| | | PGMFVQEKEAVTLDCTYDTSDPSYGLFWYKQPSSGEMIFLIYQG |
| | | SYDQQNATEGRYSLNFQKARKSANLVISASQLGDSAMYFCAMSG |
| | | GYTGGFKTIFGAGTRLFVKANIQNPDPAVYQLRDSKSSDKSVCL |
| | | FTDFDSQTNVSQSKDSDVYITDKTVLDMRSMDFKSNSAVAWSNK |
| | | SDFACANAFNNSIIPEDTFFPSSDVPCDVKLVEKSFETDTNLNF |
| | | QNLLVIVLRILLLKVAGFNLLMTLRLWSS |
| 111 | CD19.scFV-2xG4S- | DIQMTQTTSSLSASLGDRVTISCRASQDISKYLNWYQQKPDGTV |
| | TRA | KLLIYHTSRLHSGVPSRFSGSGSGTDYSLTISNLEQEDIATYFC |
| | | QQGNTLPYTFGGGTKLEITGGGGSGGGGSGGGGSEVKLQESGPG |
| | | LVAPSQSLSVTCTVSGVSLPDYGVSWIRQPPRKGLEWLGVIWGS |
| | | ETTYYNSALKSRLTIIKDNSKSQVFLKMNSLQTDDTAIYYCAKH |
| | | YYYGGSYAMDYWGQGTSVTVSSGGGGSGGGGSAQKITQTQPGMF |
| | | VQEKEAVTLDCTYDTSDPSYGLFWYKQPSSGEMIFLIYQGSYDQ |
| | | QNATEGRYSLNFQKARKSANLVISASQLGDSAMYFCAMSGGYTG |
| | | GFKTIFGAGTRLFVKANIQNPDPAVYQLRDSKSSDKSVCLFTDF |
| | | DSQTNVSQSKDSDVYITDKTVLDMRSMDFKSNSAVAWSNKSDFA |
| | | CANAFNNSIIPEDTFFPSSDVPCDVKLVEKSFETDTNLNFQNLL |
| | | VIVLRILLLKVAGFNLLMTLRLWSS |
| 112 | Furin cleavage | RAKR |
| | site | |

### EXAMPLES

### Example 1

### Evaluation of Engineered TCRs

A MAGEA4-reactive, HLA-A2-restricted T-cell receptor (TCR) was embedded with a VHH targeting human CD33 to produce an engineered dual-targeting TCR (“VHH-TCR”) (SEQ ID NO: 93) (FIGS. 1A and 1B). This was evaluated for expression and function compared to a TCR targeting MAGEA4 (SEQ ID NO: 89) and a DARIC (Dimerizing Agent Regulated Immunoreceptor Complex, a controllable and adaptable antigen recognizing system) targeting human CD33 (SEQ ID NO: 90) (the “comparators”) (FIG. 1A). Dual-targeting TCR T cells were produced in a 10 Day process using G-REX® flasks. Briefly, peripheral blood mononuclear cells (PBMC) were cultured in media containing IL-2 (CellGenix, GmbH) and antibodies specific for CD3 and CD28 (Miltenyi Biotec, Inc.). Lentiviruses encoding the test constructs were added one day after culture initiation. On Day 3, CAR T cells were transferred from a 24-well plate, to a 24 well G-REX flask, where cells were maintained until harvest on Day 10.

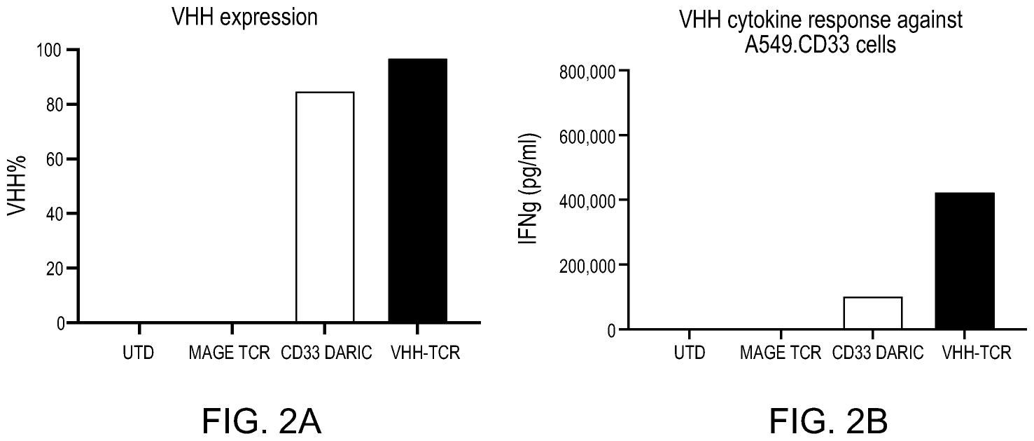

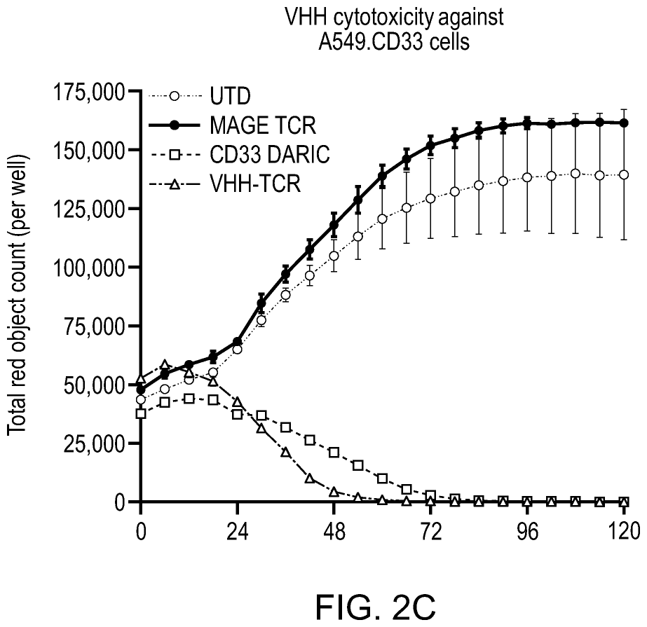

T cells were interrogated for cell surface VHH expression using flow cytometry. T cells were stained using an iFlour488 labeled anti-Camelid VHH antibody (Genscript). Surface VHH expression was higher in the VHH-TCR compared to CD33 DARIC (FIG. 2A). Additionally, the biological activity of the T cells was assessed for interferon gamma production in co-culture with tumor cell lines positive for CD33 (adherent A549 cell line that was stably transduced with CD33). As shown in FIG. 2B, the interferon gamma production of VHH-TCR is >3-fold greater than the CD33-DARIC, which was activated by including 1 nm Rapamycin during coculture. Live-cell imaging by IncuCyte was used to analyze tumor cell growth of A549.CD33 stably transduced with red reporter. The A549 cells grew normally in the presence of UTD T cells and MAGEA4 TCR-T cells. Co-culture with either VHH-TCR or CD33-DARIC resulted in tumor cell elimination, with VHH-TCR achieving elimination more rapidly than the DARIC (FIG. 2C).

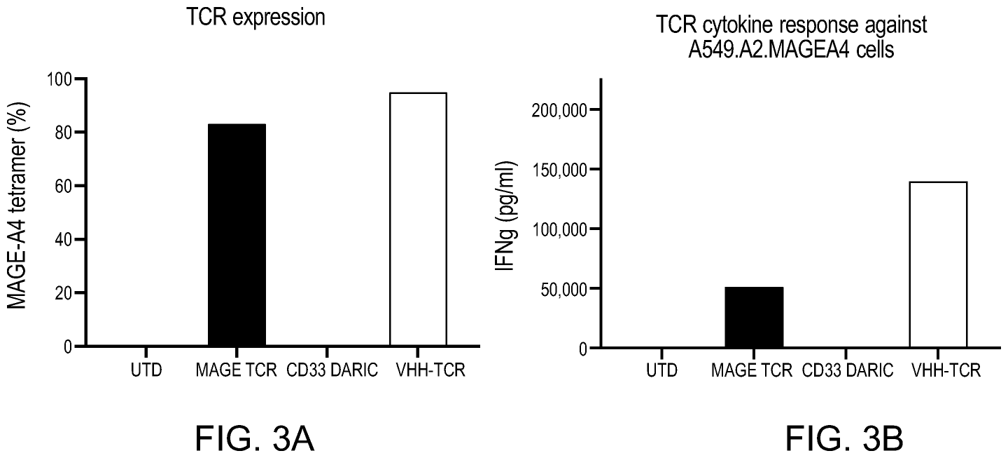

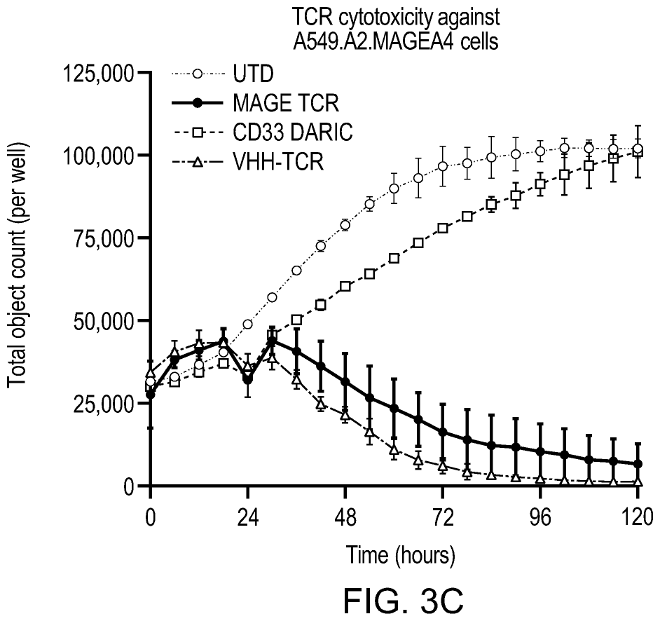

Flow cytometry was performed to evaluate MAGEA4 tetramer/HLA-multimer binding which was higher in VHH-TCR compared to the MAGEA4 TCR (FIG. 3A). Additionally, the biological activity of the T cells was assessed for interferon gamma production in co-culture with tumor cell lines positive for MAGEA4 (adherent A549 cell line that was stably transduced with MAGEA4 and HLA-A2). As shown in FIG. 3B, the interferon gamma production of VHH-TCR is ˜3-fold greater than the MAGEA4 TCR. Live-cell imaging by IncuCyte was used to analyze tumor cell growth of the adherent A549.MAGEA4.HLA-A cell line that was stably transduced with a red reporter. The A549 cells grew normally in the presence of UTD T cells and CD33-DARIC cells. Co-culture of MAGEA4 TCR resulted in complete elimination of tumor cells, whereas VHH-TCR resulted in complete and more rapid elimination of tumor cells (FIG. 3C).

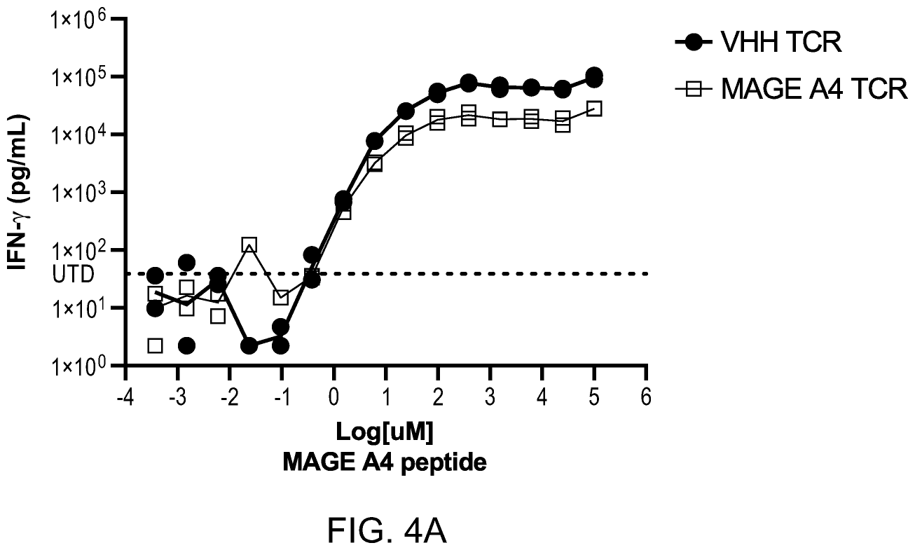

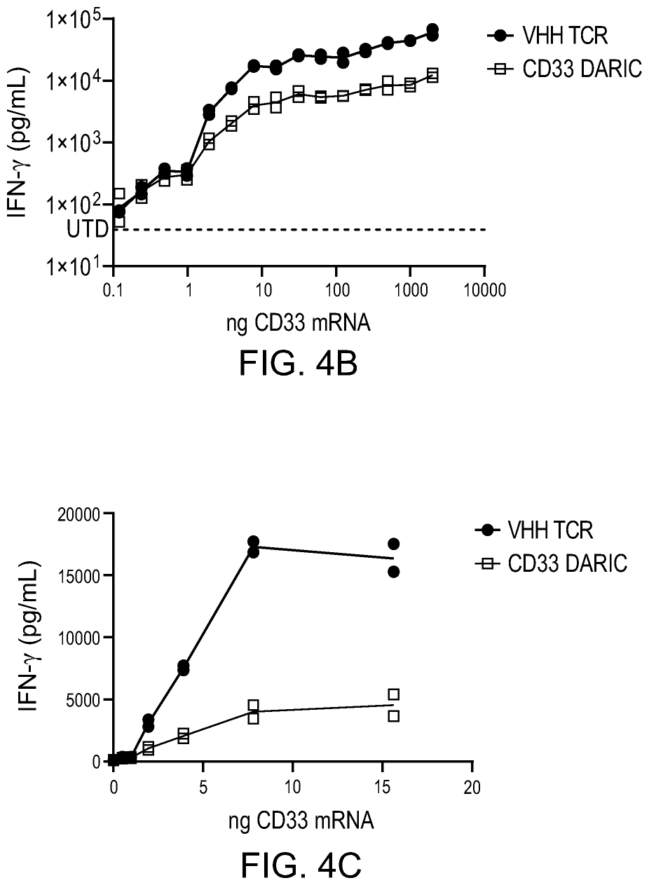

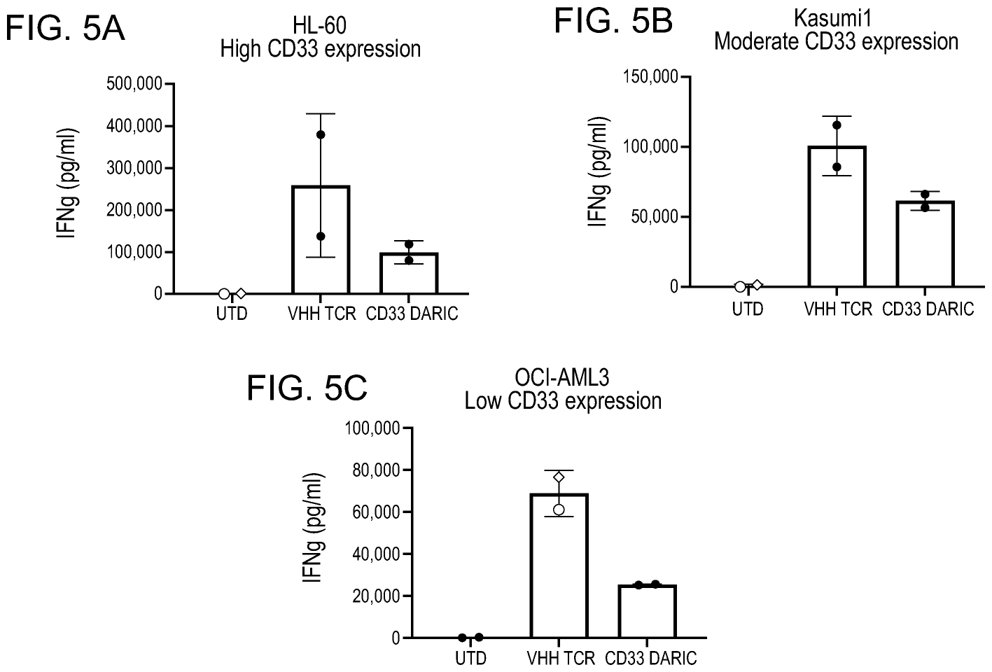

The sensitivity of VHH-TCR was compared to the MAGEA4 TCR by setting up co-cultures with A549 cells, that do not express MAGEA4, pulsed with a range of MAGEA4 peptide concentrations. As shown in FIG. 4A, compared to the MAGEA4 TCR, the VHH-TCR demonstrates similar kinetics but superior interferon gamma release in co-culture with a range of MAGEA4 peptide expression. The sensitivity of VHH-TCR was compared to the CD33 DARIC by setting up co-cultures with A549 cells, that do not express CD33, electroporated with a range of CD33 mRNA concentrations. As shown in FIGS. 4B and 4C, compared to the CD33 DARIC activated with 1 nm Rapamycin, the VHH-TCR demonstrates similar kinetics but superior interferon gamma production in co-culture with a range of CD33 mRNA expression. Additionally, cocultures were set up with cell lines endogenously expressing varying levels of CD33; HL-60 has high CD33 expression, Kasumi1 has moderate CD33 expression and OCI-AML3 has low CD33 expression. As shown in FIGS. 5A-5C, the VHH-TCR demonstrates superior interferon gamma upon coculture, most evident in OCI-AML3 that expresses low level of CD33.

### Example 2

### Evaluation of Engineered TCR Configurations

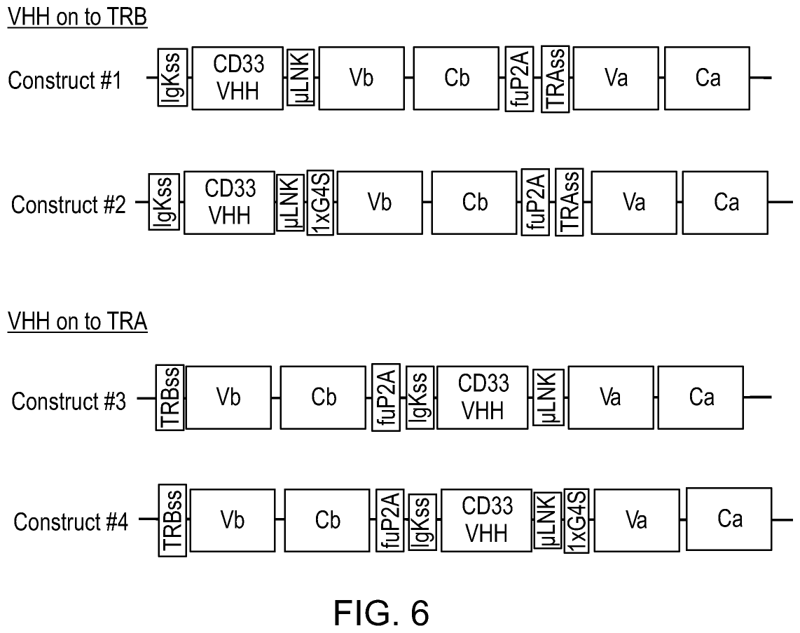

Four configurations were assessed: 1) a VHH was added to the TRB (T cell receptor beta chain) separated by a marsupial mu linker (LEKT) (SEQ ID NO: 91), 2) a VHH was added to the TRB separated by a mu linker+G4S (SEQ ID NO: 92), 3) a VHH was added to the TRA (T cell receptor alpha chain) separated by a mu linker (SEQ ID NO: 93), and 4) a VHH was added to the TRA separated by a mu linker+G4S (SEQ ID NO: 94) (FIG. 6). TCR T cells were produced as described in Example 1.

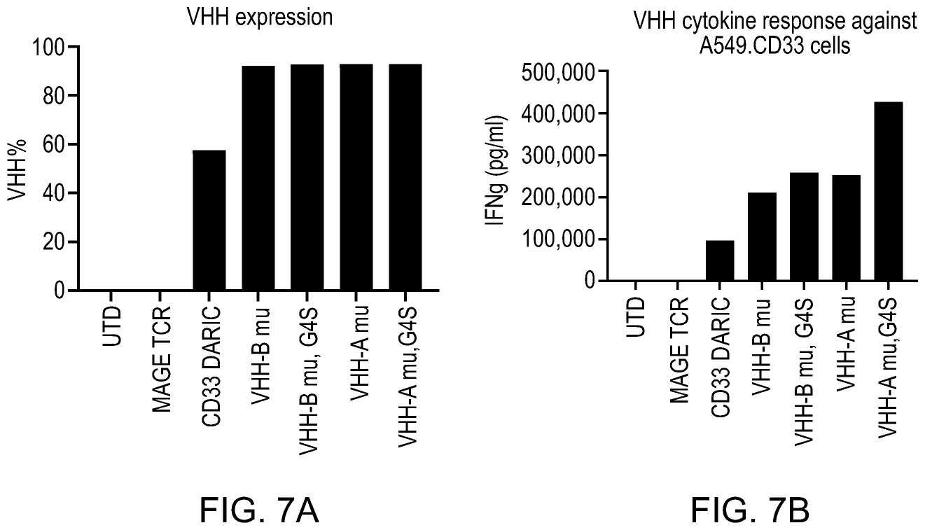

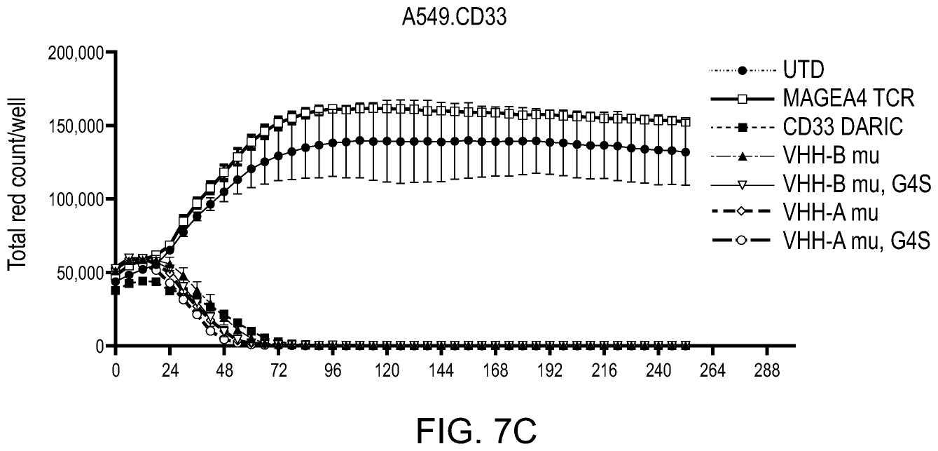

T cells were interrogated for cell surface VHH expression using flow cytometry. T cells were stained using an iFlour488 labeled anti-Camelid VHH antibody (Genscript). Surface VHH expression was higher in the VHH-TCR than the CD33 DARIC (SEQ ID NO: 90) and was comparable in all the orientations tested (FIG. 7A). Additionally, the biological activity of the T cells was assessed for interferon gamma production in co-culture with tumor cell lines positive for CD33 (adherent A549 cell line that was stably transduced with CD33). As shown in FIG. 7B, the interferon gamma production of all VHH-TCRs is >2-fold greater than the CD33-DARIC activated with 1 nm Rapamycin, and the VHH-TCR with VHH added to the TRA separated by a mu linker+G4S outperformed all the constructs assessed. Live-cell imaging by IncuCyte was used to analyze tumor cell growth of A549.CD33 stably transduced with red reporter. The A549 cells grew normally in the presence of UTD T cells and MAGEA4 TCR-T cells. Co-culture with all VHH-TCR or activated CD33-DARIC resulted in tumor cell elimination, with VHH-TCRs achieving elimination more rapidly than the DARIC (FIG. 7C).

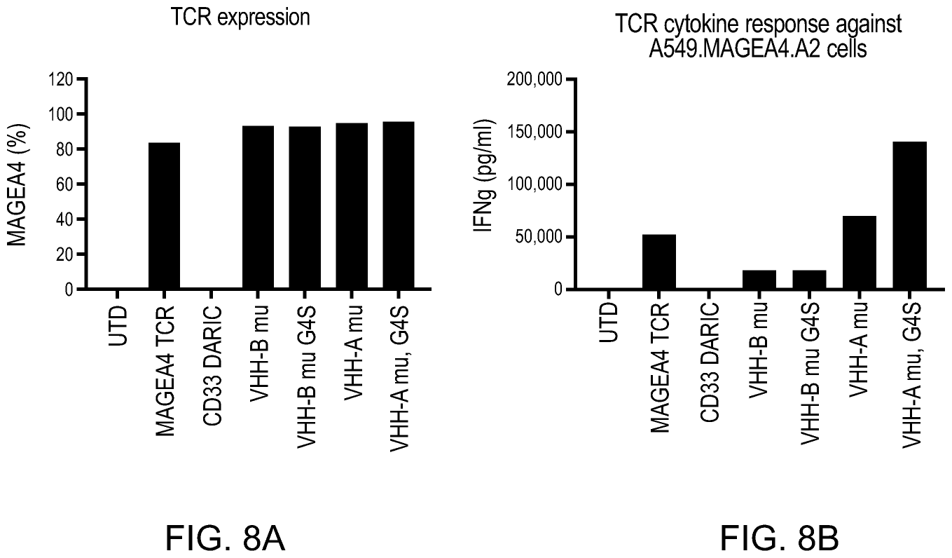

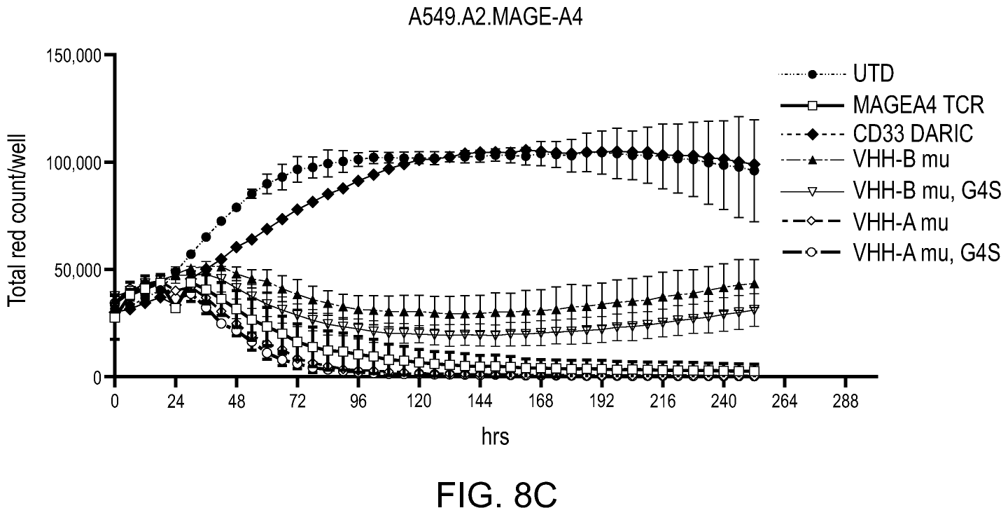

Flow cytometry was performed to evaluate MAGEA4 tetramer/HLA-multimer binding which was comparable in all VHH TCRs and to the MAGEA4 TCR (FIG. 8A). Additionally, the biological activity of the T cells was assessed for interferon gamma production in co-culture with tumor cell lines positive for MAGEA4 (adherent A549 cell line that was stably transduced with MAGEA4 and HLA-A2). As shown in FIG. 8B, in comparison with the MAGEA4 TCR, the interferon gamma production of VHH-TCR with VHH embedded in TRB is lower, but slightly greater when VHH was added to the TRA separated by a mu linker, and ˜3-fold greater when the VHH was added to the TRA separated by a mu linker+G4S. Live-cell imaging by IncuCyte was used to analyze tumor cell growth of the adherent A549.MAGEA4.HLA-A cell line that was stably transduced with a red reporter. The A549 cells grew normally in the presence of UTD T cells and CD33-DARIC cells. Co-culture with TCRs with VHH embedded in TRB had incomplete elimination of tumor cells. Co-culture with MAGEA4 TCR resulted in complete elimination of tumor cells and co-culture with VHH embedded in TRA resulted in complete and more rapid elimination of tumor cells (FIG. 8C).

### Example 3

### Further Evaluation of Linkers in Engineered TCRs

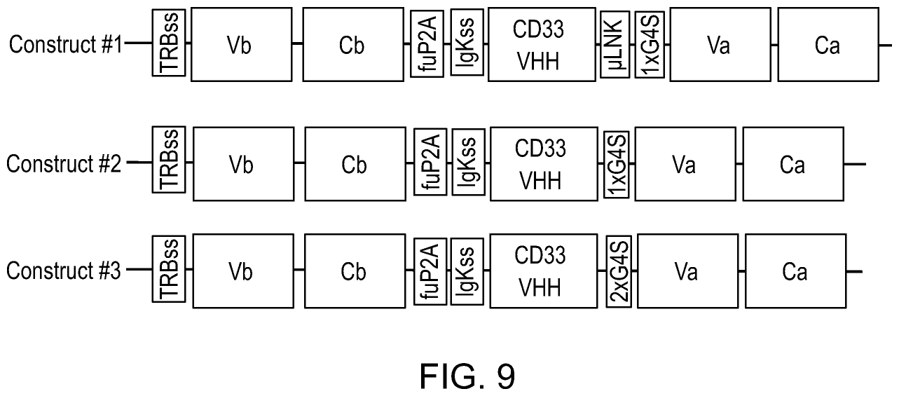

The significance of the linkers on the VHH was further assessed by comparing constructs where 1) a VHH was added to the TRA separated by a mu linker (LEKT)+G4S (SEQ ID NO: 94), 2) a VHH was added to the TRA separated by a 1×G4S (SEQ ID NO: 95), and 3) a VHH was added to the TRA separated by a 2×G4S (SEQ ID NO: 96) (FIG. 9). TCR T cells were produced as described in Example 1.

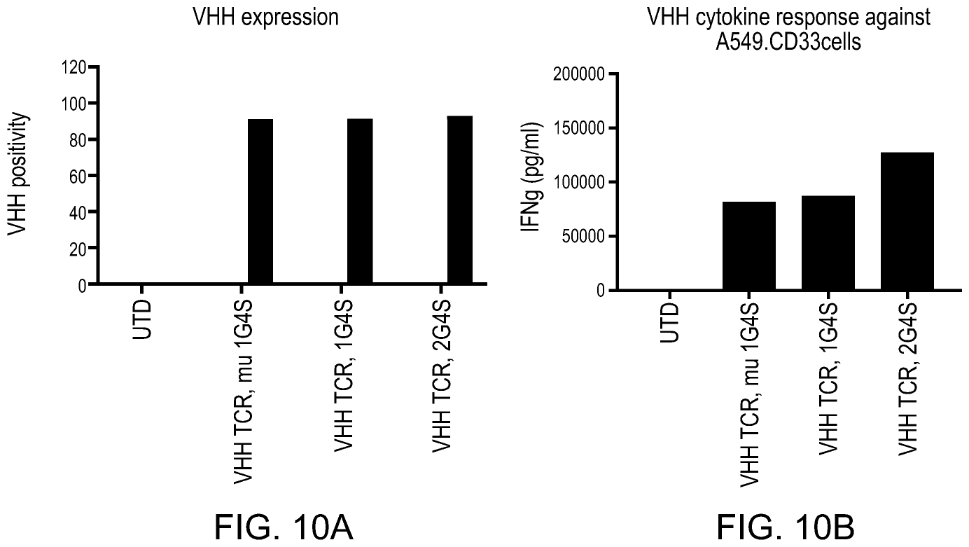

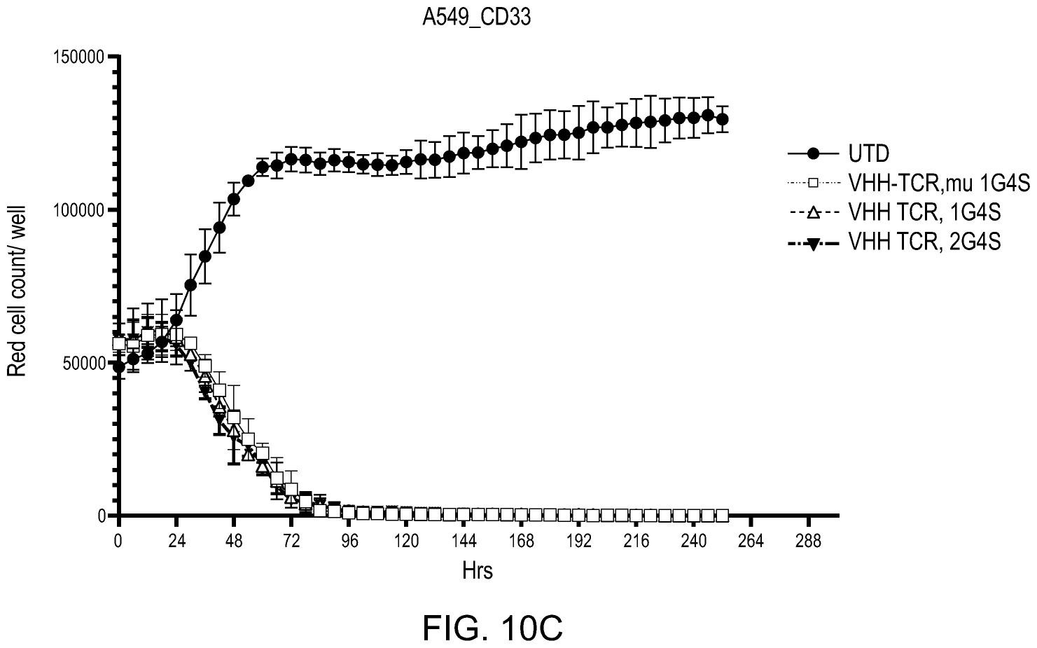

T cells were interrogated for cell surface CD33 expression using flow cytometry. T cells were stained using a His labeled CD33-Fc reagent (Acros) and secondary staining was performed with APC labeled streptavidin. Surface CD33 expression was comparable in all the three assessed formats (FIG. 10A). Additionally, the biological activity of the T cells was assessed for interferon gamma production in co-culture with tumor cell lines positive for CD33 (adherent A549 cell line that was stably transduced with CD33). As shown in FIG. 10B, the interferon gamma production of VHH-TCRs with mu linker+1G4S and 1G4S was comparable and was highest in VHH-TCR with 2G4S. Live-cell imaging by IncuCyte was used to analyze tumor cell growth of A549.CD33 stably transduced with red reporter. The A549 cells grew normally in the presence of UTD T cells. Co-culture with all VHH-TCRs resulted in tumor cell elimination (FIG. 10C).

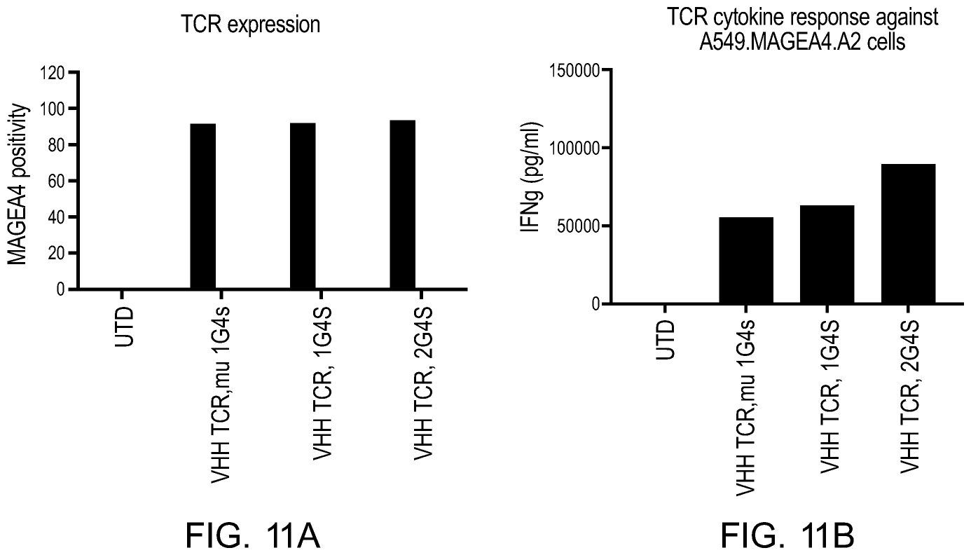

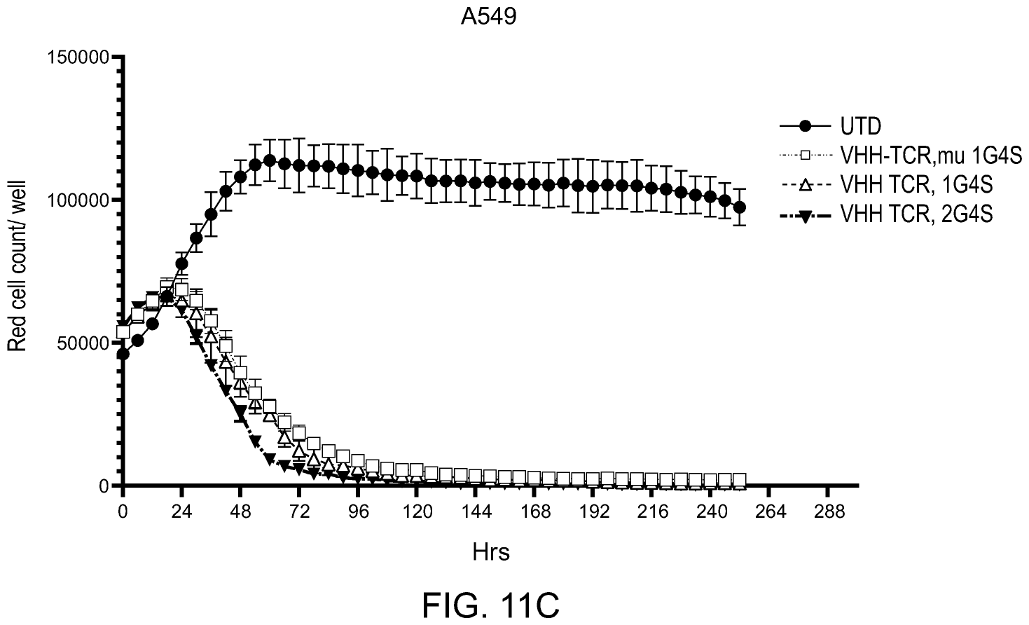

Flow cytometry was performed to evaluate MAGEA4 tetramer/HLA-multimer binding which was comparable in all three assessed formats (FIG. 11A). Additionally, the biological activity of the T cells was assessed for interferon gamma production in co-culture with tumor cell lines positive for MAGEA4 (adherent A549 cell line that was stably transduced with MAGEA4 and HLA-A2). As shown in FIG. 11B, the interferon gamma production of VHH-TCRs with mu linker+1G4S and one G4S was comparable and highest in the VHH-TCR with two G4Ss. Live-cell imaging by IncuCyte was used to analyze tumor cell growth of the adherent A549.MAGEA4.HLA-A cell line that was stably transduced with a red reporter. The A549 cells grew normally in the presence of UTD T cells. Co-culture with all VHH-TCRs had complete elimination of tumor cells and it was fastest in VHH-TCR with two G4Ss (FIG. 11C).

### Example 4

### Evaluation of Engineered Multi-Targeting TCRδ with Tandem Binders

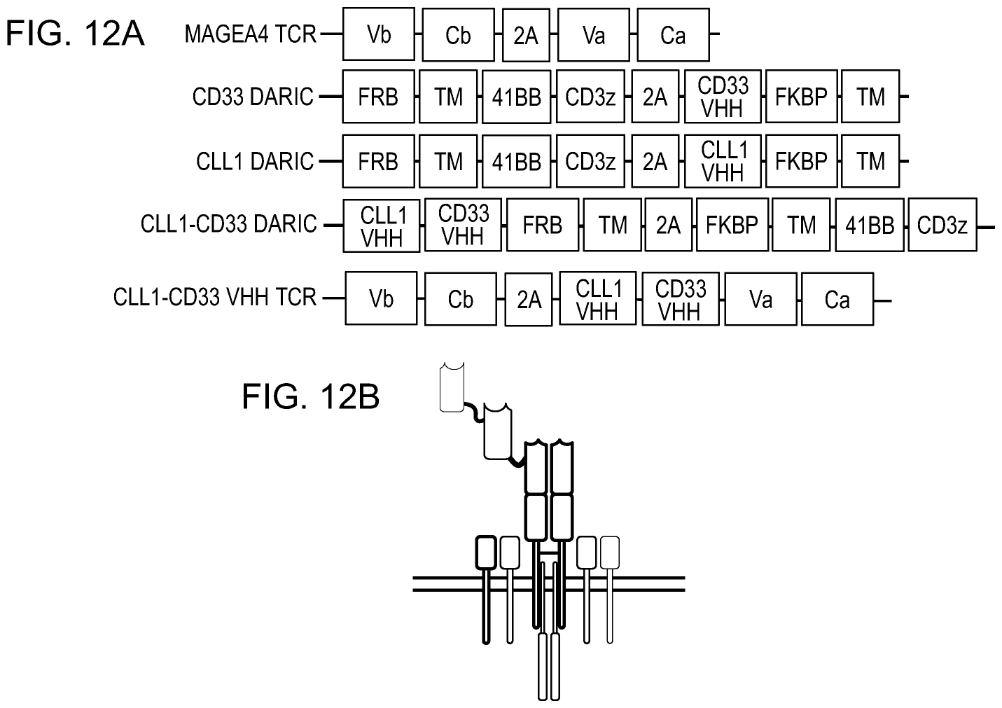

A MAGEA4-reactive, HLA-A2-restricted T-cell receptor (TCR) was embedded with tandem VHHs targeting human CD33 and CLL1 (SEQ ID NO: 97) (FIGS. 12A and 12B). This was evaluated for expression and function compared to a known TCR targeting MAGEA4 and a DARIC (Dimerizing Agent Regulated Immunoreceptor Complex, a controllable and adaptable antigen recognizing system) targeting human CD33 (SEQ ID NO: 90), CLL1 (SEQ ID NO: 98), and both CD33 and CLL1 in tandem (SEQ ID NO: 99) (the “comparators”) (FIG. 12A). Dual targeting TCR T cells were produced in a 10 Day process using G-REX® flasks using the same protocol as Example 1.

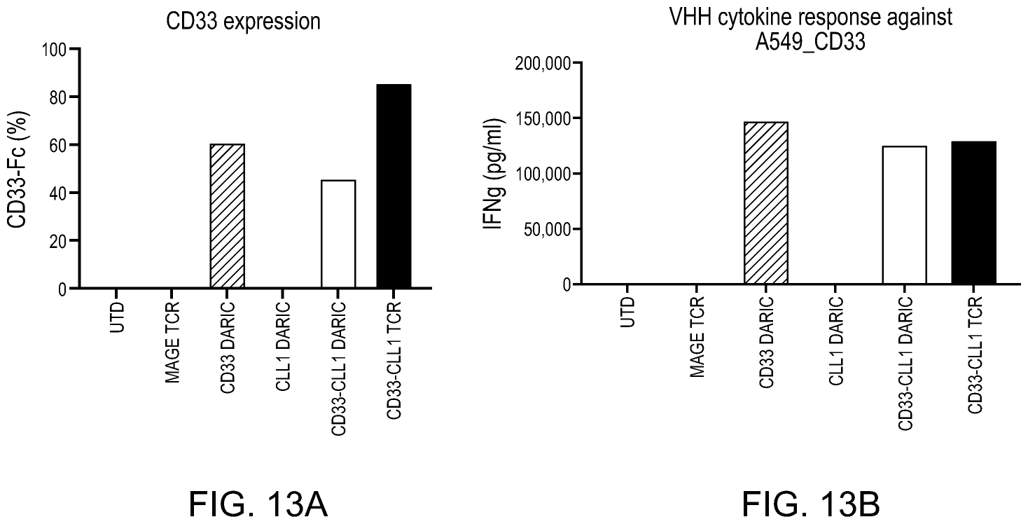

T cells were interrogated for cell surface CD33 expression using flow cytometry. T cells were stained using a His labeled CD33-Fc reagent (Acros) and secondary staining with APC labeled streptavidin. Surface CD33 expression was higher in the CD33-CLL1-TCR compared to CD33 DARIC and CD33-CLL1 DARIC (FIG. 13A). Additionally, the biological activity of the T cells was assessed for interferon gamma production in co-culture with tumor cell lines positive for CD33 (adherent A549 cell line that was stably transduced with CD33). As shown in FIG. 13B, the interferon gamma production of the CD33-CLL1-TCR is comparable to CD33-DARIC and CD33-CLL1 DARIC (the latter two were activated by addition of 1 nm Rapamycin).

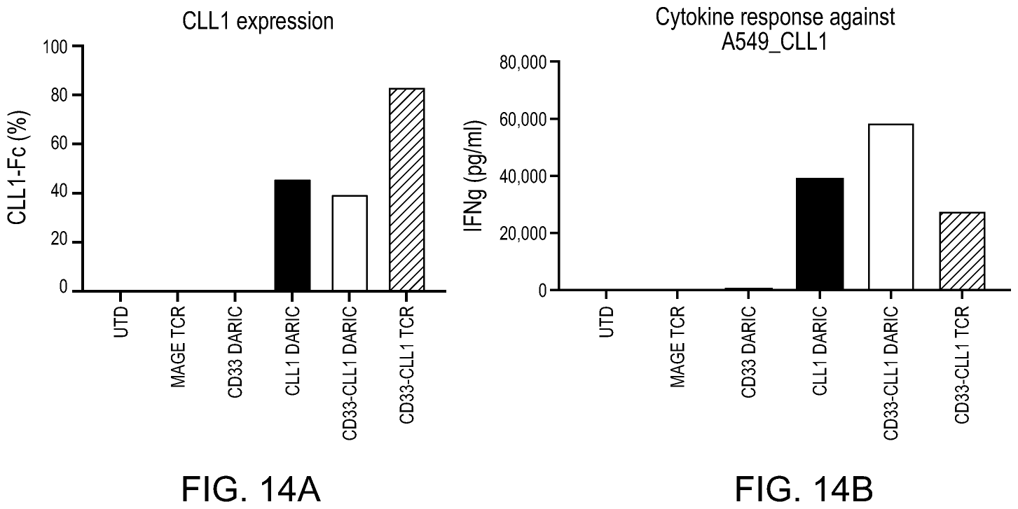

T cells were interrogated for cell surface CLL1 expression using flow cytometry. T cells were stained using a PE labeled CLL1-Fc reagent (Creative Biomart). Surface CLL1 expression was higher in the CD33-CLL1-TCR compared to CLL1 DARIC and CD33-CLL1 DARIC (FIG. 14A). Additionally, the biological activity of the T cells was assessed for interferon gamma production in co-culture with tumor cell lines positive for CLL1 (adherent A549 cell line that was stably transduced with CLL1). As shown in FIG. 14B, the CD33-CLL1 TCR produces robust interferon gamma in co-culture with a CLL1 expressing cell line.

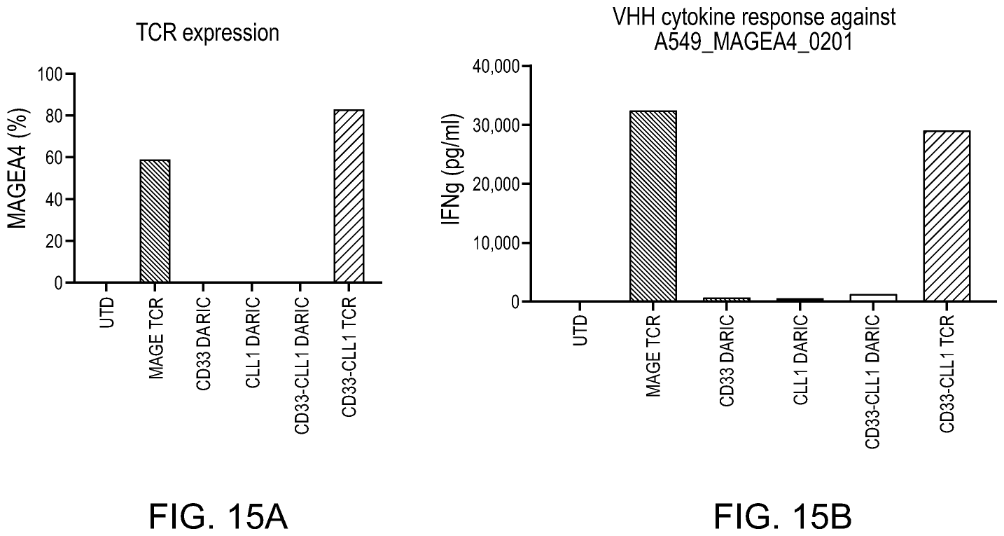

Flow cytometry was performed to evaluate MAGEA4 tetramer/HLA-multimer binding which was higher in CD33-CLL1-TCR compared to the MAGEA4 TCR (FIG. 15A). Additionally, the biological activity of the T cells was assessed for interferon gamma production in co-culture with tumor cell lines positive for MAGEA4 (adherent A549 cell line that was stably transduced with MAGEA4 and HLA-A2). As shown in FIG. 15B, the interferon gamma production of CD33-CLL1-TCR is comparable to the MAGEA4 TCR.

### Example 5

### Evaluation of VHH-Based Engineered TCRs

Two engineered TCRs were constructed, each with a MAGEA4-reactive, HLA-A2-restricted T-cell receptor (TCR) embedded with one of two anti-BCMA VHH. The same anti-BCMA VHHs were also formatted in a CAR format. These were evaluated for expression and function compared to a TCR targeting MAGEA4 and a known scFv-based CAR targeting BCMA (the “comparators”). T cells were produced in a 10 Day process using G-REX® flasks using the same protocol as Example 1.

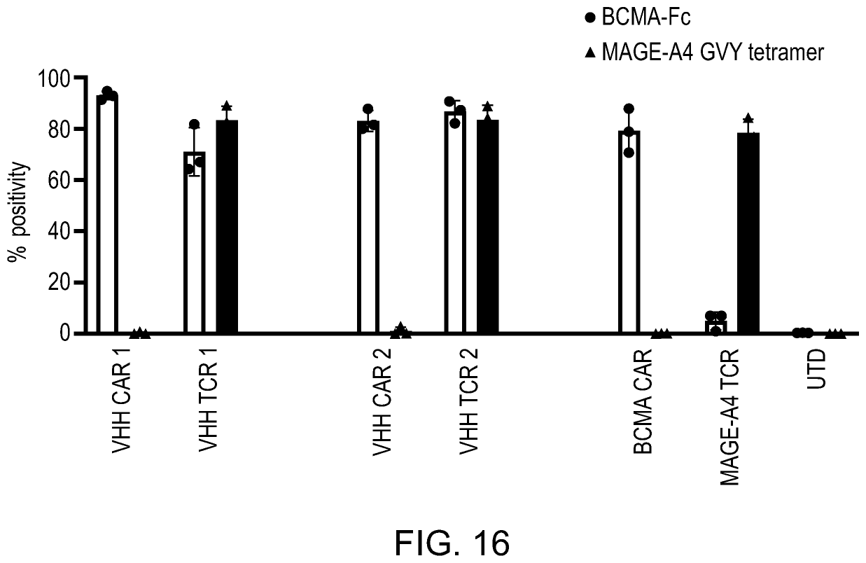

T cells were interrogated for cell surface CAR and TCR expression using flow cytometry and evaluated for MAGEA4 tetramer/HLA-multimer binding. T cells were stained using a PE labeled BCMA Fc reagent (AcroBio). Surface BCMA binder expression was detectable on all constructs having a BCMA binder (FIG. 16). Both VHH TCR were detected robustly by the MAGEA4 tetramer, and were comparable to the MAGE-A4 TCR.

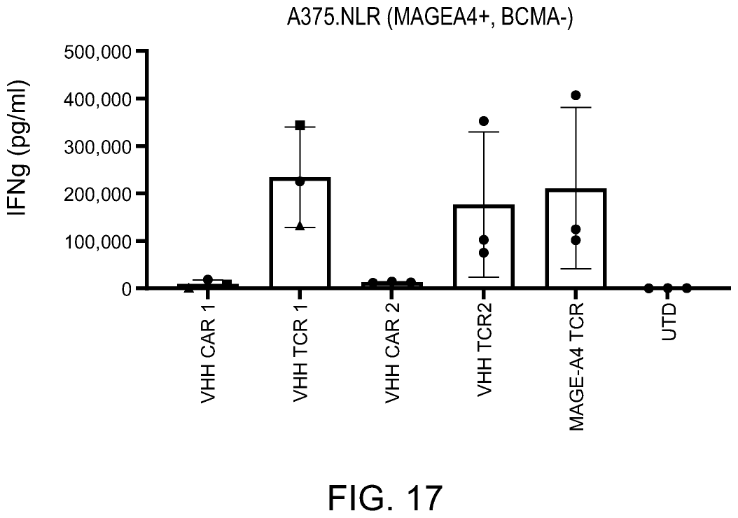

The biological activity of the T cells was assessed for interferon gamma production in co-culture with tumor cell lines positive for MAGEA4 (adherent A375 cell line that endogenously expresses MAGEA4 and HLA-A2). As shown in FIG. 17, the VHH TCRs expressed a very robust level of interferon gamma, and expression is comparable to the MAGEA4 TCR.

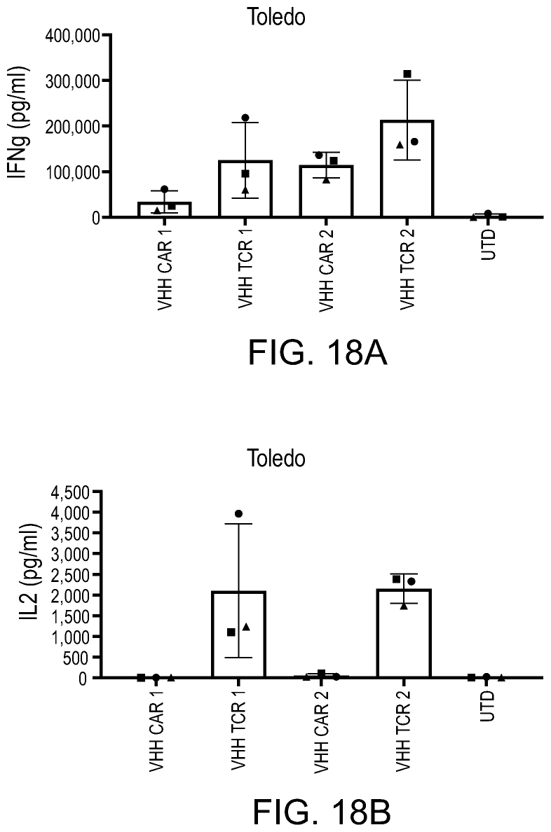

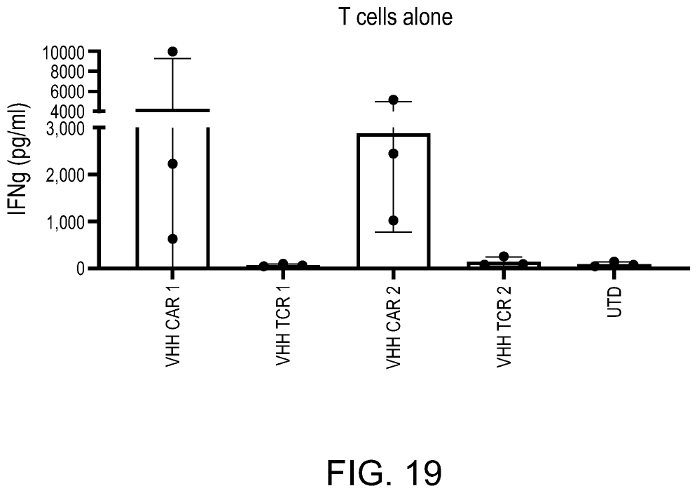

The biological activity of the T cells was also assessed for interferon gamma production in co-culture with tumor cell lines positive for BCMA (the Toledo suspension cell line endogenously expresses low levels of BCMA). As shown in FIG. 18A, the VHH TCRs produced interferon gamma comparable to or greater than the respective VHH CAR. The biological activity of the T cells in co-culture with Toledo cells was further assessed for Interleukin 2 (IL2) production, which is a more sensitive assay. As shown in FIG. 18B, none of the VHH CARs produced a detectable amount of IL2, whereas both VHH TCRs produced robust IL2. Antigen independent signaling of the T cells was assessed by interferon gamma production in co-culture without tumor cell lines. As shown in FIG. 19, the VHH CARs had detectable levels of interferon gamma production, but the VHH TCRs had low or no detectable interferon gamma production in the absence of tumor cells.

### Example 6

### Evaluation of SCFV-Based Engineered TCRs

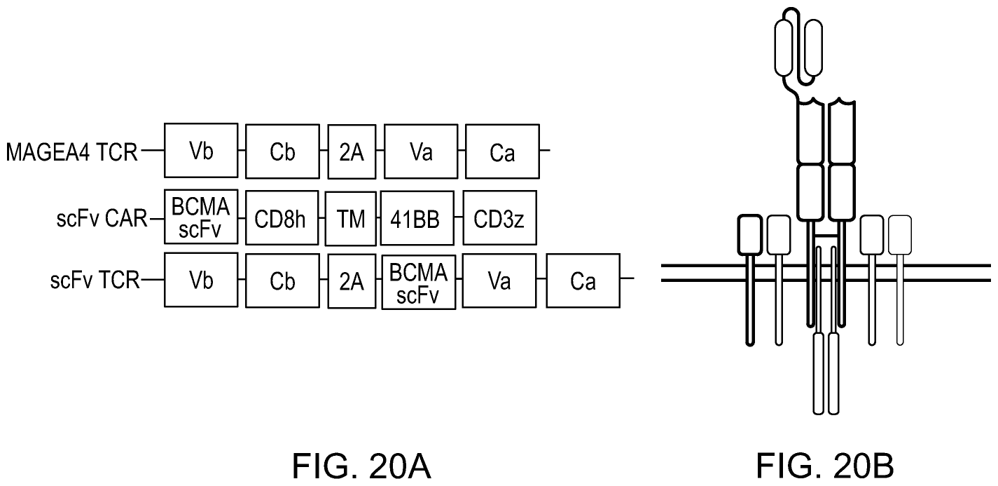

A MAGEA4-reactive, HLA-A2-restricted T-cell receptor (TCR) was embedded with an scFv targeting human BCMA (SEQ ID NO: 100) and a 2×G4S linker between the scFv and Va (FIGS. 20A and 20B). This was evaluated for expression and function compared to a TCR targeting MAGEA4 (SEQ ID NO: 89) and an scFv-based CAR targeting human BCMA (SEQ ID NO: 101) (the “comparators”) (FIG. 20A). Dual targeting TCR T cells were produced in the same manner as Example 1.

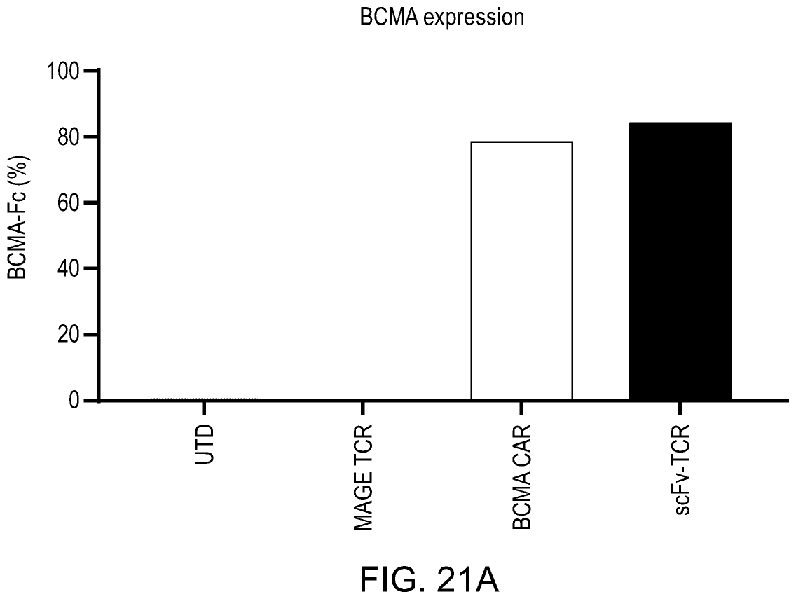

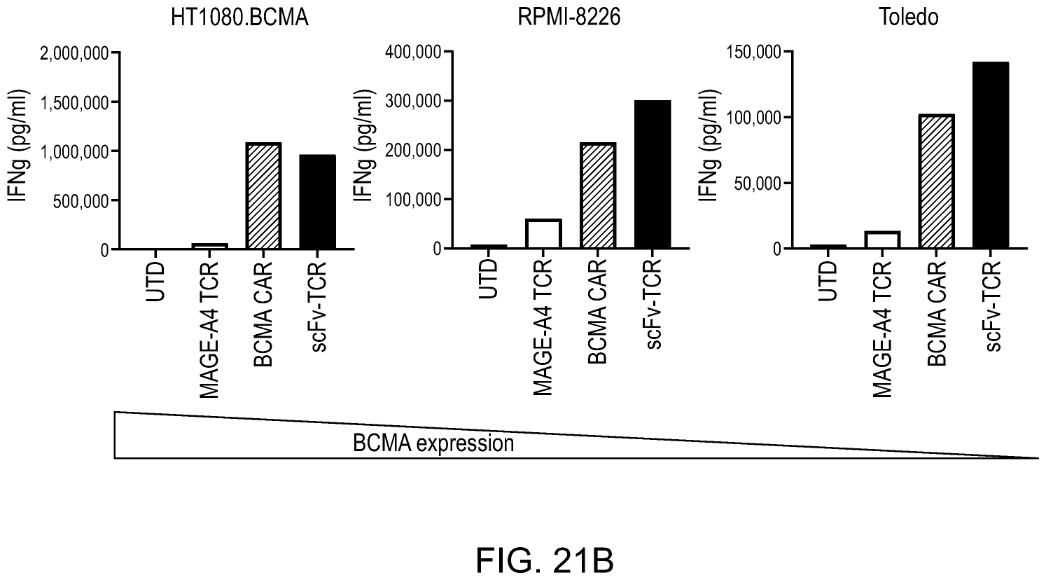

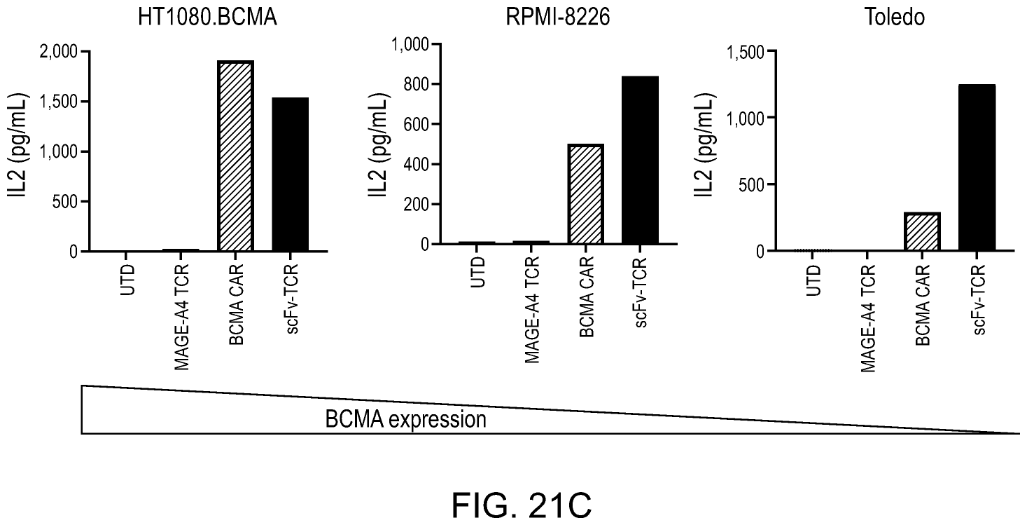

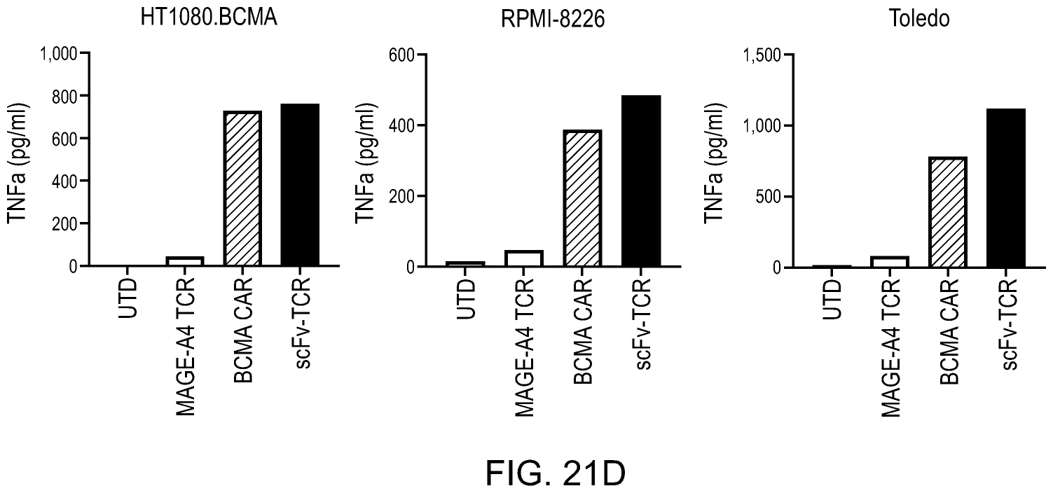

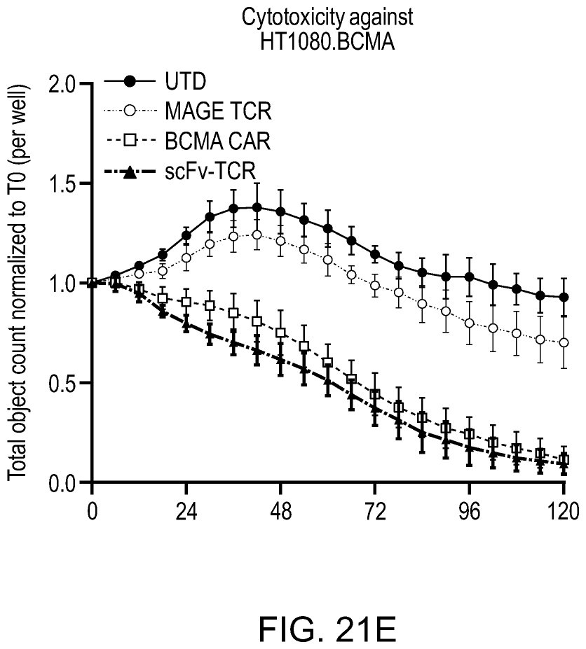

T cells were interrogated for cell surface CAR expression using flow cytometry. T cells were stained using a PE labeled BCMA Fc reagent (AcroBio). Surface BCMA binder expression was comparable between the scFv-TCR and anti-BCMA CAR (FIG. 21A). Additionally, the biological activity of the T cells was assessed for interferon gamma production in co-culture with tumor cell lines expressing varying levels of BCMA (HT1080 engineered to overexpress high levels of BCMA, RPMI-8226: medium endogenous BCMA expression, Toledo: low endogenous expression). As shown in FIG. 21B, the interferon gamma production of scFv-TCR is comparable to the anti-BCMA CAR in high BCMA expressing cell line, but greater in medium and low expressing cell lines. IL2 secretion of scFv-TCR was greater than anti-BCMA CAR in coculture with medium and low BCMA expressing cell lines (FIG. 21C). Secretion of tumor necrosis factor a, another sensitive assay, was assessed in FIG. 21D, and this was greater in RPMI-8226 and Toledo, medium and low expressing BCMA cell lines. For Live-cell imaging by IncuCyte was used to analyze tumor cell growth of HT1080.BCMA stably transduced with red reporter. The HT1080.BCMA cells grew in the presence of UTD T cells and MAGEA4 TCR-T cells. Co-culture with either scFv-TCR or anti-BCMA CAR resulted in tumor cell elimination, with the scFv-TCR achieving elimination more rapidly than the CAR (FIG. 21E).

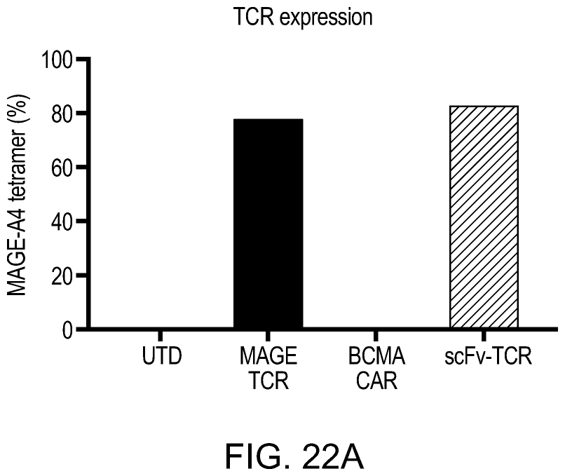

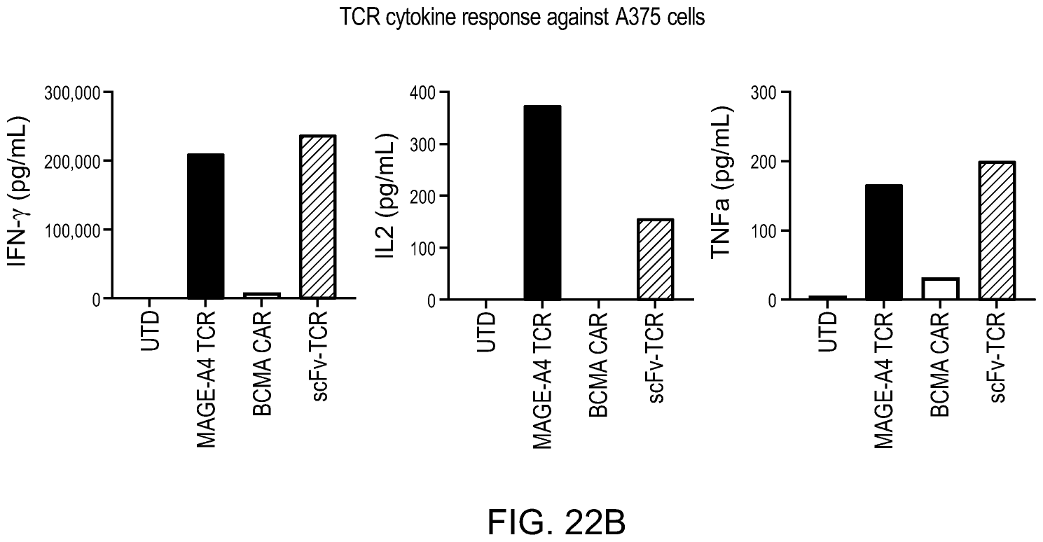

Flow cytometry was performed to evaluate MAGEA4 tetramer/HLA-multimer binding which was comparable in scFv-TCR and the MAGEA4 TCR (FIG. 22A). Additionally, the biological activity of the T cells was assessed for interferon gamma production, IL2 and tumor necrosis factor a in co-culture with tumor cell lines positive for MAGEA4 (adherent A375 cell line that endogenously expresses MAGEA4 and HLA-A2). As shown in FIG. 22B, the interferon gamma and tumor necrosis factor a production of VHH-TCR is comparable to the MAGEA4 TCR.

### Example 7

### Evaluation of Engineered TCRδ in a CD33 Antigen Only Positive Tumor Model

A MAGEA4-reactive, HLA-A2-restricted T-cell receptor (TCR) was embedded with a VHH targeting human CD33 to produce an engineered dual-targeting TCR (“VHH-TCR”) (SEQ ID NO: 93) (FIGS. 1A and 1B). Dual-targeting TCR T cells were produced in the same manner as Example 1. Engineered T cells were evaluated for expression and in vitro function in the same manner as Example 1.

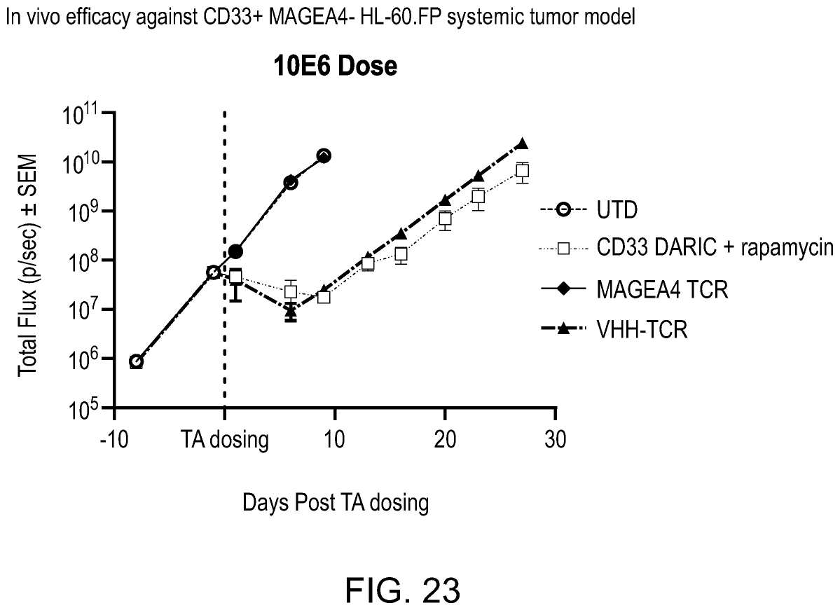

The ability of the VHH-TCR to recognize and function in the presence of VHH antigen was assessed in vivo using the systemic luciferase tagged HL-60 tumor model in NSG mice. The HL-60 model expresses CD33 but not MAGEA4, therefore any observed anti-tumor activity would be a result of the VHH-TCR signaling following VHH recognition of CD33. Luciferase tagged HL-60 cells were transplanted intravenously into naïve female NSG mice and allowed to establish for five days. Mice were randomized into groups of 5 animals with similar means on study day −1 (D−1). On DO, animals were intravenously dosed with either untransduced T cells, MAGE4 TCR T cells, CD33-DARIC T cells, or VHH-TcR T cells. T cell doses were normalized to 10E6 receptor positive cells/mouse; the untransduced T cell dose was normalized to match the highest total T cell dose. Animals treated with CD33-DARIC T cells were maintained on a Monday/Wednesday/Friday 0.1 mg/kg rapamycin schedule starting on DO. As shown in FIG. 23, tumor growth continues unchecked in animals treated with either untransduced or MAGEA4 TCR T cells. Both the CD33-DARIC and VHH-TCR T cells demonstrate comparable tumor control.

### Example 8

### Evaluation of Engineered TCRδ in a TCR Antigen Only Positive Tumor Model

A MAGEA4-reactive, HLA-A2-restricted T-cell receptor (TCR) was embedded with a VHH targeting human CD33 to produce an engineered dual-targeting TCR (“VHH-TCR”) (SEQ ID NO: 93) (FIGS. 1A and 1B). Dual-targeting TCR T cells were produced in the same manner as Example 1. Engineered T cells were evaluated for expression and in vitro function in the same manner as Example 1.

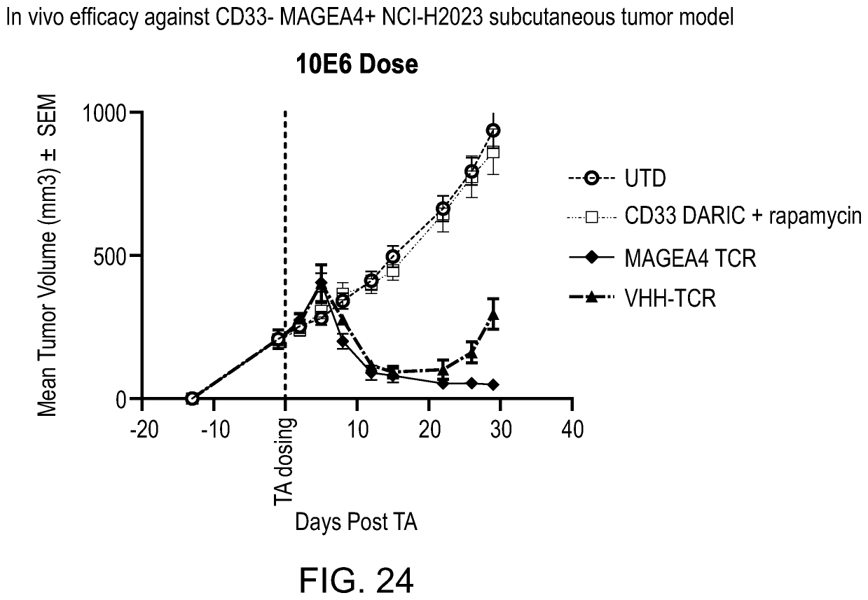

The ability of the VHH-TCR to recognize and function in the presence of TCR antigen was assessed in vivo using the subcutaneous NCI-H2023 tumor model in NSG mice. The NCI-H2023 model expresses MAGEA4 but not CD33, therefore any observed anti-tumor activity would be a result of the VHH-TCR signaling following TCR recognition of MAGEA4. NCI-H2023 cells were transplanted subcutaneously into naïve female NSG mice and allowed to establish for twenty-one days. Mice were randomized into groups of 5 animals with similar means on study day −1 (D−1). On DO, animals were intravenously dosed with either untransduced T cells, MAGE4 TCR T cells, CD33-DARIC T cells, or VHH-TCR T cells. T cell doses were normalized to 10E6 receptor positive cells/mouse; the untransduced T cell dose was normalized to match the highest total T cell dose. Animals treated with CD33-DARIC T cells were maintained on a Monday/Wednesday/Friday 0.1 mg/kg rapamycin schedule starting on DO. As shown in FIG. 24, tumor growth continues unchecked in animals treated with either untransduced or CD33-DARIC T cells. Both the MAGEA4 TCR and VHH-TCR T cells initially demonstrate comparable tumor control. Loss of tumor control occurs earlier in the animals treated with the VHH-TCR T cells.

### Example 9

### Evaluation of Engineered TCR Constructs Comprising a CD19 scFv

A MAGEA4-reactive, HLA-A2-restricted T-cell receptor (TCR) was embedded with an scFv targeting human CD19 (SEQ ID NO: 102). This was evaluated for expression and function compared to a MAGEA4-reactive, HLA-A2-restricted T-cell receptor (TCR) embedded with an scFv targeting human BCMA (SEQ ID NO: 100). Dual-targeting TCR T cells were produced as described in Example 1.

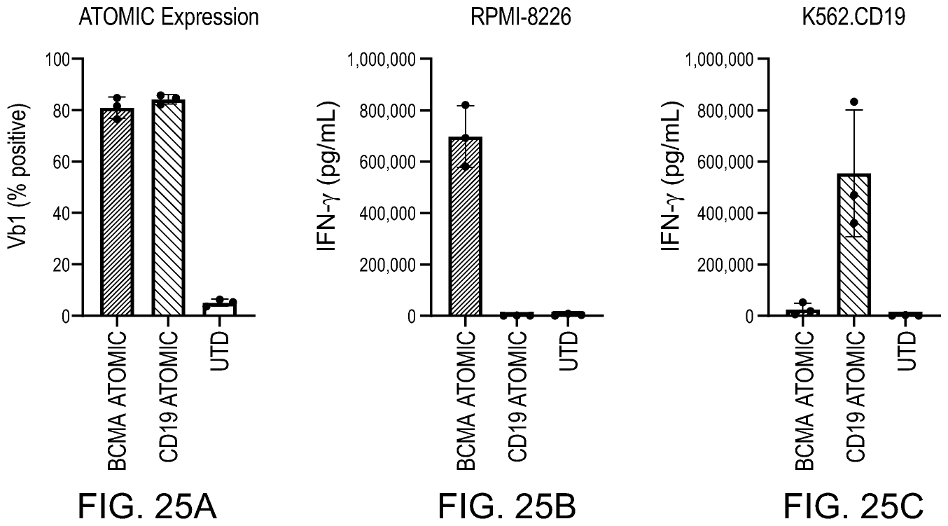

T cells were interrogated for cell surface TCR expression using flow cytometry. T cells were stained using a PE-labeled anti-TCR Vb1 antibody (Miltenyi Biotech). Surface expression of the engineered constructs was comparable (FIG. 25A). Additionally, the biological activity of the T cells was assessed by measuring interferon gamma production in co-cultures with suspension tumor cell line RPMI-8226 (endogenous BCMA expression, undetectable CD19 expression), and with suspension tumor cell line K562.CD19 (undetectable BCMA expression, stably transduced with CD19). As shown in FIG. 25B and FIG. 25C, CD19 ScFv TCR T cells produce interferon gamma in response to tumor cell lines positive for surface CD19 at comparable levels to the interferon gamma produced by BCMA ScFv TCR T cells in response to tumor cell lines positive for surface BCMA.

### Example 10

### Illustrative Engineered TCR Constructs

As contemplated herein, antigen-binding domains (also referred to herein as “binders” or “antigen binders”), polypeptide linkers, and TCRs can be surprisingly combined to produce an engineered TCR having multi-specificity. In other words, the components can be combined without destroying the functionality of either the antigen-binding domain(s) or the TCR(s). Thus, the engineered TCRs contemplated herein surprisingly provide (1) multi-specificity, (2) increased sensitivity to non-MHC presented targets, and (3) the ability to simultaneously target both intracellular and extracellular targets.

Engineered TCRs can be constructed in multiple formats, and can be designed and constructed using known components (e.g., antigen-binding domains, polypeptide linkers, and TCRα and TCRβ chains) and techniques. For example, one or more antigen-binding domains (e.g., one or more “A” components) can be linked to one or more TCR components (e.g., one or more “C” components) with or without one or more polypeptide linkers (e.g., with or without one or more “B” components) using standard cloning techniques. The “A” component can be linked to either the TCRα or TCRβ polypeptide/chain or both; or the TCRγ or TCRδ or both; of the “C” component. Illustrative general engineered TCR formulas are provided below:

A-C

A-B-C

The engineered TCRs contemplated herein can be designed and constructed using known components (e.g., TCRα and TCRβ chains, linkers, and antigen-binding domains) and techniques. Table 3 provides an illustrative list of known antigen-binding domains. Table 4 provides an illustrative list of known polypeptide linkers. Table 5 provides an illustrative list of known TCRs. However, other known antigen-binding domains, linkers, and TCRs can be found throughout the literature, e.g., including but not limited to US20120082661, WO2016014789, WO2022046730, WO2016033570, U.S. Pat. No. 8,147,832B2, WO2014026054, WO2018145649, WO2014065961, WO2020123947, WO2013049254, WO2019241685, WO2019241688, WO2016049214, WO2018236870, WO2020102240, WO2018183888, U.S. Pat. No. 6,217,866B1, WO2008119566, WO2003055917, WO2018073680, WO2014146672, WO2019200007, WO2016016859, WO2018119279, WO2020227072, WO2020227073, WO2020227071, WO2017153402, WO2007042289, WO2018028647, WO2005113595, US20180273602, WO2019067242, WO2020193767, U.S. Ser. No. 10/538,572B2, U.S. Ser. No. 11/078,252B2, WO2019140100, WO2015009606, WO2021195503, WO2007131092, US20190169260 each of which are incorporated by reference herein, in their entirety. Since other known antigen-binding domains, linkers, and TCRs are well known in the literature, the invention is not intended to be limited to the illustrative components disclosed in Tables 3-5.

TABLE 3
Illustrative Antigen-Binding Domains (“A” Components):

| COMPONENT | | | |
|---|---|---|---|
| REFERENCE | TARGET | BINDER TYPE | SEQ ID NOs: |
| A1 | BCMA | scFv | 1, 2, AND/OR 3 |
| A2 | CD19 | scFv | 4 |
| A3 | CD20 | scFv | 5, 6, AND/OR 7 |
| A4 | CD22 | scFv | 8 |
| A5 | CD33 | scFv | 9 |
| A6 | CD79A | scFv | 10 |
| A7 | CD79B | scFv | 11 AND/OR 12 |
| A8 | B7H3 | scFv | 13 |
| A9 | Muc16 | scFv | 14 |
| A10 | HER2 | scFv | 15 |
| A11 | EGFR | scFv | 16 |
| A12 | FN-EDB | scFv | 17 |
| A13 | CLDN18.2 | scFv | 18 |
| A14 | DLL3 | scFv | 19 |
| A15 | FLT3 | scFv | 20 AND/OR 21 |
| A16 | CD33 | VHH | 22, 23, AND/OR 24 |
| A17 | CLL1 | VHH | 25 AND/OR 26 |
| A18 | CD123 | VHH | 27 AND/OR 28 |
| A19 | CD20 | VHH | 29 |
| A20 | EGFR | VHH | 30 |
| A21 | BCMA | VHH | 31 AND/OR 32 |

TABLE 4
Illustrative Polypeptide Linkers (“B” Components):

| COMPONENT | | |
|---|---|---|
| REFERENCE | SEQUENCE | SEQ ID NO: |
| B1 | LEKT | 33 |
| B2 | LEKTGGGGS | 34 |
| B3 | GGGGS | 35 |
| B4 | GGGGSGGGGS | 36 |
| B5 | GGGGSGGGGSGGGGS | 37 |
| B6 | GGGGSGGGGSGGGGSGGGGS | 38 |
| B7 | GGGGSGGGGSGGGGSGGGGSGGGGS | 39 |
| B8 | DGGGS | 40 |
| B9 | TGEKP | 41 |
| B10 | GGRR | 42 |
| B11 | EGKSSGSGSESKVD | 43 |
| B12 | KESGSVSSEQLAQFRSLD | 44 |
| B13 | GGRRGGGS | 45 |
| B14 | LRQRDGERP | 46 |
| B15 | LRQKDGGGSERP | 47 |
| B16 | LRQKDGGGSGGGSERP | 48 |
| B17 | GSTSGSGKPGSGEGSTKG | 49 |
| B18 | GSTSGSGKSSEGSGSTKG | 50 |
| B19 | GSTSGSGKSSEGKG | 51 |
| B20 | GSTSGSGKPGSGEGS | 52 |
| B21 | GGGS | 53 |

TABLE 5
Illustrative TCRs (“C” Components):

| COMPONENT | | | |
|---|---|---|---|
| REFERENCE | TARGET | TCRα CHAIN SEQ ID NO: | TCRβ CHAIN SEQ ID NO: |
| C1 | NY-ESO-1 | 54 | 55 |
| C2 | NY-ESO-1 | 56 | 57 |
| C3 | PRAME | 58 | 59 |
| C4 | TP53R175H | 60 | 61 |
| C5 | MAGE-A4 | 62 | 63 |
| C6 | WT1 | 64 | 65 |
| C7 | MR1 | 66 | 67 |
| C8 | MR1 | 68 | 69 |
| C9 | CD1d + aGalCer | 70 | 71 |
| C10 | HPV16E7 | 72 | 73 |
| C11 | GP100 | 74 | 75 |
| C12 | MART-1 | 76 | 77 |
| | | TCRγ Chain SEQ ID NO: | TCRδ Chain SEQ ID NO: |
| C13 | allo-HLA | 78 | 79 |

As one example of an engineered TCR contemplated herein, an antigen-binding domain from Table 3 (e.g., an antigen-binding domain selected from Component A1) can be combined with one or more polypeptide linkers from Table 4 (e.g., Component B1) and one or both TCR variable domains of a TCR from Table 5 (e.g., Component C1), to produce a novel engineered TCR construct (e.g., ATOMIC construct #1; see below). Additionally, as further shown and contemplated herein, multiple “A” components can be combined to produce multi-specific antigen-binding domains/regions (e.g., tandem antigen-binding domains), and multiple polypeptide linkers can be combined to produce functional linkers.

Table 6 provides an illustrative, non-limiting list of engineered TCRs (i.e., ATOMIC constructs) based on the antigen-binding domains, linkers, and TCRs provided in Tables 3, 4, and 5. One of skill in the art would understand that other combinations are possible, including combinations using other antigen-binding domains, linkers, and TCRs either known to or newly developed by the skilled artisan.

TABLE 6
Illustrative Engineered TCRs (i.e., ATOMICs):

| ATOMIC | | | |
|---|---|---|---|
| CONSTRUCT # | “A” COMPONENT | “B” COMPONENT | “C” COMPONENT |
| 1 | A1 | none or B1 | C1 |
| 2 | A2 | none or B1 | C1 |
| 3 | A3 | none or B1 | C1 |
| 4 | A4 | none or B1 | C1 |
| 5 | A5 | none or B1 | C1 |
| 6 | A6 | none or B1 | C1 |
| 7 | A7 | none or B1 | C1 |
| 8 | A8 | none or B1 | C1 |
| 9 | A9 | none or B1 | C1 |
| 10 | A10 | none or B1 | C1 |
| 11 | A11 | none or B1 | C1 |
| 12 | A12 | none or B1 | C1 |
| 13 | A13 | none or B1 | C1 |
| 14 | A14 | none or B1 | C1 |
| 15 | A15 | none or B1 | C1 |
| 16 | A16 | none or B1 | C1 |
| 17 | A17 | none or B1 | C1 |
| 18 | A18 | none or B1 | C1 |
| 19 | A19 | none or B1 | C1 |
| 20 | A20 | none or B1 | C1 |
| 21 | A21 | none or B1 | C1 |
| 22 | A1 | B2 | C1 |
| 23 | A2 | B2 | C1 |
| 24 | A3 | B2 | C1 |
| 25 | A4 | B2 | C1 |
| 26 | A5 | B2 | C1 |
| 27 | A6 | B2 | C1 |
| 28 | A7 | B2 | C1 |
| 29 | A8 | B2 | C1 |
| 30 | A9 | B2 | C1 |
| 31 | A10 | B2 | C1 |
| 32 | A11 | B2 | C1 |
| 33 | A12 | B2 | C1 |
| 34 | A13 | B2 | C1 |
| 35 | A14 | B2 | C1 |
| 36 | A15 | B2 | C1 |
| 37 | A16 | B2 | C1 |
| 38 | A17 | B2 | C1 |
| 39 | A18 | B2 | C1 |
| 40 | A19 | B2 | C1 |
| 41 | A20 | B2 | C1 |
| 42 | A21 | B2 | C1 |
| 43 | A1 | B3 | C1 |
| 44 | A2 | B3 | C1 |
| 45 | A3 | B3 | C1 |
| 46 | A4 | B3 | C1 |
| 47 | A5 | B3 | C1 |
| 48 | A6 | B3 | C1 |
| 49 | A7 | B3 | C1 |
| 50 | A8 | B3 | C1 |
| 51 | A9 | B3 | C1 |
| 52 | A10 | B3 | C1 |
| 53 | A11 | B3 | C1 |
| 54 | A12 | B3 | C1 |
| 55 | A13 | B3 | C1 |
| 56 | A14 | B3 | C1 |
| 57 | A15 | B3 | C1 |
| 58 | A16 | B3 | C1 |
| 59 | A17 | B3 | C1 |
| 60 | A18 | B3 | C1 |
| 61 | A19 | B3 | C1 |
| 62 | A20 | B3 | C1 |
| 63 | A21 | B3 | C1 |
| 64 | A1 | B4 | C1 |
| 65 | A2 | B4 | C1 |
| 66 | A3 | B4 | C1 |
| 67 | A4 | B4 | C1 |
| 68 | A5 | B4 | C1 |
| 69 | A6 | B4 | C1 |
| 70 | A7 | B4 | C1 |
| 71 | A8 | B4 | C1 |
| 72 | A9 | B4 | C1 |
| 73 | A10 | B4 | C1 |
| 74 | A11 | B4 | C1 |
| 75 | A12 | B4 | C1 |
| 76 | A13 | B4 | C1 |
| 77 | A14 | B4 | C1 |
| 78 | A15 | B4 | C1 |
| 79 | A16 | B4 | C1 |
| 80 | A17 | B4 | C1 |
| 81 | A18 | B4 | C1 |
| 82 | A19 | B4 | C1 |
| 83 | A20 | B4 | C1 |
| 84 | A21 | B4 | C1 |
| 85 | A1 | Any one of B5 through B21 | C1 |
| 86 | A2 | Any one of B5 through B21 | C1 |
| 87 | A3 | Any one of B5 through B21 | C1 |
| 88 | A4 | Any one of B5 through B21 | C1 |
| 89 | A5 | Any one of B5 through B21 | C1 |
| 90 | A6 | Any one of B5 through B21 | C1 |
| 91 | A7 | Any one of B5 through B21 | C1 |
| 92 | A8 | Any one of B5 through B21 | C1 |
| 93 | A9 | Any one of B5 through B21 | C1 |
| 94 | A10 | Any one of B5 through B21 | C1 |
| 95 | A11 | Any one of B5 through B21 | C1 |
| 96 | A12 | Any one of B5 through B21 | C1 |
| 97 | A13 | Any one of B5 through B21 | C1 |
| 98 | A14 | Any one of B5 through B21 | C1 |
| 99 | A15 | Any one of B5 through B21 | C1 |
| 100 | A16 | Any one of B5 through B21 | C1 |
| 101 | A17 | Any one of B5 through B21 | C1 |
| 102 | A18 | Any one of B5 through B21 | C1 |
| 103 | A19 | Any one of B5 through B21 | C1 |
| 104 | A20 | Any one of B5 through B21 | C1 |
| 105 | A21 | Any one of B5 through B21 | C1 |
| 106 | A1 | none or B1 | C2 |
| 107 | A2 | none or B1 | C2 |
| 108 | A3 | none or B1 | C2 |
| 109 | A4 | none or B1 | C2 |
| 110 | A5 | none or B1 | C2 |
| 111 | A6 | none or B1 | C2 |
| 112 | A7 | none or B1 | C2 |
| 113 | A8 | none or B1 | C2 |
| 114 | A9 | none or B1 | C2 |
| 115 | A10 | none or B1 | C2 |
| 116 | A11 | none or B1 | C2 |
| 117 | A12 | none or B1 | C2 |
| 118 | A13 | none or B1 | C2 |
| 119 | A14 | none or B1 | C2 |
| 120 | A15 | none or B1 | C2 |
| 121 | A16 | none or B1 | C2 |
| 122 | A17 | none or B1 | C2 |
| 123 | A18 | none or B1 | C2 |
| 124 | A19 | none or B1 | C2 |
| 125 | A20 | none or B1 | C2 |
| 126 | A21 | none or B1 | C2 |
| 127 | A1 | B2 | C2 |
| 128 | A2 | B2 | C2 |
| 129 | A3 | B2 | C2 |
| 130 | A4 | B2 | C2 |
| 131 | A5 | B2 | C2 |
| 132 | A6 | B2 | C2 |
| 133 | A7 | B2 | C2 |
| 134 | A8 | B2 | C2 |
| 135 | A9 | B2 | C2 |
| 136 | A10 | B2 | C2 |
| 137 | A11 | B2 | C2 |
| 138 | A12 | B2 | C2 |
| 139 | A13 | B2 | C2 |
| 140 | A14 | B2 | C2 |
| 141 | A15 | B2 | C2 |
| 142 | A16 | B2 | C2 |
| 143 | A17 | B2 | C2 |
| 144 | A18 | B2 | C2 |
| 145 | A19 | B2 | C2 |
| 146 | A20 | B2 | C2 |
| 147 | A21 | B2 | C2 |
| 148 | A1 | B3 | C2 |
| 149 | A2 | B3 | C2 |
| 150 | A3 | B3 | C2 |
| 151 | A4 | B3 | C2 |
| 152 | A5 | B3 | C2 |
| 153 | A6 | B3 | C2 |
| 154 | A7 | B3 | C2 |
| 155 | A8 | B3 | C2 |
| 156 | A9 | B3 | C2 |
| 157 | A10 | B3 | C2 |
| 158 | A11 | B3 | C2 |
| 159 | A12 | B3 | C2 |
| 160 | A13 | B3 | C2 |
| 161 | A14 | B3 | C2 |
| 162 | A15 | B3 | C2 |
| 163 | A16 | B3 | C2 |
| 164 | A17 | B3 | C2 |
| 165 | A18 | B3 | C2 |
| 166 | A19 | B3 | C2 |
| 167 | A20 | B3 | C2 |
| 168 | A21 | B3 | C2 |
| 169 | A1 | B4 | C2 |
| 170 | A2 | B4 | C2 |
| 171 | A3 | B4 | C2 |
| 172 | A4 | B4 | C2 |
| 173 | A5 | B4 | C2 |
| 174 | A6 | B4 | C2 |
| 175 | A7 | B4 | C2 |
| 176 | A8 | B4 | C2 |
| 177 | A9 | B4 | C2 |
| 178 | A10 | B4 | C2 |
| 179 | A11 | B4 | C2 |
| 180 | A12 | B4 | C2 |
| 181 | A13 | B4 | C2 |
| 182 | A14 | B4 | C2 |
| 183 | A15 | B4 | C2 |
| 184 | A16 | B4 | C2 |
| 185 | A17 | B4 | C2 |
| 186 | A18 | B4 | C2 |
| 187 | A19 | B4 | C2 |
| 188 | A20 | B4 | C2 |
| 189 | A21 | B4 | C2 |
| 190 | A1 | Any one of B5 through B21 | C2 |
| 191 | A2 | Any one of B5 through B21 | C2 |
| 192 | A3 | Any one of B5 through B21 | C2 |
| 193 | A4 | Any one of B5 through B21 | C2 |
| 194 | A5 | Any one of B5 through B21 | C2 |
| 195 | A6 | Any one of B5 through B21 | C2 |
| 196 | A7 | Any one of B5 through B21 | C2 |
| 197 | A8 | Any one of B5 through B21 | C2 |
| 198 | A9 | Any one of B5 through B21 | C2 |
| 199 | A10 | Any one of B5 through B21 | C2 |
| 200 | A11 | Any one of B5 through B21 | C2 |
| 201 | A12 | Any one of B5 through B21 | C2 |
| 202 | A13 | Any one of B5 through B21 | C2 |
| 203 | A14 | Any one of B5 through B21 | C2 |
| 204 | A15 | Any one of B5 through B21 | C2 |
| 205 | A16 | Any one of B5 through B21 | C2 |
| 206 | A17 | Any one of B5 through B21 | C2 |
| 207 | A18 | Any one of B5 through B21 | C2 |
| 208 | A19 | Any one of B5 through B21 | C2 |
| 209 | A20 | Any one of B5 through B21 | C2 |
| 210 | A21 | Any one of B5 through B21 | C2 |
| 211 | A1 | none or B1 | C3 |
| 212 | A2 | none or B1 | C3 |
| 213 | A3 | none or B1 | C3 |
| 214 | A4 | none or B1 | C3 |
| 215 | A5 | none or B1 | C3 |
| 216 | A6 | none or B1 | C3 |
| 217 | A7 | none or B1 | C3 |
| 218 | A8 | none or B1 | C3 |
| 219 | A9 | none or B1 | C3 |
| 220 | A10 | none or B1 | C3 |
| 221 | A11 | none or B1 | C3 |
| 222 | A12 | none or B1 | C3 |
| 223 | A13 | none or B1 | C3 |
| 224 | A14 | none or B1 | C3 |
| 225 | A15 | none or B1 | C3 |
| 226 | A16 | none or B1 | C3 |
| 227 | A17 | none or B1 | C3 |
| 228 | A18 | none or B1 | C3 |
| 229 | A19 | none or B1 | C3 |
| 230 | A20 | none or B1 | C3 |
| 231 | A21 | none or B1 | C3 |
| 232 | A1 | B2 | C3 |
| 233 | A2 | B2 | C3 |
| 234 | A3 | B2 | C3 |
| 235 | A4 | B2 | C3 |
| 236 | A5 | B2 | C3 |
| 237 | A6 | B2 | C3 |
| 238 | A7 | B2 | C3 |
| 239 | A8 | B2 | C3 |
| 240 | A9 | B2 | C3 |
| 241 | A10 | B2 | C3 |
| 242 | A11 | B2 | C3 |
| 243 | A12 | B2 | C3 |
| 244 | A13 | B2 | C3 |
| 245 | A14 | B2 | C3 |
| 246 | A15 | B2 | C3 |
| 247 | A16 | B2 | C3 |
| 248 | A17 | B2 | C3 |
| 249 | A18 | B2 | C3 |
| 250 | A19 | B2 | C3 |
| 251 | A20 | B2 | C3 |
| 252 | A21 | B2 | C3 |
| 253 | A1 | B3 | C3 |
| 254 | A2 | B3 | C3 |
| 255 | A3 | B3 | C3 |
| 256 | A4 | B3 | C3 |
| 257 | A5 | B3 | C3 |
| 258 | A6 | B3 | C3 |
| 259 | A7 | B3 | C3 |
| 260 | A8 | B3 | C3 |
| 261 | A9 | B3 | C3 |
| 262 | A10 | B3 | C3 |
| 263 | A11 | B3 | C3 |
| 264 | A12 | B3 | C3 |
| 265 | A13 | B3 | C3 |
| 266 | A14 | B3 | C3 |
| 267 | A15 | B3 | C3 |
| 268 | A16 | B3 | C3 |
| 269 | A17 | B3 | C3 |
| 270 | A18 | B3 | C3 |
| 271 | A19 | B3 | C3 |
| 272 | A20 | B3 | C3 |
| 273 | A21 | B3 | C3 |
| 274 | A1 | B4 | C3 |
| 275 | A2 | B4 | C3 |
| 276 | A3 | B4 | C3 |
| 277 | A4 | B4 | C3 |
| 278 | A5 | B4 | C3 |
| 279 | A6 | B4 | C3 |
| 280 | A7 | B4 | C3 |
| 281 | A8 | B4 | C3 |
| 282 | A9 | B4 | C3 |
| 283 | A10 | B4 | C3 |
| 284 | A11 | B4 | C3 |
| 285 | A12 | B4 | C3 |
| 286 | A13 | B4 | C3 |
| 287 | A14 | B4 | C3 |
| 288 | A15 | B4 | C3 |
| 289 | A16 | B4 | C3 |
| 290 | A17 | B4 | C3 |
| 291 | A18 | B4 | C3 |
| 292 | A19 | B4 | C3 |
| 293 | A20 | B4 | C3 |
| 294 | A21 | B4 | C3 |
| 295 | A1 | Any one of B5 through B21 | C3 |
| 296 | A2 | Any one of B5 through B21 | C3 |
| 297 | A3 | Any one of B5 through B21 | C3 |
| 298 | A4 | Any one of B5 through B21 | C3 |
| 299 | A5 | Any one of B5 through B21 | C3 |
| 300 | A6 | Any one of B5 through B21 | C3 |
| 301 | A7 | Any one of B5 through B21 | C3 |
| 302 | A8 | Any one of B5 through B21 | C3 |
| 303 | A9 | Any one of B5 through B21 | C3 |
| 304 | A10 | Any one of B5 through B21 | C3 |
| 305 | A11 | Any one of B5 through B21 | C3 |
| 306 | A12 | Any one of B5 through B21 | C3 |
| 307 | A13 | Any one of B5 through B21 | C3 |
| 308 | A14 | Any one of B5 through B21 | C3 |
| 309 | A15 | Any one of B5 through B21 | C3 |
| 310 | A16 | Any one of B5 through B21 | C3 |
| 311 | A17 | Any one of B5 through B21 | C3 |
| 312 | A18 | Any one of B5 through B21 | C3 |
| 313 | A19 | Any one of B5 through B21 | C3 |
| 314 | A20 | Any one of B5 through B21 | C3 |
| 315 | A21 | Any one of B5 through B21 | C3 |
| 316 | A1 | none or B1 | C4 |
| 317 | A2 | none or B1 | C4 |
| 318 | A3 | none or B1 | C4 |
| 319 | A4 | none or B1 | C4 |
| 320 | A5 | none or B1 | C4 |
| 321 | A6 | none or B1 | C4 |
| 322 | A7 | none or B1 | C4 |
| 323 | A8 | none or B1 | C4 |
| 324 | A9 | none or B1 | C4 |
| 325 | A10 | none or B1 | C4 |
| 326 | A11 | none or B1 | C4 |
| 327 | A12 | none or B1 | C4 |
| 328 | A13 | none or B1 | C4 |
| 329 | A14 | none or B1 | C4 |
| 330 | A15 | none or B1 | C4 |
| 331 | A16 | none or B1 | C4 |
| 332 | A17 | none or B1 | C4 |
| 333 | A18 | none or B1 | C4 |
| 334 | A19 | none or B1 | C4 |
| 335 | A20 | none or B1 | C4 |
| 336 | A21 | none or B1 | C4 |
| 337 | A1 | B2 | C4 |
| 338 | A2 | B2 | C4 |
| 339 | A3 | B2 | C4 |
| 340 | A4 | B2 | C4 |
| 341 | A5 | B2 | C4 |
| 342 | A6 | B2 | C4 |
| 343 | A7 | B2 | C4 |
| 344 | A8 | B2 | C4 |
| 345 | A9 | B2 | C4 |
| 346 | A10 | B2 | C4 |
| 347 | A11 | B2 | C4 |
| 348 | A12 | B2 | C4 |
| 349 | A13 | B2 | C4 |
| 350 | A14 | B2 | C4 |
| 351 | A15 | B2 | C4 |
| 352 | A16 | B2 | C4 |
| 353 | A17 | B2 | C4 |
| 354 | A18 | B2 | C4 |
| 355 | A19 | B2 | C4 |
| 356 | A20 | B2 | C4 |
| 357 | A21 | B2 | C4 |
| 358 | A1 | B3 | C4 |
| 359 | A2 | B3 | C4 |
| 360 | A3 | B3 | C4 |
| 361 | A4 | B3 | C4 |
| 362 | A5 | B3 | C4 |
| 363 | A6 | B3 | C4 |
| 364 | A7 | B3 | C4 |
| 365 | A8 | B3 | C4 |
| 366 | A9 | B3 | C4 |
| 367 | A10 | B3 | C4 |
| 368 | A11 | B3 | C4 |
| 369 | A12 | B3 | C4 |
| 370 | A13 | B3 | C4 |
| 371 | A14 | B3 | C4 |
| 372 | A15 | B3 | C4 |
| 373 | A16 | B3 | C4 |
| 374 | A17 | B3 | C4 |
| 375 | A18 | B3 | C4 |
| 376 | A19 | B3 | C4 |
| 377 | A20 | B3 | C4 |
| 378 | A21 | B3 | C4 |
| 379 | A1 | B4 | C4 |
| 380 | A2 | B4 | C4 |
| 381 | A3 | B4 | C4 |
| 382 | A4 | B4 | C4 |
| 383 | A5 | B4 | C4 |
| 384 | A6 | B4 | C4 |
| 385 | A7 | B4 | C4 |
| 386 | A8 | B4 | C4 |
| 387 | A9 | B4 | C4 |
| 388 | A10 | B4 | C4 |
| 389 | A11 | B4 | C4 |
| 390 | A12 | B4 | C4 |
| 391 | A13 | B4 | C4 |
| 392 | A14 | B4 | C4 |
| 393 | A15 | B4 | C4 |
| 394 | A16 | B4 | C4 |
| 395 | A17 | B4 | C4 |
| 396 | A18 | B4 | C4 |
| 397 | A19 | B4 | C4 |
| 398 | A20 | B4 | C4 |
| 399 | A21 | B4 | C4 |
| 400 | A1 | Any one of B5 through B21 | C4 |
| 401 | A2 | Any one of B5 through B21 | C4 |
| 402 | A3 | Any one of B5 through B21 | C4 |
| 403 | A4 | Any one of B5 through B21 | C4 |
| 404 | A5 | Any one of B5 through B21 | C4 |
| 405 | A6 | Any one of B5 through B21 | C4 |
| 406 | A7 | Any one of B5 through B21 | C4 |
| 407 | A8 | Any one of B5 through B21 | C4 |
| 408 | A9 | Any one of B5 through B21 | C4 |
| 409 | A10 | Any one of B5 through B21 | C4 |
| 410 | A11 | Any one of B5 through B21 | C4 |
| 411 | A12 | Any one of B5 through B21 | C4 |
| 412 | A13 | Any one of B5 through B21 | C4 |
| 413 | A14 | Any one of B5 through B21 | C4 |
| 414 | A15 | Any one of B5 through B21 | C4 |
| 415 | A16 | Any one of B5 through B21 | C4 |
| 416 | A17 | Any one of B5 through B21 | C4 |
| 417 | A18 | Any one of B5 through B21 | C4 |
| 418 | A19 | Any one of B5 through B21 | C4 |
| 419 | A20 | Any one of B5 through B21 | C4 |
| 420 | A21 | Any one of B5 through B21 | C4 |
| 421 | A1 | none or B1 | C5 |
| 422 | A2 | none or B1 | C5 |
| 423 | A3 | none or B1 | C5 |
| 424 | A4 | none or B1 | C5 |
| 425 | A5 | none or B1 | C5 |
| 426 | A6 | none or B1 | C5 |
| 427 | A7 | none or B1 | C5 |
| 428 | A8 | none or B1 | C5 |
| 429 | A9 | none or B1 | C5 |
| 430 | A10 | none or B1 | C5 |
| 431 | A11 | none or B1 | C5 |
| 432 | A12 | none or B1 | C5 |
| 433 | A13 | none or B1 | C5 |
| 434 | A14 | none or B1 | C5 |
| 435 | A15 | none or B1 | C5 |
| 436 | A16 | none or B1 | C5 |
| 437 | A17 | none or B1 | C5 |
| 438 | A18 | none or B1 | C5 |
| 439 | A19 | none or B1 | C5 |
| 440 | A20 | none or B1 | C5 |
| 441 | A21 | none or B1 | C5 |
| 442 | A1 | B2 | C5 |
| 443 | A2 | B2 | C5 |
| 444 | A3 | B2 | C5 |
| 445 | A4 | B2 | C5 |
| 446 | A5 | B2 | C5 |
| 447 | A6 | B2 | C5 |
| 448 | A7 | B2 | C5 |
| 449 | A8 | B2 | C5 |
| 450 | A9 | B2 | C5 |
| 451 | A10 | B2 | C5 |
| 452 | A11 | B2 | C5 |
| 453 | A12 | B2 | C5 |
| 454 | A13 | B2 | C5 |
| 455 | A14 | B2 | C5 |
| 456 | A15 | B2 | C5 |
| 457 | A16 | B2 | C5 |
| 458 | A17 | B2 | C5 |
| 459 | A18 | B2 | C5 |
| 460 | A19 | B2 | C5 |
| 461 | A20 | B2 | C5 |
| 462 | A21 | B2 | C5 |
| 463 | A1 | B3 | C5 |
| 464 | A2 | B3 | C5 |
| 465 | A3 | B3 | C5 |
| 466 | A4 | B3 | C5 |
| 467 | A5 | B3 | C5 |
| 468 | A6 | B3 | C5 |
| 469 | A7 | B3 | C5 |
| 470 | A8 | B3 | C5 |
| 471 | A9 | B3 | C5 |
| 472 | A10 | B3 | C5 |
| 473 | A11 | B3 | C5 |
| 474 | A12 | B3 | C5 |
| 475 | A13 | B3 | C5 |
| 476 | A14 | B3 | C5 |
| 477 | A15 | B3 | C5 |
| 478 | A16 | B3 | C5 |
| 479 | A17 | B3 | C5 |
| 480 | A18 | B3 | C5 |
| 481 | A19 | B3 | C5 |
| 482 | A20 | B3 | C5 |
| 483 | A21 | B3 | C5 |
| 484 | A1 | B4 | C5 |
| 485 | A2 | B4 | C5 |
| 486 | A3 | B4 | C5 |
| 487 | A4 | B4 | C5 |
| 488 | A5 | B4 | C5 |
| 489 | A6 | B4 | C5 |
| 490 | A7 | B4 | C5 |
| 491 | A8 | B4 | C5 |
| 492 | A9 | B4 | C5 |
| 493 | A10 | B4 | C5 |
| 494 | A11 | B4 | C5 |
| 495 | A12 | B4 | C5 |
| 496 | A13 | B4 | C5 |
| 497 | A14 | B4 | C5 |
| 498 | A15 | B4 | C5 |
| 499 | A16 | B4 | C5 |
| 500 | A17 | B4 | C5 |
| 501 | A18 | B4 | C5 |
| 502 | A19 | B4 | C5 |
| 503 | A20 | B4 | C5 |
| 504 | A21 | B4 | C5 |
| 505 | A1 | Any one of B5 through B21 | C5 |
| 506 | A2 | Any one of B5 through B21 | C5 |
| 507 | A3 | Any one of B5 through B21 | C5 |
| 508 | A4 | Any one of B5 through B21 | C5 |
| 509 | A5 | Any one of B5 through B21 | C5 |
| 510 | A6 | Any one of B5 through B21 | C5 |
| 511 | A7 | Any one of B5 through B21 | C5 |
| 512 | A8 | Any one of B5 through B21 | C5 |
| 513 | A9 | Any one of B5 through B21 | C5 |
| 514 | A10 | Any one of B5 through B21 | C5 |
| 515 | A11 | Any one of B5 through B21 | C5 |
| 516 | A12 | Any one of B5 through B21 | C5 |
| 517 | A13 | Any one of B5 through B21 | C5 |
| 518 | A14 | Any one of B5 through B21 | C5 |
| 519 | A15 | Any one of B5 through B21 | C5 |
| 520 | A16 | Any one of B5 through B21 | C5 |
| 521 | A17 | Any one of B5 through B21 | C5 |
| 522 | A18 | Any one of B5 through B21 | C5 |
| 523 | A19 | Any one of B5 through B21 | C5 |
| 524 | A20 | Any one of B5 through B21 | C5 |
| 525 | A21 | Any one of B5 through B21 | C5 |
| 526 | A1 | none or B1 | C6 |
| 527 | A2 | none or B1 | C6 |
| 528 | A3 | none or B1 | C6 |
| 529 | A4 | none or B1 | C6 |
| 530 | A5 | none or B1 | C6 |
| 531 | A6 | none or B1 | C6 |
| 532 | A7 | none or B1 | C6 |
| 533 | A8 | none or B1 | C6 |
| 534 | A9 | none or B1 | C6 |
| 535 | A10 | none or B1 | C6 |
| 536 | A11 | none or B1 | C6 |
| 537 | A12 | none or B1 | C6 |
| 538 | A13 | none or B1 | C6 |
| 539 | A14 | none or B1 | C6 |
| 540 | A15 | none or B1 | C6 |
| 541 | A16 | none or B1 | C6 |
| 542 | A17 | none or B1 | C6 |
| 543 | A18 | none or B1 | C6 |
| 544 | A19 | none or B1 | C6 |
| 545 | A20 | none or B1 | C6 |
| 546 | A21 | none or B1 | C6 |
| 547 | A1 | B2 | C6 |
| 548 | A2 | B2 | C6 |
| 549 | A3 | B2 | C6 |
| 550 | A4 | B2 | C6 |
| 551 | A5 | B2 | C6 |
| 552 | A6 | B2 | C6 |
| 553 | A7 | B2 | C6 |
| 554 | A8 | B2 | C6 |
| 555 | A9 | B2 | C6 |
| 556 | A10 | B2 | C6 |
| 557 | A11 | B2 | C6 |
| 558 | A12 | B2 | C6 |
| 559 | A13 | B2 | C6 |
| 560 | A14 | B2 | C6 |
| 561 | A15 | B2 | C6 |
| 562 | A16 | B2 | C6 |
| 563 | A17 | B2 | C6 |
| 564 | A18 | B2 | C6 |
| 565 | A19 | B2 | C6 |
| 566 | A20 | B2 | C6 |
| 567 | A21 | B2 | C6 |
| 568 | A1 | B3 | C6 |
| 569 | A2 | B3 | C6 |
| 570 | A3 | B3 | C6 |
| 571 | A4 | B3 | C6 |
| 572 | A5 | B3 | C6 |
| 573 | A6 | B3 | C6 |
| 574 | A7 | B3 | C6 |
| 575 | A8 | B3 | C6 |
| 576 | A9 | B3 | C6 |
| 577 | A10 | B3 | C6 |
| 578 | A11 | B3 | C6 |
| 579 | A12 | B3 | C6 |
| 580 | A13 | B3 | C6 |
| 581 | A14 | B3 | C6 |
| 582 | A15 | B3 | C6 |
| 583 | A16 | B3 | C6 |
| 584 | A17 | B3 | C6 |
| 585 | A18 | B3 | C6 |
| 586 | A19 | B3 | C6 |
| 587 | A20 | B3 | C6 |
| 588 | A21 | B3 | C6 |
| 589 | A1 | B4 | C6 |
| 590 | A2 | B4 | C6 |
| 591 | A3 | B4 | C6 |
| 592 | A4 | B4 | C6 |
| 593 | A5 | B4 | C6 |
| 594 | A6 | B4 | C6 |
| 595 | A7 | B4 | C6 |
| 596 | A8 | B4 | C6 |
| 597 | A9 | B4 | C6 |
| 598 | A10 | B4 | C6 |
| 599 | A11 | B4 | C6 |
| 600 | A12 | B4 | C6 |
| 601 | A13 | B4 | C6 |
| 602 | A14 | B4 | C6 |
| 603 | A15 | B4 | C6 |
| 604 | A16 | B4 | C6 |
| 605 | A17 | B4 | C6 |
| 606 | A18 | B4 | C6 |
| 607 | A19 | B4 | C6 |
| 608 | A20 | B4 | C6 |
| 609 | A21 | B4 | C6 |
| 610 | A1 | Any one of B5 through B21 | C6 |
| 611 | A2 | Any one of B5 through B21 | C6 |
| 612 | A3 | Any one of B5 through B21 | C6 |
| 613 | A4 | Any one of B5 through B21 | C6 |
| 614 | A5 | Any one of B5 through B21 | C6 |
| 615 | A6 | Any one of B5 through B21 | C6 |
| 616 | A7 | Any one of B5 through B21 | C6 |
| 617 | A8 | Any one of B5 through B21 | C6 |
| 618 | A9 | Any one of B5 through B21 | C6 |
| 619 | A10 | Any one of B5 through B21 | C6 |
| 620 | A11 | Any one of B5 through B21 | C6 |
| 621 | A12 | Any one of B5 through B21 | C6 |
| 622 | A13 | Any one of B5 through B21 | C6 |
| 623 | A14 | Any one of B5 through B21 | C6 |
| 624 | A15 | Any one of B5 through B21 | C6 |
| 625 | A16 | Any one of B5 through B21 | C6 |
| 626 | A17 | Any one of B5 through B21 | C6 |
| 627 | A18 | Any one of B5 through B21 | C6 |
| 628 | A19 | Any one of B5 through B21 | C6 |
| 629 | A20 | Any one of B5 through B21 | C6 |
| 630 | A21 | Any one of B5 through B21 | C6 |
| 631 | A1 | none or B1 | C7 |
| 632 | A2 | none or B1 | C7 |
| 633 | A3 | none or B1 | C7 |
| 634 | A4 | none or B1 | C7 |
| 635 | A5 | none or B1 | C7 |
| 636 | A6 | none or B1 | C7 |
| 637 | A7 | none or B1 | C7 |
| 638 | A8 | none or B1 | C7 |
| 639 | A9 | none or B1 | C7 |
| 640 | A10 | none or B1 | C7 |
| 641 | A11 | none or B1 | C7 |
| 642 | A12 | none or B1 | C7 |
| 643 | A13 | none or B1 | C7 |
| 644 | A14 | none or B1 | C7 |
| 645 | A15 | none or B1 | C7 |
| 646 | A16 | none or B1 | C7 |
| 647 | A17 | none or B1 | C7 |
| 648 | A18 | none or B1 | C7 |
| 649 | A19 | none or B1 | C7 |
| 650 | A20 | none or B1 | C7 |
| 651 | A21 | none or B1 | C7 |
| 652 | A1 | B2 | C7 |
| 653 | A2 | B2 | C7 |
| 654 | A3 | B2 | C7 |
| 655 | A4 | B2 | C7 |
| 656 | A5 | B2 | C7 |
| 657 | A6 | B2 | C7 |
| 658 | A7 | B2 | C7 |
| 659 | A8 | B2 | C7 |
| 660 | A9 | B2 | C7 |
| 661 | A10 | B2 | C7 |
| 662 | A11 | B2 | C7 |
| 663 | A12 | B2 | C7 |
| 664 | A13 | B2 | C7 |
| 665 | A14 | B2 | C7 |
| 666 | A15 | B2 | C7 |
| 667 | A16 | B2 | C7 |
| 668 | A17 | B2 | C7 |
| 669 | A18 | B2 | C7 |
| 670 | A19 | B2 | C7 |
| 671 | A20 | B2 | C7 |
| 672 | A21 | B2 | C7 |
| 673 | A1 | B3 | C7 |
| 674 | A2 | B3 | C7 |
| 675 | A3 | B3 | C7 |
| 676 | A4 | B3 | C7 |
| 677 | A5 | B3 | C7 |
| 678 | A6 | B3 | C7 |
| 679 | A7 | B3 | C7 |
| 680 | A8 | B3 | C7 |
| 681 | A9 | B3 | C7 |
| 682 | A10 | B3 | C7 |
| 683 | A11 | B3 | C7 |
| 684 | A12 | B3 | C7 |
| 685 | A13 | B3 | C7 |
| 686 | A14 | B3 | C7 |
| 687 | A15 | B3 | C7 |
| 688 | A16 | B3 | C7 |
| 689 | A17 | B3 | C7 |
| 690 | A18 | B3 | C7 |
| 691 | A19 | B3 | C7 |
| 692 | A20 | B3 | C7 |
| 693 | A21 | B3 | C7 |
| 694 | A1 | B4 | C7 |
| 695 | A2 | B4 | C7 |
| 696 | A3 | B4 | C7 |
| 697 | A4 | B4 | C7 |
| 698 | A5 | B4 | C7 |
| 699 | A6 | B4 | C7 |
| 700 | A7 | B4 | C7 |
| 701 | A8 | B4 | C7 |
| 702 | A9 | B4 | C7 |
| 703 | A10 | B4 | C7 |
| 704 | A11 | B4 | C7 |
| 705 | A12 | B4 | C7 |
| 706 | A13 | B4 | C7 |
| 707 | A14 | B4 | C7 |
| 708 | A15 | B4 | C7 |
| 709 | A16 | B4 | C7 |
| 710 | A17 | B4 | C7 |
| 711 | A18 | B4 | C7 |
| 712 | A19 | B4 | C7 |
| 713 | A20 | B4 | C7 |
| 714 | A21 | B4 | C7 |
| 715 | A1 | Any one of B5 through B21 | C7 |
| 716 | A2 | Any one of B5 through B21 | C7 |
| 717 | A3 | Any one of B5 through B21 | C7 |
| 718 | A4 | Any one of B5 through B21 | C7 |
| 719 | A5 | Any one of B5 through B21 | C7 |
| 720 | A6 | Any one of B5 through B21 | C7 |
| 721 | A7 | Any one of B5 through B21 | C7 |
| 722 | A8 | Any one of B5 through B21 | C7 |
| 723 | A9 | Any one of B5 through B21 | C7 |
| 724 | A10 | Any one of B5 through B21 | C7 |
| 725 | A11 | Any one of B5 through B21 | C7 |
| 726 | A12 | Any one of B5 through B21 | C7 |
| 727 | A13 | Any one of B5 through B21 | C7 |
| 728 | A14 | Any one of B5 through B21 | C7 |
| 729 | A15 | Any one of B5 through B21 | C7 |
| 730 | A16 | Any one of B5 through B21 | C7 |
| 731 | A17 | Any one of B5 through B21 | C7 |
| 732 | A18 | Any one of B5 through B21 | C7 |
| 733 | A19 | Any one of B5 through B21 | C7 |
| 734 | A20 | Any one of B5 through B21 | C7 |
| 735 | A21 | Any one of B5 through B21 | C7 |
| 736 | A1 | none or B1 | C8 |
| 737 | A2 | none or B1 | C8 |
| 738 | A3 | none or B1 | C8 |
| 739 | A4 | none or B1 | C8 |
| 740 | A5 | none or B1 | C8 |
| 741 | A6 | none or B1 | C8 |
| 742 | A7 | none or B1 | C8 |
| 743 | A8 | none or B1 | C8 |
| 744 | A9 | none or B1 | C8 |
| 745 | A10 | none or B1 | C8 |
| 746 | A11 | none or B1 | C8 |
| 747 | A12 | none or B1 | C8 |
| 748 | A13 | none or B1 | C8 |
| 749 | A14 | none or B1 | C8 |
| 750 | A15 | none or B1 | C8 |
| 751 | A16 | none or B1 | C8 |
| 752 | A17 | none or B1 | C8 |
| 753 | A18 | none or B1 | C8 |
| 754 | A19 | none or B1 | C8 |
| 755 | A20 | none or B1 | C8 |
| 756 | A21 | none or B1 | C8 |
| 757 | A1 | B2 | C8 |
| 758 | A2 | B2 | C8 |
| 759 | A3 | B2 | C8 |
| 760 | A4 | B2 | C8 |
| 761 | A5 | B2 | C8 |
| 762 | A6 | B2 | C8 |
| 763 | A7 | B2 | C8 |
| 764 | A8 | B2 | C8 |
| 765 | A9 | B2 | C8 |
| 766 | A10 | B2 | C8 |
| 767 | A11 | B2 | C8 |
| 768 | A12 | B2 | C8 |
| 769 | A13 | B2 | C8 |
| 770 | A14 | B2 | C8 |
| 771 | A15 | B2 | C8 |
| 772 | A16 | B2 | C8 |
| 773 | A17 | B2 | C8 |
| 774 | A18 | B2 | C8 |
| 775 | A19 | B2 | C8 |
| 776 | A20 | B2 | C8 |
| 777 | A21 | B2 | C8 |
| 778 | A1 | B3 | C8 |
| 779 | A2 | B3 | C8 |
| 780 | A3 | B3 | C8 |
| 781 | A4 | B3 | C8 |
| 782 | A5 | B3 | C8 |
| 783 | A6 | B3 | C8 |
| 784 | A7 | B3 | C8 |
| 785 | A8 | B3 | C8 |
| 786 | A9 | B3 | C8 |
| 787 | A10 | B3 | C8 |
| 788 | A11 | B3 | C8 |
| 789 | A12 | B3 | C8 |
| 790 | A13 | B3 | C8 |
| 791 | A14 | B3 | C8 |
| 792 | A15 | B3 | C8 |
| 793 | A16 | B3 | C8 |
| 794 | A17 | B3 | C8 |
| 795 | A18 | B3 | C8 |
| 796 | A19 | B3 | C8 |
| 797 | A20 | B3 | C8 |
| 798 | A21 | B3 | C8 |
| 799 | A1 | B4 | C8 |
| 800 | A2 | B4 | C8 |
| 801 | A3 | B4 | C8 |
| 802 | A4 | B4 | C8 |
| 803 | A5 | B4 | C8 |
| 804 | A6 | B4 | C8 |
| 805 | A7 | B4 | C8 |
| 806 | A8 | B4 | C8 |
| 807 | A9 | B4 | C8 |
| 808 | A10 | B4 | C8 |
| 809 | A11 | B4 | C8 |
| 810 | A12 | B4 | C8 |
| 811 | A13 | B4 | C8 |
| 812 | A14 | B4 | C8 |
| 813 | A15 | B4 | C8 |
| 814 | A16 | B4 | C8 |
| 815 | A17 | B4 | C8 |
| 816 | A18 | B4 | C8 |
| 817 | A19 | B4 | C8 |
| 818 | A20 | B4 | C8 |
| 819 | A21 | B4 | C8 |
| 820 | A1 | Any one of B5 through B21 | C8 |
| 821 | A2 | Any one of B5 through B21 | C8 |
| 822 | A3 | Any one of B5 through B21 | C8 |
| 823 | A4 | Any one of B5 through B21 | C8 |
| 824 | A5 | Any one of B5 through B21 | C8 |
| 825 | A6 | Any one of B5 through B21 | C8 |
| 826 | A7 | Any one of B5 through B21 | C8 |
| 827 | A8 | Any one of B5 through B21 | C8 |
| 828 | A9 | Any one of B5 through B21 | C8 |
| 829 | A10 | Any one of B5 through B21 | C8 |
| 830 | A11 | Any one of B5 through B21 | C8 |
| 831 | A12 | Any one of B5 through B21 | C8 |
| 832 | A13 | Any one of B5 through B21 | C8 |
| 833 | A14 | Any one of B5 through B21 | C8 |
| 834 | A15 | Any one of B5 through B21 | C8 |
| 835 | A16 | Any one of B5 through B21 | C8 |
| 836 | A17 | Any one of B5 through B21 | C8 |
| 837 | A18 | Any one of B5 through B21 | C8 |
| 838 | A19 | Any one of B5 through B21 | C8 |
| 839 | A20 | Any one of B5 through B21 | C8 |
| 840 | A21 | Any one of B5 through B21 | C8 |
| 841 | A1 | none or B1 | C9 |
| 842 | A2 | none or B1 | C9 |
| 843 | A3 | none or B1 | C9 |
| 844 | A4 | none or B1 | C9 |
| 845 | A5 | none or B1 | C9 |
| 846 | A6 | none or B1 | C9 |
| 847 | A7 | none or B1 | C9 |
| 848 | A8 | none or B1 | C9 |
| 849 | A9 | none or B1 | C9 |
| 850 | A10 | none or B1 | C9 |
| 851 | A11 | none or B1 | C9 |
| 852 | A12 | none or B1 | C9 |
| 853 | A13 | none or B1 | C9 |
| 854 | A14 | none or B1 | C9 |
| 855 | A15 | none or B1 | C9 |
| 856 | A16 | none or B1 | C9 |
| 857 | A17 | none or B1 | C9 |
| 858 | A18 | none or B1 | C9 |
| 859 | A19 | none or B1 | C9 |
| 860 | A20 | none or B1 | C9 |
| 861 | A21 | none or B1 | C9 |
| 862 | A1 | B2 | C9 |
| 863 | A2 | B2 | C9 |
| 864 | A3 | B2 | C9 |
| 865 | A4 | B2 | C9 |
| 866 | A5 | B2 | C9 |
| 867 | A6 | B2 | C9 |
| 868 | A7 | B2 | C9 |
| 869 | A8 | B2 | C9 |
| 870 | A9 | B2 | C9 |
| 871 | A10 | B2 | C9 |
| 872 | A11 | B2 | C9 |
| 873 | A12 | B2 | C9 |
| 874 | A13 | B2 | C9 |
| 875 | A14 | B2 | C9 |
| 876 | A15 | B2 | C9 |
| 877 | A16 | B2 | C9 |
| 878 | A17 | B2 | C9 |
| 879 | A18 | B2 | C9 |
| 880 | A19 | B2 | C9 |
| 881 | A20 | B2 | C9 |
| 882 | A21 | B2 | C9 |
| 883 | A1 | B3 | C9 |
| 884 | A2 | B3 | C9 |
| 885 | A3 | B3 | C9 |
| 886 | A4 | B3 | C9 |
| 887 | A5 | B3 | C9 |
| 888 | A6 | B3 | C9 |
| 889 | A7 | B3 | C9 |
| 890 | A8 | B3 | C9 |
| 891 | A9 | B3 | C9 |
| 892 | A10 | B3 | C9 |
| 893 | A11 | B3 | C9 |
| 894 | A12 | B3 | C9 |
| 895 | A13 | B3 | C9 |
| 896 | A14 | B3 | C9 |
| 897 | A15 | B3 | C9 |
| 898 | A16 | B3 | C9 |
| 899 | A17 | B3 | C9 |
| 900 | A18 | B3 | C9 |
| 901 | A19 | B3 | C9 |
| 902 | A20 | B3 | C9 |
| 903 | A21 | B3 | C9 |
| 904 | A1 | B4 | C9 |
| 905 | A2 | B4 | C9 |
| 906 | A3 | B4 | C9 |
| 907 | A4 | B4 | C9 |
| 908 | A5 | B4 | C9 |
| 909 | A6 | B4 | C9 |
| 910 | A7 | B4 | C9 |
| 911 | A8 | B4 | C9 |
| 912 | A9 | B4 | C9 |
| 913 | A10 | B4 | C9 |
| 914 | A11 | B4 | C9 |
| 915 | A12 | B4 | C9 |
| 916 | A13 | B4 | C9 |
| 917 | A14 | B4 | C9 |
| 918 | A15 | B4 | C9 |
| 919 | A16 | B4 | C9 |
| 920 | A17 | B4 | C9 |
| 921 | A18 | B4 | C9 |
| 922 | A19 | B4 | C9 |
| 923 | A20 | B4 | C9 |
| 924 | A21 | B4 | C9 |
| 925 | A1 | Any one of B5 through B21 | C9 |
| 926 | A2 | Any one of B5 through B21 | C9 |
| 927 | A3 | Any one of B5 through B21 | C9 |
| 928 | A4 | Any one of B5 through B21 | C9 |
| 929 | A5 | Any one of B5 through B21 | C9 |
| 930 | A6 | Any one of B5 through B21 | C9 |
| 931 | A7 | Any one of B5 through B21 | C9 |
| 932 | A8 | Any one of B5 through B21 | C9 |
| 933 | A9 | Any one of B5 through B21 | C9 |
| 934 | A10 | Any one of B5 through B21 | C9 |
| 935 | A11 | Any one of B5 through B21 | C9 |
| 936 | A12 | Any one of B5 through B21 | C9 |
| 937 | A13 | Any one of B5 through B21 | C9 |
| 938 | A14 | Any one of B5 through B21 | C9 |
| 939 | A15 | Any one of B5 through B21 | C9 |
| 940 | A16 | Any one of B5 through B21 | C9 |
| 941 | A17 | Any one of B5 through B21 | C9 |
| 942 | A18 | Any one of B5 through B21 | C9 |
| 943 | A19 | Any one of B5 through B21 | C9 |
| 944 | A20 | Any one of B5 through B21 | C9 |
| 945 | A21 | Any one of B5 through B21 | C9 |
| 946 | A1 | none or B1 | C10 |
| 947 | A2 | none or B1 | C10 |
| 948 | A3 | none or B1 | C10 |
| 949 | A4 | none or B1 | C10 |
| 950 | A5 | none or B1 | C10 |
| 951 | A6 | none or B1 | C10 |
| 952 | A7 | none or B1 | C10 |
| 953 | A8 | none or B1 | C10 |
| 954 | A9 | none or B1 | C10 |
| 955 | A10 | none or B1 | C10 |
| 956 | A11 | none or B1 | C10 |
| 957 | A12 | none or B1 | C10 |
| 958 | A13 | none or B1 | C10 |
| 959 | A14 | none or B1 | C10 |
| 960 | A15 | none or B1 | C10 |
| 961 | A16 | none or B1 | C10 |
| 962 | A17 | none or B1 | C10 |
| 963 | A18 | none or B1 | C10 |
| 964 | A19 | none or B1 | C10 |
| 965 | A20 | none or B1 | C10 |
| 966 | A21 | none or B1 | C10 |
| 967 | A1 | B2 | C10 |
| 968 | A2 | B2 | C10 |
| 969 | A3 | B2 | C10 |
| 970 | A4 | B2 | C10 |
| 971 | A5 | B2 | C10 |
| 972 | A6 | B2 | C10 |
| 973 | A7 | B2 | C10 |
| 974 | A8 | B2 | C10 |
| 975 | A9 | B2 | C10 |
| 976 | A10 | B2 | C10 |
| 977 | A11 | B2 | C10 |
| 978 | A12 | B2 | C10 |
| 979 | A13 | B2 | C10 |
| 980 | A14 | B2 | C10 |
| 981 | A15 | B2 | C10 |
| 982 | A16 | B2 | C10 |
| 983 | A17 | B2 | C10 |
| 984 | A18 | B2 | C10 |
| 985 | A19 | B2 | C10 |
| 986 | A20 | B2 | C10 |
| 987 | A21 | B2 | C10 |
| 988 | A1 | B3 | C10 |
| 989 | A2 | B3 | C10 |
| 990 | A3 | B3 | C10 |
| 991 | A4 | B3 | C10 |
| 992 | A5 | B3 | C10 |
| 993 | A6 | B3 | C10 |
| 994 | A7 | B3 | C10 |
| 995 | A8 | B3 | C10 |
| 996 | A9 | B3 | C10 |
| 997 | A10 | B3 | C10 |
| 998 | A11 | B3 | C10 |
| 999 | A12 | B3 | C10 |
| 1000 | A13 | B3 | C10 |
| 1001 | A14 | B3 | C10 |
| 1002 | A15 | B3 | C10 |
| 1003 | A16 | B3 | C10 |
| 1004 | A17 | B3 | C10 |
| 1005 | A18 | B3 | C10 |
| 1006 | A19 | B3 | C10 |
| 1007 | A20 | B3 | C10 |
| 1008 | A21 | B3 | C10 |
| 1009 | A1 | B4 | C10 |
| 1010 | A2 | B4 | C10 |
| 1011 | A3 | B4 | C10 |
| 1012 | A4 | B4 | C10 |
| 1013 | A5 | B4 | C10 |
| 1014 | A6 | B4 | C10 |
| 1015 | A7 | B4 | C10 |
| 1016 | A8 | B4 | C10 |
| 1017 | A9 | B4 | C10 |
| 1018 | A10 | B4 | C10 |
| 1019 | A11 | B4 | C10 |
| 1020 | A12 | B4 | C10 |
| 1021 | A13 | B4 | C10 |
| 1022 | A14 | B4 | C10 |
| 1023 | A15 | B4 | C10 |
| 1024 | A16 | B4 | C10 |
| 1025 | A17 | B4 | C10 |
| 1026 | A18 | B4 | C10 |
| 1027 | A19 | B4 | C10 |
| 1028 | A20 | B4 | C10 |
| 1029 | A21 | B4 | C10 |
| 1030 | A1 | Any one of B5 through B21 | C10 |
| 1031 | A2 | Any one of B5 through B21 | C10 |
| 1032 | A3 | Any one of B5 through B21 | C10 |
| 1033 | A4 | Any one of B5 through B21 | C10 |
| 1034 | A5 | Any one of B5 through B21 | C10 |
| 1035 | A6 | Any one of B5 through B21 | C10 |
| 1036 | A7 | Any one of B5 through B21 | C10 |
| 1037 | A8 | Any one of B5 through B21 | C10 |
| 1038 | A9 | Any one of B5 through B21 | C10 |
| 1039 | A10 | Any one of B5 through B21 | C10 |
| 1040 | A11 | Any one of B5 through B21 | C10 |
| 1041 | A12 | Any one of B5 through B21 | C10 |
| 1042 | A13 | Any one of B5 through B21 | C10 |
| 1043 | A14 | Any one of B5 through B21 | C10 |
| 1044 | A15 | Any one of B5 through B21 | C10 |
| 1045 | A16 | Any one of B5 through B21 | C10 |
| 1046 | A17 | Any one of B5 through B21 | C10 |
| 1047 | A18 | Any one of B5 through B21 | C10 |
| 1048 | A19 | Any one of B5 through B21 | C10 |
| 1049 | A20 | Any one of B5 through B21 | C10 |
| 1050 | A21 | Any one of B5 through B21 | C10 |
| 1051 | A1 | none or B1 | C11 |
| 1052 | A2 | none or B1 | C11 |
| 1053 | A3 | none or B1 | C11 |
| 1054 | A4 | none or B1 | C11 |
| 1055 | A5 | none or B1 | C11 |
| 1056 | A6 | none or B1 | C11 |
| 1057 | A7 | none or B1 | C11 |
| 1058 | A8 | none or B1 | C11 |
| 1059 | A9 | none or B1 | C11 |
| 1060 | A10 | none or B1 | C11 |
| 1061 | A11 | none or B1 | C11 |
| 1062 | A12 | none or B1 | C11 |
| 1063 | A13 | none or B1 | C11 |
| 1064 | A14 | none or B1 | C11 |
| 1065 | A15 | none or B1 | C11 |
| 1066 | A16 | none or B1 | C11 |
| 1067 | A17 | none or B1 | C11 |
| 1068 | A18 | none or B1 | C11 |
| 1069 | A19 | none or B1 | C11 |
| 1070 | A20 | none or B1 | C11 |
| 1071 | A21 | none or B1 | C11 |
| 1072 | A1 | B2 | C11 |
| 1073 | A2 | B2 | C11 |
| 1074 | A3 | B2 | C11 |
| 1075 | A4 | B2 | C11 |
| 1076 | A5 | B2 | C11 |
| 1077 | A6 | B2 | C11 |
| 1078 | A7 | B2 | C11 |
| 1079 | A8 | B2 | C11 |
| 1080 | A9 | B2 | C11 |
| 1081 | A10 | B2 | C11 |
| 1082 | A11 | B2 | C11 |
| 1083 | A12 | B2 | C11 |
| 1084 | A13 | B2 | C11 |
| 1085 | A14 | B2 | C11 |
| 1086 | A15 | B2 | C11 |
| 1087 | A16 | B2 | C11 |
| 1088 | A17 | B2 | C11 |
| 1089 | A18 | B2 | C11 |
| 1090 | A19 | B2 | C11 |
| 1091 | A20 | B2 | C11 |
| 1092 | A21 | B2 | C11 |
| 1093 | A1 | B3 | C11 |
| 1094 | A2 | B3 | C11 |
| 1095 | A3 | B3 | C11 |
| 1096 | A4 | B3 | C11 |
| 1097 | A5 | B3 | C11 |
| 1098 | A6 | B3 | C11 |
| 1099 | A7 | B3 | C11 |
| 1100 | A8 | B3 | C11 |
| 1101 | A9 | B3 | C11 |
| 1102 | A10 | B3 | C11 |
| 1103 | A11 | B3 | C11 |
| 1104 | A12 | B3 | C11 |
| 1105 | A13 | B3 | C11 |
| 1106 | A14 | B3 | C11 |
| 1107 | A15 | B3 | C11 |
| 1108 | A16 | B3 | C11 |
| 1109 | A17 | B3 | C11 |
| 1110 | A18 | B3 | C11 |
| 1111 | A19 | B3 | C11 |
| 1112 | A20 | B3 | C11 |
| 1113 | A21 | B3 | C11 |
| 1114 | A1 | B4 | C11 |
| 1115 | A2 | B4 | C11 |
| 1116 | A3 | B4 | C11 |
| 1117 | A4 | B4 | C11 |
| 1118 | A5 | B4 | C11 |
| 1119 | A6 | B4 | C11 |
| 1120 | A7 | B4 | C11 |
| 1121 | A8 | B4 | C11 |
| 1122 | A9 | B4 | C11 |
| 1123 | A10 | B4 | C11 |
| 1124 | A11 | B4 | C11 |
| 1125 | A12 | B4 | C11 |
| 1126 | A13 | B4 | C11 |
| 1127 | A14 | B4 | C11 |
| 1128 | A15 | B4 | C11 |
| 1129 | A16 | B4 | C11 |
| 1130 | A17 | B4 | C11 |
| 1131 | A18 | B4 | C11 |
| 1132 | A19 | B4 | C11 |
| 1133 | A20 | B4 | C11 |
| 1134 | A21 | B4 | C11 |
| 1135 | A1 | Any one of B5 through B21 | C11 |
| 1136 | A2 | Any one of B5 through B21 | C11 |
| 1137 | A3 | Any one of B5 through B21 | C11 |
| 1138 | A4 | Any one of B5 through B21 | C11 |
| 1139 | A5 | Any one of B5 through B21 | C11 |
| 1140 | A6 | Any one of B5 through B21 | C11 |
| 1141 | A7 | Any one of B5 through B21 | C11 |
| 1142 | A8 | Any one of B5 through B21 | C11 |
| 1143 | A9 | Any one of B5 through B21 | C11 |
| 1144 | A10 | Any one of B5 through B21 | C11 |
| 1145 | A11 | Any one of B5 through B21 | C11 |
| 1146 | A12 | Any one of B5 through B21 | C11 |
| 1147 | A13 | Any one of B5 through B21 | C11 |
| 1148 | A14 | Any one of B5 through B21 | C11 |
| 1149 | A15 | Any one of B5 through B21 | C11 |
| 1150 | A16 | Any one of B5 through B21 | C11 |
| 1151 | A17 | Any one of B5 through B21 | C11 |
| 1152 | A18 | Any one of B5 through B21 | C11 |
| 1153 | A19 | Any one of B5 through B21 | C11 |
| 1154 | A20 | Any one of B5 through B21 | C11 |
| 1155 | A21 | Any one of B5 through B21 | C11 |
| 1156 | A1 | none or B1 | C12 |
| 1157 | A2 | none or B1 | C12 |
| 1158 | A3 | none or B1 | C12 |
| 1159 | A4 | none or B1 | C12 |
| 1160 | A5 | none or B1 | C12 |
| 1161 | A6 | none or B1 | C12 |
| 1162 | A7 | none or B1 | C12 |
| 1163 | A8 | none or B1 | C12 |
| 1164 | A9 | none or B1 | C12 |
| 1165 | A10 | none or B1 | C12 |
| 1166 | A11 | none or B1 | C12 |
| 1167 | A12 | none or B1 | C12 |
| 1168 | A13 | none or B1 | C12 |
| 1169 | A14 | none or B1 | C12 |
| 1170 | A15 | none or B1 | C12 |
| 1171 | A16 | none or B1 | C12 |
| 1172 | A17 | none or B1 | C12 |
| 1173 | A18 | none or B1 | C12 |
| 1174 | A19 | none or B1 | C12 |
| 1175 | A20 | none or B1 | C12 |
| 1176 | A21 | none or B1 | C12 |
| 1177 | A1 | B2 | C12 |
| 1178 | A2 | B2 | C12 |
| 1179 | A3 | B2 | C12 |
| 1180 | A4 | B2 | C12 |
| 1181 | A5 | B2 | C12 |
| 1182 | A6 | B2 | C12 |
| 1183 | A7 | B2 | C12 |
| 1184 | A8 | B2 | C12 |
| 1185 | A9 | B2 | C12 |
| 1186 | A10 | B2 | C12 |
| 1187 | A11 | B2 | C12 |
| 1188 | A12 | B2 | C12 |
| 1189 | A13 | B2 | C12 |
| 1190 | A14 | B2 | C12 |
| 1191 | A15 | B2 | C12 |
| 1192 | A16 | B2 | C12 |
| 1193 | A17 | B2 | C12 |
| 1194 | A18 | B2 | C12 |
| 1195 | A19 | B2 | C12 |
| 1196 | A20 | B2 | C12 |
| 1197 | A21 | B2 | C12 |
| 1198 | A1 | B3 | C12 |
| 1199 | A2 | B3 | C12 |
| 1200 | A3 | B3 | C12 |
| 1201 | A4 | B3 | C12 |
| 1202 | A5 | B3 | C12 |
| 1203 | A6 | B3 | C12 |
| 1204 | A7 | B3 | C12 |
| 1205 | A8 | B3 | C12 |
| 1206 | A9 | B3 | C12 |
| 1207 | A10 | B3 | C12 |
| 1208 | A11 | B3 | C12 |
| 1209 | A12 | B3 | C12 |
| 1210 | A13 | B3 | C12 |
| 1211 | A14 | B3 | C12 |
| 1212 | A15 | B3 | C12 |
| 1213 | A16 | B3 | C12 |
| 1214 | A17 | B3 | C12 |
| 1215 | A18 | B3 | C12 |
| 1216 | A19 | B3 | C12 |
| 1217 | A20 | B3 | C12 |
| 1218 | A21 | B3 | C12 |
| 1219 | A1 | B4 | C12 |
| 1220 | A2 | B4 | C12 |
| 1221 | A3 | B4 | C12 |
| 1222 | A4 | B4 | C12 |
| 1223 | A5 | B4 | C12 |
| 1224 | A6 | B4 | C12 |
| 1225 | A7 | B4 | C12 |
| 1226 | A8 | B4 | C12 |
| 1227 | A9 | B4 | C12 |
| 1228 | A10 | B4 | C12 |
| 1229 | A11 | B4 | C12 |
| 1230 | A12 | B4 | C12 |
| 1231 | A13 | B4 | C12 |
| 1232 | A14 | B4 | C12 |
| 1233 | A15 | B4 | C12 |
| 1234 | A16 | B4 | C12 |
| 1235 | A17 | B4 | C12 |
| 1236 | A18 | B4 | C12 |
| 1237 | A19 | B4 | C12 |
| 1238 | A20 | B4 | C12 |
| 1239 | A21 | B4 | C12 |
| 1240 | A1 | Any one of B5 through B21 | C12 |
| 1241 | A2 | Any one of B5 through B21 | C12 |
| 1242 | A3 | Any one of B5 through B21 | C12 |
| 1243 | A4 | Any one of B5 through B21 | C12 |
| 1244 | A5 | Any one of B5 through B21 | C12 |
| 1245 | A6 | Any one of B5 through B21 | C12 |
| 1246 | A7 | Any one of B5 through B21 | C12 |
| 1247 | A8 | Any one of B5 through B21 | C12 |
| 1248 | A9 | Any one of B5 through B21 | C12 |
| 1249 | A10 | Any one of B5 through B21 | C12 |
| 1250 | A11 | Any one of B5 through B21 | C12 |
| 1251 | A12 | Any one of B5 through B21 | C12 |
| 1252 | A13 | Any one of B5 through B21 | C12 |
| 1253 | A14 | Any one of B5 through B21 | C12 |
| 1254 | A15 | Any one of B5 through B21 | C12 |
| 1255 | A16 | Any one of B5 through B21 | C12 |
| 1256 | A17 | Any one of B5 through B21 | C12 |
| 1257 | A18 | Any one of B5 through B21 | C12 |
| 1258 | A19 | Any one of B5 through B21 | C12 |
| 1259 | A20 | Any one of B5 through B21 | C12 |
| 1260 | A21 | Any one of B5 through B21 | C12 |
| 1261 | A1 | none or B1 | C13 |
| 1262 | A2 | none or B1 | C13 |
| 1263 | A3 | none or B1 | C13 |
| 1264 | A4 | none or B1 | C13 |
| 1265 | A5 | none or B1 | C13 |
| 1266 | A6 | none or B1 | C13 |
| 1267 | A7 | none or B1 | C13 |
| 1268 | A8 | none or B1 | C13 |
| 1269 | A9 | none or B1 | C13 |
| 1270 | A10 | none or B1 | C13 |
| 1271 | A11 | none or B1 | C13 |
| 1272 | A12 | none or B1 | C13 |
| 1273 | A13 | none or B1 | C13 |
| 1274 | A14 | none or B1 | C13 |
| 1275 | A15 | none or B1 | C13 |
| 1276 | A16 | none or B1 | C13 |
| 1277 | A17 | none or B1 | C13 |
| 1278 | A18 | none or B1 | C13 |
| 1279 | A19 | none or B1 | C13 |
| 1280 | A20 | none or B1 | C13 |
| 1281 | A21 | none or B1 | C13 |
| 1282 | A1 | B2 | C13 |
| 1283 | A2 | B2 | C13 |
| 1284 | A3 | B2 | C13 |
| 1285 | A4 | B2 | C13 |
| 1286 | A5 | B2 | C13 |
| 1287 | A6 | B2 | C13 |
| 1288 | A7 | B2 | C13 |
| 1289 | A8 | B2 | C13 |
| 1290 | A9 | B2 | C13 |
| 1291 | A10 | B2 | C13 |
| 1292 | A11 | B2 | C13 |
| 1293 | A12 | B2 | C13 |
| 1294 | A13 | B2 | C13 |
| 1295 | A14 | B2 | C13 |
| 1296 | A15 | B2 | C13 |
| 1297 | A16 | B2 | C13 |
| 1298 | A17 | B2 | C13 |
| 1299 | A18 | B2 | C13 |
| 1300 | A19 | B2 | C13 |
| 1301 | A20 | B2 | C13 |
| 1302 | A21 | B2 | C13 |
| 1303 | A1 | B3 | C13 |
| 1304 | A2 | B3 | C13 |
| 1305 | A3 | B3 | C13 |
| 1306 | A4 | B3 | C13 |
| 1307 | A5 | B3 | C13 |
| 1308 | A6 | B3 | C13 |
| 1309 | A7 | B3 | C13 |
| 1310 | A8 | B3 | C13 |
| 1311 | A9 | B3 | C13 |
| 1312 | A10 | B3 | C13 |
| 1313 | A11 | B3 | C13 |
| 1314 | A12 | B3 | C13 |
| 1315 | A13 | B3 | C13 |
| 1316 | A14 | B3 | C13 |
| 1317 | A15 | B3 | C13 |
| 1318 | A16 | B3 | C13 |
| 1319 | A17 | B3 | C13 |
| 1320 | A18 | B3 | C13 |
| 1321 | A19 | B3 | C13 |
| 1322 | A20 | B3 | C13 |
| 1323 | A21 | B3 | C13 |
| 1324 | A1 | B4 | C13 |
| 1325 | A2 | B4 | C13 |
| 1326 | A3 | B4 | C13 |
| 1327 | A4 | B4 | C13 |
| 1328 | A5 | B4 | C13 |
| 1329 | A6 | B4 | C13 |
| 1330 | A7 | B4 | C13 |
| 1331 | A8 | B4 | C13 |
| 1332 | A9 | B4 | C13 |
| 1333 | A10 | B4 | C13 |
| 1334 | A11 | B4 | C13 |
| 1335 | A12 | B4 | C13 |
| 1336 | A13 | B4 | C13 |
| 1337 | A14 | B4 | C13 |
| 1338 | A15 | B4 | C13 |
| 1339 | A16 | B4 | C13 |
| 1340 | A17 | B4 | C13 |
| 1341 | A18 | B4 | C13 |
| 1342 | A19 | B4 | C13 |
| 1343 | A20 | B4 | C13 |
| 1344 | A21 | B4 | C13 |
| 1345 | A1 | Any one of B5 through B21 | C13 |
| 1346 | A2 | Any one of B5 through B21 | C13 |
| 1347 | A3 | Any one of B5 through B21 | C13 |
| 1348 | A4 | Any one of B5 through B21 | C13 |
| 1349 | A5 | Any one of B5 through B21 | C13 |
| 1350 | A6 | Any one of B5 through B21 | C13 |
| 1351 | A7 | Any one of B5 through B21 | C13 |
| 1352 | A8 | Any one of B5 through B21 | C13 |
| 1353 | A9 | Any one of B5 through B21 | C13 |
| 1354 | A10 | Any one of B5 through B21 | C13 |
| 1355 | A11 | Any one of B5 through B21 | C13 |
| 1356 | A12 | Any one of B5 through B21 | C13 |
| 1357 | A13 | Any one of B5 through B21 | C13 |
| 1358 | A14 | Any one of B5 through B21 | C13 |
| 1359 | A15 | Any one of B5 through B21 | C13 |
| 1360 | A16 | Any one of B5 through B21 | C13 |
| 1361 | A17 | Any one of B5 through B21 | C13 |
| 1362 | A18 | Any one of B5 through B21 | C13 |
| 1363 | A19 | Any one of B5 through B21 | C13 |
| 1364 | A20 | Any one of B5 through B21 | C13 |
| 1365 | A21 | Any one of B5 through B21 | C13 |

As would be apparent to one skilled in the art, certain engineered TCR constructs (ATOMICs) comprising multiple A and B components are contemplated, and are surprisingly effective (see Examples 2-9).

Additionally, the engineered TCRs (ATOMICs) contemplated herein may also include a native or engineered TCR constant domain. For example, the constant domain can be a native or engineered TCRα, TCRβ, TCRγ, or TCRδ constant domain. Moreover, any TCR variable domain can be combined with any TCR constant domain. For example, a TCRα variable domain can be combined with any one of the TCRα, TCRβ, TCRγ, or TCRδ constant domains; a TCRβ variable domain can be combined with any one of the TCRα, TCRβ, TCRγ, or TCRδ constant domains; a TCRγ variable domain can be combined with any one of the TCRα, TCRβ, TCRγ, or TCRδ constant domains; and a TCRδ variable domain can be combined with any one of the TCRα, TCRβ, TCRγ, or TCRδ constant domains. Illustrative native and pairing enhanced TCR constant domains are provided in Table 7 below. For other examples of TCR constant domains, see also WO2021195503A1, which is incorporated by reference herein, in its entirety.

TABLE 7
TCR constant domains:

| NAME | SEQ ID NO: |
|---|---|
| human beta constant 1 (huTRBC1) | 80 |
| human beta constant 2 (huTRBC2) | 81 |
| human alpha constant (huTRAC) | 82 |
| human gamma constant 1 (huTRGC1) | 83 |
| human gamma constant 2 (huTRGC2) | 84 |
| human delta constant (huTRDC) | 85 |
| pairing enhanced beta constant 1 (mmTRBC1) | 86 |
| pairing enhanced beta constant 2 (mmTRBC2) | 87 |
| pairing enhanced alpha constant (mmTRAC) | 88 |

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. An engineered T cell receptor (TCR) comprising:
a) a TCRα polypeptide comprising a TCRα variable domain;
b) a TCRβ polypeptide comprising a TCRβ variable domain; and
c) one or more antigen-binding domains linked to the TCRα variable domain and/or TCRβ variable domain.

2. An engineered T cell receptor (TCR) comprising:
a) a TCRγ polypeptide comprising a TCRγ variable domain;
b) a TCRδ polypeptide comprising a TCRδ variable domain; and
c) one or more antigen-binding domains linked to the TCRγ variable domain and/or TCRδ variable domain.

3. The engineered TCR of claim 1, wherein the TCRα polypeptide comprises a TCRα constant domain and the TCRβ polypeptide comprises a TCRβ constant domain.

4. The engineered TCR of claim 2, wherein the TCRγ polypeptide comprises a TCRγ constant domain and the TCRδ polypeptide comprises a TCRδ constant domain.

5. The engineered TCR of any one of claims 1-4, wherein the one or more antigen-binding domains comprises a first antigen-binding domain linked to the TCRα or TCRγ variable domain.

6. The engineered TCR of any one of claims 1-5, wherein the one or more antigen-binding domains comprises a first antigen-binding domain linked to the TCRβ or TCRδ variable domain.

7. The engineered TCR of any one of claims 1-6, wherein the one or more antigen-binding domains comprise: (i) a first antigen-binding domain linked to the TCRα or TCRγ variable domain, and (ii) a first antigen-binding domain linked to the TCRβ or TCRδ variable domain.

8. The engineered TCR of any one of claims 5-7, wherein the first antigen-binding domains are linked to the N-terminus of the variable domains.

9. The engineered TCR of any one of claims 5-8, wherein the first antigen-binding domains are the same or different, and/or bind to the same or different target antigens.

10. The engineered TCR of any one of claims 5-9, wherein the one or more antigen-binding domains comprises a second antigen-binding domain linked to the first antigen-binding domain linked to the TCRα or TCRγ variable domain.

11. The engineered TCR of any one of claims 5-10, wherein the one or more antigen-binding domains comprises a second antigen-binding domain linked to the first antigen-binding domain linked to the TCRβ or TCRδ variable domain.

12. The engineered TCR of any one of claims 5-11, wherein the one or more antigen-binding domains comprises: (i) a second antigen-binding domain linked to the first antigen-binding domain linked to the TCRα or TCRγ variable domain, and (ii) a second antigen-binding domain linked to the first antigen-binding domain linked to the TCRβ or TCRδ variable domain.

13. The engineered TCR of any one of claims 10-12, wherein the second antigen-binding domains are linked to the N-terminus of the first antigen-binding domain.

14. The engineered TCR of claim 12 or claim 13, wherein the second antigen-binding domains are the same or different, and/or bind to the same or different target antigens.

15. The engineered TCR of any one of claims 5-14, wherein the first and second antigen-binding domains are the same or different, and/or bind to the same or different target antigens.

16. The engineered TCR of any one of claims 1-15, wherein the one or more antigen-binding domains bind a target antigen selected from the group consisting of: alpha folate receptor (FRα), αᵥβ₆ integrin, ADGRE2, BACE2, B cell maturation antigen (BCMA), B7-H3 (CD276), B7-H4, B7-H6, CA19.9, carbonic anhydrase IX (CAIX), CCR1, CD7, CD16, CD19, CD20, CD22, CD30, CD33, CD3γ, CD38, CD44, CD44v6, CD44v7/8, CD70, CD79a, CD79b, CD123, CD133, CD138, CD171, CD244, carcinoembryonic antigen (CEA), C-type lectin-like molecule-1 (CLL-1), CD2 subset 1 (CS-1), CLDN6, cMET, chondroitin sulfate proteoglycan 4 (CSPG4), CLDN18.2, cutaneous T cell lymphoma-associated antigen 1 (CTAGE1), DLL3, epidermal growth factor receptor (EGFR), epidermal growth factor receptor variant III (EGFRvIII), EGFR806, epithelial glycoprotein 2 (EGP2), epithelial glycoprotein 40 (EGP40), EPHB2, ERBB4, epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), FLT3, FN, FN-EDB, FRBeta, ganglioside G2 (GD2), ganglioside G3 (GD3), Glypican-3 (GPC3), EGFR family including ErbB2 (HER2), HER2p95, EGFRv3, IL-10Rα, IL-13Rα2, Kappa, cancer/testis antigen 2 (LAGE-1A), K-Ras, K-Ras G12C, K-Ras G12D, K-Ras G12V, Lambda, Lewis-Y (LeY), L1 cell adhesion molecule (L1-CAM), LILRB2, LY6G6GD, melanoma antigen recognized by T cells 1 (MelanA or MART1), Mesothelin (MSLN), MMP10, MUC1, MUC16, MHC class I chain related proteins A (MICA), MHC class I chain related proteins B (MICB), neural cell adhesion molecule (NCAM), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), transmembrane activator and CAML interactor (TACI), tumor endothelial marker 1 (TEM1/CD248), tumor endothelial marker 7-related (TEM7R), TIM3, trophoblast glycoprotein (TPBG), UL16-binding protein (ULBP) 1, ULBP2, ULBP3, ULBP4, ULBP5, ULBP6, and vascular endothelial growth factor receptor 2 (VEGFR2).

17. The engineered TCR of any one of claims 1-16, wherein the one or more antigen-binding domains bind a target polypeptide derived from a protein selected from the group consisting of: α-fetoprotein (AFP), ASCL2, B Melanoma Antigen (BAGE) family members, Brother of the regulator of imprinted sites (BORIS), Cancer-testis antigens, Cancer-testis antigen 83 (CT-83), Carbonic anhydrase IX (CAIX), Carcinoembryonic antigen (CEA), Cytomegalovirus (CMV) antigens, Cytotoxic T cell (CTL)-recognized antigen on melanoma (CAMEL), Epstein-Barr virus (EBV) antigens, EPHB2, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human papillomavirus (HPV)-E6, HPV-E7, Human telomerase reverse transcriptase (hTERT), IGF2BP3/A3, IGF2BP1, K-Ras, K-Ras G12C, K-Ras G12D, K-Ras G12V, Latent membrane protein 2 (LMP2), LY6G6D, Melanoma antigen family A, 1 (MAGE-A1), MAGE-A2, MAGE-A3, MAGE-A4, MAGE-A6, MAGE-A10, MAGE-A12, Melanoma antigen recognized by T cells (MART-1), Mesothelin (MSLN), Mucin 1 (MUC1), Mucin 16 (MUC16), New York esophageal squamous cell carcinoma-1 (NYESO-1), P53,P antigen (PAGE) family members, PAP, PIK3CA, PIK3CA H1047R, Placenta-specific 1 (PLAC1), Preferentially expressed antigen in melanoma (PRAME), Prostate specific antigen PSA, Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, TP53 R175H, Tyrosinase, Tyrosinase related protein (TRP)1, TRP2, UBD, Wilms tumor protein (WT-1), Wnt10A, X Antigen Family Member 1 (XAGE1), and X Antigen Family Member 2 (XAGE2).

18. The engineered TCR of any one of claims 1-17, wherein the one or more antigen-binding domains bind CD33, CLL1, CD19, CD20, CD22, CD79A, CD79B, or BCMA.

19. The engineered TCR of any one of claims 1-17, wherein the one or more antigen-binding domains bind CD19, CD20, CD22, CD33, CD79A, CD79B, B7H3, Muc16, Her2, EGFR, FN-EDB, CLDN18.2, DLL3, FLT3, CLL1, CD123, or BCMA.

20. The engineered TCR of any one of claims 1-17, wherein the one or more antigen-binding domains comprises an amino acid sequence at least 95% identical to an amino acid sequence as set forth in any one of SEQ ID NOs: 1-32.

21. The engineered TCR of any one of claims 1-20, wherein the one or more antigen-binding domains comprise an antibody or antigen binding fragment thereof selected from the group consisting of: a Camel Ig, a Llama Ig, an Alpaca Ig, Ig NAR, a Fab′ fragment, a F(ab′)₂ fragment, a bispecific Fab dimer (Fab2), a trispecific Fab trimer (Fab3), an Fv, an single chain Fv protein (“scFv”), a bis-scFv, (scFv)₂, a minibody, a diabody, a triabody, a tetrabody, a disulfide stabilized Fv protein (“dsFv”), and a single-domain antibody (sdAb, a camelid VHH, Nanobody).

22. The engineered TCR of any one of claims 1-21, wherein the one or more antigen-binding domains comprise one or more single-chain variable fragments (scFv).

23. The engineered TCR of any one of claims 1-22, wherein the one or more antigen-binding domains comprise one or more single domain antibodies (sdAb).

24. The engineered TCR of claim 23, wherein the sdAb is a camelid VHH, nanobody, or heavy chain-only antibody (HcAb).

25. The engineered TCR of claim 23, wherein the sdAb is a camelid VHH.

26. The engineered TCR of any one of claims 21-25, wherein antibody or antigen binding fragment thereof is human or humanized.

27. The engineered TCR of any one of claims 1-19, wherein the one or more antigen-binding domains comprise a ligand.

28. The engineered TCR of any one of claims 1-27, wherein the one or more antigen-binding domain are linked to the TCR variable domains by one or more polypeptide linkers.

29. The engineered TCR of claim 28, wherein the one or more polypeptide linkers comprise a linker from about 2 to about 25 amino acids long.

30. The engineered TCR of claim 28 or claim 29, wherein the one or more polypeptide linkers comprise a linker from about 4 to about 15 amino acids long.

31. The engineered TCR of any one of claims 28-30, wherein the one or more polypeptide linkers comprise a linker from about 4 to about 10 amino acids long.

32. The engineered TCR of any one of claims 28-31, wherein the one or more polypeptide linkers comprise a linker of about 4, about 5, about 6, about 7, about 8, about 9, about 10, about 11, about 12, about 13, about 14, or about 15 amino acids long.

33. The engineered TCR of any one of claims 28-32, wherein the one or more polypeptide linkers comprise a linker of about 9 or about 10 amino acids long.

34. The engineered TCR of any one of claims 28-33 wherein the one or more polypeptide linkers comprise a linker selected from the group consisting of: GG, GS, SG, SS, GSS, SSG, GSG, SGS, SGG, GGS, GGGS (SEQ ID NO: 53), (GGGGS)₁₋₅ polypeptide (SEQ ID NOs: 35-39), a linker from a marsupial γμTCR (e.g., LEKT; SEQ ID NO: 33), and any combination thereof.

35. The engineered TCR of claims 28-34, wherein the one or more polypeptide linkers comprises a linker from a marsupial YTCR, comprising an amino acid sequence as set forth in SEQ ID NO: 33.

36. The engineered TCR of claims 28-35, wherein the one or more polypeptide linkers comprise a GGGGS (SEQ ID NO: 35) linker (G4S).

37. The engineered TCR of claims 28-36, wherein the one or more polypeptide linkers comprise a marsupial γμTCR linker and a G4S linker as set forth in SEQ ID NO: 34.

38. The engineered TCR of claims 28-37, wherein the one or more polypeptide linkers comprise two GGGGS linkers (2×G4S) (SEQ ID NO: 36).

39. The engineered TCR of claims 28-38, wherein the one or more polypeptide linkers comprise three GGGGS linkers (3×G4S) (SEQ ID NO: 37).

40. The engineered TCR of claims 28-39, wherein the one or more polypeptide linkers comprise an amino acid sequence as set forth in any one of SEQ ID NOs: 33-53.

41. The engineered TCR of any one of claims 10-40, wherein the first and second antigen-binding domains are separated by a second polypeptide linker.

42. The engineered TCR of claim 41, wherein the second polypeptide linker is about 2 to about 25 amino acids long.

43. The engineered TCR of claim 41 or claim 42, wherein the second polypeptide linker is about 4 to about 15 amino acids long.

44. The engineered TCR of any one of claims 41-43, wherein the second polypeptide linker comprises a linker selected from the group consisting of: GG, GS, SG, SS, GSS, SSG, GSG, SGS, SGG, GGS, GGGS (SEQ ID NO: 53), (GGGGS)₁₋₅ polypeptide (SEQ ID NOs: 35-39), and any combination thereof.

45. The engineered TCR of any one of claims 41-44, wherein the second polypeptide linker comprises an amino acid sequence as set forth in any one of SEQ ID NOs: 33-53.

46. The engineered TCR of any one of claims 1-45, wherein the TCR variable domains bind a target polypeptide presented by an MHC complex.

47. The engineered TCR of any one of the claims 1-46, wherein the TCR variable domains bind a target polypeptide derived from a protein selected from the group consisting of: α-fetoprotein (AFP), ASCL2, B Melanoma Antigen (BAGE) family members, Brother of the regulator of imprinted sites (BORIS), Cancer-testis antigens, Cancer-testis antigen 83 (CT-83), Carbonic anhydrase IX (CAIX), Carcinoembryonic antigen (CEA), Cytomegalovirus (CMV) antigens, Cytotoxic T cell (CTL)-recognized antigen on melanoma (CAMEL), Epstein-Barr virus (EBV) antigens, EPHB2, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human papillomavirus (HPV)-E6, HPV-E7, Human telomerase reverse transcriptase (hTERT), IGF2BP3/A3, IGF2BP1, K-Ras, K-Ras G12C, K-Ras G12D, K-Ras G12V, Latent membrane protein 2 (LMP2), LY6G6D, Melanoma antigen family A, 1 (MAGE-A1), MAGE-A2, MAGE-A3, MAGE-A4, MAGE-A6, MAGE-A10, MAGE-A12, Melanoma antigen recognized by T cells (MART-1), Mesothelin (MSLN), Mucin 1 (MUC1), Mucin 16 (MUC16), New York esophageal squamous cell carcinoma-1 (NYESO-1), P53,P antigen (PAGE) family members, PAP, PIK3CA, PIK3CA H1047R, Placenta-specific 1 (PLAC1), Preferentially expressed antigen in melanoma (PRAME), Prostate specific antigen PSA, Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, TP53 R175H, Tyrosinase, Tyrosinase related protein (TRP)1, TRP2, UBD, Wilms tumor protein (WT-1), Wnt10A, X Antigen Family Member 1 (XAGE1), and X Antigen Family Member 2 (XAGE2).

48. The engineered TCR of any one of the claims 1-47, wherein the TCR variable domains bind a target polypeptide derived from MAGE-A4, PRAME, K-Ras, TP53R175H, PSA, or IGF2BP3.

49. The engineered TCR of any one of the claims 1-48, wherein the TCR variable domains bind a target polypeptide derived from MAGE-A4.

50. The engineered TCR of any one of claims 1-49, wherein the TCRα constant domain comprises an amino acid sequence at least 90% identical to an amino acid sequence as set forth in SEQ ID NOs: 82 or 88, and/or the TCRβ constant domain comprises an amino acid sequence at least 90% identical to an amino acid sequence as set forth in any one of SEQ ID NOs: 80, 81, 86, or 87.

51. The engineered TCR of any one of claims 1-49, wherein the TCRγ constant domain comprises an amino acid sequence at least 90% identical to an amino acid sequence as set forth in SEQ ID NO: 83 or 84, and/or the TCRδ constant domain comprises an amino acid sequence at least 90% identical to an amino acid sequence as set forth in any one of SEQ ID NO: 85.

52. The engineered TCR of any one of claims 1-51, wherein the TCRα or TCRγ polypeptide comprises (i) an amino acid sequence as set forth in any one of SEQ ID NOs: 105-111, or (ii) a TCRα or TCRγ variable domain comprising an amino acid sequence as set forth in any one of SEQ ID NOs: 62, 64, 66, 68, 70, 72, 74, 76, and 78.

53. The engineered TCR of any one of claims 1-52, wherein the TCRβ or TCRδ polypeptide comprises (i) an amino acid sequence as set forth in SEQ ID NO: 103 or 104, or (ii) a TCRβ or TCRδ variable domain comprising an amino acid sequence as set forth in any one of SEQ ID NOs: 63, 65, 67, 69, 71, 73, 75, 77, and 79.

54. A fusion polypeptide comprising:
a) a TCRβ polypeptide comprising a TCRβ variable domain;
b) a polypeptide cleavage signal; and
c) a TCRα polypeptide comprising one or more antigen-binding domains, a polypeptide linker, and a TCRα variable domain.

55. A fusion polypeptide comprising:
a) a TCRβ polypeptide comprising one or more antigen-binding domains, a polypeptide linker, and a TCRβ variable domain;
b) a polypeptide cleavage signal; and
c) a TCRα polypeptide comprising a TCRα variable domain.

56. A fusion polypeptide comprising:
a) a TCRβ polypeptide comprising one or more antigen-binding domains, a polypeptide linker, and a TCRβ variable domain;
b) a polypeptide cleavage signal; and
c) a TCRα polypeptide comprising one or more antigen-binding domains, a polypeptide linker, and a TCRα variable domain.

57. A fusion polypeptide comprising:
a) a TCRγ polypeptide comprising a TCRγ variable domain;
b) a polypeptide cleavage signal; and
c) a TCRδ polypeptide comprising one or more antigen-binding domains, a polypeptide linker, and a TCRδ variable domain.

58. A fusion polypeptide comprising:
a) a TCRγ polypeptide comprising one or more antigen-binding domains, a polypeptide linker, and a TCRγ variable domain;
b) a polypeptide cleavage signal; and
c) a TCRδ polypeptide comprising a TCRδ variable domain.

59. A fusion polypeptide comprising:
a) a TCRγ polypeptide comprising one or more antigen-binding domains, a polypeptide linker, and a TCRγ variable domain;
b) a polypeptide cleavage signal; and
c) a TCRδ polypeptide comprising one or more antigen-binding domains, a polypeptide linker, and a TCRδ variable domain.

60. The fusion polypeptide of any one of claims 54-56, wherein the TCRβ polypeptide comprises TCRβ constant domain, and the TCRα polypeptide comprises a TCRα constant domain.

61. The fusion polypeptide of any one of claims 57-59, wherein the TCRγ polypeptide comprises TCRγ constant domain, and the TCRδ polypeptide comprises a TCRδ constant domain.

62. The fusion polypeptide of any one of claims 54-61, wherein the one or more antigen-binding domains comprises a first antigen-binding domain linked to the TCRα or TCRγ variable domain.

63. The fusion polypeptide of any one of claims 54-62, wherein the one or more antigen-binding domains comprises a first antigen-binding domain linked to the TCRβ or TCRδ variable domain.

64. The fusion polypeptide of any one of claims 54-63, wherein the one or more antigen-binding domains comprise: (i) a first antigen-binding domain linked to the TCRα or TCRγ variable domain, and (ii) a first antigen-binding domain linked to the TCRβ or TCRδ variable domain.

65. The fusion polypeptide of any one of claims 62-64, wherein the first antigen-binding domains are linked to the N-terminus of the variable domains.

66. The fusion polypeptide of any one of claims 62-65, wherein the first antigen-binding domains are the same or different, and/or bind to the same or different target antigens.

67. The fusion polypeptide of any one of claims 62-66, wherein the one or more antigen-binding domains comprises a second antigen-binding domain linked to the first antigen-binding domain linked to the TCRα or TCRγ variable domain.

68. The fusion polypeptide of any one of claims 62-67, wherein the one or more antigen-binding domains comprises a second antigen-binding domain is linked to the first antigen-binding domain linked to the TCRβ or TCRδ variable domain.

69. The fusion polypeptide of any one of claims 62-68, wherein the one or more antigen-binding domains comprises: (i) a second antigen-binding domain linked to the first antigen-binding domain linked to the TCRα or TCRγ variable domain, and (ii) a second antigen-binding domain linked to the first antigen-binding domain linked to the TCRβ or TCRδ variable domain.

70. The fusion polypeptide of any one of claims 67-69, wherein the second antigen-binding domains are linked to the N-terminus of the first antigen-binding domain.

71. The fusion polypeptide of claim 69 or claim 70, wherein the second antigen-binding domains are the same or different, and/or bind to the same or different target antigens.

72. The fusion polypeptide of any one of claims 67-71, wherein the first and second antigen-binding domains are the same or different, and/or bind to the same or different target antigens.

73. The fusion polypeptide of any one of claims 54-72, wherein the one or more antigen-binding domains bind a target antigen selected from the group consisting of: alpha folate receptor (FRα), αᵥβ₆ integrin, ADGRE2, BACE2, B cell maturation antigen (BCMA), B7-H3 (CD276), B7-H4, B7-H6, CA19.9, carbonic anhydrase IX (CAIX), CCR1, CD7, CD16, CD19, CD20, CD22, CD30, CD33, CD37, CD38, CD44, CD44v6, CD44v7/8, CD70, CD79a, CD79b, CD123, CD133, CD138, CD171, CD244, carcinoembryonic antigen (CEA), C-type lectin-like molecule-1 (CLL-1), CD2 subset 1 (CS-1), CLDN6, cMET, chondroitin sulfate proteoglycan 4 (CSPG4), CLDN18.2, cutaneous T cell lymphoma-associated antigen 1 (CTAGE1), DLL3, epidermal growth factor receptor (EGFR), epidermal growth factor receptor variant III (EGFRvIII), EGFR806, epithelial glycoprotein 2 (EGP2), epithelial glycoprotein 40 (EGP40), EPHB2, ERBB4, epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), FLT3, FN, FN-EDB, FRBeta, ganglioside G2 (GD2), ganglioside G3 (GD3), Glypican-3 (GPC3), EGFR family including ErbB2 (HER2), HER2p95, EGFRv3, IL-10Rα, IL-13Rα2, Kappa, cancer/testis antigen 2 (LAGE-1A), K-Ras, K-Ras G12C, K-Ras G12D, K-Ras G12V, Lambda, Lewis-Y (LeY), L1 cell adhesion molecule (L1-CAM), LILRB2, LY6G6GD, melanoma antigen recognized by T cells 1 (MelanA or MART1), Mesothelin (MSLN), MMP10, MUC1, MUC16, MHC class I chain related proteins A (MICA), MHC class I chain related proteins B (MICB), neural cell adhesion molecule (NCAM), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), transmembrane activator and CAML interactor (TACI), tumor endothelial marker 1 (TEM1/CD248), tumor endothelial marker 7-related (TEM7R), TIM3, trophoblast glycoprotein (TPBG), UL16-binding protein (ULBP) 1, ULBP2, ULBP3, ULBP4, ULBP5, ULBP6, and vascular endothelial growth factor receptor 2 (VEGFR2).

74. The fusion polypeptide of any one of claims 54-73, wherein the one or more antigen-binding domains bind a target polypeptide derived from a protein selected from the group consisting of: α-fetoprotein (AFP), ASCL2, B Melanoma Antigen (BAGE) family members, Brother of the regulator of imprinted sites (BORIS), Cancer-testis antigens, Cancer-testis antigen 83 (CT-83), Carbonic anhydrase IX (CAIX), Carcinoembryonic antigen (CEA), Cytomegalovirus (CMV) antigens, Cytotoxic T cell (CTL)-recognized antigen on melanoma (CAMEL), Epstein-Barr virus (EBV) antigens, EPHB2, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human papillomavirus (HPV)-E6, HPV-E7, Human telomerase reverse transcriptase (hTERT), IGF2BP3/A3, IGF2BP1, K-Ras, K-Ras G12C, K-Ras G12D, K-Ras G12V, Latent membrane protein 2 (LMP2), LY6G6D, Melanoma antigen family A, 1 (MAGE-A1), MAGE-A2, MAGE-A3, MAGE-A4, MAGE-A6, MAGE-A10, MAGE-A12, Melanoma antigen recognized by T cells (MART-1), Mesothelin (MSLN), Mucin 1 (MUC1), Mucin 16 (MUC16), New York esophageal squamous cell carcinoma-1 (NYESO-1), P53,P antigen (PAGE) family members, PAP, PIK3CA, PIK3CA H1047R, Placenta-specific 1 (PLAC1), Preferentially expressed antigen in melanoma (PRAME), Prostate specific antigen PSA, Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, TP53 R175H, Tyrosinase, Tyrosinase related protein (TRP)1, TRP2, UBD, Wilms tumor protein (WT-1), Wnt10A, X Antigen Family Member 1 (XAGE1), and X Antigen Family Member 2 (XAGE2).

75. The fusion polypeptide of claims 54-74, wherein the one or more antigen-binding domains bind CD33, CLL1, CD19, CD20, CD22, CD79A, CD79B, or BCMA.

76. The engineered TCR of any one of claims 54-74, wherein the one or more antigen-binding domains bind CD19, CD20, CD22, CD33, CD79A, CD79B, B7H3, Muc16, Her2, EGFR, FN-EDB, CLDN18.2, DLL3, FLT3, CLL1, CD123, or BCMA.

77. The engineered TCR of any one of claims 54-74, wherein the one or more antigen-binding domains comprises an amino acid sequence at least 95% identical to an amino acid sequence as set forth in any one of SEQ ID NOs: 1-32.

78. The fusion polypeptide of any one of claims 54-77, wherein the one or more antigen-binding domains comprise an antibody or antigen binding fragment thereof selected from the group consisting of: a Camel Ig, a Llama Ig, an Alpaca Ig, Ig NAR, a Fab′ fragment, a F(ab′)₂ fragment, a bispecific Fab dimer (Fab2), a trispecific Fab trimer (Fab3), an Fv, an single chain Fv protein (“scFv”), a bis-scFv, (scFv)₂, a minibody, a diabody, a triabody, a tetrabody, a disulfide stabilized Fv protein (“dsFv”), and a single-domain antibody (sdAb, a camelid VHH, Nanobody).

79. The fusion polypeptide of any one of claims 54-78, wherein the one or more antigen-binding domains comprise one or more single-chain variable fragments (scFv).

80. The fusion polypeptide of any one of claims 54-79, wherein the one or more antigen-binding domains comprise one or more single domain antibodies (sdAb).

81. The fusion polypeptide of claim 80, wherein the sdAb is a camelid VHH, nanobody, or heavy chain-only antibody (HcAb).

82. The fusion polypeptide of claim 80, wherein the sdAb is a camelid VHH.

83. The fusion polypeptide of any one of claims 74-82, wherein the antibody or antigen binding fragment thereof is human or humanized.

84. The fusion polypeptide of any one of claims 54-76, wherein the one or more antigen-binding domains comprise a ligand.

85. The fusion polypeptide of any one of claims 54-84, wherein the one or more antigen-binding domains are linked to the TCR variable domains by one or more polypeptide linkers.

86. The fusion polypeptide of claim 85, wherein the one or more polypeptide linkers comprise a linker from about 2 to about 25 amino acids long.

87. The fusion polypeptide of claim 85 or claim 86, wherein the one or more polypeptide linkers comprise a linker from about 4 to about 15 amino acids long.

88. The fusion polypeptide of any one of claims 85-87, wherein the one or more polypeptide linkers comprise a linker from about 4 to about 10 amino acids long.

89. The fusion polypeptide of any one of claims 85-88, wherein the one or more polypeptide linkers comprise a linker of about 4, about 5, about 6, about 7, about 8, about 9, about 10, about 11, about 12, about 13, about 14, or about 15 amino acids long.

90. The fusion polypeptide of any one of claims 85-89, wherein the one or more polypeptide linkers comprise a linker of about 9 or about 10 amino acids long.

91. The fusion polypeptide of any one of claims 85-90 wherein the one or more polypeptide linkers comprise a linker selected from the group consisting of: GG, GS, SG, SS, GSS, SSG, GSG, SGS, SGG, GGS, GGGS (SEQ ID NO: 53), (GGGGS)₁₋₅ polypeptide (SEQ ID NOs: 35-39), a linker from a marsupial γμTCR (e.g., LEKT; SEQ ID NO: 33), and any combination thereof.

92. The fusion polypeptide of claims 85-91, wherein the one or more polypeptide linkers comprises a linker from a marsupial YTCR, comprising an amino acid sequence as set forth in SEQ ID NO: 33.

93. The fusion polypeptide of claims 85-92, wherein the one or more polypeptide linkers comprise a GGGGS (SEQ ID NO: 35) linker (G4S).

94. The fusion polypeptide of claims 85-93, wherein the one or more polypeptide linkers comprise a marsupial γμTCR linker and a G4S linker as set forth in SEQ ID NO: 34.

95. The fusion polypeptide of claims 85-94, wherein the one or more polypeptide linkers comprise two GGGGS linkers (2×G4S) (SEQ ID NO: 36).

96. The fusion polypeptide of claims 85-95, wherein the one or more polypeptide linkers comprise three GGGGS linkers (3×G4S) (SEQ ID NO: 37).

97. The fusion polypeptide of claims 85-96, wherein the one or more polypeptide linkers comprise an amino acid sequence as set forth in any one of SEQ ID NOs: 33-53.

98. The fusion polypeptide of any one of claims 67-97, wherein the first and second antigen-binding domains are separated by a second polypeptide linker.

99. The fusion polypeptide of claim 98, wherein the second polypeptide linker is about 2 to about 25 amino acids long.

100. The fusion polypeptide of claim 98 or claim 99, wherein the one or more polypeptide linkers comprise a linker from about 4 to about 15 amino acids long.

101. The fusion polypeptide of any one of claims 98-100, wherein the second polypeptide linker comprises a linker selected from the group consisting of: GG, GS, SG, SS, GSS, SSG, GSG, SGS, SGG, GGS, GGGS (SEQ ID NO: 53), (GGGGS)₁₋₅ polypeptide (SEQ ID NOs: 35-39), and any combination thereof.

102. The fusion polypeptide of any one of claims 98-101, wherein the second polypeptide linker comprises an amino acid sequence as set forth in any one of SEQ ID NOs: 33-53.

103. The fusion polypeptide of any one of claims 54-102, wherein the TCR variable domains bind a target polypeptide presented by an MHC complex.

104. The fusion polypeptide of any one of the claims 54-103, wherein the TCR variable domains bind a target polypeptide derived from a protein selected from the group consisting of: α-fetoprotein (AFP), ASCL2, B Melanoma Antigen (BAGE) family members, Brother of the regulator of imprinted sites (BORIS), Cancer-testis antigens, Cancer-testis antigen 83 (CT-83), Carbonic anhydrase IX (CAIX), Carcinoembryonic antigen (CEA), Cytomegalovirus (CMV) antigens, Cytotoxic T cell (CTL)-recognized antigen on melanoma (CAMEL), Epstein-Barr virus (EBV) antigens, EPHB2, G antigen 1 (GAGE-1), GAGE-2, GAGE-3, GAGE-4, GAGE-5, GAGE-6, GAGE-7B, GAGE-8, Glycoprotein 100 (GP100), Hepatitis B virus (HBV) antigens, Hepatitis C virus (HCV) non-structure protein 3 (NS3), Human papillomavirus (HPV)-E6, HPV-E7, Human telomerase reverse transcriptase (hTERT), IGF2BP3/A3, IGF2BP1, K-Ras, K-Ras G12C, K-Ras G12D, K-Ras G12V, Latent membrane protein 2 (LMP2), LY6G6D, Melanoma antigen family A, 1 (MAGE-A1), MAGE-A2, MAGE-A3, MAGE-A4, MAGE-A6, MAGE-A10, MAGE-A12, Melanoma antigen recognized by T cells (MART-1), Mesothelin (MSLN), Mucin 1 (MUC1), Mucin 16 (MUC16), New York esophageal squamous cell carcinoma-1 (NYESO-1), P53,P antigen (PAGE) family members, PAP, PIK3CA, PIK3CA H1047R, Placenta-specific 1 (PLAC1), Preferentially expressed antigen in melanoma (PRAME), Prostate specific antigen PSA, Survivin, Synovial sarcoma X 1 (SSX1), Synovial sarcoma X 2 (SSX2), Synovial sarcoma X 3 (SSX3), Synovial sarcoma X 4 (SSX4), Synovial sarcoma X 5 (SSX5), Synovial sarcoma X 8 (SSX8), Thyroglobulin, TP53 R175H, Tyrosinase, Tyrosinase related protein (TRP)1, TRP2, UBD, Wilms tumor protein (WT-1), Wnt10A, X Antigen Family Member 1 (XAGE1), and X Antigen Family Member 2 (XAGE2).

105. The fusion polypeptide of any one of the claims 54-104, wherein the TCR variable domains bind a target polypeptide derived from MAGE-A4, PRAME, K-Ras, TP53R175H, PSA, or IGF2BP3.

106. The fusion polypeptide of any one of the claims 54-105, wherein the TCR variable domains bind a target polypeptide derived from MAGE-A4.

107. The engineered TCR of any one of claims 54-106, wherein the TCRα constant domain comprises an amino acid sequence at least 90% identical to an amino acid sequence as set forth in SEQ ID NOs: 82 or 88, and/or the TCRβ constant domain comprises an amino acid sequence at least 90% identical to an amino acid sequence as set forth in any one of SEQ ID NOs: 80, 81, 86, or 87.

108. The engineered TCR of any one of claims 54-106, wherein the TCRγ constant domain comprises an amino acid sequence at least 90% identical to an amino acid sequence as set forth in SEQ ID NO: 83 or 84, and/or the TCRδ constant domain comprises an amino acid sequence at least 90% identical to an amino acid sequence as set forth in any one of SEQ ID NO: 85.

109. The fusion polypeptide of any one of claims 54-108, wherein the TCRα or TCRγ polypeptide comprises (i) an amino acid sequence as set forth in any one of SEQ ID NOs: 105-111, or (ii) a TCRα or TCRγ variable domain comprising an amino acid sequence as set forth in any one of SEQ ID NOs: 62, 64, 66, 68, 70, 72, 74, 76, and 78.

110. The fusion polypeptide of any one of claims 54-109, wherein the TCRβ or TCRδ polypeptide comprises an amino acid sequence as set forth in any one of SEQ ID NOs: 103 or 104, or (ii) a TCRβ or TCRδ variable domain comprising an amino acid sequence as set forth in any one of SEQ ID NOs: 63, 65, 67, 69, 71, 73, 75, 77, and 79.

111. The fusion polypeptide of any one of claims 54-110, wherein the polypeptide cleavage signal is a viral self-cleaving peptide or ribosomal skipping sequence.

112. The fusion polypeptide of any one of claims 54-111, wherein the polypeptide cleavage signal is a viral 2A peptide.

113. The fusion polypeptide of any one of claims 54-112, wherein the polypeptide cleavage signal is an aphthovirus 2A peptide, a potyvirus 2A peptide, or a cardiovirus 2A peptide.

114. The fusion polypeptide of any one of claims 54-113, wherein the polypeptide cleavage signal is a viral 2A peptide selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

115. The fusion polypeptide of any one of claims 111-114, wherein the polypeptide cleavage signal comprises a furin recognition site upstream of the self-cleaving peptide, optionally wherein the furin recognition site comprises the amino acid sequence as set forth in SEQ ID NO: 112.

116. The fusion polypeptide of any one of claims 54-115, wherein the polypeptide cleavage signal comprises an amino acid sequence as set forth in any one of SEQ ID NOs: 113-137.

117. The fusion polypeptide of any one of claims 54-116, wherein the TCRβ or TCRδ polypeptide is N-terminal of the TCRα or TCRγ polypeptide.

118. The fusion polypeptide of any one of claims 54-116, wherein the TCRα or TCRγ polypeptide is N-terminal of the TCRβ or TCRδ polypeptide.

119. The fusion polypeptide of any one of claims 54-118, wherein the TCRα and TCRβ polypeptides each comprise an N-terminal signal sequence.

120. The fusion polypeptide of any one of claims 54-119, wherein the TCRγ and TCRδ polypeptides each comprises an N-terminal signal sequence.

121. The fusion polypeptide of claim 119 or claim 120, wherein the signal sequences are the same or different.

122. The fusion polypeptide of any one of claims 119-121, wherein the signal sequence is an IgK or TCRα signal sequence.

123. The fusion polypeptide of any one of claims 54-122, wherein the fusion polypeptide comprises an amino acid sequence at least 90% identical to an amino acid sequence as set forth in any one of SEQ ID NOs: 91-97, 100, and 102.

124. A polynucleotide encoding the TCR polypeptides of the engineered TCR according to any one of claims 1-51, or the fusion polypeptide of any one of claims 54-123.

125. A vector comprising the polynucleotide of claim 124.

126. The vector of claim 125, wherein the vector is an expression vector, retroviral vector, or a lentiviral vector.

127. A cell comprising the engineered TCR according to any one of claims 1-53, the fusion polypeptide of any one of claims 54-123, the polynucleotide of claim 124, or the vector of claim 125 or claim 126.

128. The cell of claim 127, wherein the cell is a hematopoietic cell.

129. The cell of claim 127 or claim 128, wherein the cell is a T cell, an αβ-T cell, or a γδ-T cell.

130. The cell of any one of claims 127-129, wherein the cell is a CD3⁺, CD4⁺, and/or CD8⁺ cell.

131. The cell of any one of claims 127-130, wherein the cell is an immune effector cell.

132. The cell of any one of claims 127-131, wherein the cell is a cytotoxic T lymphocytes (CTLs), a tumor infiltrating lymphocytes (TILs), or a helper T cell.

133. The cell of any one of claims 127-132, wherein the cell is a T cell, a natural killer (NK) cell, or a natural killer T (NKT) cell.

134. The cell of any one of claims 127-133, wherein the source of the cell is peripheral blood mononuclear cells, bone marrow, lymph nodes tissue, cord blood, thymus issue, tissue from a site of infection, ascites, pleural effusion, spleen tissue, or tumors.

135. The cell of any one of claims 127-134, wherein the cell is an isolated non-natural cell.

136. The cell of any one of claims 127-135, wherein the cell is obtained from a subject.

137. The cell of any one of claims 127-136, wherein the cell is a human cell.

138. A composition comprising the engineered TCR according to any one of claims 1-53, the fusion polypeptide of any one of claims 54-123, the polynucleotide of claim 124, or the vector of claim 125 or claim 126, or the cell of any one of claims 127-137.

139. A pharmaceutical composition comprising a pharmaceutically acceptable carrier and the engineered TCR according to any one of claims 1-53, the fusion polypeptide of any one of claims 54-123, the polynucleotide of claim 124, or the vector of claim 125 or claim 126, or the cell of any one of claims 127-137.

140. A method of treating a subject in need thereof comprising administering the subject an effective amount of the cell of any one of claims 127-137, the composition of claim 138, or the pharmaceutical composition of claim 139.

141. A method of treating, preventing, or ameliorating at least one symptom of a cancer, infectious disease, autoimmune disease, inflammatory disease, and immunodeficiency, or condition associated therewith, comprising administering to the subject an effective amount of the cell of any one of claims 127-137, the composition of claim 138, or the pharmaceutical composition of claim 139.

142. A method of treating a solid cancer comprising administering to the subject an effective amount of the cell of any one of claims 127-137, the composition of claim 138, or the pharmaceutical composition of claim 139.

143. The method of claim 142, wherein the solid cancer is selected from the group consisting of: lung cancer, squamous cell carcinoma, colorectal cancer, pancreatic cancer, breast cancer, thyroid cancer, bladder cancer, cervical cancer, esophageal cancer, ovarian cancer, gastric cancer endometrial cancer, brain cancer, or sarcoma.

144. The method of claim 142, wherein the solid cancer is a non-small cell lung carcinoma (NSCLC), small cell lung cancer (SCLC), head and neck squamous cell carcinoma, colorectal cancer, pancreatic cancer, breast cancer, thyroid cancer, bladder cancer, cervical cancer, esophageal cancer, ovarian cancer, gastric cancer endometrial cancer, gliomas, glioblastomas, oligodendroglioma, sarcoma, or osteosarcoma.

145. A method of treating a hematological malignancy comprising administering to the subject an effective amount of the cell of any one of claims 127-137, the composition of claim 138, or the pharmaceutical composition of claim 139.

146. The method of claim 145, wherein the hematological malignancy is a leukemia, lymphoma, or multiple myeloma.

147. The method of claim 145 or claim 146, wherein the hematological malignancy is selected from the group consisting of non-Hodgkin's lymphoma, acute myeloid leukemia (AML), acute lymphoblastic leukemia (ALL).


SEQUENCE LISTING (139 sequences)

SEQ ID NO: 1 - protein, 246 aa, synthetic construct
  REGION 1..246: note = Synthetic: anti-BCMA.1 scFV
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 2 - protein, 246 aa, synthetic construct
  REGION 1..246: note = Synthetic: anti-BCMA.2 scFV
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 3 - protein, 247 aa, synthetic construct
  REGION 1..247: note = Synthetic: anti-BCMA.3 scFV
DIQMTQSPSSLSASVGDRITITCRASQDIRNYLGWYQQKPGKAPKVLIFAASSLQSGVPSRFSGSGSGTDFTLTISSLQPEDFATYYCLQDYIYPWTFAQGTKVEIKGGGGSGGGGSGGGGSQVQLVESGGGVVQPGRSLRLSCAASGFTFSSYGMHWVRQAPGKGLEWVAVISYDGRNKNYADSVKGRFTISRDNSKNTLYLQMNSLRAEDTAVYYCAREGEATYYDILTGPFDYWGQGTLVTVSS

SEQ ID NO: 4 - protein, 242 aa, synthetic construct
  REGION 1..242: note = Synthetic: anti-CD19 scFV
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 5 - protein, 243 aa, synthetic construct
  REGION 1..243: note = Synthetic: anti-CD20.1 scFV
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 6 - protein, 243 aa, synthetic construct
  REGION 1..243: note = Synthetic: anti-CD20.2 scFV
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKR

SEQ ID NO: 7 - protein, 249 aa, synthetic construct
  REGION 1..249: note = Synthetic: anti-CD20.3 scFV
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSGGGGSGGGGSGGGGSDIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 8 - protein, 236 aa, synthetic construct
  REGION 1..236: note = Synthetic: anti-CD22 scFV
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 9 - protein, 245 aa, synthetic construct
  REGION 1..245: note = Synthetic: anti-CD33 scFV
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKGGGGSGGGGSGGGGSQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 10 - protein, 248 aa, synthetic construct
  REGION 1..248: note = Synthetic: anti-CD79A scFV
DVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIKRGGGGSGGGGSGGGGSQVQLQQSGPELVKPGASVKISCKASGYTFSTSWMNWVKQRPGQGLEWIGRIYPGDGDTNYNGKFKGKATLTADKSSNTAYMQLSSLTSVDSAVYFCERFYYGNTFAMDYWGQGTSVTVSS

SEQ ID NO: 11 - protein, 238 aa, synthetic construct
  REGION 1..238: note = Synthetic: anti-CD79B.1 scFV
QIQLVQSGPELKKPGETVKISCKASGYTFTDYSMHWVKQAPGEGLKWMGWINTETGEPTYADDFKGRFAFSLETSASTAYLQINNLKNEDTATYFCYYGSYWGQGTLVTVSAGGGGSGGGGSGGGGSDIVLTQSPASLAVSLGQRATISCKASQSVDYDGDGYMDWYQQKPGQPPKLLIFAASNLKSGIPARFSGSGSGTDFTLNIHPVEEEDAATYYCQQTNEYPWTFGGGTKLEIK

SEQ ID NO: 12 - protein, 244 aa, synthetic construct
  REGION 1..244: note = Synthetic: anti-CD79B.2 scFV
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRGGGGSGGGGSGGGGSEVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSS

SEQ ID NO: 13 - protein, 242 aa, synthetic construct
  REGION 1..242: note = Synthetic: anti-B7H3 scFV
DIVMTQSHKFMSTSIGARVSITCKASQDVRTAVAWYQQKPGQSPKLLIYSASYRYTGVPDRFTGSGSGTDFTFTISSVQAEDLAVYYCQQHYGTPPWTFGGGTKLEIKGGGGSGGGGSGGGGSEVQLVESGGGLVKPGGSLKLSCEASRFTFSSYAMSWVRQTPEKRLEWVAAISGGGRYTYYPDSMKGRFTISRDNAKNFLYLQMSSLRSEDTAMYYCARHYDGYLDYWGQGTTLTVSSTR

SEQ ID NO: 14 - protein, 250 aa, synthetic construct
  REGION 1..250: note = Synthetic: anti-Muc16 scFV
VKLQESGGGFVKPGGSLKVSCAASGFTFSSYAMSWVRLSPEMRLEWVATISSAGGYIFYSDSVQGRFTISRDNAKNTLHLQMGSLRSGDTAMYYCARQGFGNYGDYYAMDYWGQGTTVTVSSGGGGSGGGGSGGGGSDIELTQSPSSLAVSAGEKVTMSCKSSQSLLNSRTRKNQLAWYQQKPGQSPELLIYWASTRQSGVPDRFTGSGSGTDFTLTISSVQAEDLAVYYCQQSYNLLTFGPGTKLEVKR

SEQ ID NO: 15 - protein, 243 aa, synthetic construct
  REGION 1..243: note = Synthetic: anti-HER2 scFV
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGSGKPGSGEGSGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 16 - protein, 241 aa, synthetic construct
  REGION 1..241: note = Synthetic: anti-EGFR scFV
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSS

SEQ ID NO: 17 - protein, 236 aa, synthetic construct
  REGION 1..236: note = Synthetic: anti-FN-EDB scFV
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 18 - protein, 246 aa, synthetic construct
  REGION 1..246: note = Synthetic: anti-CLDN18.2 scFV
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 19 - protein, 241 aa, synthetic construct
  REGION 1..241: note = Synthetic: anti-DLL3 scFV
QVQLQESGPGLVKPSETLSLTCTVSGDSISSYYWTWIRQPPGKGLEWIGYIYYSGTTNYNPSLKSRVTISVDTSKSQFSLKLSSVTAADTAVYYCASIAVRGFFFDYWGQGTLVTVSSGGGGSGGGGSGGGGSEIVLTQSPGTLSLSPGERATLSCRASQSVSSSYLAWYQQKPGQAPRLLIYGASTRATGIPDRFSGSGSGTDFTLTISRLEPEDFAVYYCQQYGTSPLTFGGGTKVEIK

SEQ ID NO: 20 - protein, 249 aa, synthetic construct
  REGION 1..249: note = Synthetic: anti-FLT3.1 scFV
QVTLKESGPVLVKPTETLTLTCTVSGFSLINARMGVSWIRQPPGKALEWLAHIFSNAEKSYRTSLKSRLTISKDTSKSQVVLTMTNMDPVDTATYYCARIPGYGGNGDYHYYGMDVWGQGTTVTVSSGGGGSGGGGSGGGGSDIQMTQSPSSLSASLGDRVTITCRASQGIRNDLGWYQQKPGKAPKRLIYASSTLQSGVPSRFSGSGSGTEFTLTISSLQPEDFATYYCLQHNNFPWTFGQGTKVEIK

SEQ ID NO: 21 - protein, 243 aa, synthetic construct
  REGION 1..243: note = Synthetic: anti-FLT3.2 scFV
EVQLVESGGGLVQPGGSLRLSCAASGFTFSSYGMHWVRQAPGKGLEWVAVISYDGSNKYYADSVKGRFTISRDNSKNTLYLQMNSLRAEDTAVYYCANLAPWAAYWGQGTLVTVSSGGGGSGGGGSGGGGSEIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPHTFGQGTKLEIK

SEQ ID NO: 22 - protein, 116 aa, synthetic construct
  REGION 1..116: note = Synthetic: anti-CD33.1 VHH
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKP

SEQ ID NO: 23 - protein, 120 aa, synthetic construct
  REGION 1..120: note = Synthetic: anti-CD33.2 VHH
EVQLVESGGGEVQPGGSLRLSCAASGRTFSGYIMGWFRQAPGKERELVARISGNNLSTEYAESVKGRFTISRDNAKNTLYLQMSSLRAEDTAVYYCAAEYDYSSGDFVYWGQGTLVTVKP

SEQ ID NO: 24 - protein, 120 aa, synthetic construct
  REGION 1..120: note = Synthetic: anti-CD33.3 VHH
EVQLVESGGGEVQPGGSLRLSCAASGSTLNIDHIGWYRQAPGKERELVGVISSGAGPNYAESVKGRFTISRDNAKNTVYLQMSSLRAEDTAVYYCNAWIDYGSGLPQNYWGQGTLVTVKP

SEQ ID NO: 25 - protein, 120 aa, synthetic construct
  REGION 1..120: note = Synthetic: anti-CLL1.1 VHH
EVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKP

SEQ ID NO: 26 - protein, 116 aa, synthetic construct
  REGION 1..116: note = Synthetic: anti-CLL1.2 VHH
EVQLVESGGGEVQPGGSLRLSCAASGLLFSIYDMNWYRQAPGKEREWVAGITNNGYSTAYAESVKGRFTISRDNAKNTVYLQMSSLRAEDTAVYYCHTDEWGREYWGQGTLVTVKP

SEQ ID NO: 27 - protein, 125 aa, synthetic construct
  REGION 1..125: note = Synthetic: anti-CD123.1 VHH
EVQLVESGGGEVQPGGSLRLSCTASGRAINMYAMGWFRQAPGKEREFVAAINWNGAYTQYAESVKGRFTISRDNAKNTLYLQMSSLRAEDTAVYYCSADADYNTYVSPNKRVSYWGQGTLVTVKP

SEQ ID NO: 28 - protein, 126 aa, synthetic construct
  REGION 1..126: note = Synthetic: anti-CD123.2 VHH
EVQLVESGGGLVQPGGSLRLSCAASGRAINAYNMGWFRQAPGKGREFVSAINWNAARTYYAESVKGRFTISRDNAKNTLYLQMSSLRAEDTAVYYCAASGRWSAAVPSGEDQYNFWGQGTLVTVKP

SEQ ID NO: 29 - protein, 121 aa, synthetic construct
  REGION 1..121: note = Synthetic: anti-CD20 VHH
QVQLQESGGGLVQAGGSLRLSCAASGRTFSNYNMGWFRQAPGKEREFVAAIDWSGGSPYYAASVRGRFTISRDNAENTVYLQMNSLKPEDTAVYYCAAPLSYGSTWLADYWGQGTQVTVSS

SEQ ID NO: 30 - protein, 124 aa, synthetic construct
  REGION 1..124: note = Synthetic: anti-EGFR VHH
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 31 - protein, 119 aa, synthetic construct
  REGION 1..119: note = Synthetic: anti-BCMA.1 VHH
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSS

SEQ ID NO: 32 - protein, 118 aa, synthetic construct
  REGION 1..118: note = Synthetic: anti-BCMA.2 VHH
EVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSS

SEQ ID NO: 33 - protein, 4 aa, synthetic construct
  REGION 1..4: note = Synthetic: yuTCR linker (uLNK)
LEKT

SEQ ID NO: 34 - protein, 9 aa, synthetic construct
  REGION 1..9: note = Synthetic: uLNK + G4S linker
LEKTGGGGS

SEQ ID NO: 35 - protein, 5 aa, synthetic construct
  REGION 1..5: note = Synthetic: 1xG4S linker
GGGGS

SEQ ID NO: 36 - protein, 10 aa, synthetic construct
  REGION 1..10: note = Synthetic: 2xG4S linker
GGGGSGGGGS

SEQ ID NO: 37 - protein, 15 aa, synthetic construct
  REGION 1..15: note = Synthetic: 3xG4S linker
GGGGSGGGGSGGGGS

SEQ ID NO: 38 - protein, 20 aa, synthetic construct
  REGION 1..20: note = Synthetic: 4xG4S linker
GGGGSGGGGSGGGGSGGGGS

SEQ ID NO: 39 - protein, 25 aa, synthetic construct
  REGION 1..25: note = Synthetic: 5xG4S linker
GGGGSGGGGSGGGGSGGGGSGGGGS

SEQ ID NO: 40 - protein, 5 aa, synthetic construct
  REGION 1..5: note = Synthetic: linker
DGGGS

SEQ ID NO: 41 - protein, 5 aa, synthetic construct
  REGION 1..5: note = Synthetic: linker
TGEKP

SEQ ID NO: 42 - protein, 4 aa, synthetic construct
  REGION 1..4: note = Synthetic: linker
GGRR

SEQ ID NO: 43 - protein, 14 aa, synthetic construct
  REGION 1..14: note = Synthetic: linker
EGKSSGSGSESKVD

SEQ ID NO: 44 - protein, 18 aa, synthetic construct
  REGION 1..18: note = Synthetic: linker
KESGSVSSEQLAQFRSLD

SEQ ID NO: 45 - protein, 8 aa, synthetic construct
  REGION 1..8: note = Synthetic: linker
GGRRGGGS

SEQ ID NO: 46 - protein, 9 aa, synthetic construct
  REGION 1..9: note = Synthetic: linker
LRQRDGERP

SEQ ID NO: 47 - protein, 12 aa, synthetic construct
  REGION 1..12: note = Synthetic: linker
LRQKDGGGSERP

SEQ ID NO: 48 - protein, 16 aa, synthetic construct
  REGION 1..16: note = Synthetic: linker
LRQKDGGGSGGGSERP

SEQ ID NO: 49 - protein, 18 aa, synthetic construct
  REGION 1..18: note = Synthetic: linker
GSTSGSGKPGSGEGSTKG

SEQ ID NO: 50 - protein, 18 aa, synthetic construct
  REGION 1..18: note = Synthetic: linker
GSTSGSGKSSEGSGSTKG

SEQ ID NO: 51 - protein, 14 aa, synthetic construct
  REGION 1..14: note = Synthetic: linker
GSTSGSGKSSEGKG

SEQ ID NO: 52 - protein, 15 aa, synthetic construct
  REGION 1..15: note = Synthetic: linker
GSTSGSGKPGSGEGS

SEQ ID NO: 53 - protein, 4 aa, synthetic construct
  REGION 1..4: note = Synthetic: linker
GGGS

SEQ ID NO: 54 - protein, 114 aa, synthetic construct
  REGION 1..114: note = Synthetic: NY-ESO-1.1 TCRalpha
QEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPY

SEQ ID NO: 55 - protein, 112 aa, synthetic construct
  REGION 1..112: note = Synthetic: NY-ESO-1.1 TCRbeta
GVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLE

SEQ ID NO: 56 - protein, 112 aa, synthetic construct
  REGION 1..112: note = Synthetic: NY-ESO-1.2 TCRalpha
QEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVH

SEQ ID NO: 57 - protein, 112 aa, synthetic construct; identical to SEQ ID NO: 55
  REGION 1..112: note = Synthetic: NY-ESO-1.2 TCRbeta

SEQ ID NO: 58 - protein, 108 aa, synthetic construct
  REGION 1..108: note = Synthetic: PRAME TCRalpha
VEQSPQSLHVQEGDSTNFTCSFPSSNFYALHWYRWETAKSPEALFVMTLNGDEKKKGRISATLNTKEGYSYLYIKGSQPEDSATYLCALYNNNDMRFGAGTRLTVKPN

SEQ ID NO: 59 - protein, 112 aa, synthetic construct
  REGION 1..112: note = Synthetic: PRAME TCRbeta
GVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGSTDTQYFGPGTRLTVLE

SEQ ID NO: 60 - protein, 112 aa, synthetic construct
  REGION 1..112: note = Synthetic: TP53R175H TCRalpha
QRKEVEQDPGPFNVPEGATVAFNCTYSNSASQSFFWYRQDCRKEPKLLMSVYSSGNEDGRFTAQLNRASQYISLLIRDSKLSDSATYLCVVQPGGYQKVTFGTGTKLQVIPD

SEQ ID NO: 61 - protein, 114 aa, synthetic construct
  REGION 1..114: note = Synthetic: TP53R175H TCRbeta
GVTQTPKFQVLKTGQSMTLQCAQDMNHNSMYWYRQDPGMGLRLIYYSASEGTTDKGEVPNGYNVSRLNKREFSLRLESAAPSQTSVYFCASSEGLWQVGDEQYFGPGTRLTVTE

SEQ ID NO: 62 - protein, 116 aa, synthetic construct
  REGION 1..116: note = Synthetic: MAGE-A4 TCRalpha
QKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKAN

SEQ ID NO: 63 - protein, 111 aa, synthetic construct
  REGION 1..111: note = Synthetic: MAGE-A4 TCRbeta
GVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLE

SEQ ID NO: 64 - protein, 107 aa, synthetic construct
  REGION 1..107: note = Synthetic: WT1 TCRalpha
VEQHPSTLSVQEGDSAVIKCTYSDSASNYFPWYKQELGKRPQLIIDIRSNVGEKKDQRIAVTLNKTAKHFSLHITETQPEDSAVYFCAATEDYQLIWGAGTKLIIKP

SEQ ID NO: 65 - protein, 110 aa, synthetic construct
  REGION 1..110: note = Synthetic: WT1 TCRbeta
GITQSPKYLFRKEGQNVTLSCEQNLNHDAMYWYRQDPGQGLRLIYYSQIVNDFQKGDIAEGYSVSREKKESFPLTVTSAQKNPTAFYLCASSPGALYEQYFGPGTRLTVT

SEQ ID NO: 66 - protein, 110 aa, synthetic construct
  REGION 1..110: note = Synthetic: MR1.1 TCRalpha
GQNIDQPTEMTATEGAIVQINCTYQTSGFNGLFWYQQHAGEAPTFLSYNVLDGLEEKGRFSSFLSRSKGYSYLLLKELQMKDSASYLCAVKDSNYQLIWGAGTKLIIKPD

SEQ ID NO: 67 - protein, 116 aa, synthetic construct
  REGION 1..116: note = Synthetic: MR1.1 TCRbeta
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSVWTGEGSGELFFGEGSRLTVLE

SEQ ID NO: 68 - protein, 114 aa, synthetic construct
  REGION 1..114: note = Synthetic: MR1.2 TCRalpha
QTVTQSQPEMSVQEAETVTLSCTYDTSESDYYLFWYKQPPSRQMILVIRQEAYKQQNATENRFSVNFQKAAKSFSLKISDSQLGDAAMYFCAYRSAVNARLMFGDGTQLVVKPN

SEQ ID NO: 69 - protein, 116 aa, synthetic construct
  REGION 1..116: note = Synthetic: MR1.2 TCRbeta
DIYQTPRYLVIGTGKKITLECSQTMGHDKMYWYQQDPGMELHLIHYSYGVNSTEKGDLSSESTVSRIRTEHFPLTLESARPSHTSQYLCASSEARGLAEFTDTQYFGPGTRLTVLE

SEQ ID NO: 70 - protein, 113 aa, synthetic construct
  REGION 1..113: note = Synthetic: CD1d+aGalCer TCRalpha
QVEQSPQSLIILEGKNCTLQCNYTVSPFSNLRWYKQDTGRGPVSLTIMTFSENTKSNGRYTATLDADTKQSSLHITASQLSDSASYICVVSDRGSTLGRLYFGRGTQLTVWPD

SEQ ID NO: 71 - protein, 114 aa, synthetic construct
  REGION 1..114: note = Synthetic: CD1d+aGalCer TCRbeta
DIYQTPRYLVIGTGKKITLECSQTMGHDKMYWYQQDPGMELHLIHYSYGVNSTEKGDLSSESTVSRIRTEHFPLTLESARPSHTSQYLCASSGLRDRGLYEQYFGPGTRLTVTE

SEQ ID NO: 72 - protein, 132 aa, synthetic construct
  REGION 1..132: note = Synthetic: HPV16E7 TCRalpha
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGHPSSNSGYALNFGKGTSLLVTPH

SEQ ID NO: 73 - protein, 136 aa, synthetic construct
  REGION 1..136: note = Synthetic: HPV16E7 TCRbeta
MGTSLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSSQTGARTDTQYFGPGTRLTVLE

SEQ ID NO: 74 - protein, 133 aa, synthetic construct
  REGION 1..133: note = Synthetic: GP100 TCRalpha
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAASLIQGAQKLVFGQGTRLTINPN

SEQ ID NO: 75 - protein, 132 aa, synthetic construct
  REGION 1..132: note = Synthetic: GP100 TCRbeta
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGGNEQFFGPGTRLTVLE

SEQ ID NO: 76 - protein, 128 aa, synthetic construct
  REGION 1..128: note = Synthetic: MART-1 TCRalpha
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGGTGNQFYFGTGTSLTVIPN

SEQ ID NO: 77 - protein, 133 aa, synthetic construct
  REGION 1..133: note = Synthetic: MART-1 TCRbeta
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISEVGVGQPQHFGDGTRLSILE

SEQ ID NO: 78 - protein, 136 aa, synthetic construct
  REGION 1..136: note = Synthetic: allo-HLA TCRgamma
MGWALLVLLAFLSPASQKSSNLEGGTKSVTRPTRSSAEITCDLTVINAFYIHWYLHQEGKAPQRLLYYDVSNSKDVLESGLSPGKYYTHTPRRWSWILILRNLIENDSGVYYCATWDRPEIYYKKLFGSGTTLVVT

SEQ ID NO: 79 - protein, 140 aa, synthetic construct
  REGION 1..140: note = Synthetic: allo-HLA TCRdelta
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGDSYGGGPLYTDKLIFGKGTRVTVEPR

SEQ ID NO: 80 - protein, 176 aa, synthetic construct
  REGION 1..176: note = Synthetic: huTRBC1
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 81 - protein, 178 aa, synthetic construct
  REGION 1..178: note = Synthetic: huTRBC2
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 82 - protein, 140 aa, synthetic construct
  REGION 1..140: note = Synthetic: huTRAC
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 83 - protein, 173 aa, synthetic construct
  REGION 1..173: note = Synthetic: huTRGC1
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 84 - protein, 189 aa, synthetic construct
  REGION 1..189: note = Synthetic: huTRGC2
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKS

SEQ ID NO: 85 - protein, 153 aa, synthetic construct
  REGION 1..153: note = Synthetic: huTRDC
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 86 - protein, 176 aa, synthetic construct
  REGION 1..176: note = Synthetic: mmTRBC1
DLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 87 - protein, 178 aa, synthetic construct
  REGION 1..178: note = Synthetic: mmTRBC2
DLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 88 - protein, 140 aa, synthetic construct
  REGION 1..140: note = Synthetic: mmTRAC
IQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 89 - protein, 613 aa, synthetic construct
  REGION 1..613: note = Synthetic: MAGEA4 TCR
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGRAKRGSGATNFSLLKQAGDVEENPGPMSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 90 - protein, 608 aa, synthetic construct
  REGION 1..608: note = Synthetic: CD33 DARIC
MALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKASAGTGSDIYIWAPLAGTCGVLLLSLVITMHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGATNFSLLKQAGDVEENPGPSMETDTLLLWVLLLWVPGSTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 91 - protein, 734 aa, synthetic construct
  REGION 1..734: note = Synthetic: CD33.VHH-mLNK-TRB-P2A-TRA
METPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPLEKTDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGRAKRGSGATNFSLLKQAGDVEENPGPMSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 92 - protein, 739 aa, synthetic construct
  REGION 1..739: note = Synthetic: CD33.VHH-mLNK.1xG4S-TRB-P2A-TRA
METPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPLEKTGGGGSDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGRAKRGSGATNFSLLKQAGDVEENPGPMSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 93 - protein, 733 aa, synthetic construct
  REGION 1..733: note = Synthetic: TRB-P2A-CD33.VHH-mLNK-TRA
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGRAKRGSGATNFSLLKQAGDVEENPGPMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPLEKTAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 94 - protein, 738 aa, synthetic construct
  REGION 1..738: note = Synthetic: TRB-P2A-CD33.VHH-mLNK.1xG4S-TRA
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGRAKRGSGATNFSLLKQAGDVEENPGPMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPLEKTGGGGSAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 95 - protein, 734 aa, synthetic construct
  REGION 1..734: note = Synthetic: TRB-P2A-CD33.VHH-1xG4S-TRA
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGRAKRGSGATNFSLLKQAGDVEENPGPMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 96 - protein, 739 aa, synthetic construct
  REGION 1..739: note = Synthetic: TRB-P2A-CD33.VHH-2xG4S-TRA
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGRAKRGSGATNFSLLKQAGDVEENPGPMETPAQLLFLLLLWLPDTTGEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGGGGSAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 97 - protein, 868 aa, synthetic construct
  REGION 1..868: note = Synthetic: TRB-P2A-CLL1-CD33.VHH-mLNK.1xG4S-TRA
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGRAKRGSGATNFSLLKQAGDVEENPGPMETPAQLLFLLLLWLPDTTGEVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKPGGGGSGGGGSEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPLEKTGGGGSAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 98 - protein, 612 aa, synthetic construct
  REGION 1..612: note = Synthetic: CLL1 DARIC
MALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKASAGTGSDIYIWAPLAGTCGVLLLSLVITMHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGATNFSLLKQAGDVEENPGPSMETDTLLLWVLLLWVPGSTGEVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 99 - protein, 743 aa, synthetic construct
  REGION 1..743: note = Synthetic: CLL1-CD33 tandem DARIC
MALPVTALLLPLALLLHAARPGSILWHEMWHEGLEEASRLYFGERNVKGMFEVLEPLHAMMERGPQTLKETSFNQAYGRDLMEAQEWCRKYMKSGNVKDLLQAWDLYYHVFRRISKASAGTGSDIYIWAPLAGTCGVLLLSLVITMHKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGSGATNFSLLKQAGDVEENPGPSMETDTLLLWVLLLWVPGSTGEVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKPGGGGSGGGGSGGGGSEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLEGGRMALIVLGGVAGLLLFIGLGIFFCVRCRHRRRQ

SEQ ID NO: 100 - protein, 869 aa, synthetic construct
  REGION 1..869: note = Synthetic: TRB-P2A-BCMA.scFv-2xG4S-TRA
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGRAKRGSGATNFSLLKQAGDVEENPGPMETPAQLLFLLLLWLPDTTGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 101 - protein, 493 aa, synthetic construct
  REGION 1..493: note = Synthetic: BCMA scFv CAR
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 865 aa, synthetic construct
  REGION 1..865: note = Synthetic: TRB-P2A-CD19.scFv-2xG4S-TRA
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRGRAKRGSGATNFSLLKQAGDVEENPGPMETPAQLLFLLLLWLPDTTGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 103 - protein, 411 aa, synthetic construct
  REGION 1..411: note = Synthetic: CD33.VHH-mLNK-TRB
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPLEKTDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 104 - protein, 416 aa, synthetic construct
  REGION 1..416: note = Synthetic: CD33.VHH-mLNK.1xG4S-TRB
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPLEKTGGGGSDSGVTQTPKHLITATGQRVTLRCSPRSGDLSVYWYQQSLDQGLQFLIQYYNGEERAKGNILERFSAQQFPDLHSELNLSSLELGDSALYFCASSGGDGDEQFFGPGTRLTVLEDLKNVFPPEVAVFEPSKAEIAHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDSRG

SEQ ID NO: 105 - protein, 377 aa, synthetic construct
  REGION 1..377: note = Synthetic: CD33.VHH-mLNK-TRA
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPLEKTAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 106 - protein, 382 aa, synthetic construct
  REGION 1..382: note = Synthetic: CD33.VHH-mLNK.1xG4S-TRA
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPLEKTGGGGSAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 107 - protein, 378 aa, synthetic construct
  REGION 1..378: note = Synthetic: CD33.VHH-1xG4S-TRA
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 108 - protein, 383 aa, synthetic construct
  REGION 1..383: note = Synthetic: CD33.VHH-2xG4S-TRA
EVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPGGGGSGGGGSAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 109 - protein, 512 aa, synthetic construct
  REGION 1..512: note = Synthetic: CLL1-CD33.VHH-mLNK.1xG4S-TRA
EVQLVESGGGEVQPGGSLRLSCAASGFLFSIYDMNWYRQAPGKEREWVAGITNNGYSTAYAESVKGRFTISRDNAKNTIYLQMSSLRAEDTAVYYCHADLTKAYDVEYAWGQGTLVTVKPGGGGSGGGGSEVQLVESGGGEVQPGGSLRLSCAASRSSGIDVMGWYRQAPGKERELVAEISGVGDTNYAASLADRFTVSRDNAKNTVYLQMSSLRAEDTAVYYCNAHSFLDLVGAWGQGTLVTVKPLEKTGGGGSAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 110 - protein, 513 aa, synthetic construct
  REGION 1..513: note = Synthetic: BCMA.scFv-2xG4S-TRA
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 111 - protein, 509 aa, synthetic construct
  REGION 1..509: note = Synthetic: CD19.ScFv-2xG4S-TRA
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSAQKITQTQPGMFVQEKEAVTLDCTYDTSDPSYGLFWYKQPSSGEMIFLIYQGSYDQQNATEGRYSLNFQKARKSANLVISASQLGDSAMYFCAMSGGYTGGFKTIFGAGTRLFVKANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 112 - protein, 4 aa, synthetic construct
  REGION 1..4: note = Synthetic: Furin cleavage site
RAKR

SEQ ID NO: 113 - skipped

SEQ ID NO: 114 - protein, 7 aa, synthetic construct
  REGION 1..7: note = Synthetic: protease cleavage site
ENLYFQG

SEQ ID NO: 115 - protein, 7 aa, synthetic construct
  REGION 1..7: note = Synthetic: protease cleavage site
ENLYFQS

SEQ ID NO: 116 - protein, 22 aa, synthetic construct
  REGION 1..22: note = Synthetic: polypeptide cleavage signal
GSGATNFSLLKQAGDVEENPGP

SEQ ID NO: 117 - protein, 19 aa, synthetic construct
  REGION 1..19: note = Synthetic: polypeptide cleavage signal
ATNFSLLKQAGDVEENPGP

SEQ ID NO: 118 - protein, 14 aa, synthetic construct
  REGION 1..14: note = Synthetic: polypeptide cleavage signal
LLKQAGDVEENPGP

SEQ ID NO: 119 - protein, 21 aa, synthetic construct
  REGION 1..21: note = Synthetic: polypeptide cleavage signal
GSGEGRGSLLTCGDVEENPGP

SEQ ID NO: 120 - protein, 18 aa, synthetic construct
  REGION 1..18: note = Synthetic: polypeptide cleavage signal
EGRGSLLTCGDVEENPGP

SEQ ID NO: 121 - protein, 13 aa, synthetic construct
  REGION 1..13: note = Synthetic: polypeptide cleavage signal
LLTCGDVEENPGP

SEQ ID NO: 122 - protein, 23 aa, synthetic construct
  REGION 1..23: note = Synthetic: polypeptide cleavage signal
GSGQCTNYALLKLAGDVESNPGP

SEQ ID NO: 123 - protein, 20 aa, synthetic construct
  REGION 1..20: note = Synthetic: polypeptide cleavage signal
QCTNYALLKLAGDVESNPGP

SEQ ID NO: 124 - protein, 14 aa, synthetic construct
  REGION 1..14: note = Synthetic: polypeptide cleavage signal
LLKLAGDVESNPGP

SEQ ID NO: 125 - protein, 25 aa, synthetic construct
  REGION 1..25: note = Synthetic: polypeptide cleavage signal
GSGVKQTLNFDLLKLAGDVESNPGP

SEQ ID NO: 126 - protein, 22 aa, synthetic construct
  REGION 1..22: note = Synthetic: polypeptide cleavage signal
VKQTLNFDLLKLAGDVESNPGP

SEQ ID NO: 127 - protein, 14 aa, synthetic construct
  REGION 1..14: note = Synthetic: polypeptide cleavage signal
LLKLAGDVESNPGP

SEQ ID NO: 128 - protein, 19 aa, synthetic construct
  REGION 1..19: note = Synthetic: polypeptide cleavage signal
LLNFDLLKLAGDVESNPGP

SEQ ID NO: 129 - protein, 19 aa, synthetic construct
  REGION 1..19: note = Synthetic: polypeptide cleavage signal
TLNFDLLKLAGDVESNPGP

SEQ ID NO: 130 - protein, 14 aa, synthetic construct
  REGION 1..14: note = Synthetic: polypeptide cleavage signal
LLKLAGDVESNPGP

SEQ ID NO: 131 - protein, 17 aa, synthetic construct
  REGION 1..17: note = Synthetic: polypeptide cleavage signal
NFDLLKLAGDVESNPGP

SEQ ID NO: 132 - protein, 20 aa, synthetic construct
  REGION 1..20: note = Synthetic: polypeptide cleavage signal
QLLNFDLLKLAGDVESNPGP

SEQ ID NO: 133 - protein, 24 aa, synthetic construct
  REGION 1..24: note = Synthetic: polypeptide cleavage signal
APVKQTLNFDLLKLAGDVESNPGP

SEQ ID NO: 134 - protein, 40 aa, synthetic construct
  REGION 1..40: note = Synthetic: polypeptide cleavage signal
VTELLYRMKRAETYCPRPLLAIHPTEARHKQKIVAPVKQT

SEQ ID NO: 135 - protein, 18 aa, synthetic construct
  REGION 1..18: note = Synthetic: polypeptide cleavage signal
LNFDLLKLAGDVESNPGP

SEQ ID NO: 136 - protein, 40 aa, synthetic construct
  REGION 1..40: note = Synthetic: polypeptide cleavage signal
LLAIHPTEARHKQKIVAPVKQTLNFDLLKLAGDVESNPGP

SEQ ID NO: 137 - protein, 33 aa, synthetic construct
  REGION 1..33: note = Synthetic: polypeptide cleavage signal
EARHKQKIVAPVKQTLNFDLLKLAGDVESNPGP

SEQ ID NO: 138 - protein, 5 aa, synthetic construct
  REGION 1..5: note = Synthetic: peptide that generally precedes CDRH2
LEWIG

SEQ ID NO: 139 - DNA, 10 bp, synthetic construct
  misc_feature 1..10: note = Synthetic: consensus Kozak sequence
  source 1..10: mol_type = other DNA
gccrccatgg